(19) United States
(12) Patent Application Publication
(10) Pub. No.: US 2026/0325618 A1
(43) Pub. Date: Oct. 22, 2026

(54) A MAPS VACCINE TARGETING GROUP B STREPTOCOCCUS (GBS)

(71) Applicants: Claudette THOMPSON, North Reading, MA (US); THE CHILDREN'S MEDICAL CENTER CORPORATION, BOSTON, MA (US)

(72) Inventors: CLaudette THOMPSON, North Reading, MA (US); Richard MALLEY, Beverly, MA (US); Fan ZHANG, West Roxbury, MA (US); Yingjie LU, West Roxbury, MA (US)

(73) Assignee: THE CHILDREN'S MEDICAL CENTER CORPORATION, BOSTON, MA (US)

(21) Appl. No.: 19/120,118

(22) PCT Filed: Oct. 13, 2023
(86) PCT No.: PCT/US2023/076878

§ 371 Date: Apr. 10, 2025

Related U.S. Application Data
(60) Provisional application No. 63/416,482, filed on Oct. 14, 2022.

(51) Int. Cl. A61K 39/09; A61K 39/00; A61K 39/39; A61P 37/04; C07K 14/315

(52) U.S. Cl. CPC A61K 39/092; A61K 39/39; A61P 37/04; C07K 14/315; A61K 2039/55505; A61K 2039/6087; A61K 2039/625; A61K 2039/70; C07K 2319/40

(57) ABSTRACT

Technologies for the prevention and/or treatment of GBS infections. The technology relates to compositions, including vaccines compositions and methods comprising an immunogenic complex that is a GBS multiple antigen presenting system (MAPS-GBS), where two or more biotinylated GBS polysaccharide antigens are joined together by non-covalent associations with one or more bifunctional fusion proteins comprising, in any order, (i) a sialic acid binding protein (SBD), a GBS polypeptide antigen and (iii) a biotin-binding moiety (BBD), thereby facilitating the linking of multiple GBS polysaccharide antigens together in the complex to form a MAPS-GBS immunogenic complex. The polysaccharide antigens that are linked can be on the same polysaccharide macromolecule or on distinct polysaccharide macromolecules.

### CROSS-REFERENCED TO RELATED APPLICATIONS

This application claims benefit under 35 U.S.C. § 119(e) of U.S. Provisional Application No. 63/416,482 filed Oct. 14, 2022, the contents of which is incorporated herein by reference in its entirety.

### FIELD OF THE INVENTION

The present invention relates to technologies, compositions, and methods for the prevention and/or treatment of Group B Streptococcus agalactiae (GBS) infections.

### SEQUENCE LISTING

The instant application contains a Sequence Listing which was created on Oct. 12, 2023 with the filename “701039-000102WOPT_SL.xml” and has a file size of 249,404 bytes.

### BACKGROUND OF INVENTION

Streptococci are catalase negative Gram positive cocci. They may be classified by the type of hemolysis exhibited on blood agar, by the serologic detection of carbohydrate antigens, or by certain biochemical reactions. Medically important streptococci include Groups A, B, D, S. pneumoniae and the viridans group of streptococci.

Streptococcus agalactiae are Gram positive polysaccharide encapsulated organisms that are also known as group B streptococcus (GBS). Group B Streptococcus agalactiae are the most common cause of serious bacterial infections in newborns, and important pathogens in pregnant women and nonpregnant adults with underlying medical problems such as diabetes and cardiovascular disease.

Group B streptococci (GBS) are the most common cause of serious bacterial disease in neonates and are important pathogens in pregnant women and adults with underlying illnesses.

Common manifestations of these infections include bacteremia, pneumonia, meningitis, sepsis, endocarditis, and osteoarticular infections. The incidence of invasive GBS disease is approximately 2.6 in 1000 live births and 7.7 in 100,000 in the overall population, with mortality rates that vary from 6 to 30%. They are a common commensal of the human gastrointestinal and genital tract and also a cause of serious disease in infants and older adults. The main risk factor for GBS infection in infants is maternal colonization. As much as one in four women carry GBS recto-vaginally, which can infect the amniotic fluid or baby before or during delivery causing sepsis, pneumonia, and meningitis. Twenty five percent of infants who survive GBS meningitis suffer from neurologic impairment with 19% experiencing cognitive delay, cerebral palsy, blindness, and hearing loss. GBS can also cause miscarriages and preterm deliveries and is linked to stillbirths. Very low birth weight infants are at much higher risk of infection, with up to 3% infected and mortality rates of up to 30%, even with immediate antibiotic treatment. Although much neonatal disease is preventable by administration of prophylactic antibiotics to women in labor, antibiotic prophylaxis programs can be inefficient, suffer from poor compliance, or fail if antibiotic resistance emerges. No effective prophylaxis strategy for adult infections has been established.

During childbirth, GBS can pass from the mother to the newborn. By one estimate, up to 30% of pregnant women carry GBS at least temporarily in the vagina or rectum without symptoms. Infants born to these women become colonized with GBS during delivery. Aspiration of infected amniotic fluid or vaginal secretions allow GBS to gain access to the lungs. Adhesion to, and invasion of, respiratory epithelium and endothelium appear to be critical factors in early onset neonatal infection. Subsequent steps in infection, such as blood stream invasion and the establishment of metastatic local infections have not been clarified. The pathogenesis of neonatal infection occurring after the first week of life is also not well understood. Gastrointestinal colonization may be more important than a respiratory focus in late onset neonatal disease. Considerable evidence suggests that invasion of brain microvascular endothelial cells by GBS is the initial step in the pathogenesis of meningitis. GBS are able to invade human brain microvascular endothelial cells and type III GBS, which are responsible for the majority of meningitis, accomplish this 2-6 times more efficiently than other serotypes.

In some high-income countries, because GBS is widely distributed among the population and is an important pathogen in newborns, pregnant women are commonly tested for GBS at 35-37 weeks of pregnancy. Much of GBS neonatal disease is preventable by administration of prophylactic antibiotics during labor to women who test positive or display known risk factors. However, these antibiotics programs do not prevent all GBS disease. The programs are deficient for a number of reasons. First, the programs can be inefficient. Second, it is difficult to ensure that all healthcare providers and patients comply with the testing and treatment. Third, a small but significant proportion of individuals may suffer potentially lethal anaphylactic reactions to the antibiotic. And finally, if new serotypes or antibiotic resistance emerges, the antibiotic programs may fail altogether. Currently available tests for GBS are inefficient. These tests may provide false negatives. Furthermore, the tests are not specific to virulent strains of GBS. Thus, antibiotic treatment may be given unnecessarily and add to the problem of antibiotic resistance.

Traditionally, GBS are divided into 10 serotypes according to the immunologic reactivity of the polysaccharide capsule. Serotype III GBS are particularly important in human neonates, causing 60-70% of all infections and almost all meningitis. Type III GBS can be subdivided into three groups of related strains based on the analysis of restriction digest patterns (RDPs) produced by digestion of chromosomal DNA with Hind III and Sse8387. Over 90% of invasive type III GBS neonatal disease in Tokyo, Japan and in Salt Lake City, Utah is caused by bacteria from one of three RDP types, termed RDP type III-3, while RDP type III-2 are significantly more likely to be isolated from vagina than from blood or CSF. These results suggest that this genetically-related cluster of type III-3 GBS are more virulent than III-2 strains and could be responsible for the majority of invasive type III disease globally.

Another population at risk for GBS disease is the elderly. Risk factors include chronic medical problems such diabetes mellitus, cancer, heart failure, neurologic, and urologic conditions. According to CDC ABC surveillance data, the annual U.S. incidence of invasive GBS in 2013 was 0.28/1,000 adults or 12,400 cases/year in adults >65 years of age. This rate approaches the incidence of invasive pneumococcal disease in the elderly (vs. 0.30/1,000 for >65). These rates are expected to continue to increase in both the U.S. and in Europe. One approach to prevent GBS disease among infants and the elderly is the use of a polysaccharide-based vaccine. The implementation of a maternal GBS prophylactic vaccine has the potential to prevent GBS disease among infants.

Pneumococcal conjugate vaccines for prevention of invasive pneumococcal disease, including bacteremia and meningitis have been developed, however and in spite of intrapartum antibiotic prophylaxis (IAP) for prevention of GBS disease, GBS has become the single most common cause of neonatal sepsis (EOD) and meningitis (<2 mo) in infants in the U.S.

Although polysaccharides can be immunogenic on their own, conjugation of polysaccharides to protein carriers has been used to improve immunogenicity, particularly in infants and the elderly. Polysaccharide-protein conjugate vaccines are made using polysaccharides, generally from the surface coat of bacteria, linked to protein carriers. The covalent linkage (e.g., formation of a chemical bond) between the polysaccharide and protein carrier induces an immune response against bacteria displaying the polysaccharide contained within the vaccine on their surface, thus preventing disease. Accordingly, vaccination using polysaccharides from pathogenic bacteria is a potential strategy for boosting host immunity.

Preliminary vaccines for GBS used unconjugated purified polysaccharide. GBS poly- and oligosaccharides are poorly immunogenic and fail to elicit significant memory and booster responses. Overall, only 57% of women with low levels of specific antibody responded to the vaccine. The poor immunogenicity of purified polysaccharide antigen was further demonstrated in a study in which thirty adult volunteers were immunized with a tetravalent vaccine composed of purified polysaccharide from serotypes Ia, Ib, II, and III. Although safe, this vaccine was only modestly immunogenic, with only 13% of subjects responding to type Ib, 17% to type II, 33% responding to type Ia, and 70% responding to type III polysaccharide. The poor immunogenicity of polysaccharide antigens prompted efforts to develop polysaccharide conjugate vaccines, whereby these poly- or oligosaccharides are conjugated to protein carriers. Ninety percent of healthy adult women immunized with a type III polysaccharide-tetanus toxoid conjugate vaccine responded with a 4-fold rise in antibody concentration, compared to 50% immunized with plain polysaccharide. A type Ia/Ib polysaccharide-tetanus toxoid conjugate vaccine was similarly more immunogenic in healthy adults than plain polysaccharide.

Individual monovalent polysaccharide-protein conjugates of GBS serotypes Ia, Ib, II, III, and V have been evaluated in phase 1 and 2 clinical trials in non-pregnant adults. Bivalent II-TT and III-TT glycoconjugate vaccines and a trivalent vaccine comprising Ia-CRM-197, Ib-CRM₁₉₇ and III-CRM-₁₉₇ glycoconjugates have also been studied (Clinicaltrials.gov NCT01 193920, NCT01412801, and NCT01446289). A tri-valent vaccine covers >90% of invasive strains causing neonatal disease in South Africa, but these same serotypes represent only 62% and 66% of invasive isolates in North America and Europe, respectively, based on surveillance of recent neonatal isolates from a global collection of 901 samples collected between 2004-2013 from the Tigecycline Evaluation and Surveillance Trial (T.E.S.T.).

Analysis of the strains obtained from the T.E.S.T. samples showed that 95% of the strains collected belonged to one of the five documented major serotypes (Ia, Ib, II, III, and V) and a further 3% were serotype IV. A series of publications have also confirmed the appearance of serotype IV over the last decade in the Americas and in Europe (Diedrick, M. J., et al., J. Clin. Microbiol., 48(9):3100-3104 (2010); Teatero (2014); Meehan, M. et al., European Journal of Clinical Microbiology & Infectious Diseases, 33(7): 1 155-1 162 (2014); Florindo, C, et al., Euro Surveillance: Bulletin European sur les Maladies Transmissibles (European Communicable Disease Bulletin), 19(23) (2014); Palmiero, J. K., et al., Journal of Clinical Microbiology, 48(12):4397-4403 (2010)). A study surveying recto/vaginal carriage in adults, which is a risk factor for transmission of GBS to the infant, also found 97% of isolates belonging to one of these six serotypes, with serotype IV representing a frequency of about 4%. The study was designed to monitor carriage of beta-hemolytic streptococci (which includes GBS), Clostridium difficile, and Staphylococcus aureus in healthy U.S. adults. Similarly, analysis of T.E.S.T. samples showed 98% of U.S. blood isolates from older adults >65 years of age belong to the same six predominant serogroups. The most noticeable difference between the elderly isolates and the other populations is the serogroup distribution. For the isolates from elderly patients, serotype V strains constitute the largest group (34% vs. 18% for neonatal or 18% for adult carriage strains). Other studies have found that there is a geographic variance of serotype prevalence. For instance, serotype VI and VIII isolates have been shown to be predominant colonizers of healthy pregnant women in Japan (Lachenauer, C. S., et al., JID 179(4): 1030-1033 (1999).

The polysaccharides that cover bacteria vary greatly, even within a single species of bacteria. For example, in GBS there are ten different serotypes due to variation in the bacterial polysaccharide capsule. Therefore, it is desirable for GBS vaccines to consist of a panel of polysaccharides to ensure breadth of coverage against different circulating strains.

However, GBS capsular polysaccharides (CP) comprise sialic acid, which are a monosaccharide that is also widely present in animals and, to a lesser extent, in microorganisms, including fungi, bacteria and virus. In some cases, such bacteria and viruses, including GBS, incorporate sialic acid onto their surface capsule (e.g., polysaccharides in the case of bacteria or glycoproteins in the case of virus), so that they can evade host immune defense during infection by using sialic acid (a self-antigen for the host) as a shield. As a result, it is difficult to generate immune responses to sialic acid-containing bacterial capsular polysaccharides or viral glycoproteins when used as vaccine targets. There prior reports that decreasing the levels of salic acid on GBS, e.g., by desialylation (see International Applications WO 2012/035519) or to reduce sialic acid content >50% for serotype V (Int'l Patent Appl. Pub. No. WO 2014/053612, see International Application WO2016178123A1) enhances immunogenicity. Alternatively, de-O-acetylated polysaccharides have also been used (see International Application WO2016178123A1)

Thus, there is a need to develop a method to overcome the “immune suppression” caused by sialic acid modification in bacterial or viral components and induce robust immune responses to these domains during vaccination. Moreover, a need exists for polyvalent GBS vaccines that have enhanced immunogenicity to a broad range of GBS subtypes, and that can confer passive immunity to prevent or treat GBS diseases, among broad populations worldwide.

### SUMMARY OF THE INVENTION

The present disclosure addresses the lack of suitable vaccines for the prevention and/or treatment of diseases caused by Streptococcus agalactiae also known as group B streptococcus (GBS). Among other things, the present disclosure addresses challenges in providing vaccines with sufficient immunogenicity to protect against GBS. Technologies described herein can induce a T- and B-cell response and/or provide immunity against a broad range of GBS serotypes, including but not limited to subtypes IIa, Ib, II, III, IV, V or VII.

In some embodiments, a vaccine comprising plurality of MAPS-GBS immunogenic complexes, wherein each MAPS-GBS immunogenic complex comprises: (a) a biotinylated GBS polysaccharide antigen; and (b) at least one fusion protein selected from (i) a biotin-binding moiety (BBM) fusion protein, or (ii) a sialic acid binding domain (SBD) fusion protein, or (iii) a bifunctional SBD-BBM fusion protein; wherein the fusion protein further comprises at least one polypeptide antigen, wherein the biotinylated GBS polysaccharide antigen comprises a polysaccharide from GBS, and further wherein the biotinylated polysaccharide antigen is non-covalently associated with the biotin-binding moiety of the fusion protein to form an immunogenic complex.

The technology disclosed herein relates to a polyvalent and multi-component Group B Streptococcus (GBS) immunogenic composition (MAPS-GBS) that is modified from Multiple Antigen Presenting System (MAPS), previously disclosed in U.S. Pat. No. 10,766,932, which is incorporated herein in its entirety by reference. More specifically, the inventors have improved upon the MAPS disclosed in the U.S. Pat. No. 10,766,932, in that, in some embodiments, the MAPS-GBS immunogenic complex disclosed herein comprises a fusion protein comprising a sialic acid-binding domain (SBD) fused to a GBS polypeptide antigen, and where the SBD binds to salic acid on a GBS polysaccharide of the immunogenic complex. In some embodiments, as an added benefit, the non-covalent association of the SBD to the sialic acid on the antigenic polysaccharide blocks the immune suppressive function of sialic acids on the antigenic GBS polysaccharide. That is, without being limited to theory, a sialic acid-binding protein domain (SBD) can bind to a sialic acid on the GBS polysaccharide and mask (e.g., shield) the immune tolerance of a the GBS antigenic polysaccharide. Stated differently, the binding of the SBD of the fusion protein to sialic acid on GBS polysaccharides reduces the hosts' exposure to the sialic acids, which are normally recognized by the host as a self-antigen, and thus increases the immunogenicity of the polysaccharide to the host. Herein the inventors have demonstrated that a SBD-[GBSAg]-BBM fusion protein in a MAPS-GBS complex as disclosed herein can quench the ability of sialic acids on the surface of GBS capsular polysaccharides to evade a host immune system, and therefore improve the host's immune response to the GBS polysaccharide when administered to the subject. That is, the technology disclosed herein describes a MAPS-GBS immunogenic complex that comprises a sialic acid-binding domain (SBD) to overcome the immune suppression of native sialic acid on the surface of a GBS polysaccharide.

Disclosed herein is data demonstrating use of a bi-functional fusion protein that contains a sialic acid-binding domain (SBD) and a biotin-binding moiety (BBM), with the application that the SBD binds sialic acid on the polysaccharide antigens and blocks its exposure to (and immune-suppressive or -tolerizing effect) antigen-presenting cells until the antigens are internalized and processed for proper epitope-presentation, while the BBM can bind biotinylated scaffold, ligands, or other molecules that could potentiate the immune response generation.

Aspects of the technology described herein relate to methods, compositions and vaccines comprising a MAPS-GBS immunogenic complex comprising a bi-functional fusion protein which comprises a sialic acid-binding domain (SBD) and a biotin-binding moiety (BBM), referred to herein as SBD-BBM fusion protein, where the fusion protein binds to at least one antigenic biotinylated polysaccharide that comprises biotin and sialic acids, such that the biotinylated antigenic polysaccharide form a MAPS-GBS immunogenic complex with the fusion protein (see, e.g., FIG. 1B).

Aspects of the technology disclosed herein relates to a bi-functional fusion protein comprising, in any order, (i) a sialic acid-binding domain (SBD), (ii) a biotin-binding moiety (BBM), and (iii) at least 1, or at least 2, or at least 3, at least 4, at least 5, or more than 5 polypeptide antigens, e.g., polypeptide antigens from GBS, and is broadly referred to herein as SBD-[GBS-Ag]n-BBM fusion protein, where n is the number of antigenic polypeptide located between the SBD and BBM. It is envisioned that the antigen in the SBD-[GBS-Ag]n-BBM fusion protein can be located at the N-terminal, or the C-terminal of the fusion protein, or between the SBD and the BBM. For example, exemplary fusion proteins are disclosed in Tables 2a, 2B and 3 herein.

Accordingly, aspects of the technology disclosed herein relates to methods, compositions and vaccines comprising a MAPS-GBS immunogenic complex using a bi-functional fusion protein comprising a sialic acid-binding domain (SBD) and a biotin-binding moiety (BBM), where the bi-functional fusion protein further comprises at least one polypeptide from Group B Streptococcus (GBS) or Streptococcus agalactiae. It is envisioned that at least one, or at least two of the GBS polypeptide antigens as disclosed herein can be substituted for any polypeptide antigen, for example including, but not limited to any antigen disclosed in U.S. Pat. Nos. 10,766,932, 11,235,047, 11,013,793, 11,305,001, 9,499,593, 10,017,548, 10,611,805 or U.S. patent application Ser. No. 14/766,252, 16/616,258, 16/568,646, the contents of each are incorporated herein in their entirety by reference.

In some embodiments, the bi-functional fusion protein comprises at least one antigenic polypeptide which can be arranged in any order in the fusion protein with the sialic acid-binding domain (SBD) and a biotin-binding moiety (BBM), wherein the one antigenic polypeptide is from Group B Streptococcus (GBS). In some embodiments, the bi-functional fusion protein comprises at least two antigenic polypeptides located between the sialic acid-binding domain (SBD) and a biotin-binding moiety (BBM), wherein both the antigenic polypeptides is from Group B Streptococcus (GBS) or Streptococcus agalactiae. In some embodiments, the fusion protein can further comprise other antigenic polypeptides located between the SBD and the BBM, for example, any polypeptide antigen, for example including, but not limited to any antigen disclosed in U.S. Pat. Nos. 10,766,932, 11,235,047, 11,013,793, 11,305,001, 9,499,593, 10,017,548, 10,611,805 or U.S. patent application Ser. Nos. 14/766,252, 16/616,258, 16/568,646, the contents of each are incorporated herein in their entirety by reference.

In some embodiments, a fusion protein useful in the MAPS-GBS immunogenic complex as disclosed herein can comprise at least one antigenic polypeptide from GBS is selected from at least one of: Rib, Sip, AlpC, Alp1 or Alp3, as disclosed herein. Exemplary fusion proteins are disclosed in Table 2A, 2B and 3. In some embodiments, a fusion protein useful in the MAPS-GBS immunogenic complex is selected from any of: SBD-[Rib]-BBM fusion protein, SBD-[Sip]-BBM fusion protein, SBD-[Rib-Sip]-BBM fusion protein, BBM-[Rib]-SBD fusion protein, BBM-[Sip]-SBD fusion protein, BBM-[Rib-Sip]-SBD fusion protein, or variations thereof. In some embodiments, Rib and Sip, either individually, or together can be readily replaced with alternative GBS antigens known in the art, such as AlpC, Alp1, Alp3 or a Alp3/1 as disclosed herein.

In some embodiments, a fusion protein useful in the MAPS-GBS immunogenic complex as disclosed herein comprises, in any order (i) at least one antigenic polypeptide from GBS and (ii) SBD having an amino acid sequence with at least 50%, at least 60%, at least 70%, at least 80%, or at least 85%, or at least 90%, or at least 95% sequence identity to SEQ ID NO: 2 (SBD1). In some embodiments, a GBS fusion protein useful in the MAPS-GBS immunogenic complex as disclosed herein comprises, in any order (i) at least one antigenic polypeptide from GBS and (ii) SBD having an amino acid sequence with at least 50%, at least 60%, at least 70%, at least 80%, or at least 85%, or at least 90%, or at least 95% sequence identity to SEQ ID NO: 112 (SBD2). In some embodiments, a GBS fusion protein useful in the MAPS-GBS immunogenic complex as disclosed herein comprises, in any order (i) at least one antigenic polypeptide from GBS and (ii) SBD selected from any of SBD1, SBD2, SBD3, SBD4, NanH, NanH2, VcNanH, or having an amino acid sequence with at least 80%, or at least 85%, or at least 90%, or at least 95% sequence identity to any of: SEQ ID NOs: 2 or 112-120.

In some embodiments, a fusion protein useful in the MAPS-GBS immunogenic complex as disclosed herein comprises, in any order (i) at least one antigenic polypeptide from GBS and (ii) BBM which is Rhizavidin having an amino acid sequence with at least 80%, or at least 85%, or at least 90%, or at least 95% sequence identity to SEQ ID NO: 1 or SEQ ID NO: 8.

In some embodiments, a fusion protein useful in the MAPS-GBS immunogenic complex as disclosed herein comprises, in any order, (i) at least one antigenic polypeptide from GBS, (ii) SBD having an amino acid sequence with at least 80%, or at least 85%, or at least 90%, or at least 95% sequence identity to SEQ ID NO: 2 (SBD1), and (iii) a BBM which is Rhizavidin, having an amino acid sequence with at least 80%, or at least 85%, or at least 90%, or at least 95% sequence identity to SEQ ID NO: 1 or SEQ ID NO: 8. In some embodiments, a fusion protein useful in the MAPS-GBS immunogenic complex as disclosed herein comprises, in any order, (i) at least one antigenic polypeptide from GBS, (ii) SBD having an amino acid sequence with at least 80%, or at least 85%, or at least 90%, or at least 95% sequence identity to SEQ ID NO: 112 (SBD2), and (iii) a BBM which is Rhizavidin, having an amino acid sequence with at least 80%, or at least 85%, or at least 90%, or at least 95% sequence identity to SEQ ID NO: 1 or SEQ ID NO: 8. In some embodiments, a fusion protein useful in the MAPS-GBS immunogenic complex as disclosed herein comprises, in any order, (i) at least one antigenic polypeptide from GBS, (ii) a SBD selected from any of SBD1, SBD2, SBD3, SBD4, NanH, NanH2, VcNanH, or having an amino acid sequence with at least 80%, or at least 85%, or at least 90%, or at least 95% sequence identity to any of: SEQ ID NOs: 2 or 112-120 and (iii) a BBM which is Rhizavidin, having an amino acid sequence with at least 80%, or at least 85%, or at least 90%, or at least 95% sequence identity to SEQ ID NO: 1 or SEQ ID NO: 8.

In some embodiments, a fusion protein useful in the MAPS-GBS immunogenic complex as disclosed herein comprises a Rib polypeptide, where the Rib polypeptide comprises at least an amino acid sequence of SEQ ID NO: 4, or a polypeptide having at least 50%, at least 60%, at least 70%, as least 80%, or at least 85%, or at least 90%, or at least 95% sequence identity to the amino acid sequence of SEQ ID NO: 4 (Rib). In some embodiments, a fusion protein useful in the MAPS-GBS immunogenic complex as disclosed herein comprises a Sip polypeptide, where the Sip polypeptide comprises at least an amino acid sequence of SEQ ID NO: 5, or a polypeptide having at least 50%, at least 60%, at least 70%, at least 80%, or at least 85%, or at least 90%, or at least 95% sequence identity to SEQ ID NO: 5 (Sip). In some embodiments, a fusion protein useful in the MAPS-GBS immunogenic complex as disclosed herein comprises a AlpC polypeptide, where the AlpC polypeptide comprises at least an amino acid sequence of SEQ ID NO: 11, or a polypeptide having at least 50%, at least 60%, at least 70%, at least 80%, or at least 85%, or at least 90%, or at least 95% sequence identity to SEQ ID NO: 11 (AlpC). In some embodiments, a fusion protein useful in the MAPS-GBS immunogenic complex as disclosed herein comprises a Alp1 polypeptide, where the Alp1 polypeptide comprises at least an amino acid sequence of SEQ ID NO: 12, or a polypeptide having at least 50%, at least 60%, at least 70%, t least 80%, or at least 85%, or at least 90%, or at least 95% sequence identity to SEQ ID NO: 12 (Alp1). In some embodiments, a fusion protein useful in the MAPS-GBS immunogenic complex as disclosed herein comprises a Alp3 polypeptide, where the Alp3 polypeptide comprises at least an amino acid sequence of SEQ ID NO: 13, or a polypeptide having at least 50%, at least 60%, at least 70%, at least 80%, or at least 85%, or at least 90%, or at least 95% sequence identity to SEQ ID NO: 13 (Alp3). In some embodiments, a fusion protein useful in the MAPS-GBS immunogenic complex as disclosed herein comprises a Alp3/1 polypeptide, where the Alp3/1 polypeptide comprises at least an amino acid sequence of SEQ ID NO: 14, or a polypeptide having at least 50%, at least 60%, at least 70%, at least 80%, or at least 85%, or at least 90%, or at least 95% sequence identity to SEQ ID NO: 14 (Alp3/1). In some embodiments, a fusion protein useful in the MAPS-GBS immunogenic complex as disclosed herein comprises an amino acid sequence having at least 50%, at least 60%, at least 70%, at least 80%, or at least 85%, or at least 90%, or at least 95% sequence identity to SEQ ID NO: 15 (AlpC-Rib).

In some embodiments, a fusion protein useful in the MAPS-GBS immunogenic complex as disclosed herein comprises at least one, or at least two, or at least 3 antigenic polypeptides selected from SEQ ID NOS: 4, 5, 11-15, or least one, or at least two, or at least 3 antigenic polypeptides having an amino acid sequence having at least 80%, or at least 85%, or at least 90%, or at least 95% sequence identity to SEQ ID NOS: SEQ ID NO: 4, 5, 11-15. In some embodiments, a fusion protein useful in the MAPS-GBS immunogenic complex as disclosed herein comprises an amino acid sequence having at least 80%, or at least 85%, or at least 90%, or at least 95% sequence identity to SEQ ID NO: 15 (AlpC-Rib).

One aspect of the present invention relates a fusion protein comprising a sialic acid-binding domain (SBD) and a GBS antigenic polypeptide, referred to as a SBD-Antigen fusion protein, wherein the antigenic GBS polypeptide is selected from one or more selected from: Rib, Sip, AlpC, Alp1, Alp3 or a Alp3/1 as disclosed herein. Other aspects relate to the use of such a SBD-Antigen fusion protein in methods, compositions and vaccines comprising a MAPS-GBS immunogenic complex comprising for the treatment and or prevention of infection with Group B streptococci (GBS), including the prevention and treatment of common manifestations of GBS infections, including but not limited to bacteremia, pneumonia, meningitis, endocarditis, and osteoarticular infections

In some embodiments, the fusion protein comprising a sialic acid-binding domain (SBD) and an antigenic polypeptide from Group B Streptococcus (GBS) or Streptococcus agalactiae. In some embodiments, the antigenic polypeptide from GBS is selected from Rib, or Sip, or Rib and Sip, as disclosed herein. In some embodiments, a SBD-Antigen fusion protein is selected from any of: SBD-[Rib] fusion protein, SBD-[Sip]-fusion protein, SBD-[Rib-Sip] fusion protein, [Rib]-SBD fusion protein, [Sip]-SBD fusion protein, [Rib-Sip]-SBD fusion protein. In some embodiments, Rib and Sip, either individually, or together can be readily replaced with alternative GBS antigens known in the art.

It is envisioned that at least one, or at least two of the GBS polypeptide antigens in a SBD-Antigen fusion as disclosed herein can be substituted for any polypeptide antigen, for example including, but not limited to any antigen disclosed in U.S. Pat. Nos. 10,766,932, 11,235,047, 11,013,793, 11,305,001, 9,499,593, 10,017,548, 10,611,805 or U.S. patent application Ser. Nos. 14/766,252, 16/616,258, 16/568,646, the contents of each are incorporated herein in their entirety by reference.

The MAPS-GBS complex as disclosed herein comprises biotinylated polysaccharides and a bifunctional SBD-[GBS-Ag]-BBM fusion protein. In brief, a bifunctional SBD-[GBS-Ag]-BBM serves as both a carrier protein and a linking protein, which can link two polysaccharide chains together, thereby forming a cross-linked MAPS-GBS complex. Stated differently, a MAPS-GBS complex comprises at least two polysaccharide antigens, which can be (i) on the same polysaccharide macromolecule or (ii) on different polysaccharide macromolecules, where the two polysaccharide antigens are linked together via the SBD-[GBS-Ag]-BBM fusion protein which non-covalently associates with each of the two polysaccharide antigens. As such, the SBD-[GBS-Ag]-BBM fusion protein serves as a linking protein that is also functions as a carrier protein to link two polypeptide antigens.

In some embodiments, if the two polypeptide antigens, referred herein and throughout the specification as PS1 and PS2 respectively, are located on the same macromolecule, e.g., on the same polymer chain, or on different branches of a polymer of the same macromolecule, the SBD-[GBS-Ag]-BBM fusion protein serves an intra-macromolecule linkage role to form a MAPS-GBS immunogenic complex. In alternative embodiments, if the two polypeptide antigens are located on a different macromolecule, e.g., one PS1 is from a distinct polysaccharide macromolecule, and the other PS2 is a distinct polysaccharide macromolecule (e.g., from a different pathogen and/or a specific serotype from the macromolecule used in P1), the SBD-[GBS-Ag]-BBM fusion protein serves an inter-macromolecule linkage role, to form a MAPS-GBS immunogenic complex with more than one polysaccharide macromolecule.

Accordingly, term “polysaccharide antigen” as used herein refers to a region/portion of a polysaccharide macromolecule that comprises biotin and/or sialic acid molecules. Thus, two “polysaccharide antigens” can be present either (i) on the same polysaccharide molecule (e.g., the same macromolecule of PS polymer chain, including any branches), thereby forming links within the same PS macromolecule, or (ii) each “polysaccharide antigen” can be present in two distinct polymer macromolecules, thereby joining different PS macromolecules. As such, in some embodiments, a MAPS-GBS complex can comprise intra-linkage of polysaccharide antigens within the same polysaccharide macromolecule, and/or inter-linkage of polysaccharide antigens from two or more polysaccharide macromolecules.

For illustrative purposes only, referring to FIG. 1B, another aspect of the present invention relates to methods, compositions and vaccines comprising a MAPS-GBS immunogenic complex, wherein a composition comprises at least one species of MAPS-GBS immunogenic complex, wherein each species of the MAPS-GBS immunogenic complex comprises; (i) at least a first biotinylated polysaccharide antigen (PS1) comprising at least one sialic acid domain and at least one biotin molecule, (ii) at least a second biotinylated polysaccharide antigen (PS2) comprising at least one sialic acid domain and at least one biotin molecule, and (iii) at least one fusion protein, each fusion protein comprises at least a biotin-biotin moiety (BBM) and a sialic acid binding domain (SBD), and wherein a BBM of at least one fusion protein non-covalently associates with at least one biotin molecule on the PS1 and the SBD non-covalently associates with at least one sialic acid on the PS2, and/or wherein a SBD of at least one fusion protein non-covalently associates with at least one sialic acid domain on the PS1 and the BBM non-covalently associates with at least biotin domain on the PS2, to result in the PS1 and PS2 being indirectly linked by the fusion protein form an MAPS-GBS immunogenic complex, as the fusion protein non-covalently associates with the PS1 and PS2. Where the first (PS1) and second polysaccharide antigen (PS2) are located on the same macromolecule, e.g., on the same polymer chain, or on different branches of a polymer of the same macromolecule, the SBD-[GBS-Ag]-BBM fusion protein serves an intra-macromolecule linkage role to form a MAPS-GBS immunogenic complex. Where the PS1 and PS2 are located on a different macromolecule, e.g., PS1 is located on a specific polysaccharide macromolecule, and the other PS2 is located a distinct polysaccharide macromolecule (e.g., from a different pathogen and/or a specific serotype to the PS1 macromolecule), the SBD-[GBS-Ag]-BBM fusion protein serves an inter-macromolecule linkage role, to form a MAPS-GBS immunogenic complex with more than one polysaccharide macromolecule.

In some embodiments, the MAPS-GBS immunogenic complex can comprise the following non-covalent associations; PS1-(SBD-BBM fusion protein)-PS2, where the BBM of the SBD-BBM fusion protein non-covalently associates with a biotin on the first polysaccharide (PS1), and the SBD of the SBD-BBM fusion protein non-covalently associates with a sialic acid on the second polysaccharide (PS2), to form an immunogenic complex. In some embodiments, the MAPS-GBS immunogenic complex can comprise the following non-covalent associations: PS1-(SBD-BBM fusion protein)-PS2, where the SBD of the SBD-BBM fusion protein non-covalently associates with a sialic acid on the first polysaccharide (PS1), and the BBM of the SBD-BBM fusion protein non-covalently associates with a biotin on the second polysaccharide (PS2), to form an immunogenic complex. As the first (PS1) and second antigenic polysaccharides (PS2) both have biotin molecules and sialic acid domains on their surface, the immune complex can comprise both the following non-covalent associations: PS1:SBD-BBM:PS2 and PS1:BBM-SBD:PS2, where: represents a non-covalent interaction (see, e.g., FIG. 1B). It is envisioned that the SBD-BBM fusion protein further comprises at least one, or at least 2, or at least 3 or more GBS polypeptide antigens, and can optionally be located between a BBM and a SBD polypeptide, or can be selected from any of the fusion proteins in Tables 2A, 2B or Table 3 as disclosed herein. In some embodiments, the PS1 and PS2 for each MAPS-GBS immunogenic complex of each species is from the same GBS serotype. In some embodiments, the PS1 and PS2 for each MAPS-GBS immunogenic complex for each species is from a different GBS serotype. For example, PS1 and PS2 for a particular species of a MAPS-GBS immunogenic complex can be from, e.g., a specific serotype of Group B Streptococcus (GBS) or Streptococcus agalactiae. In alternative embodiments, PS1 and PS2 for a particular species of a MAPS-GBS immunogenic complex can be from, e.g., different serotypes of Group B Streptococcus (GBS) or Streptococcus agalactiae.

Aspects of the technology described herein relate to a composition, which is a polyvalent immune composition, and comprises at least one species of MAPS-GBS immunogenic complex. That is, each MAPS-GBS species is distinct from the other MAPS-GBS species in the immunogenic complex. Without being limited to theory, for example, a composition can comprise, e.g., two MAPS-GBS immunogenic complexes as disclosed herein, where the first species of MAPS-GBS immunogenic complex disclosed herein comprises a PS1 and PS2 from Streptococcus agalactiae serotype Ia, and the second species of MAPS-GBS immunogenic complex can comprise a PS1 and PS2 from Streptococcus agalactiae serotype 1b. In such an embodiment, a composition would be considered a two valent (2V) MAPS-GBS immunogenic composition or vaccine. It is envisioned that a MAPS-GBS polyvalent immune composition as disclosed herein comprises MAPS-GBS immunogenic complexes that comprise polysaccharides from at least 2, or at least 3, or at least 4, or at least 5, or at least 6, or at least 7, or at least 8, or at least 9, or at least 10, or 2-5, or 5-6, or 6-7, or 7-8, or 8-9 or 9-10, or more than 10 different serotypes of GBS bacteria.

In some embodiments, as disclosed herein, the first biotinylated polysaccharide (PS1), or second biotinylated polysaccharide (PS2), or both, is from any of serotypes IIa, Ib, II, III, IV, V, VI, VII, VIII, and IX of Streptococcus agalactiae. In some embodiments, as disclosed herein, the first biotinylated polysaccharide, or second biotinylated polysaccharide, or both, is from any of serotypes IIa, Ib, II, III, IV, V or VII of Streptococcus agalactiae (7VMAPS-GBS). In some embodiments, as disclosed herein, the first biotinylated polysaccharide, or second biotinylated polysaccharide, or both, is from any of serotypes IIa, Ib, II, III, V or VII of Streptococcus agalactiae (6VMAPS-GBS).

It also is envisioned a MAPS-GBS polyvalent immune composition as disclosed herein, which comprises at least 1, or at least 2, or at least 3, or at least 4, or at least 5, or at least 6., or at least 7, or at least 8 or more species of MAPS-GBS immunogenic complexes can be combined with other non-GBS MAPS immunogenic complexes, e.g., other MAPS immunogenic complexes comprising polysaccharides from different bacteria, e.g., such as those from as disclosed herein and further comprise one or more Multiple presenting antigen systems (MAPS), such as those disclosed in in International Applications: WO/2020/056127, WO/2020/056202, WO/2014/124228, WO/2023/039223, U.S. Ser. No. 11/560,410B2, WO/2018/183475, WO/2018/217564, WO/2023/102359, WO/2023/102359A9, WO/2012/155007, WO/2023/192997A2WO/2013/134656, WO/2023/039108, WO/2012/155053, WO/2023/172741A2, WO/2014/124228, WO/2020/056127, WO/2017/192801, WO/2020/056202, WO/2018/237221, WO/2023/039223, U.S. Ser. No. 11/305,001, US20220362367, US20210008192, US20200121777, US20140154287, U.S. Ser. No. 10/766,932, US20210332090, US20230233667, U.S. Ser. No. 11/560,410, U.S. Ser. No. 10/611,805, US20160090404, U.S. Ser. No. 10/017,548, U.S. Pat. No. 9,499,593, US20140154286, US20200407404, US20190119335, US20150374811, U.S. Ser. No. 11/576,958, US20230081705, US20230089151, US20200087361, US20190119332, U.S. Ser. No. 11/013,793, U.S. Ser. No. 11/701,416, US20210346487, US20200222522, U.S. Ser. No. 11/612,647, US20220072118, US20230091255, or US20150374811, or WO2023/102359 or PCT application: PCT/2023/76822, each of which are incorporated herein in their entirety by reference.

Another aspect of the present invention relates to, methods of making or producing the immune composition as disclosed herein, a pharmaceutical composition comprising the immune compositions as disclosed herein and/or a fusion protein disclosed herein, a vaccine, e.g., a polyvalent vaccine, comprising the immune composition as disclosed herein, a method to induce an immune response in a subject by administering any one or more of: the immune compositions as disclosed herein and/or a fusion protein as disclosed herein, wherein the immune response is an antibody or B cell response.

### BRIEF DESCRIPTION OF THE DRAWINGS

FIG. 1A-1F are schematic drawings that shows an exemplary MAPS-GBS immunogenic complexes using the bi-functional SBD-BBM fusion protein, including, but not limited to a SBD-[GBS-Ag]n-BBM fusion protein. FIG. 1A shows an exemplary bifunctional SBD-BBM fusion protein comprising a Rhizavidin-[GBS antigen]-SBD fusion protein and a biotinylated polysaccharide, e.g., a biotinylated GBS polysaccharide. GBS capsular polysaccharides comprise sialic acids, which are optimal for capsule polymerization and expression, as well as an epitope for opsonic antibodies. FIG. 1B shows the formation of a multiple antigen presenting system (MAPS) complex comprising at least a first polysaccharide antigen (PS1) and a second polysaccharide antigen (PS2) and at least one SBD-[GBS-Ag]-BBM fusion proteins, where the SBD of a first fusion protein interacts with and non-covalently associates to sialic acid on a first polysaccharide antigen (PS1), and the Rhavi of the same first fusion protein interacts with and non-covalently associates to a biotin located on a second polysaccharide antigen (PS2), and vice versa (e.g., the Rhavi of a second fusion protein interacts with and non-covalently associates to biotin on a first polysaccharide antigen (PS1), and the SBD of the same second fusion protein interacts with and non-covalently associates, or binds to a sialic acid located on a second polysaccharide antigen (PS2)) to form a MAPS-GBS immunogenic complex. In some embodiments, the PS1 and PS2 can be the same or different GBS polysaccharides, for example, the PS1 and PS2 can be selected from one of the different Streptococcus agalactiae serotypes, as disclosed herein, e.g., 6 or 7 of serotypes Ia, Ib, II, III, IV, V or VII of Streptococcus agalactiae. In between the SBD and Rhavi, there can be one, or two polypeptide antigens, e.g., GBS antigens, such as, but not limited to Rib and/or Sip, as disclosed herein. FIG. 1C is a schematic showing exemplary embodiments of a MAPS-GBS immunogenic complex, showing (i) a single bifunctional SBD-[GBS-Ag]-BBM fusion protein non-covalently associating with the PS1 and PS2, (ii) two (or a plurality of) bifunctional SBD-[GBS-Ag]-BBM fusion proteins non-covalently associating with the PS1 and PS2, and (iii) two (or a plurality of) different species of bifunctional SBD-[GBS-Ag]-BBM fusion proteins non-covalently associating with the PS1 and PS2. FIG. 1D is a schematic showing exemplary embodiments of MAPS-GBS immunogenic complex species, with FIG. 1D(ii) showing the MAPS-GBS complex of FIG. 1C(ii) or FIG. 1C(iii) further comprising a plurality of BBM-[GBS-Ag] fusion proteins (which can be the same or different species), which can non-covalently associate with one PS. FIG. 1D(ii) shows a MAPS-GBS complex of FIG. 1C(ii) or FIG. 1C(iii) further comprising a plurality of SBD-[GBS-Ag] fusion proteins (which can be the same or different species), which can non-covalently associate with only one PS at a time. FIG. 1D(iii) shows the MAPS-GBS complex of FIG. 1C(ii) or FIG. 1C(iii) further comprising a plurality of both BBM-[GBS-Ag] fusion proteins and SBD-[GBS-Ag] fusion proteins, where these non-bifunctional fusion proteins (e.g., only bind to one polysaccharide at a time) can comprise the same GBS antigen, or a different GBS antigen to that present in the bifunctional SBD-[GBS-Ag]-BBM fusion protein. FIG. 1E is a schematic showing an exemplary embodiment of MAPS-GBS immunogenic complex species, which forms a larger complex as compared to a MAPS complex that does not comprise a bifunctional SBD-[GBS-Ag]-BBM fusion protein. For illustrative purposes only, shown is a MAPS-GBS complex of 5 polysaccharides (PS1-PS5), a first SBD-BBM fusion protein (FP1) can associate with PS1 and PS2, and a second SBD-BBM fusion protein (FP2) can associate with PS2 and PS3, and a third SBD-BBM fusion protein (FP3) can associate with, e.g., PS3 and PS4, and a fourth SBD-BBM fusion protein (FP2) can associate with, e.g., PS4 and PS5, etc. thereby forming a multi-bifunctional fusion protein and GBS polysaccharide complex (e.g., a MAPS-GBS immunogenic complex). Some SBD-BBM fusion proteins can also associate with polysaccharides already existing in the complex, for example, a fifth SBD-BBM fusion protein (FP5) can associate with PS5 and PS1, a sixth SBD-BBM fusion protein (FP6) can associate with PS5 and PS3, strengthening the MAPS-GBS complex. The polysaccharides, e.g., PS1-PS5 be from the same subtype of GBS. In alternative embodiments, each PS (e.g., PS1, PS2, PS3, PS4, PS5, PS6 etc.) can be from a different subtype of GBS. Accordingly, due to the presence of a plurality of bifunctional SBD-[GBS-Ag]-BBM fusion proteins which can non-covalently associate with two polysaccharides, a MAPS-GBS immunogenic complex as disclosed herein can cross-link a plurality of polysaccharides in the complex, therefore forming larger complexes than prior MAPS complexes which comprise biotin-binding fusion proteins that can non-covalently associate with only one polysaccharide at a time. FIG. 1F is a schematic showing an exemplary embodiment of a 3 valent (3V) MAPS-GBS vaccine composition comprising three different MAPS-GBS immunogenic complex species, each species comprising a different biotinylated polysaccharide, which can be selected from serotypes any of: Ia, Ib, II, III, IV, V, VI, VII and VIII. Each species of the MAPS-GBS immunogenic complex can comprise the same, or a different SBD-[GBS-Ag]-BBM fusion protein as the other species of MAPS-GBS immunogenic complex in the vaccine composition. Additionally, each MAPS-GBS immunogenic complex within the composition can comprise the same species of SBD-[GBS-Ag]-BBM fusion protein, or different species within the complex (e.g., see FIGS. 1C(ii) and 1C(ii)).

FIG. 2. shows that incorporation of SBD in the carrier protein significantly enhances antibody to GBS polysaccharide. The antibody response to GBS polysaccharides from serotypes Ib, II and III polysaccharides (GBS1b, GBSII or GBSIII) was assessed in a MAPS complex comprising Rhavi-antigen (e.g., a fusion protein comprising Rhavi-0435 antigen) or a MAPS complex comprising a SBD-[GBS-Ag]-BBM fusion protein (e.g., Rhavi-0435-SBD fusion protein), showing that incorporation of Rhavi-(GBS-Ag)-SBD fusion protein in the MAPS complex, as opposed to Rhavi-antigen significantly enhances antibody responses to the GBS polysaccharides.

FIG. 3A-3B shows immunization with 6V MAPS-GBS immunogenic composition induces a robust functional antibody response to GBS polysaccharides from serotypes Ia, Ib, II, III, V and VII. FIG. 3A shows the antibody response to GBS polysaccharides from serotypes Ia, Ib, II, III, V and VII after immunization with a MAPS complex comprising the SBD-Rhavi fusion protein. Six rabbits were immunized with 6V MAPS-GBS vaccine complex (0.4 ug PS/dose). OPK titer of pre-immune sera (P0) was below the lower limit of detection (20) for all serotypes. FIG. 3B shows the OPK titer on post-2 immunization (P2) sera.

FIG. 4 shows candidate protective GBS polypeptide antigens for incorporating into a SBD-Rhavi fusion protein, with candidate protein antigens selected from: PI-2a, Sip, Rib, AlpC and Alp3/1.

FIG. 5 shows surface exposure of selected GBS protein antigens on different GBS strains. Flow cytometry analysis (MFI) on heat-killed GBS was used to show the binding of antibodies against GBS polypeptide antigens, PI-2a, Sip, Rib and AlpC antigens on serotypes Ia, Ib, II, III, V and VII of Streptococcus agalactiae.

FIG. 6 shows OPA (opsonic killing assay) of anti-GBS antibodies to specific antigen polypeptides PI-2a, Sip, Rib, AlpC and Alp3/1 on serotypes Ia, Ib, II, III, V and VII of Streptococcus agalactiae.

FIG. 7A-7B shows results of passive immunization with anti-Rib serum protects adult mice against type Ia or type III GBS infection. FIG. 7A-7B shows a Kaplan meier curve for immunization with anti-Rib serum protects adult mice from infection with serotypes Ia (FIG. 7A) or III (FIG. 7B) Streptococcus agalactiae.

FIG. 8 shows results of passive maternal immunization with anti-Rib serum protects infant mice against type 3 GBS infection. A Kaplan Meier curve for immunization of pregnant mice 3-days before delivery with anti-Rib serum protects infant mice from infection with serotype III Streptococcus agalactiae.

FIG. 9A-9B shows results of passive maternal immunization with anti-Rib/Sip sera protects infant mice against type II or III GBS infection. FIG. 9A-9B show Kaplan Meier curves for immunization with anti-Rib/Sip serum protects infant mice from infection with serotype II (FIG. 9A) or III (FIG. 9B) Streptococcus agalactiae.

FIG. 10 shows that different GBS strains (also known as clones) of the same serotype have variable sensitivity to anti-protein antigens. Clones analyzed are: S12, SA1, SA3, S28, SA7, SA9, M781, SA10, SA12, S35, SA45, SA65, SA13, ATTC, S3, SA14, S5, S25, S88.

FIG. 11 shows that different GBS strains from the same serotype have different amounts of capsule polysaccharides. PS content is measured on paraformaldehyde-fixed GBS by inhibition ELISA using purified GBS CPS as standard. Comparison between different serotypes may not be valid as the size of purified serotypes vary (which impacts inhibition efficiency).

FIG. 12 shows that combinations of anti-antigenic polysaccharides (anti-PS) and antibodies to the antigenic polypeptides (anti-protein) results in synergy (e.g., the combined effect is greater than the sum of each individually). Sip is present in 90% of sequenced GBS strains. Rib is present in most type III GBS strains which is the dominant GBS serotype causing LOD.

FIG. 13 shows that in vitro synergy is even seen when GBS isolates are not susceptible to anti-protein antibody-mediated killing.

FIG. 14 shows that in vitro synergy is even seen when GBS isolates are not susceptible to anti-protein antibody-mediated killing.

FIG. 15A-15B shows that Rhavi-Sip-Rib-SBD MAPS-GBS immunogenic complex induces functional anti-polysaccharide and anti-Sip and anti-Rib protein antibody responses after 1 dose in rabbits. FIG. 15A shows anti-PS responses to GBS polysaccharides from serotypes Ia, Ib, II, III, V and VII. FIG. 15B shows anti-Rib or anti-Sip antibody responses.

FIG. 16 shows that P1 sera from rabbits immunized with Rhavi-Sip-Rib-SBD MAPS have enhanced killing activity against serotypes II (strains 28 and SA9), III, and V GBS stains.

FIG. 17 shows P3 antibody titer of mice immunized with an exemplary 7V MAPS-GBS immunogenic composition induced an anti-polysaccharide IgG immune response to polysaccharides from serotypes Ia, Ib, II, III, IV, V and VII of Streptococcus agalactiae. The 7V MAPS-GBS immunogenic composition comprises an admixture of 7 different MAPS-GBS immunogenic complexes, each MAPS-GBS immunogenic complex comprising a biotinylated polysaccharide selected from any of the serotypes Ia, Ib, II, III, IV, V and VII of Streptococcus agalactiae, and non-covalently bound to each biotinylated polysaccharide a Rhavi-Sip-Rib-SBD fusion protein.

FIG. 18 shows mice administered the 7V MAPS-GBS immunogenic composition induced a robust immune response to Rib and Sip proteins, as determined by the anti-Rib and anti-Sip IgG responses.

FIG. 19A-19D shows the anti-polysaccharide IgG responses (anti-PS IgG responses) of rabbits immunized with MAPS-GBS immunogenic complexes comprising different fusion proteins selected from rhavi-SBD vs. rhavi-Rib-SBD vs. rhavi-Sip-SBD. FIG. 19A shows anti-GBS 1b IgG in P0, P1 and P2 sera from rabbits immunized with MAPS-GBS immunogenic complexes comprising any of the fusion proteins selected from rhavi-SBD vs. rhavi-Rib-SBD vs. rhavi-Sip-SBD. FIG. 19B shows anti-GBS II IgG in P0, P1 and P2 sera from rabbits immunized with MAPS-GBS immunogenic complexes comprising any of the fusion proteins selected from rhavi-SBD vs. rhavi-Rib-SBD vs. rhavi-Sip-SBD. FIG. 19C shows anti-GBS III IgG in P0, P1 and P2 sera from rabbits immunized with MAPS-GBS immunogenic complexes comprising any of the fusion proteins selected from rhavi-SBD vs. rhavi-Rib-SBD vs. rhavi-Sip-SBD. FIG. 19D shows anti-GBS IV IgG in P0, P1 and P2 sera from rabbits immunized with MAPS-GBS immunogenic complexes comprising any of the fusion proteins selected from rhavi-SBD vs. rhavi-Rib-SBD vs. rhavi-Sip-SBD.

FIG. 20A-20B show OPK titer of P2 sera of rabbits immunized with MAPS-GBS immunogenic complexes comprising different fusion proteins selected from rhavi-SBD vs. rhavi-Rib-SBD vs. rhavi-Sip-SBD. FIG. 20A shows type Ib GBS killing titers of rabbits immunized with MAPS-GBS immunogenic complexes comprising any of the fusion proteins selected from rhavi-SBD vs. rhavi-Rib-SBD vs. rhavi-Sip-SBD. FIG. 20B shows type III GBS killing titers of rabbits immunized with MAPS-GBS immunogenic complexes comprising any of the fusion proteins selected from rhavi-SBD vs. rhavi-Rib-SBD vs. rhavi-Sip-SBD.

FIG. 21A-21D show the anti-polysaccharide IgG responses (anti-PS IgG responses) of rabbits immunized with different amounts of MAPS-GBS immunogenic complexes comprising different fusion proteins selected from rhavi-SBD, rhavi-Rib-Sip-SBD, or rhavi-Sip-Rib-SBD. FIG. 21A shows anti-GBS 1b IgG in P0, P1 and P2 sera from rabbits immunized with 1 or 0.5 g MAPS-GBS immunogenic complexes comprising any of the fusion proteins selected from rhavi-SBD, rhavi-Rib-Sip-SBD, or rhavi-Sip-Rib-SBD. FIG. 21B shows anti-GBS II IgG in P0, P1 and P2 sera from rabbits immunized with 1 or 0.5 g MAPS-GBS immunogenic complexes comprising any of the fusion proteins selected from rhavi-SBD, rhavi-Rib-Sip-SBD, or rhavi-Sip-Rib-SBD. FIG. 21C shows anti-GBS III IgG in P0, P1 and P2 sera from rabbits immunized with 1 or 0.5 g MAPS-GBS immunogenic complexes comprising any of the fusion proteins selected from rhavi-SBD, rhavi-Rib-Sip-SBD, or rhavi-Sip-Rib-SBD. FIG. 21D shows anti-GBS IV IgG in P0, P1 and P2 sera from rabbits immunized with 1 or 0.5 g MAPS-GBS immunogenic complexes comprising any of the fusion proteins selected from rhavi-SBD, rhavi-Rib-Sip-SBD, or rhavi-Sip-Rib-SBD.

FIG. 22A-22C show OPK titer of P2 sera of rabbits immunized with MAPS-GBS immunogenic complexes comprising different fusion proteins selected from rhavi-SBD vs. rhavi-Rib-Sip-SBD. FIG. 22A shows type 1b GBS killing titers of rabbits immunized with MAPS-GBS immunogenic complexes comprising any of the fusion proteins selected from rhavi-SBD vs. rhavi-Rib-Sip-SBD. FIG. 22B shows type II GBS killing titers of rabbits immunized with MAPS-GBS immunogenic complexes comprising a fusion protein selected from rhavi-SBD vs. rhavi-Rib-Sip-SBD. FIG. 22C shows type III GBS killing titers of rabbits immunized with MAPS-GBS immunogenic complexes comprising a fusion protein selected from rhavi-SBD vs. rhavi-Rib-Sip-SBD.

FIG. 23A-23B shows that rhavi-Rib-Sip-SBD or rhavi-Sip-Rib-SBD MAPS-GBS immunogenic complex induces a functional anti-Sip and anti-Rib IgG antibody responses to antigenic polypeptides Sip and Rib after 1 dose in rabbits. FIG. 23A shows anti-Rib IgG antibody responses to MAPS-GBS comprising fusion proteins Rhavi-Rib-Sip-SBD or Rhavi-Sip-Rib-SBD. FIG. 23B shows anti-Sip IgG antibody responses to MAPS-GBS comprising fusion proteins Rhavi-Rib-Sip-SBD or Rhavi-Sip-Rib-SBD.

FIG. 24A-24D show that MAPS-GBS immunogenic complexes comprising different fusion proteins selected from: Rhavi-SBD, Rhavi-SBD-Rib-Sip, and Rib-Sip-Rhavi-SBD induces a functional anti-polysaccharide antibody response in rabbits. FIG. 24A shows anti-PS IgG responses to GBS polysaccharide serotype II in P0, P1 and P2 sera from rabbits immunized with MAPS-GBS immunogenic complexes comprising any of the fusion proteins selected from: Rhavi-SBD, Rhavi-SBD-Rib-Sip and Rib-Sip-Rhavi-SBD. FIG. 24B shows anti-PS IgG responses to GBS polysaccharide serotype III in P0, P1 and P2 sera from rabbits immunized with MAPS-GBS immunogenic complexes comprising any of the fusion proteins selected from: Rhavi-SBD, Rhavi-SBD-Rib-Sip and Rib-Sip-Rhavi-SBD. FIG. 24C shows anti-PS IgG responses to GBS polysaccharide serotype IV in P0, P1 and P2 sera from rabbits immunized with MAPS-GBS immunogenic complexes comprising any of the fusion proteins selected from: Rhavi-SBD, Rhavi-SBD-Rib-Sip and Rib-Sip-Rhavi-SBD. FIG. 24D shows anti-PS IgG responses to GBS polysaccharide serotype VII in P0, P1 and P2 sera from rabbits immunized with MAPS-GBS immunogenic complexes comprising any of the fusion proteins selected from: Rhavi-SBD, Rhavi-SBD-Rib-Sip and Rib-Sip-Rhavi-SBD.

FIG. 25A-25B show that MAPS-GBS immunogenic complexes comprising different fusion proteins selected from: Rhavi-SBD-Rib-Sip and Rib-Sip-Rhavi-SBD induces a functional anti-Rib or Anti-Sip IgG antibody response in rabbits. FIG. 25A shows anti-Rib IgG responses to GBS polysaccharide serotype II in P0, P1 and P2 sera from rabbits immunized with MAPS-GBS immunogenic complexes comprising any of the fusion proteins selected from: Rhavi-SBD-Rib-Sip and Rib-Sip-Rhavi-SBD. FIG. 25B shows anti-Sip IgG responses to GBS polysaccharide serotype II in P0, P1 and P2 sera from rabbits immunized with MAPS-GBS immunogenic complexes comprising any of the fusion proteins selected from: Rhavi-SBD-Rib-Sip and Rib-Sip-Rhavi-SBD.

FIG. 26 shows the OPK titer of P2 sera of rabbits immunized with MAPS-GBS immunogenic complexes comprising different fusion proteins selected from rhavi-SBD vs. Rib-Sip-rhavi-SBD. FIG. 26 shows type II, III, IV GBS killing titers of rabbit sera after two immunizations with MAPS-GBS immunogenic complexes comprising any of the fusion proteins selected from rhavi-SBD vs. Rib-Sip-rhavi-SBD.

FIG. 27A-27F shows results of immunization with 7V MAPS-GBS immunogenic complexes comprising a fusion protein rhavi-Rib-Sip-SBD protects adult mice against types 1b, II, III, IV and IV GBS infection. FIG. 27A shows a Kaplan meier survival curve of CD1 mice infected with SA5 (serotypes Ib of Streptococcus agalactiae) after immunization with 7V MAPS-GBS immunogenic complexes comprising a fusion protein rhavi-Rib-Sip-SBD. FIG. 27B shows a Kaplan meier survival curve of CD1 mice infected with type 1b GBS Streptococcus agalactiae after immunization with 7V MAPS-GBS immunogenic complexes comprising a fusion protein rhavi-Rib-Sip-SBD. FIG. 27C shows a Kaplan meier survival curve of CD1 mice infected with type II GBS Streptococcus agalactiae after immunization with 7V MAPS-GBS immunogenic complexes comprising a fusion protein rhavi-Rib-Sip-SBD. FIG. 27D shows a Kaplan meier survival curve of CD1 mice infected with type III GBS Streptococcus agalactiae after immunization with 7V MAPS-GBS immunogenic complexes comprising a fusion protein rhavi-Rib-Sip-SBD. FIG. 27E shows a Kaplan meier survival curve of CD1 mice infected with type IV GBS Streptococcus agalactiae after immunization with 7V MAPS-GBS immunogenic complexes comprising a fusion protein rhavi-Rib-Sip-SBD. FIG. 27F shows a Kaplan meier survival curve of CD1 mice infected with type VII GBS Streptococcus agalactiae after immunization with 7V MAPS-GBS immunogenic complexes comprising a fusion protein rhavi-Rib-Sip-SBD. In each experiment for FIG. 27A-27F, CD1 mice received three immunizations with a 7V rhavi-Rib-Sip-SBD MAPS vaccine or a pneumococcal protein (SP_1826, negative control), both were adjuvanted with aluminum phosphate.

FIG. 28A-28H shows results of passive immunization of mice with sera from rabbits immunized with 7V MAPS-GBS immunogenic complexes comprising a fusion protein selected from rhavi-Rib-Sip-SBD, or Rhavi-SBD, or administration of the rhavi-Rib-Sip-SBD protein alone, protects adult mice against multiple subtypes of GBS infection. FIG. 28A shows a Kaplan meier survival curve of CD1 mice infected with type Ia GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the 7V MAPS-GBS immunogenic complexes comprising the fusion protein rhavi-Rib-Sip-SBD or Rhavi-SBD had statistically significant survival rates compared to the negative control. FIG. 28B shows a Kaplan meier survival curve of CD1 mice infected with type 1b GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the rhavi-Rib-Sip-SBD protein alone, or 7V MAPS-GBS immunogenic complexes comprising the fusion protein rhavi-Rib-Sip-SBD or Rhavi-SBD had statistically significant survival rates as compared to the negative control. FIG. 28C shows a Kaplan meier survival curve of CD1 mice infected with type II (strain 1) GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the rhavi-Rib-Sip-SBD protein alone, or 7V MAPS-GBS immunogenic complexes comprising the fusion protein rhavi-Rib-Sip-SBD or Rhavi-SBD had statistically significant survival rates as compared to the negative control. FIG. 28D shows a Kaplan meier survival curve of CD1 mice infected with type II (strain 2) GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the 7V MAPS-GBS immunogenic complexes comprising the fusion protein rhavi-Rib-Sip-SBD or Rhavi-SBD had statistically significant survival rates as compared to negative control or mice received anti-sera from rabbits immunized with the rhavi-Rib-Sip-SBD protein alone. FIG. 28E shows a Kaplan meier survival curve of CD1 mice infected with type III GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the 7V MAPS-GBS immunogenic complexes comprising the fusion protein rhavi-Rib-Sip-SBD or Rhavi-SBD had statistically significant survival rates as compared to negative control or mice received anti-sera from rabbits immunized with the rhavi-Rib-Sip-SBD protein alone. FIG. 28F shows a Kaplan meier survival curve of CD1 mice infected with type IV GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the 7V MAPS-GBS immunogenic complexes comprising the fusion protein rhavi-Rib-Sip-SBD or Rhavi-SBD had statistically significant survival rates as compared to negative control or mice received anti-sera from rabbits immunized with the rhavi-Rib-Sip-SBD protein alone. FIG. 28G shows a Kaplan meier survival curve of CD1 mice infected with type V GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the rhavi-Rib-Sip-SBD protein alone, or 7V MAPS-GBS immunogenic complexes comprising the fusion protein rhavi-Rib-Sip-SBD or Rhavi-SBD had statistically significant survival rates as compared to the negative control. FIG. 28H shows a Kaplan meier survival curve of CD1 mice infected with type VII GBS Streptococcus agalactiae after passive immunization with sera from rabbits immunized with (i) 7V MAPS-GBS immunogenic complexes comprising the fusion protein rhavi-Rib-Sip-SBD or Rhavi-SBD, or immunization with (ii) the rhavi-Rib-Sip-SBD protein alone, or (iii) no immunization (negative control), showing that mice received anti-sera from rabbits immunized with the 7V MAPS-GBS immunogenic complexes comprising the fusion protein rhavi-Rib-Sip-SBD or Rhavi-SBD had statistically significant survival rates as compared to negative control or mice received anti-sera from rabbits immunized with the rhavi-Rib-Sip-SBD protein alone. In each experiment for FIG. 28A-28H, mice received IP injection with 200 μl serum from naïve rabbits (negative control), or rabbits immunized with 7V rhavi-Rib-Sip-SBD MAPS, 7V rhavi-SBD MAPS, or rhavi-Rib-Sip-SBD protein 1-day prior infection with indicated GBS strains.

FIG. 29 shows results of enhanced protection mediated by combined anti-PS and anti-protein antibodies compared to each antibody individually against type IV GBS infection. Mice received passive immunization with serum from rabbits immunized with rhavi-Rib-Sip-SBD protein alone (serum 3), or 7V rhavi-SBD MAPS-GBS immunogenic complexes (serum 1 and serum 2), or a combo sera comprising serum 1 and serum 3, or serum 2 and serum 3. The negative control group received naïve rabbit serum. Mice received the combo sera had better survival rate compared to the control group, or the groups received single serum.

### DETAILED DESCRIPTION OF CERTAIN EMBODIMENTS

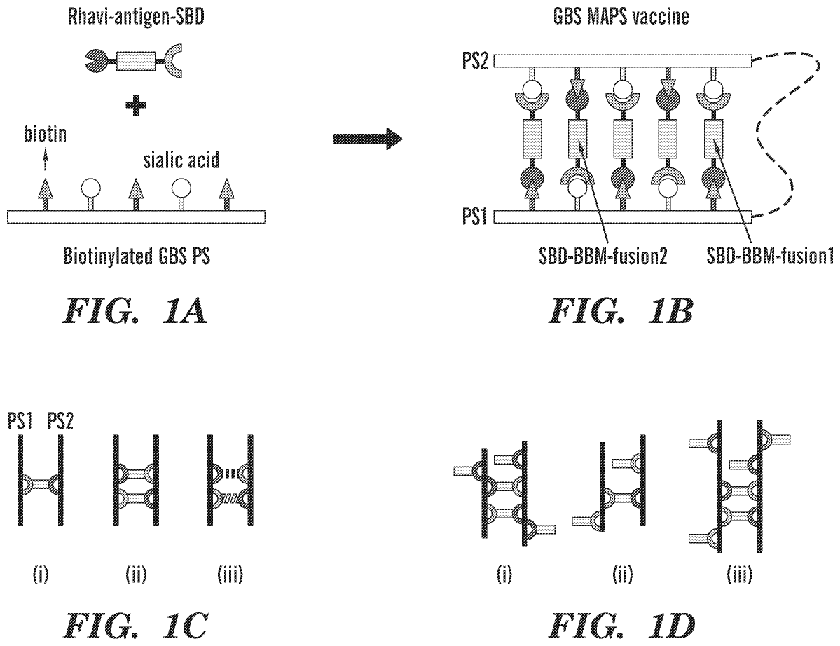

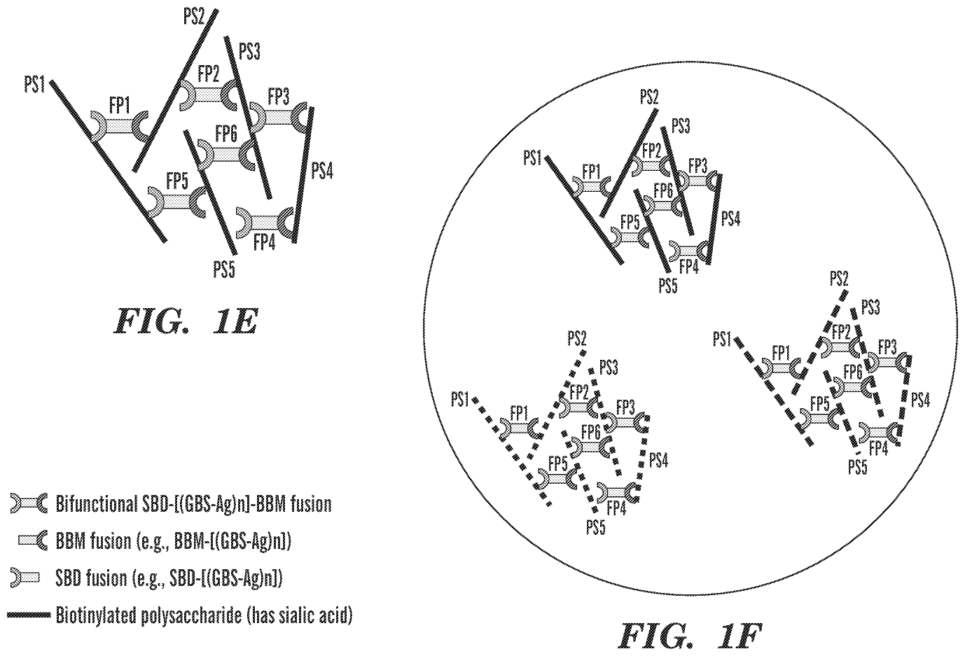

The present disclosure relates, generally, to compositions, systems, and methods of use thereof, of a multiple antigen presenting system (MAPS) immunogenic complex, comprising fusion proteins and GBS polysaccharides. Some aspects of the technology relate to compositions, including, a vaccines, comprising a plurality of GBS-MAPS immunogenic complexes, wherein each MAPS-GBS immunogenic complex comprises: (a) at least one biotinylated GBS polysaccharide antigen; and (b) at least one fusion protein selected from (i) a biotin-binding moiety (BBM) fusion protein, or (ii) a sialic acid binding domain (SBD) fusion protein, or (iii) a bifunctional SBD-BBM fusion protein; wherein the fusion protein further comprises at least one GBS polypeptide antigen, wherein the biotinylated GBS polysaccharide antigen comprises a polysaccharide from GBS, and further wherein the biotinylated polysaccharide antigen is non-covalently associated with at least one fusion protein selected from any of (i)-(iii) to form a MAPS-GBS immunogenic complex. In some embodiments, the MAPS-GBS immunogenic complex comprises at least two biotinylated polysaccharides, e.g. PS1 and PS2, e.g., as illustrated in an exemplary MAPS-GBS in FIG. 1B.

Such complexes can be used, e.g., to induce and/or increase an immunoprotective response in subjects at risk of or suffering from Group B Streptococcus (GBS) infection. Aspects of the present disclosure relates, generally, to novel immunogenic fusion proteins of Streptococcus agalactiae that can be used, e.g., to induce and/or increase an immunoprotective response, or to reduce Streptococcus agalactiae infection or colonization in subjects at risk of or suffering from Streptococcus agalactiae infection. The presently disclosed MAPS-GBS immunogenic complexes represent a substantial advance over the currently available options for immunizing patients against Streptococcus agalactiae infection. Such MAPS-GBS immunogenic complexes can be used, e.g., to induce and/or increase an immunoprotective response or to reduce Streptococcus agalactiae colonization in subjects, such as those at risk of or suffering from Streptococcus agalactiae infection.

Accordingly, aspects of the technology disclosed herein relates to methods, compositions and vaccines comprising a MAPS-GBS immunogenic complex using a bi-functional fusion protein comprising a sialic acid-binding domain (SBD) and a biotin-binding moiety (BBM), where the bi-functional fusion protein further comprises at least one polypeptide from Group B Streptococcus (GBS) or Streptococcus agalactiae.

### I. MAPS-GBS Immunogenic Complexes

Aspects of the technology relate to a MAPS-GBS immunogenic complex, wherein each MAPS-GBS immunogenic complex comprises: (a) a biotinylated GBS polysaccharide antigen (PS1); and (b) at least one fusion protein selected from (i) a biotin-binding moiety (BBM) fusion protein (e.g., BBM-[GBS-Ag]n fusion protein), or (ii) a sialic acid binding domain (SBD) fusion protein (e.g., SBD-[GBS-Ag]n fusion protein), or (iii) a bifunctional SBD-BBM fusion protein (e.g., SBD-[GBS-Ag]n-BBM fusion protein); wherein the fusion protein further comprises at least one GBS polypeptide antigen, and wherein the biotinylated polysaccharide antigen is non-covalently associated with at least two fusion proteins form an immunogenic complex.

As disclosed herein, The MAPS-GBS complex as disclosed herein comprises biotinylated polysaccharides and a bifunctional SBD-[GBS-Ag]-BBM fusion protein. In brief, a bifunctional SBD-[GBS-Ag]-BBM serves as both a carrier protein and a linking protein, which can link two polysaccharide chains together, thereby forming a cross-linked MAPS-GBS complex. Stated differently, a MAPS-GBS complex comprises at least two polysaccharide antigens, which can be (i) on the same polysaccharide macromolecule or (ii) on different polysaccharide macromolecules, where the two polysaccharide antigens are linked together via the SBD-[GBS-Ag]-BBM fusion protein which non-covalently associates with each of the two polysaccharide antigens. As such, the SBD-[GBS-Ag]-BBM fusion protein serves as a linking protein that is also functions as a carrier protein to link two polypeptide antigens.

In some embodiments, if the two polypeptide antigens, referred herein and throughout the specification as PS1 and PS2 respectively, are located on the same macromolecule, e.g., on the same polymer chain, or on different branches of a polymer of the same macromolecule, the SBD-[GBS-Ag]-BBM fusion protein serves an intra-macromolecule linkage role to form a MAPS-GBS immunogenic complex. In alternative embodiments, if the two polypeptide antigens are located on a different macromolecule, e.g., one PS1 is from a distinct polysaccharide macromolecule, and the other PS2 is a distinct polysaccharide macromolecule (e.g., from a different pathogen and/or a specific serotype from the macromolecule used in P1), the SBD-[GBS-Ag]-BBM fusion protein serves an inter-macromolecule linkage role, to form a MAPS-GBS immunogenic complex with more than one polysaccharide macromolecule.

For illustrative purposes only, referring to FIG. 1B, a MAPS-GBS immunogenic complex comprises; (i) at least a first biotinylated polysaccharide antigen (PS1) comprising at least one sialic acid domain and at least one biotin molecule, (ii) at least a second biotinylated polysaccharide antigen (PS2) comprising at least one sialic acid domain and at least one biotin molecule, and (iii) at least one fusion protein, selected from a biotin-binding moiety (BBM) fusion protein (e.g., BBM-[GBS-Ag]n fusion protein), or (ii) a sialic acid binding domain (SBD) fusion protein (e.g., SBD-[GBS-Ag]n fusion protein), or (iii) a bifunctional SBD-BBM fusion protein (e.g., SBD-[GBS-Ag]n-BBM fusion protein).

In some embodiments where at least two bifunctional SBD-BBM fusion protein are used, a BBM of at least a first fusion protein (SBD-BBM fusion1) non-covalently associates with at least one biotin molecule on the first biotinylated polysaccharide antigen (PS1) and the SBD non-covalently associates with at least one sialic acid on the second biotinylated polysaccharide antigen (PS2), and the SBD of a second SBD-BBM fusion protein (SBD-BBM fusion2) non-covalently associates with at least one sialic acid domain on the first biotinylated polysaccharide antigen (PS1) and the BBM of the second fusion protein non-covalently associates with at least biotin molecule on the second biotinylated polysaccharide antigen (PS2). This results in the PS1 and PS2 forming a MAPS-complex by the non-covalent association via the first and second bifunctional SBD-BBM fusion proteins. In some embodiments, where the first (PS1) and second polysaccharide antigen (PS2) are located on the same macromolecule, e.g., on the same polymer chain, or on different branches of a polymer of the same macromolecule, the SBD-[GBS-Ag]-BBM fusion protein serves an intra-macromolecule linkage role to form a MAPS-GBS immunogenic complex. Where the PS1 and PS2 are located on a different macromolecule, e.g., PS1 is located on a specific polysaccharide macromolecule, and the other PS2 is located a distinct polysaccharide macromolecule (e.g., from a different pathogen and/or a specific serotype to the PS1 macromolecule), the SBD-[GBS-Ag]-BBM fusion protein serves an inter-macromolecule linkage role, to form a MAPS-GBS immunogenic complex with more than one polysaccharide macromolecule.

In some embodiments, the MAPS-GBS immunogenic complex can comprise the following non-covalent associations; PS1-(SBD-BBM fusion protein1)-PS2, where the BBM of the first SBD-BBM fusion protein non-covalently associates with a biotin on the first polysaccharide (PS1), and the SBD of the first SBD-BBM fusion protein non-covalently associates with a sialic acid on the second polysaccharide (PS2), to form a MAPS-GBS immunogenic complex. In some embodiments, a MAPS-GBS immunogenic complex can also comprise the following non-covalent associations: PS1-(SBD-BBM fusion protein2)-PS2, where the SBD of the second SBD-BBM fusion protein non-covalently associates with a sialic acid on the first polysaccharide (PS1), and the BBM of the second SBD-BBM fusion protein non-covalently associates with a biotin on the second polysaccharide (PS2). As the first (PS1) and second antigenic polysaccharides (PS2) both have biotin molecules and sialic acid domains on their surface, the MAPS-GBS immune complex can comprise the following non-covalent associations: PS1-(SBD-BBM fusion protein1)-PS2 and PS1-(SBD-BBM fusion protein2)-PS2 (see, e.g., FIG. 1B and FIG. 1E). It is envisioned that the SBD-BBM fusion protein further comprises at least one, or at least 2, or at least 3 or more GBS polypeptide antigens. Exemplary GBS fusion proteins for use as SBD-BBM fusion proteins are disclosed in Tables 2A, 2B and Table 3, herein. The position of the GSB antigen in the fusion protein is flexible, and can optionally be located anywhere between a BBM and a SBD polypeptide, or at the N- or C-terminus, as disclosed in the fusion proteins listed in Table 3. It is envisioned that as a plurality of SBD-BBM fusion protein are used in each MAPS-GBS complex, each polysaccharide can be non-covalently attached to more than 1 (e.g., 1, 2, 3, 4, 5, 6, 7, 8 or more) different SBD-BBM fusion proteins, therefore cross-linking the polysaccharides in the complex via the bifunctional SBD-BBM fusion proteins. Accordingly, due to the presence of a plurality of bifunctional SBD-[GBS-Ag]-BBM fusion proteins, each of which can non-covalently associate with two polysaccharides, a MAPS-GBS immunogenic complex as disclosed herein can cross-link a plurality of polysaccharides in the MAPS-GBS immunogenic complex, forming larger complexes than prior MAPS complexes which comprise biotin-binding fusion proteins that can non-covalently associate with only one polysaccharide at a time. Such cross-linking and generation of larger MAPS-GBS immunogenic complex induces a larger immunological response to both the polysaccharide and/or GBS antigen on administration to a subject.

### II. GBS Antigen Fusion Proteins

Aspects of the technology disclosed herein relate to fusion protein comprising a sialic acid-binding domain (SBD) and an antigenic polypeptide from Group B Streptococcus (GBS) or Streptococcus agalactiae. In some embodiments, the antigenic polypeptide from GBS is selected from Rib, or Sip, or Rib and Sip, AlpC, Alp3/1 as disclosed herein. In some embodiments, a SBD-Antigen fusion protein is selected from any of: SBD-[Rib] fusion protein, SBD-[Sip]-fusion protein, SBD-[Rib-Sip] fusion protein, [Rib]-SBD fusion protein, [Sip]-SBD fusion protein, [Rib-Sip]-SBD fusion protein. In some embodiments, Rib and Sip, either individually, or together can be readily replaced with alternative GBS antigens known in the art.

Aspects of the technology disclosed herein relate to GBS fusion proteins comprising (i) at least one GBS antigenic polypeptide and (ii) a sialic acid binding domain (SBD) and/or a biotin-binding moiety (BBM). For example, in some embodiments, a fusion protein can comprise a sialic acid binding domain (SBD) and one or more GBS antigenic polypeptides as described herein, referred to herein as SBD-[GBS-Ag]n fusion protein, where “[GBS-Ag]” refers to an immunogenic GBS polypeptide as disclosed herein, and n represents the number of GBS polypeptide antigens. N can be 1, 2, 3, or more than 3 GBS polypeptide antigens as disclosed herein. In some embodiments, a fusion protein can comprise a biotin-binding moiety (BBM) and one or more GBS antigenic polypeptides as described herein, referred to herein as BBM-[GBS-Ag]n fusion protein. Alternatively, in some embodiments, a fusion protein can comprise a biotin-binding moiety (BBM), one or more GBS antigenic polypeptides as described herein, and at least one SBD, referred to herein as SBD-[GBS-Ag]n-BBM fusion protein. It is envisioned that the fusion protein can have any order of SBD, GBS-Ag and BBM.

In some embodiments, a GBS fusion protein useful in a GBS-MAPS immunogenic complex has carrier properties. In some embodiments, a fusion protein disclosed herein useful in a MAPS-GBS immunogenic complex has antigenic properties. In some embodiments, a fusion protein useful in a MAPS-GBS in the immunogenic complex has carrier properties and antigenic properties.

Exemplary antigenic GBS polypeptides useful in the fusion proteins are selected from one or more selected from: Rib, Sip, AlpC, Alp1, Alp3 or a Alp3/1 as disclosed herein. In some embodiments, a GBS fusion protein useful in the MAPS-GBS immunogenic complex as disclosed herein comprises at least one, or at least two, or at least 3 antigenic polypeptides selected from SEQ ID NOS: 4, 5, 11-15, or least one, or at least two, or at least 3 antigenic polypeptides having an amino acid sequence having at least 80%, or at least 85%, or at least 90%, or at least 95% sequence identity to SEQ ID NOS: SEQ ID NO: 4, 5, 11-15.

### Exemplary GBS Fusion Proteins

The present disclosure describes novel immunogenic fusion proteins of Streptococcus agalactiae (also known as Group B Streptococcus or GBS). Fusion proteins described and/or utilized herein provide improved immunogenicity and IL-17 response to protein stimulation, as well as further reduction of Streptococcus agalactiae colonization and protection from invasive diseases.

A fusion protein includes one, two, or more polypeptides that elicit (e.g., primarily elicit) a T cell response, or that elicit both a T cell and a B cell response. In some embodiments, the fusion protein comprises one or more of the polypeptides listed in Table 1. In some embodiments, the fusion protein comprises two of the polypeptides listed in Table 1. In some embodiments, the fusion protein comprises three of the polypeptides listed in Table 1. In some embodiments, the fusion protein comprises one or more of polypeptides encoded by one or more of the genes listed in Table 1. In some embodiments, the fusion protein comprises two of polypeptides encoded by two or more of the genes listed in Table 1. In some embodiments, the fusion protein comprises three polypeptides encoded by three of the genes listed in Table 1.

TABLE 1
Exemplary Polypeptide Components of GBS Fusion Proteins

| | Protein | |
|---|---|---|
| | SEQ | DNA SEQ |
| Component | ID NO: | ID NO: |
| Rhavi | 1 | 16 |
| Rhavi(A5T) | 8 | |
| SBD1 NanA (121-305) | 2 | 170 |
| SBD2 NanL (81-272) | 112 | 171 or 172 (codon- |
| | | optimized) |
| SBD3 NanB (40-230) | 113 | 173 |
| SBD4 NanC 82-270 | 114 | 174 |
| NanH (Salmonella) | 115 | |
| NanH2(Salmonella) | 116 | |
| NanH3 Trypanosoma cruzi 4-399 | 117 | |
| SBD NanA (full-length) | 118 | |
| NanH (Vibrio cholera) | 119 | |
| NanH-Truncated (25-216aa) | 120 | |
| (Vibrio cholera) | | |
| Rib | 4 | |
| Sip | 5 | |
| AlpC | 11 | |
| Alp1 | 12 | |
| Alp3 | 13 | |
| Alp3-Alp1 (“Alp3/1”) | 14 | |
| AlpC-Rib | 15 | |

In some embodiments, the present disclosure discloses fusion proteins as disclosed in Table 2A and 2B, although such fusion proteins are representative of exemplary components of the GBS fusion proteins disclosed herein, and are not to be limited to the specific order of proteins shown, rather the proteins components of the fusion protein can be in any order.

TABLE 2A
Exemplary GBS fusion proteins for use in a MAPS-GBS immunogenic complex,
where the fusion protein comprises at least one or two GBS polypeptide
antigens. SBD refers to any SBD protein as disclosed herein, including
but not limited to SBD1, SBD2, SBD3, SBD4, NanH, NanH2 or VcNanH,
or having an amino acid sequence with at least 80%, or at least 85%,
or at least 90%, or at least 95% sequence identity to any of SEQ
ID NOs: 2 or SEQ ID NO: 112-120 disclosed herein.

| BBM-[GBS-Ag]n fusion | SBD-[GBS-Ag]n fusion | BBM-[GBS-Ag]n-SBD fusion |
|---|---|---|
| proteins: Rhavi fused to | proteins: Rhavi fused to | proteins: Rhavi fused to |
| 1 or 2 GBS antigens | 1 or 2 GBS antigens | 1 or 2 GBS antigens |
| Rhavi-Rib | SBD-Rib | Rhavi-Rib-SBD |
| Rhavi-Sip | SBD-Sip | Rhavi-Sip-SBD |
| Rhavi-AlpC | SBD-AlpC | Rhavi-AlpC-SBD |
| Rhavi-Alp3/1 | SBD-Alp3/1 | Rhavi-Alp3/1-SBD |
| Rhavi-Rib-Sip | SBD-Rib-Sip | Rhavi-Rib-Sip-SBD |
| Rhavi-Rib-AlpC | SBD-Rib-AlpC | Rhavi-Rib-AlpC-SBD |
| Rhavi-Rib-Alp3/1 | SBD-Rib-Alp3/1 | Rhavi-Rib-Alp3/1-SBD |
| Rhavi-Sip-AlpC | SBD-Sip-AlpC | Rhavi-Sip-AlpC-SBD |
| Rhavi-Sip-Alp3/1 | SBD-Sip-Alp3/1 | Rhavi-Sip-Alp3/1-SBD |
| Rhavi-AlpC-Alp3/1 | SBD-AlpC-Alp3/1 | Rhavi-AlpC-Alp3/1-SBD |

TABLE 2B
Exemplary fusion proteins for MAPS-GBS immunogenic complex, where the fusion
protein comprises at least three GBS polypeptide antigens. SBD refers to
any SBD protein as disclosed herein, including but not limited to SBD1, SBD2,
SBD3, SBD4, NanH, NanH2 or VcNanH, or having an amino acid sequence with
at least 80%, or at least 85%, or at least 90%, or at least 95% sequence
identity to any of SEQ ID NOs: 2 or SEQ ID NO: 112-120 disclosed herein.
The arrangement of the proteins is illustrative and it is envisioned that
any order of the proteins in the GBS fusion protein is encompassed.

| BBM-[GBS-Ag]n fusion | BBM-[GBS-Ag]n-SBD fusion | BBM-[GBS-Ag]n-SBD fusion |
|---|---|---|
| proteins: Rhavi fused | proteins: Rhavi fused | proteins: Rhavi fused |
| to 3 GBS antigens | to 3 GBS antigens | to 3 GBS antigens |
| Rhavi-Rib-Sip-AlpC | Rib-Sip-AlpC-SBD | Rhavi-Rib-Sip-AlpC-SBD |
| Rhavi-Rib-Sip-Alp1-3 | Rib-Sip-Alp3/1-SBD | Rhavi-Rib-Sip-Alp3/1-SBD |
| Rhavi-Rib-AlpC-Alp3/1 | Rib-AlpC-Alp3/1-SBD | Rhavi-Rib-AlpC-Alp3/1-SBD |
| Rhavi-Sip-AlpC-Alp3/1 | Sip-AlpC-Alp3/1-SBD | Rhavi-Sip-AlpC-Alp3/1-SBD |

In some embodiments, the present disclosure provides fusion proteins with at least 80%, 85%, 90%, 95%, 96%, 97%, 98%, 99%, 99.5%, or 100% sequence identity to a fusion protein listed in Table 2A or 2B, or Table 3.

### (a) SBD-[GBS-Ag] Fusion Proteins

In some embodiments, a GBS fusion protein comprises a sialic acid binding domain (SBD). In some embodiments, a GBS fusion protein of a MAPS-GBS immunogenic complex comprises a SBD, and one or more polypeptide antigens. In some embodiments, a GBS fusion protein comprises a SBD and two or more polypeptide antigens.

As used herein, a “sialic acid binding domain (SBD)” or “sialic acid binding domain (SBM)” are used interchangeably, and refers to a portion, fragment of variant of a sialic acid binding protein that binds or has affinity for a sialic acid moiety on the surface of a polysaccharide. It should be understood that any polypeptide or molecules which exhibit an affinity for sialic acid, bind to or otherwise couple to or associate with sialic acid moieties is encompassed in the term SBD. Thus the term “sialic acid binding domain” may encompass any fragment, which retains an ability to bind to or otherwise couple or associate with a sialic acid moiety.

In some embodiments, a GBS fusion protein useful in the MAPS-GBS immunogenic complex as disclosed herein comprises, in any order (i) at least one antigenic polypeptide from GBS and (ii) a SBD. A SBD of a GBS fusion protein as disclosed herein may comprise a single molecule capable of binding sialic acid (a monomeric or monovalent molecule, for example) or, alternatively, two or more sialic acid binding molecules (which may all be the same or different—a polymeric or multivalent molecule, for example).

In some embodiments, a GBS fusion protein useful in the MAPS-GBS immunogenic complex as disclosed herein comprises, in any order (i) at least one antigenic polypeptide from GBS and (ii) SBD having an amino acid sequence with at least 80%, or at least 85%, or at least 90%, or at least 95% sequence identity to SEQ ID NO: 2 (SBD1). In some embodiments, a GBS fusion protein useful in the MAPS-GBS immunogenic complex as disclosed herein comprises, in any order (i) at least one antigenic polypeptide from GBS and (ii) SBD having an amino acid sequence with at least 80%, or at least 85%, or at least 90%, or at least 95% sequence identity to SEQ ID NO: 112 (SBD2). In some embodiments, a GBS fusion protein useful in the MAPS-GBS immunogenic complex as disclosed herein comprises, in any order (i) at least one antigenic polypeptide from GBS and (ii) SBD selected from any of SBD1, SBD2, SBD3, SBD4, NanH, NanH2, VcNanH, or having an amino acid sequence with at least 80%, or at least 85%, or at least 90%, or at least 95% sequence identity to any of: SEQ ID NOs: 2 or 112-120.

The term “sialic acid” as used herein, embraces all forms of N- or O-substituted neuraminic acid and includes all synthetic, naturally occurring and/or modified forms thereof. Sialic acids may be found as components of cell surface molecules, glycoproteins and glycolipids. Sialic acids are present at the end (terminal regions) of sugar chains connected to cell membranes and/or proteins. The sialic acid family encompasses a number (approximately 50) of derivatives that may result from acetylation, glycolylation, lactonisation and methylation at C4, C5, C7, C8 and C9. All such derivatives are to be embraced by the term “sialic acid”. Sialic acids are found linked α(2,3) or α(2,6) to Gal and GalNAc or α(2,8) or α(2,9) to another sialic acid. Accordingly, while the term “sialic acid” is used throughout this specification, it encompasses all derivatives, analogues or variants (either naturally occurring or synthetically generated) thereof as well as monomers, dimers, trimers, oligomers, polymers or concatamers comprising the same.

### Sialic Acid Binding Domain (SBD):

A sialic acid binding domain (SBD) useful as a component of a GBS-fusion protein as disclosed herein exhibits an affinity for sialic acid-including all forms of sialic acid described above and, in particular sialic acid present on the surface of mammalian cells. For example, the molecules of this invention may exhibit an affinity for cell membrane receptors, which comprise sialic acid. Cell receptors of this type may be present on the surface of epithelial cells-including epithelial cells of the mucosal and respiratory tracts. A number of pathogens, including viral and/or bacterial pathogens may express molecules, which exhibit an affinity for sialic acid. Pathogens of this type have evolved to exploit cell surface sialic acid moieties as a means to bind to host cells. Once bound to a cell via, for example a host cell surface bound sialic acid moiety, a pathogen may colonise the cell surface and/or infect/enter the cell.

In particular embodiments, a sialic acid binding domain (SBD) that is a component of a GBS-fusion protein as disclosed herein exhibits affinity for sialic acid present on GBS.

In some embodiments, a SBD of a GBS fusion protein as disclosed herein may exhibit an affinity for α-2,6-linked sialic acid receptors predominantly present on cells of the human upper respiratory tract. Additionally or alternatively, the sialic acid binding receptors may exhibit an affinity for α-2,3-linked sialic acid receptors present on cells of the upper and lower respiratory tracts.

### Exemplary SBDs:

A SBD of a GBS fusion protein as disclosed herein can comprise one or more moieties that exhibit an affinity for sialic acid.

SBD1 (NanA): In some embodiments, an exemplary SBD for use in a GBS fusion protein is a fragment of Streptococcus pneumoniae NanA sialidase (NanA), where the amino acid sequence is SEQ ID NO: 118 (1035 amino acids) and has been deposited under accession number P62575. In some embodiments, a SBD of a GBS fusion protein as disclosed herein comprises amino acids 121-305 the full length NanA polypeptide of amino acids of SEQ ID NO: 118. In some embodiment, a SBD for use in a GBS fusion protein as disclosed herein comprises an amino acid sequence of SEQ ID NO: 2 (180aa), or an immunogenic variant or fragment thereof.

In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of a SBD polypeptide of SEQ ID NO: 2. In some embodiments, a SBD of a GBS fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 2. In some embodiments, a SBD polypeptide of the fusion protein is a SBD1 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 2.

SBD2 (NanL): In some embodiments, an exemplary SBD for use in a GBS fusion protein is a fragment of NanL sialidase (NanL). In some embodiments, a SBD is a fragment of amino acids 81-272 the full length NanL polypeptide. In some embodiment, a SBD for use in a GBS fusion protein as disclosed herein comprises an amino acid sequence of SEQ ID NO: 112 (192aa), or an immunogenic variant or fragment thereof.

In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185 or 191 consecutive amino acids of a SBD2 polypeptide of SEQ ID NO: 112. In some embodiments, a SBD of a GBS fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185 or 191 consecutive amino acids of the sequence shown in SEQ ID NO: 112. In some embodiments, a SBD polypeptide of the fusion protein is a SBD2 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185 or 191 consecutive amino acids of SBD2 amino acid sequence shown in SEQ ID NO: 112.

SBD3 (NanB): In some embodiments, an exemplary SBD for use in a GBS fusion protein is a fragment of NanB sialidase (NanB). In some embodiments, a SBD is a fragment of amino acids 40-230 the full length NanB polypeptide. In some embodiment, a SBD for use in a GBS fusion protein as disclosed herein comprises an amino acid sequence of SEQ ID NO: 113 (191aa), or an immunogenic variant or fragment thereof.

In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of a SBD3 polypeptide of SEQ ID NO: 113. In some embodiments, a SBD of a GBS fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of the sequence shown in SEQ ID NO: 113. In some embodiments, a SBD polypeptide of the fusion protein is a SBD3 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of SBD3 amino acid sequence shown in SEQ ID NO: 113.

SBD4 (NanC): In some embodiments, an exemplary SBD for use in a GBS fusion protein is a fragment of NanC sialidase (NanC). In some embodiments, a SBD is a fragment of amino acids 82-270 the full length NanC polypeptide. In some embodiment, a SBD for use in a GBS fusion protein as disclosed herein comprises an amino acid sequence of SEQ ID NO: 114 (189aa), or an immunogenic variant or fragment thereof.

In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of a SBD4 polypeptide of SEQ ID NO: 114. In some embodiments, a SBD of a GBS fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 187 or 189 consecutive amino acids of the sequence shown in SEQ ID NO: 114. In some embodiments, a SBD polypeptide of the fusion protein is a SBD4 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 187 or 189 consecutive amino acids of SBD4 amino acid sequence shown in SEQ ID NO: 114.

NanH: In some embodiments, an exemplary SBD for use in a GBS fusion protein is a fragment of Salmonella NanH sialidase (NanH). In some embodiments, a SBD is a fragment of the full length NanH polypeptide. In some embodiment, a SBD for use in a GBS fusion protein as disclosed herein comprises NanH having an amino acid sequence of SEQ ID NO: 115 (381aa), or an immunogenic variant or fragment thereof.

In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH polypeptide of SEQ ID NO: 115. In some embodiments, a SBD of a GBS fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH polypeptide of SEQ ID NO: 115. In some embodiments, a SBD polypeptide of the fusion protein is a NanH immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH polypeptide of of SEQ ID NO: 115.

NanH2: In some embodiments, an exemplary SBD for use in a GBS fusion protein is a fragment of Salmonella NanH2 sialidase (NanH2). In some embodiments, a SBD is a fragment of the full length NanH2 polypeptide. In some embodiment, a SBD for use in a GBS fusion protein as disclosed herein comprises NanH2 having an amino acid sequence of SEQ ID NO: 116 (404aa), or an immunogenic variant or fragment thereof.

In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH2 polypeptide of SEQ ID NO: 116. In some embodiments, a SBD of a GBS fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH2 polypeptide of SEQ ID NO: 116. In some embodiments, a SBD polypeptide of the fusion protein is a NanH2 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH2 polypeptide of SEQ ID NO: 116.

NanH3: In some embodiments, an exemplary SBD for use in a GBS fusion protein is a fragment of NanH3 sialidase (NanH3) from Trypanosoma cruzi. In some embodiments, a SBD is a fragment of amino acids 4-399 the full length NanH3 polypeptide. In some embodiment, a SBD for use in a GBS fusion protein as disclosed herein comprises an amino acid sequence of SEQ ID NO: 117 (396aa), or an immunogenic variant or fragment thereof.

In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 380, 390 or 396 consecutive amino acids of a NanH2 polypeptide of SEQ ID NO: 117. In some embodiments, a SBD of a GBS fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 380, 390 or 396 consecutive amino acids of a NanH2 polypeptide of SEQ ID NO: 117. In some embodiments, a SBD polypeptide of the fusion protein is a NanH3 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 380, 390 or 396 consecutive amino acids of a NanH2 polypeptide of SEQ ID NO: 117.

Similar or homologous sialic acid binding modules present in other organisms are to be encompassed within the scope of the term “SBD” herein. In some embodiments, additional exemplary SBD for use in the fusion proteins as disclosed herein comprise the sialic acid binding domain (SBD) of Vibrio cholerae NanH sialidase (VcNanH). Accordingly, in some embodiments, an exemplary SBD for use in a GBS fusion protein is a fragment of Vibrio cholerae NanH sialidase (VcNanH sialidase), where the amino acid sequence is deposited under accession umber A5F7A4 and is as SEQ ID NO: 113 (781 amino acids). In some embodiments, a SBD region of VcNanA is from amino acid residue 25 to 216 of SEQ ID NO: 119, and corresponds to amino acid sequence SEQ ID NO: 120.

In some embodiments, a SBD for use in a GBS fusion protein is a protein or binding moiety which binds to a modified sialic acid, where modifications of sialic acids include diverse forms differing in position 5 of an amino group of neuraminic acid derivatives or an hydroxyl group of 3-deoxy-D-glycero-D-galactononulosonic acid (Kdn), different acylations of the NH₂ at position 5 (glycolyl, acetyl), and various substituent of the different hydroxyl groups including phosphate, sulfate, methyl, acetyl, etc. >50 different derivatives of sialic acids are disclosed in Table 1 of Ghosh, S. (2020). Sialic acids and sialoglycoconjugates in the biology of life, health and disease. Academic Press, which is incorporated herein in its entirety. Two most commonly expressed members of sialic acid family are Neu5Ac and Neu5Gc followed by KDN (2-keto-3-deoxy-nononic acid) and Neu (neuraminic acid). Accordingly, a SBD for use in a GBS fusion protein is a protein or binding moiety which has affinity for, and binds to any of: Neu5Ac, Neu5Gc, KDN, Neu. In some embodiments, a SBD for use in a GBS fusion protein is a protein or binding moiety which has affinity for sialic acid that have modifications to core structures of sialic acid, including modifications such as O-acetylation, O-methylation, or introduction of O-lactyl groups, sulfate, or phosphate esters at positions 4, 7, 8, and/or 9.

Streptococcus agalactiae (S. agalactiae) is a Gram-positive bacteria causing serious infections in newborns, and can produce sialic acid-containing capsule by using sialyltransferase (CpsK) adding terminal α-2,3-linked Neu5Ac to galactose within the capsule's oligosaccharide repeat. Neu5Ac can be modified by 0-acetylation. Accordingly, a SBD for use in a GBS fusion protein is a protein or binding moiety which has affinity for, and binds to a sialic acid that has an added terminal α-2,3-linked Neu5Ac to galactose within the capsule's oligosaccharide repeat, and/or where Neu5Ac is modified by O-acetylation.

In some embodiment, a SBD for use in a GBS fusion protein is a protein or binding moiety which has affinity for, and binds to a sialic acid present on the GBS capsular polysaccharide selected from any GBS serotype of: Ia, 1b, II, III, IV, V, VI, VII, VIII, IX. Such sialic acid domains on the GBS capsular polysaccharides are disclosed below.

### (a) GBS Capsular Polysaccharide of Serotype Type I

In some embodiments, a GBS fusion protein as disclosed herein comprises a SBD that is a binding moiety that has affinity for, or binds to a sialic acid present on the capsular polysaccharide of serotype Ia GBS. The structure of serotype 1a GBS capsular polysaccharide can be depicted as follows:

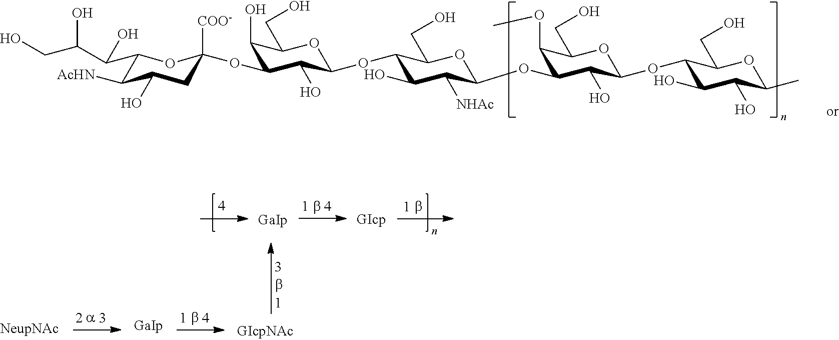

Serotype 1a capsular polysaccharides are less than about 5% O-acetylated. Some exemplary strains of serotype 1a capsular polysaccharides of the invention include 090, A909 (ATCC Accession No. BAA-1 138), 515 (ATCC Accession No. BAA-1 177), B523, CJB524, and MB 4052 (ATCC Accession No. 31574).

### (b) GBS Capsular Polysaccharide of Serotype Type Ib

In some embodiments, a GBS fusion protein as disclosed herein comprises a SBD that is a binding moiety that has affinity for, or binds to a sialic acid present on the capsular polysaccharide of serotype 1b GBS. The structure of serotype 1b GBS capsular polysaccharide can be depicted as follows:

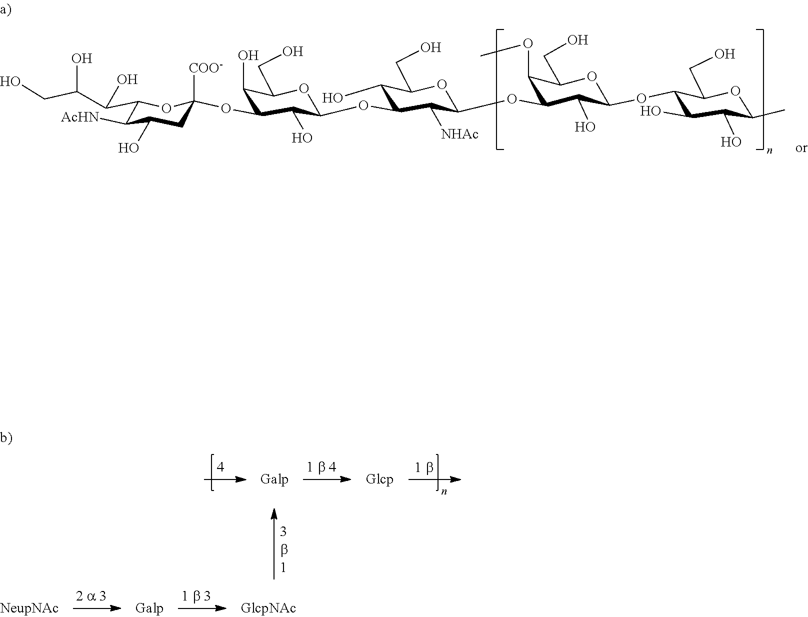

Serotype 1b capsular polysaccharides are between about 0% and about 40% O-acetylated. In one embodiment of the invention, the polysaccharide is de-O-acetylated (i.e., less than about 5% O-acetylated). Some exemplary strains of serotype 1b capsular polysaccharides of the invention include H36B (ATCC Accession No. 12401B), S40, S42, MB 4053 (ATCC Accession No. 31575), M709,133, 7357, and PFEGBST0267.

### (c) GBS Capsular Polysaccharide of Serotype Type II

In some embodiments, a GBS fusion protein as disclosed herein comprises a SBD that is a binding moiety that has affinity for, or binds to a sialic acid present on the capsular polysaccharide of serotype II GBS. The structure of serotype II GBS capsular polysaccharide can be depicted as follows:

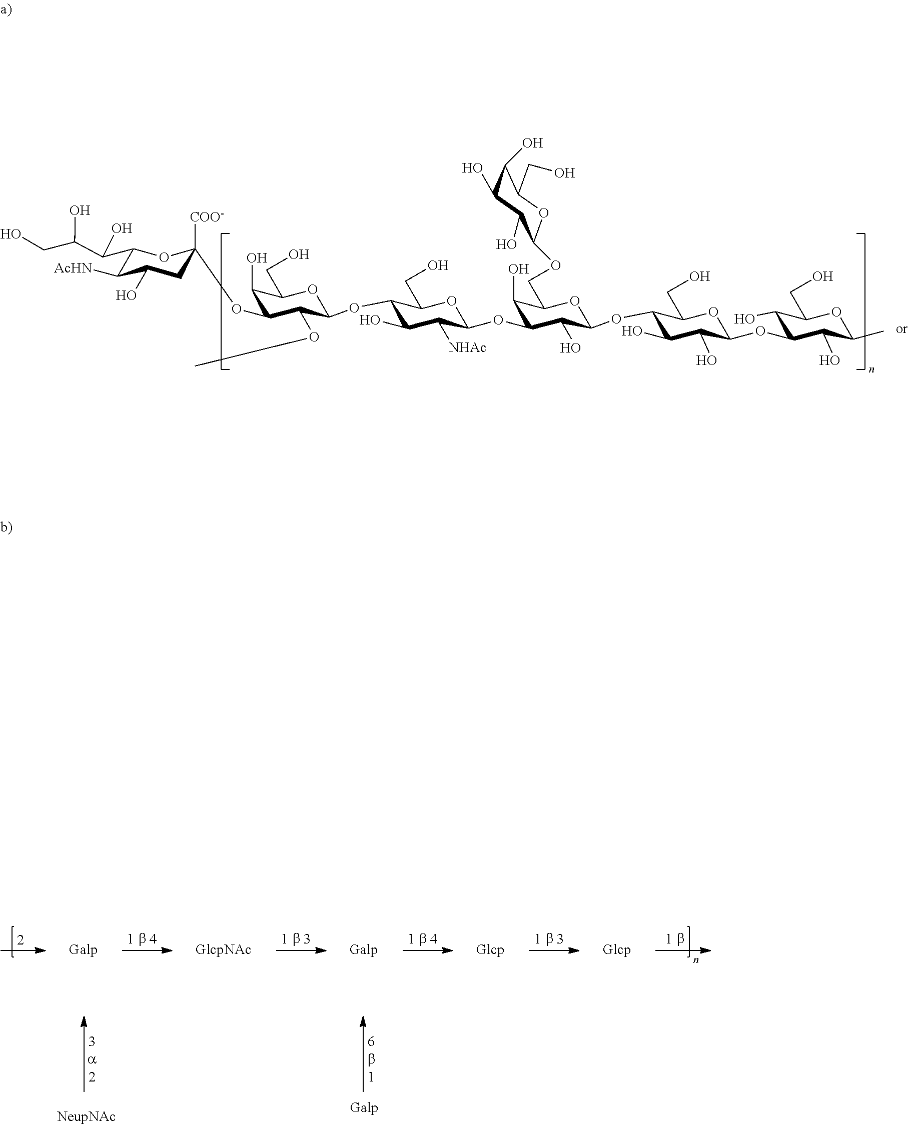

Serotype II capsular polysaccharides are less than about 5% O-acetylated. Some exemplary strains of serotype II capsular polysaccharides of the invention include MB 4055 (ATCC Accession No. 31576), 18RS21 (ATCC Accession No. BAA-1 175), S16, S20, V8 (ATCC Accession No. 12973), DK21, DK23, UAB, 5401, and PFEGBST0708.

### (d) GBS Capsular Polysaccharide of Serotype Type III

In some embodiments, a GBS fusion protein as disclosed herein comprises a SBD that is a binding moiety that has affinity for, or binds to a sialic acid present on the capsular polysaccharide of serotype III GBS. The structure of serotype III GBS capsular polysaccharide can be depicted as follows:

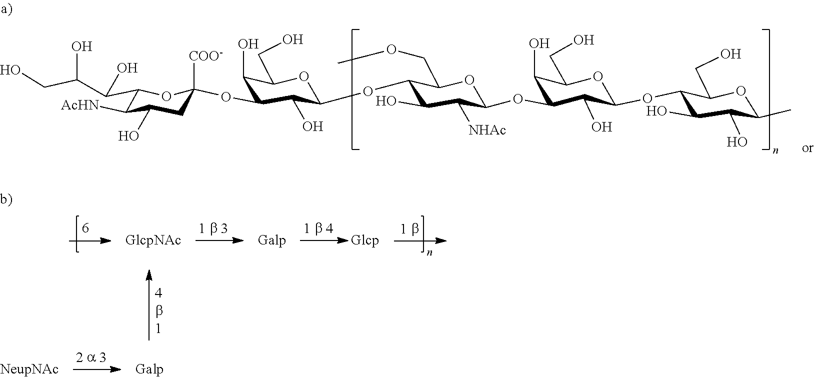

Serotype III capsular polysaccharides are between about 0% and about 40% O-acetylated. In one embodiment of the invention, the polysaccharide is de-O-acetylated (i.e., less than about 5% 0-acetylated). Some exemplary strains of serotype III capsular polysaccharides of the invention include MB 4082 (ATCC Accession No. 31577), M132, 1 10, M781 (ATCC Accession No. BAA-22), D136C(3) (ATCC Accession No. 12403), M782, S23, 120, MB 4316 (M-732; ATCC Accession No. 31475), M132, K79, COH1 (ATCC Accession No. BAA-1 176), and PFEGBST0563

### (e) GBS Capsular Polysaccharide of Serotype Type IV

In some embodiments, a GBS fusion protein as disclosed herein comprises a SBD that is a binding moiety that has affinity for, or binds to a sialic acid present on the capsular polysaccharide of serotype IV GBS. The structure of serotype IV GBS capsular polysaccharide can be depicted as follows:

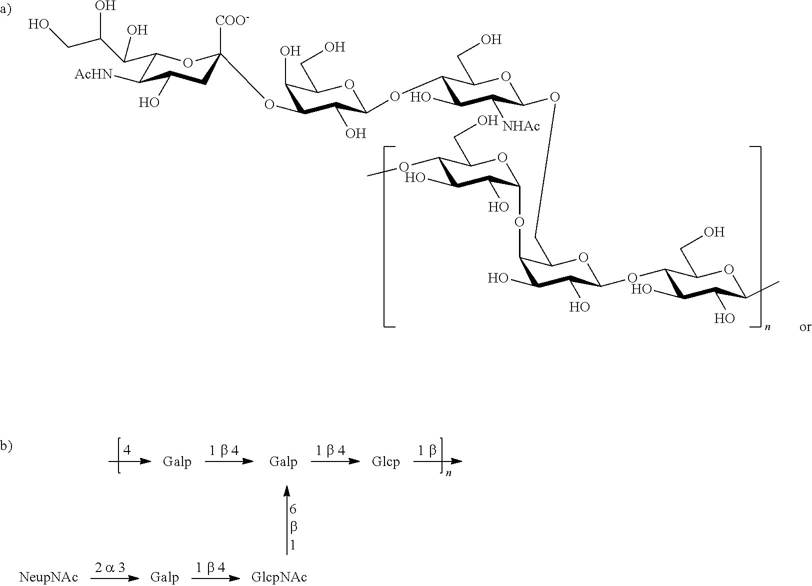

Serotype IV capsular polysaccharides are between about 0% and about 40% O-acetylated. In one embodiment of the invention, the polysaccharide is de-O-acetylated (i.e., less than about 5% O-acetylated). Some exemplary strains of serotype IV capsular polysaccharides of the invention include 3139 (ATCC Accession No. 49446), CZ-NI-016, and PFEGBST0961

### (f) GBS Capsular Polysaccharide of Serotype Type V

In some embodiments, a GBS fusion protein as disclosed herein comprises a SBD that is a binding moiety that has affinity for, or binds to a sialic acid present on the capsular polysaccharide of serotype V GBS. The structure of serotype V GBS capsular polysaccharide can be depicted as follows:

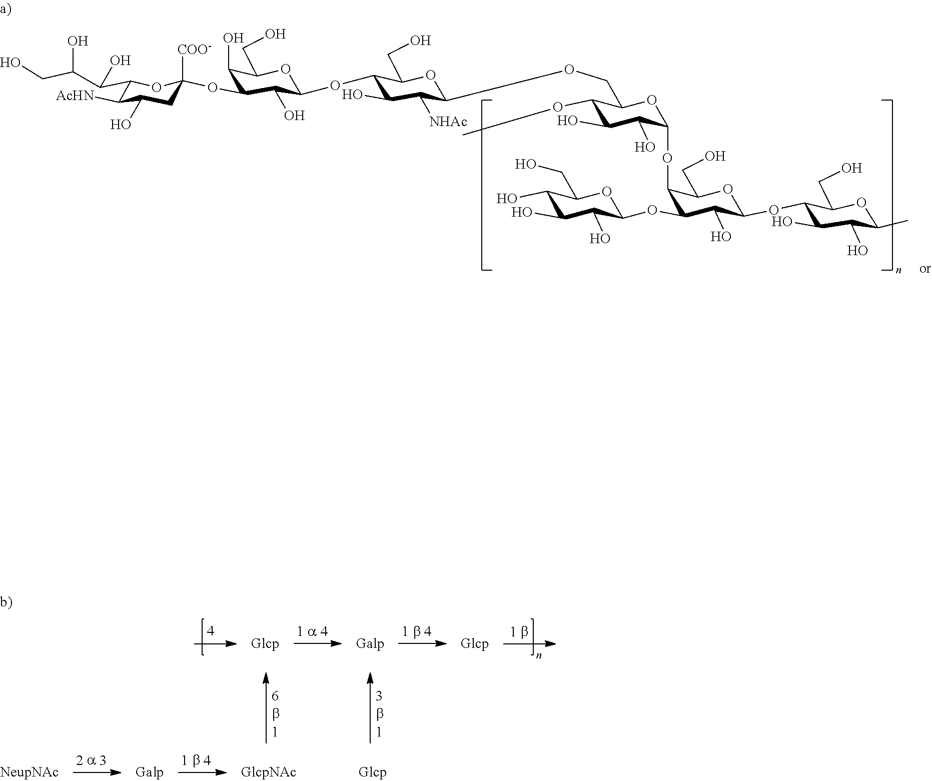

Serotype V capsular polysaccharides are between about 0% and about 40% O-acetylated. In one embodiment of the invention, the polysaccharide is de-O-acetylated (i.e., less than about 5% O-acetylated). Some exemplary strains of serotype V capsular polysaccharides of the invention include 1 169-NT1, CJB1 1 1 (ATCC Accession No. BAA-23), CJB1 12, 2603 V/R (ATCC Accession No. BAA-61 1), NCTC 10/81, CJ1 1, and PFEGBST0837.

### (g) GBS Capsular Polysaccharide of Serotype Type VI

In some embodiments, a GBS fusion protein as disclosed herein comprises a SBD that is a binding moiety that has affinity for, or binds to a sialic acid present on the capsular polysaccharide of serotype VI GBS. The structure of serotype VI GBS capsular polysaccharide can be depicted as follows:

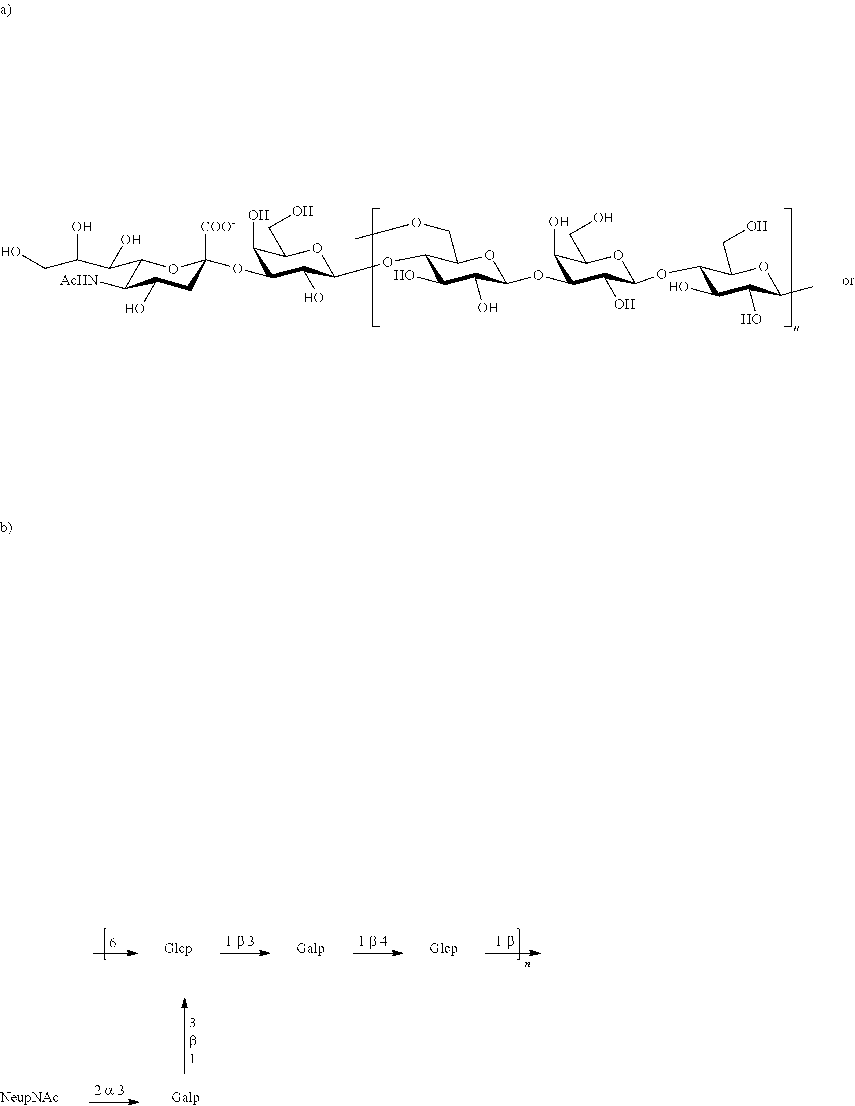

Serotype VI capsular polysaccharides are between about 0% and about 40% O-acetylated. In one embodiment of the invention, the polysaccharide is de-O-acetylated (i.e., less than about 5% O-acetylated). Some exemplary strains of serotype Nil capsular polysaccharides of the invention include 1 18754, 1 14852, 1 14862, 1 14866, 1 18775, B 4589, B 4645, SS1214, and CZ-PW-1 19.

### (h) GBS Capsular Polysaccharide of Serotype Type VII

In some embodiments, a GBS fusion protein as disclosed herein comprises a SBD that is a binding moiety that has affinity for, or binds to a sialic acid present on the capsular polysaccharide of serotype VII GBS. The repeating unit of structure of serotype VII GBS capsular polysaccharide can be depicted as follows:

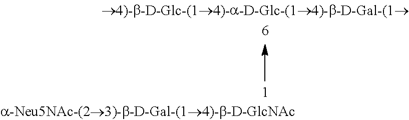

Serotype VII capsular polysaccharides are described by Kogan, G., et al., Carbohydrate Research, 277(1): 1-9 (1995), the disclosures of which are hereby incorporated by reference in their entirety. Serotype VII capsular polysaccharides are less than about 5% O-acetylated. Some exemplary strains of serotype VII capsular polysaccharides of the invention include 7271 and CZ-PW-045.

### (i) GBS Capsular Polysaccharide of Serotype Type VIII

In some embodiments, a GBS fusion protein as disclosed herein comprises a SBD that is a binding moiety that has affinity for, or binds to a sialic acid present on the capsular polysaccharide of serotype VIII GBS. The repeating unit of structure of serotype VIII GBS capsular polysaccharide can be depicted as follows:

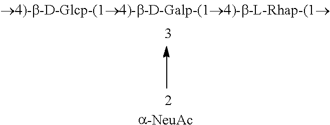

GBS Serotype VIII capsular polysaccharides are described by Kogan, G., et al., The Journal of Biological Chemistry, 271 (15):8786-8790 (1996), the disclosures of which are hereby incorporated by reference in their entirety. Serotype VIII capsular polysaccharides are between about 0% and about 40% O-acetylated. In one embodiment of the invention, the polysaccharide is de-O-acetylated (i.e., less than about 5% O-acetylated). Some exemplary strains of serotype VIII capsular polysaccharides of the invention include JM9130013 and JM9130672.

### (j) GBS Capsular Polysaccharide of Serotype Type IX

In some embodiments, a GBS fusion protein as disclosed herein comprises a SBD that is a binding moiety that has affinity for, or binds to a sialic acid present on the capsular polysaccharide of serotype IX GBS. The repeating unit of structure of serotype IX GBS capsular polysaccharide can be depicted as follows:

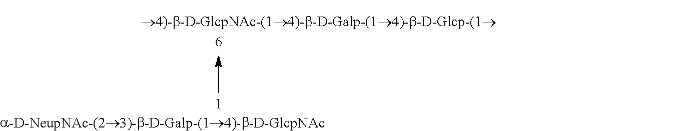

GBS Serotype IX capsular polysaccharides are described by Berti, F., et al., The Journal of Biological Chemistry, 289(34):23437-2348 (2014), the disclosures of which are hereby incorporated by reference in their entirety. Serotype IX capsular polysaccharides are between about 0% and about 40% O-acetylated. In one embodiment of the invention, the polysaccharide is de-O-acetylated (i.e., less than about 5% O-acetylated). Some exemplary strains of serotype IX capsular polysaccharides of the invention include IT-NI-016, IT-PW-62, and IT-PW-64.

### (c) Rhavi-[GBS-Ag] Fusion Proteins

In some embodiments, a GBS fusion protein comprises a biotin-binding moiety (BBM). In some embodiments, a GBS fusion protein of the immunogenic complex comprises a biotin-binding moiety, and one or more polypeptide antigens. In some embodiments, a GBS fusion protein comprises a biotin-binding moiety and two or more polypeptide antigens. As used herein, a “biotin-binding moiety” refers to a biotin-binding protein, a biotin-binding fragment thereof, or a biotin-binding molecule thereof.

In some embodiments, the biotin-binding moiety of a GBS-fusion protein comprises rhizavidin or a biotin-binding molecule or biotin-binding fragment thereof, as further described in WO 2012/155053, the contents of which are herein incorporated by reference in their entirety. In some embodiments, a biotin-binding moiety is or comprises a polypeptide having at least 80%, 85%, 90%, 95%, 96%, 97%, 98%, or 99% identity to rhizavidin, or a biotin-binding molecule or biotin-binding fragment thereof. In some embodiments, the biotin-binding moiety comprises a polypeptide of SEQ ID NO: 1 or a biotin-binding molecule or biotin-binding fragment thereof. In some embodiments, the biotin-binding moiety is or comprises a polypeptide having at least 80%, 85%, 90%, 95%, 96%, 97%, 98%, or 99% identity to the sequence of SEQ ID NO: 1, or biotin-binding molecule or biotin-binding fragment thereof.

In some embodiments, a GBS fusion protein useful in the MAPS-GBS immunogenic complex as disclosed herein comprises, in any order (i) at least one antigenic polypeptide from GBS and (ii) BBM, e.g., Rhizavidin having an amino acid sequence with at least 80%, or at least 85%, or at least 90%, or at least 95% sequence identity to SEQ ID NO: 1 or SEQ ID NO: 8.

In some embodiments, a fusion protein useful in the MAPS-GBS immunogenic complex as disclosed herein comprises, in any order, (i) at least one antigenic polypeptide from GBS, (ii) SBD having an amino acid sequence with at least 80%, or at least 85%, or at least 90%, or at least 95% sequence identity to SEQ ID NO: 2 (SBD1), and (iii) a BBM which is Rhizavidin, having an amino acid sequence with at least 80%, or at least 85%, or at least 90%, or at least 95% sequence identity to SEQ ID NO: 1 or SEQ ID NO: 8. In some embodiments, a fusion protein useful in the MAPS-GBS immunogenic complex as disclosed herein comprises, in any order, (i) at least one antigenic polypeptide from GBS, (ii) SBD having an amino acid sequence with at least 80%, or at least 85%, or at least 90%, or at least 95% sequence identity to SEQ ID NO: 112 (SBD2), and (iii) a BBM which is Rhizavidin, having an amino acid sequence with at least 80%, or at least 85%, or at least 90%, or at least 95% sequence identity to SEQ ID NO: 1 or SEQ ID NO: 8. In some embodiments, a fusion protein useful in the MAPS-GBS immunogenic complex as disclosed herein comprises, in any order, (i) at least one antigenic polypeptide from GBS, (ii) a SBD selected from any of SBD1, SBD2, SBD3, SBD4, NanH, NanH2, VcNanH, or having an amino acid sequence with at least 80%, or at least 85%, or at least 90%, or at least 95% sequence identity to any of: SEQ ID NOs: 2 or 112-120 and (iii) a BBM which is Rhizavidin, having an amino acid sequence with at least 80%, or at least 85%, or at least 90%, or at least 95% sequence identity to SEQ ID NO: 1 or SEQ ID NO: 8.

### Rhizavidin

Rhizavidin is a naturally occurring dimeric protein in the avidin protein family, was first discovered in Rhizobium etli, a symbiotic bacterium of the common bean. Rhizavidin has only a 22% amino acid identity with chicken avidin, a protein commonly found in eggs, but with high conservation of amino acid residues involved in biotin binding [Helppolainen et al, 2007]. In some embodiments, the nucleotide sequence of rhizavidin is set forth in SEQ ID NO: 16. In some embodiments, the amino acid sequence of rhizavidin is set forth in SEQ ID NO: 1. SEQ ID NO: 1 has N-terminal 1-44 amino acids removed of the full-length protein, which are predicted to be a signal sequence(s) of rhizavidin. In some embodiments, a GBS fusion protein comprises rhizavidin. In some embodiments, a GBS-fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of a rhizavidin polypeptide of SEQ ID NO: 1 or SEQ ID NO: 8.

In some embodiments, a rhizavidin polypeptide of a GBS fusion protein comprises at least 7, 8,9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 1. In some embodiments, a rhizavidin polypeptide of the fusion protein comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 1. In some embodiments, a rhizavidin polypeptide of the fusion protein comprises an amino acid sequence of SEQ ID NO: 8, which comprises 5 different A>T amino acid modification.

In some embodiments the Rhizavidin of the GSB fusion protein is lipidated (i.e., a lipidated-Rhaviavin in a GBS Ag and/or SBD fusion protein). In some embodiments, a rhizavidin fusion protein comprising at least one or more GBS antigen can comprise a lipidation sequence at the N-terminus, e.g., MKKVAAFVALSLLMAGC (SEQ ID NO: 175) or an amino acid 85% identity thereto. In some embodiments, the MAPS-GBS composition can also comprise a rhizavidin protein comprising SEQ ID NO: 1 or a protein with 85% sequence identity thereto, that comprises a lipidation sequence at the N-terminus, e.g., MKKVAAFVALSLLMAGC (SEQ ID NO: 175) or an amino acid 85% identity thereto, but the rhizavidin protein is not fused to a GBS antigen or other antigen (e.g., Rhavi is not part of a Rhavi-antigen fusion protein). Lipidated rhizavidin proteins and lapidated rhizavidin fusion proteins are disclosed in US application US2016/0090404, entitled “Modified biotin-binding protein, fusion proteins thereof and applications”, which is incorporated herein in its entirety by reference. As used herein, the term “lipidated biotin-binding protein” refers to a biotin-binding protein that is covalently linked with a lipid. The lipidated biotin-binding proteins are ligands or agonists of Toll like receptor 2. Accordingly, also provided herein are methods for inducing an immune response in subject. The method comprising administering to the subject a composition comprising a lipidated biotin-binding protein.

In another aspect provided herein is a lipidated biotin-binding protein, e.g., a lipidated rhizavidin fusion protein comprising a GBS antigen for use in the MAPS-GBS immunogenic composition as disclosed herein. As used herein, the term “lipidated biotin-binding protein” refers to a biotin-binding protein that is covalently conjugated with a lipid. The lipid moieties could be a diacyl or triacyl lipid.

In some embodiments, a rhizavidin fusion protein comprising a GBS antigen for use in the MAPS-GBS immunogenic composition as disclosed herein comprises a lipidation sequence. As used herein, the term “lipidation sequence” refers to an amino acid sequence that facilitates lipidation in bacteria, e.g., E. coli, of a polypeptide carrying the lipidating sequence. The lipidation sequence can be present at the N-terminus or the C-terminus of the protein. The lipidation sequence can be linked to the recombinant biotin-binding protein to form a fusion protein, which is in lipidated form when expressed in E. coli by conventional recombinant technology. In some embodiments, a lipidation sequence is located at the N-terminus of the biotin-binding protein.

Any lipidation sequence known to one of ordinary skill in the art can be used. In some embodiments, the lipidating sequence is MKKVAAFVALSLLMAGC (SEQ ID NO: 175) or a derivative or functional portion thereof. Other exemplary lipidating sequences include, but are not limited to, MNSKKLCCICVLFSLLAGCAS (SEQ ID NO: 176), MRYSKLTMLIPCALLLSAC (SEQ ID NO: 177), MFVTSKKMTAAVLAITLAMSLSAC (SEQ ID NO: 178), MIKRVLVVSMVGLSLVGC (SEQ ID NO: 179), and derivatives or functional portions thereof.

In some embodiments, the lipidation sequence can be fused to a rhizavidin fusion protein comprising a GBS antigen via a peptide linker, wherein the peptide linker attaches the lipidating sequence to the biotin-binding protein. In some embodiment, the peptide linker comprises the amino acid sequence sequence VSDP (SEQ ID NO: 180) or AQDP (SEQ ID NO: 181).

In some embodiments, a rhizavidin fusion protein comprising a GBS antigen for use in the MAPS-GBS immunogenic composition as disclosed herein that is a lipoprotein as described herein have enhanced immunogenicity. Without wishing to be bound by a theory, lipid moieties at the N-terminals of the lipoproteins or lipopeptides contribute to the adjuvant activity. Accordingly, additional embodiments provide immunogenic or vaccine compositions for inducing an immunological response, comprising the isolated biotin-binding lipoprotein, or a suitable vector for in vivo expression thereof, or both, and a suitable carrier, as well as to methods for eliciting an immunological or protective response comprising administering to a host the isolated recombinant biotin-binding lipoprotein, the vector expressing the recombinant biotin-binding lipoprotein, or a composition containing the recombinant lipoprotein or vector, in an amount sufficient to elicit the response.

A MAPS-GBS immunogenic composition comprising a rhizavidin fusion protein comprising a GBS antigen that is a lipoprotein elicits an immunological response-local or systemic. The response can, but need not, be protective.

### Alternative Biotin-Binding Moieties to Replace Rhizavidin

In some embodiments, a GBS fusion protein described herein comprises a BBM and is a component of non-covalent Multiple Antigen Presenting System (MAPS) GBS immunogenic complexes. In some embodiments, MAPS-GBS complexes disclosed herein utilize the high affinity (dissociation constant [KD]≈10-15M) non-covalent binding between biotin and rhizavidin, a biotin-binding protein that has no significant predicted homology with human proteins. In some embodiments, it is envisioned that Rhizavidin polypeptide of a GBS fusion protein comprising a BBM can be readily replaced with any other biotin-binding protein.

In some embodiments, the BBM is selected from any of rhizavidin, avidin, streptavidin, bradavidin, tamavidin, lentiavidin, zebavidin, NeutrAvidin, CaptAvidin™, or a biotin-binding molecule or biotin-binding fragment thereof, or a combination thereof. In some embodiments, the biotin-binding moiety is rhizavidin, or a biotin-binding molecule or biotin-binding fragment thereof.

### (c) Bifunctional SBD-[GBS-Ag]-Rhavi Fusion Proteins

One aspect of the technology disclosed herein relates to a bi-functional fusion protein comprising, in any order, (i) a sialic acid-binding domain (SBD), (ii) a biotin-binding moiety (BBM), and (iii) at least 1, or at least 2, or at least 3, at least 4, at least 5, or more than 5 polypeptide antigens, e.g., polypeptide antigens from GBS, and is broadly referred to herein as SBD-[GBS-Ag]n-BBM fusion protein, where n is the number of antigenic polypeptide located between the SBD and BBM. It is envisioned that the antigen in the SBD-[GBS-Ag]n-BBM fusion protein can be located at the N-terminal, or the C-terminal of the fusion protein, or between the SBD and the BBM. For example, exemplary fusion proteins are disclosed in Table 3 herein.

In some embodiments, the bi-functional fusion protein comprises at least one antigenic GBS polypeptide located between the sialic acid-binding domain (SBD) and a biotin-binding moiety (BBM), wherein the one antigenic polypeptide is from Group B Streptococcus (GBS) or Streptococcus agalactiae. In some embodiments, the bi-functional fusion protein comprises at least two antigenic polypeptides located between the sialic acid-binding domain (SBD) and a biotin-binding moiety (BBM), wherein both the antigenic polypeptides is from Group B Streptococcus (GBS) or Streptococcus agalactiae.

In some embodiments, a GBS fusion protein useful in the MAPS-GBS immunogenic complex as disclosed herein can comprise at least one antigenic polypeptide from GBS selected from at least one of: Rib, Sip, AlpC, Alp1 or Alp3, as disclosed herein. Exemplary fusion proteins are disclosed in Tables 2A, 2B and 3. In some embodiments, a fusion protein useful in the MAPS-GBS immunogenic complex is selected from any of: SBD-[Rib]-BBM fusion protein, SBD-[Sip]-BBM fusion protein, SBD-[Rib-Sip]-BBM fusion protein, BBM-[Rib]-SBD fusion protein, BBM-[Sip]-SBD fusion protein, BBM-[Rib-Sip]-SBD fusion protein, or variations thereof. In some embodiments, Rib and Sip, either individually, or together can be readily replaced with alternative GBS antigens known in the art, such as AlpC, Alp1, Alp3 or a Alp3/1 as disclosed herein.

### III. GBS Immunogenic Polypeptides and Variants Thereof

### (a) Rib Polypeptide or Immunogenic Fragments Thereof

Rib is a conserved GBS protein. Rib is a surface proteins belonging to the family of alpha-like proteins (Alps), are presented in S. agalactiae and is involved in binding to host cells and invasion via interacting with glycosaminoglycan on the surface of epithelial cells. In some embodiments, a Rib polypeptide is or comprises a full-length Rib polypeptide. For example, in some embodiments, a full-length Rib polypeptide has 175 amino acids and is represented by the amino acid sequence as set forth in SEQ ID NO: 4. In some embodiments, a fusion protein comprises a Rib polypeptide of S. agalactiae. In some embodiments, a fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 160, 170 or 175 consecutive amino acids of a Rib polypeptide.

In some embodiments, a Rib polypeptide of the fusion protein comprises an amino acid sequence that is at least 60% or more (including, e.g., at least 65%, 70%, 75%, 80%, 85%, 90%, 95%, 96%, 97%, 98%, 99%, or 100%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 160, 170 or 175 consecutive amino acids of the sequence shown in SEQ ID NO: 4. In some embodiments, the Rib polypeptide does not include a signal sequence. In some embodiments, the Rib polypeptide does include a signal sequence at the N-terminus of the amino acid sequence of SEQ ID NO: 4.

In some embodiments, a Rib polypeptide contains one or more amino acid alterations (e.g., deletion, substitution, and/or insertion) from a naturally-occurring wild-type Rib polypeptide sequence. For example, an Rib polypeptide may contain an amino acid sequence that is at least 60% or more (e.g., at least 65%, 70%, 75%, 80%, 85%, 90%, 95%, or 98%) identical to SEQ ID NO: 4 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or more consecutive amino acids of the sequence shown in SEQ ID NO: 4). Alternatively, an Rib polypeptide may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or more consecutive amino acids) of a sequence that is at least 60% or more (e.g., at least 65%, 70%, 75%, 80%, 85%, 90%, 95%, or 98%) identical to SEQ ID NO: 4

In some embodiments, a GBS fusion protein useful in the MAPS-GBS immunogenic complex as disclosed herein comprises an amino acid sequence having at least 80%, or at least 85%, or at least 90%, or at least 95% sequence identity to SEQ ID NO: 4 (Rib). In some embodiments, a GBS fusion protein comprises a Rib polypeptide comprising an amino acid sequence having at least 80%, 85%, 90%, 95%, 96%, 97%, 98%, 99%, or 100% identity to the sequence of SEQ ID NO: 4 (Rib).

In some embodiments, a GBS fusion protein is, e.g., a SBD-Rib fusion protein, and comprises, in any order, a sialic acid binding domain (SBD) as described herein, a polypeptide comprising an amino acid sequence having at least 80%, 85%, 90%, 95%, 96%, 97%, 98%, 99%, or 100% identity to the sequence of SEQ ID NO: 4 (Rib) or an antigenic fragment thereof. In some embodiments, the fusion protein comprises (i) a SBD having an amino acid sequence with at least 80%, or at least 85%, or at least 90%, or at least 95% sequence identity to a SBD selected from any of SBD1, SBD2, SBD3, SBD4, NanH, NanH2, VcNanH, or having an amino acid sequence with at least 80%, or at least 85%, or at least 90%, or at least 95% sequence identity to any of: SEQ ID NOs: 2 or 112-120, and (ii) a polypeptide comprising an amino acid sequence having at least 80%, 85%, 90%, 95%, 96%, 97%, 98%, 99%, or 100% identity to the sequence of SEQ ID NO: 4 (Rib) or an antigenic fragment thereof. In some embodiments, a SBD-Rib fusion protein can further comprise at least 1, or at least 2 or more additional GBS polypeptides, e.g., selected from any of Sip, AlpC, Alp1, Alp3 or Alp3/1 as disclosed herein.

In some embodiments, a GBS fusion protein is Rhavi-Rib fusion protein, comprising, in any order, biotin-binding moiety (BBM) as described herein, e.g., Rhizavidin and a polypeptide comprising an amino acid sequence having at least 80%, 85%, 90%, 95%, 96%, 97%, 98%, 99%, or 100% identity to the sequence of SEQ ID NO: 4 (Rib) or an antigenic fragment thereof. In some embodiments, the fusion protein comprises a Rhizavidin having an amino acid sequence with at least 80%, or at least 85%, or at least 90%, or at least 95% sequence identity to SEQ ID NO: 1 or SEQ ID NO: 8, and a polypeptide comprising an amino acid sequence having at least 80%, 85%, 90%, 95%, 96%, 97%, 98%, 99%, or 100% identity to the sequence of SEQ ID NO: 4 (Rib) or an antigenic fragment thereof. In some embodiments, a Rhavi-Rib fusion protein can further comprise at least 1, or at least 2 or more additional GBS polypeptides, e.g., selected from any of Sip, AlpC, Alp1, Alp3 or Alp3/1 as disclosed herein.

In some embodiments, a GBS fusion protein is, e.g., a SBD-Rib-Rhavi fusion protein, and comprises, in any order, (i) a SBD, e.g., but not limited to a SBD selected from any of SBD1, SBD2, SBD3, SBD4, NanH, NanH2, VcNanH, or having an amino acid sequence with at least 80%, or at least 85%, or at least 90%, or at least 95% sequence identity to any of: SEQ ID NOs: 2, 112-120, a (ii) biotin-binding moiety as described herein, e.g., but not limited to, Rhizavidin having an amino acid sequence with at least 80%, or at least 85%, or at least 90%, or at least 95% sequence identity to SEQ ID NO: 1 or SEQ ID NO: 8, and (iii) a polypeptide comprising an amino acid sequence having at least 80%, 85%, 90%, 95%, 96%, 97%, 98%, 99%, or 100% identity to the sequence of SEQ ID NO: 4 (Rib) or an antigenic fragment thereof. In some embodiments, a SBD-Rib-Rhavi fusion protein can further comprise at least 1, or at least 2 or more additional GBS polypeptides, e.g., selected from any of Sip, AlpC, Alp1, Alp3 or Alp3/1 as disclosed herein, which can be in any order.

### (b) Sip

In some embodiments, a fusion protein useful in the MAPS-GBS immunogenic complex as disclosed herein comprises an amino acid sequence having at least 80%, or at least 85%, or at least 90%, or at least 95% sequence identity to SEQ ID NO: 5 (Sip). In some embodiments, a GBS fusion protein comprises a Sip polypeptide comprising an amino acid sequence having at least 80%, 85%, 90%, 95%, 96%, 97%, 98%, 99%, or 100% identity to the sequence of SEQ ID NO: 5 (Sip).

In some embodiments, a GBS fusion protein is, e.g., a SBD-Sip fusion protein, and comprises, in any order, a sialic acid binding domain (SBD) as described herein, a polypeptide comprising an amino acid sequence having at least 80%, 85%, 90%, 95%, 96%, 97%, 98%, 99%, or 100% identity to the sequence of SEQ ID NO: 5 (Sip) or an antigenic fragment thereof. In some embodiments, the fusion protein comprises (i) a SBD selected from any of SBD1, SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 2 or 112-120, and (ii) a polypeptide comprising an amino acid to the sequence of SEQ ID NO: 5 (Sip) or an antigenic fragment thereof. In some embodiments, a SBD-Sip fusion protein can further comprise at least 1, or at least 2 or more additional GBS polypeptides, e.g., selected from any of Sip, AlpC, Alp1, Alp3 or Alp3/1 as disclosed herein.

In some embodiments, a GBS fusion protein is Rhavi-Sip fusion protein, comprising, in any order, biotin-binding moiety (BBM) as described herein, e.g., Rhizavidin and a polypeptide comprising an amino acid sequence having at least 80%, 85%, 90%, 95%, 96%, 97%, 98%, 99%, or 100% identity to the sequence of SEQ ID NO: 5 (Sip) or an antigenic fragment thereof. In some embodiments, the fusion protein comprises a Rhizavidin having an amino acid sequence with at least 80%, or at least 85%, or at least 90%, or at least 95% sequence identity to SEQ ID NO: 1 or SEQ ID NO: 8, and a polypeptide comprising an amino acid sequence having at least 80%, 85%, 90%, 95%, 96%, 97%, 98%, 99%, or 100% identity to the sequence of SEQ ID NO: 5 (Sip) or an antigenic fragment thereof. In some embodiments, a Rhavi-Sip fusion protein can further comprise at least 1, or at least 2 or more additional GBS polypeptides, e.g., selected from any of Rib, AlpC, Alp1, Alp3 or Alp3/1 as disclosed herein.

In some embodiments, a GBS fusion protein is, e.g., a SBD-Sip-Rhavi fusion protein, and comprises, in any order, (i) a SBD selected from any of SBD1, SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 2 or 112-120, and (ii) a biotin-binding moiety as described herein, e.g., but not limited to, Rhizavidin having an amino acid sequence with at least 80%, or at least 85%, or at least 90%, or at least 95% sequence identity to SEQ ID NO: 1 or SEQ ID NO: 8, and (iii) a polypeptide comprising an amino acid sequence having at least 80%, 85%, 90%, 95%, 96%, 97%, 98%, 99%, or 100% identity to the sequence of SEQ ID NO: 5 (Sip) or an antigenic fragment thereof. In some embodiments, a SBD-Sip-Rhavi fusion protein can further comprise at least 1, or at least 2 or more additional GBS polypeptides, e.g., selected from any of Rib, AlpC, Alp1, Alp3 or Alp3/1 as disclosed herein, which can be in any order.

### (c) AlpC

In some embodiments, a fusion protein useful in the MAPS-GBS immunogenic complex as disclosed herein comprises an amino acid sequence having at least 80%, or at least 85%, or at least 90%, or at least 95% sequence identity to SEQ ID NO: 11 (AlpC). In some embodiments, a GBS fusion protein comprises a AlpC polypeptide comprising an amino acid sequence having at least 80%, 85%, 90%, 95%, 96%, 97%, 98%, 99%, or 100% identity to the sequence of SEQ ID NO: 11 (AlpC).

In some embodiments, a GBS fusion protein is, e.g., a SBD-AlpC fusion protein, and comprises, in any order, a sialic acid binding domain (SBD) as described herein, a polypeptide comprising an amino acid sequence having at least 80%, 85%, 90%, 95%, 96%, 97%, 98%, 99%, or 100% identity to the sequence of SEQ ID NO: 11 (AlpC) or an antigenic fragment thereof. In some embodiments, the fusion protein comprises (i) a SBD selected from any of SBD1, SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 2 or 112-120, and (ii) a polypeptide comprising an amino acid to the sequence of SEQ ID NO: 11 (AlpC) or an antigenic fragment thereof. In some embodiments, a SBD-AlpC fusion protein can further comprise at least 1, or at least 2 or more additional GBS polypeptides, e.g., selected from any of Rib, Sip, Alp1, Alp3 or Alp3/1 as disclosed herein.

In some embodiments, a GBS fusion protein is Rhavi-AlpC fusion protein, comprising, in any order, (i) a biotin-binding moiety (BBM) as described herein, e.g., Rhizavidin, and (ii) a polypeptide comprising an amino acid sequence having at least 80%, 85%, 90%, 95%, 96%, 97%, 98%, 99%, or 100% identity to the sequence of SEQ ID NO: 11 (AlpC) or an antigenic fragment thereof. In some embodiments, the fusion protein comprises a Rhizavidin having an amino acid sequence with at least 80%, or at least 85%, or at least 90%, or at least 95% sequence identity to SEQ ID NO: 1 or SEQ ID NO: 8, and a polypeptide comprising an amino acid sequence having at least 80%, 85%, 90%, 95%, 96%, 97%, 98%, 99%, or 100% identity to the sequence of SEQ ID NO: 11 (AlpC) or an antigenic fragment thereof. In some embodiments, a Rhavi-AlpC fusion protein can further comprise at least 1, or at least 2 or more additional GBS polypeptides, e.g., selected from any of Rib, Sip, Alp1, Alp3 or Alp3/1 as disclosed herein.

In some embodiments, a GBS fusion protein is, e.g., a SBD-AlpC-Rhavi fusion protein, and comprises, in any order, (i) a SBD selected from any of SBD1, SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 2 or 112-120, and (ii) a biotin-binding moiety as described herein, e.g., but not limited to, Rhizavidin having an amino acid sequence with at least 80%, or at least 85%, or at least 90%, or at least 95% sequence identity to SEQ ID NO: 1 or SEQ ID NO: 8, and (iii) a polypeptide comprising an amino acid sequence having at least 80%, 85%, 90%, 95%, 96%, 97%, 98%, 99%, or 100% identity to the sequence of SEQ ID NO: 11 (AlpC) or an antigenic fragment thereof. In some embodiments, a SBD-AlpC-Rhavi fusion protein can further comprise at least 1, or at least 2 or more additional GBS polypeptides, e.g., selected from any of Rib, Sip, Alp1, Alp3 or Alp3/1 as disclosed herein, which can be in any order.

### (d) Alp1, Alp 3 and Alp3/1

In some embodiments, a fusion protein useful in the MAPS-GBS immunogenic complex as disclosed herein comprises an amino acid sequence having at least 80%, or at least 85%, or at least 90%, or at least 95% sequence identity to SEQ ID NO: 12 (Alp1). In some embodiments, a fusion protein useful in the MAPS-GBS immunogenic complex as disclosed herein comprises an amino acid sequence having at least 80%, or at least 85%, or at least 90%, or at least 95% sequence identity to SEQ ID NO: 13 (Alp3). In some embodiments, a fusion protein useful in the MAPS-GBS immunogenic complex as disclosed herein comprises an amino acid sequence having at least 80%, or at least 85%, or at least 90%, or at least 95% sequence identity to SEQ ID NO: 14 (Alp3/1).

In some embodiments, a GBS fusion protein comprises a Alp polypeptide selected from Alp1, Alp3 or Alp3/1, comprising an amino acid sequence having at least 80%, 85%, 90%, 95%, 96%, 97%, 98%, 99%, or 100% identity to the sequence of SEQ ID NO: 12 (Alp1), SEQ ID NO: 13 (Alp3) or SEQ ID NO: 14 (Alp3/1).

In some embodiments, a GBS fusion protein is, e.g., a SBD-Alp fusion protein, and comprises, in any order, a sialic acid binding domain (SBD) as described herein, a polypeptide comprising an amino acid sequence having at least 80%, 85%, 90%, 95%, 96%, 97%, 98%, 99%, or 100% identity to any of the sequences of SEQ ID NO: 12 (Alp1), SEQ ID NO: 13 (Alp3) or SEQ ID NO: 14 (Alp3/1), or an antigenic fragment thereof. In some embodiments, the fusion protein comprises (i) a SBD selected from any of SBD1, SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 2 or 112-120, and (ii) a polypeptide comprising an amino acid to the sequence selected from any of SEQ ID NO: 12 (Alp1), SEQ ID NO: 13 (Alp3) or SEQ ID NO: 14 (Alp3/1) or an antigenic fragment or variant thereof. In some embodiments, a SBD-Alp3/1 or SBD-Alp1 or SBD-Alp3 fusion protein can further comprise at least 1, or at least 2 or more additional GBS polypeptides, e.g., selected from any of Rib, Sip, or AlpC as disclosed herein.

In some embodiments, a GBS fusion protein is Rhavi-Alp fusion protein comprises a Alp polypeptide selected from Alp1, Alp3 or Alp3/1, and comprises in any order, (i) a biotin-binding moiety (BBM) as described herein, e.g., Rhizavidin, and (ii) a polypeptide comprising an amino acid sequence having at least 80%, 85%, 90%, 95%, 96%, 97%, 98%, 99%, or 100% identity to any sequence selected from any of SEQ ID NO: 12 (Alp1), SEQ ID NO: 13 (Alp3) or SEQ ID NO: 14 (Alp3/1) or an antigenic fragment thereof. In some embodiments, the fusion protein comprises a Rhizavidin having an amino acid sequence with at least 80%, or at least 85%, or at least 90%, or at least 95% sequence identity to SEQ ID NO: 1 or SEQ ID NO: 8, and a polypeptide comprising an amino acid sequence having at least 80%, 85%, 90%, 95%, 96%, 97%, 98%, 99%, or 100% identity to the any sequence selected from any of SEQ ID NO: 12 (Alp1), SEQ ID NO: 13 (Alp3) or SEQ ID NO: 14 (Alp3/1) or an antigenic fragment thereof. In some embodiments, a Rhavi-Alp fusion protein can further comprise at least 1, or at least 2 or more additional GBS polypeptides, e.g., selected from any of Rib, Sip or AlpC as disclosed herein.

In some embodiments, a GBS fusion protein is, e.g., a SBD-AlpC-Rhavi fusion protein, and comprises, in any order, (i) a SBD selected from any of SBD1, SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 2 or 112-120, and (ii) a biotin-binding moiety as described herein, e.g., but not limited to, Rhizavidin having an amino acid sequence with at least 80%, or at least 85%, or at least 90%, or at least 95% sequence identity to SEQ ID NO: 1 or SEQ ID NO: 8, and (iii) a polypeptide comprising an amino acid sequence having at least 80%, 85%, 90%, 95%, 96%, 97%, 98%, 99%, or 100% identity to any sequence selected from any of SEQ ID NO: 12 (Alp1), SEQ ID NO: 13 (Alp3) or SEQ ID NO: 14 (Alp3/1) or an antigenic fragment thereof. In some embodiments, a SBD-Alp-Rhavi fusion protein can further comprise at least 1, or at least 2 or more additional GBS polypeptides, e.g., selected from any of Rib, Sip, or AlpC as disclosed herein, which can be in any order.

### (e) Exemplary GBS Fusion Proteins for Use in a MAPS-GBS Immunogenic Complex

In some embodiments, the GBS-fusion proteins described herein comprise one or more polypeptides listed in Table 1. In some embodiments, GBS-fusion proteins can be selected from the group listed in Table 2A and 2B, but are not limited to the specific arrangement of the SBD, GBS-Ag or BBM proteins in the GBS fusion protein.

In some embodiments, a MAPS-GBS immunogenic complex can comprise one or more fusion proteins selected from the group listed in Table 3. In some embodiments, a vaccine composition comprises one or more fusion proteins selected from the group listed in Table 3.

TABLE 3
Exemplary GBS fusion proteins for use
in a MAPS-GBS immunogenic complex.

| | Amino | | Amino |
|---|---|---|---|
| | acid | | acid |
| | SEQ | | SEQ |
| GBS-Fusion protein | ID NO: | GBS-Fusion protein | ID NO: |
| Rhavi-SBD1 | 3 | Rib-Rhavi-SBD2 | 164 |
| Rhavi-Rib | 17 | Sip-Rhavi-SBD2 | 165 |
| Rhavi-Sip | 18 | Rib-Rhavi-SBD3 | 166 |
| Rhavi-Rib-Sip | 19 | Sip-Rhavi-SBD3 | 167 |
| Rhavi-Sip-Rib | 20 | Rib-Rhavi-SBD4 | 168 |
| Rib-Sip-SBD1 | 21 | Sip-Rhavi-SBD4 | 169 |
| Sip-Rib-SBD1 | 22 | Rhavi-AlpC | 27 |
| Rhavi-Rib-Sip-SBD1 | 6 | Rhavi-AlpC-SBD1 | 28 |
| Rhavi-Sip-Rib-SBD1 | 7 | AlpC-SBD1 | 29 |
| Rib-Sip-Rhavi-SBD1 | 9 | Rhavi-Rib-AlpC | 30 |
| Sip-Rib-Rhavi-SBD1 | 10 | Rhavi-Sip-AlpC | 31 |
| Rhavi-SBD1-Rib-Sip | 23 | Rhavi-AlpC-Rib | 32 |
| Rhavi-SBD1-Sip-Rib | 24 | Rhavi-AlpC-Sip | 33 |
| Rib-SBD1 | 25 | Rhavi-AlpC-Alp3/1 | 34 |
| Sip-SBD1 | 26 | Rhavi-Alp3/1-AlpC | 35 |
| Rhavi-Rib-SBD1 | 127 | Rhavi-Rib-AlpC-SBD1 | 36 |
| Rhavi-Sip-SBD1 | 128 | Rhavi-Sip-AlpC-SBD1 | 70 |
| Rib-Rhavi-SBD1 | 162 | Rhavi-AlpC-Rib-SBD1 | 71 |
| Sip-Rhavi-SBD1 | 163 | Rhavi-AlpC-Sip-SBD1 | 72 |
| Rib-Sip-Rhavi-SBD2 | 121 | Rhavi-AlpC-Alp3/1-SBD1 | 73 |
| Sip-Rib-Rhavi-SBD2 | 122 | Rhavi-Alp3/1-AlpC-SBD1 | 74 |
| Rib-Sip-Rhavi-SBD3 | 123 | Rib-AlpC-SBD1 | 75 |
| Sip-Rib-Rhavi-SBD3 | 124 | Sip-AlpC-SBD1 | 76 |
| Rib-Sip-Rhavi-SBD4 | 125 | AlpC-Rib-SBD1 | 77 |
| Sip-Rib-Rhavi-SBD4 | 126 | AlpC-Sip-SBD1 | 78 |
| Rhavi-SBD2 | 129 | Alp3/1-AlpC-SBD1 | 79 |
| Rib-Sip-SBD2 | 130 | AlpC-Alp3/1-SBD1 | 80 |
| Sip-Rib-SBD2 | 131 | Rhavi-SBD1-Rib-AlpC | 81 |
| Rhavi-Rib-Sip-SBD2 | 132 | Rhavi-SBD1-Sip-AlpC | 82 |
| Rhavi-Sip-Rib-SBD2 | 133 | Rhavi-SBD1-AlpC-Rib | 83 |
| Rhavi-Rib-SBD2 | 134 | Rhavi-SBD1-AlpC-Sip | 84 |
| Rhavi-Sip-SBD2 | 135 | Rhavi-SBD1-AlpC-Alp3/1 | 85 |
| Rhavi-SBD2-Rib-Sip | 136 | Rhavi-SBD1-Alp3/1-AlpC | 86 |
| Rhavi-SBD2-Sip-Rib | 137 | Rib-AlpC-Rhavi-SBD1 | 87 |
| Rib-SBD2 | 138 | Sip-AlpC-Rhavi-SBD1 | 88 |
| Sip-SBD2 | 139 | AlpC-Rib-Rhavi-SBD1 | 89 |
| Rhavi-SBD3 | 140 | AlpC-Sip-Rhavi-SBD1 | 90 |
| Rib-Sip-SBD3 | 141 | AlpC-Alp3/1-Rhavi-SBD1 | 91 |
| Sip-Rib-SBD3 | 142 | Alp3/1-AlpC-Rhavi-SBD1 | 92 |
| Rhavi-Rib-Sip-SBD3 | 143 | Rhavi-Alp3/1 | 93 |
| Rhavi-Sip-Rib-SBD3 | 144 | Rhavi-Alp3/1-SBD1 | 94 |
| Rhavi-Rib-SBD3 | 145 | Alp3/1-SBD1 | 95 |
| Rhavi-Sip-SBD3 | 146 | Rhavi-Rib-Alp3/1 | 96 |
| Rhavi-SBD3-Rib-Sip | 147 | Rhavi-Sip-Alp3/1 | 97 |
| Rhavi-SBD3-Sip-Rib | 148 | Rhavi-Alp3/1-Rib | 98 |
| Rib-SBD3 | 149 | Rhavi-Alp3/1-Sip | 99 |
| Sip-SBD3 | 150 | Rhavi-Rib-Alp3/1-SBD1 | 100 |
| Rhavi-SBD4 | 151 | Rhavi-Sip-Alp3/1-SBD1 | 101 |
| Rib-Sip-SBD4 | 152 | Rhavi-Alp3/1-Rib-SBD1 | 102 |
| Sip-Rib-SBD4 | 153 | Rhavi-Alp3/1-Sip-SBD1 | 103 |
| Rhavi-Rib-Sip-SBD4 | 154 | Rhavi-SBD1-Rib-Alp3/1 | 104 |
| Rhavi-Sip-Rib-SBD4 | 155 | Rhavi-SBD1-Sip-Alp3/1 | 105 |
| Rhavi-Rib-SBD4 | 156 | Rhavi-SBD1-Alp3/1-Rib | 106 |
| Rhavi-Sip-SBD4 | 157 | Rhavi-SBD1-Alp3/1-Sip | 107 |
| Rhavi-SBD4-Rib-Sip | 158 | Rib-Alp3/1-Rhavi-SBD1 | 108 |
| Rhavi-SBD4-Sip-Rib | 159 | Sip-Alp3/1-Rhavi-SBD1 | 109 |
| Rib-SBD4 | 160 | Alp3/1-Rib-Rhavi-SBD1 | 110 |
| Sip-SBD4 | 161 | Alp3/1-Sip-Rhavi-SBD1 | 111 |

In some embodiments, a GBS fusion protein useful in the MAPS-GBS immunogenic complex as disclosed herein comprises at least one, or at least two, or at least 3 antigenic polypeptides selected from SEQ ID NOS: 4, 5, 11-15, or least one, or at least two, or at least 3 antigenic polypeptides having an amino acid sequence having at least 80%, or at least 85%, or at least 90%, or at least 95% sequence identity to SEQ ID NOS: SEQ ID NO: 4, 5, 11-15.

In some embodiments, a fusion protein useful in the MAPS-GBS immunogenic complex as disclosed herein comprises an amino acid sequence having at least 80%, or at least 85%, or at least 90%, or at least 95% sequence identity to any of the GBS fusion proteins listed in Table 3. In some embodiments, a fusion protein useful in the MAPS-GBS immunogenic complex as disclosed herein comprises an amino acid sequence having at least 80%, or at least 85%, or at least 90%, or at least 95% sequence identity to any of the GBS fusion proteins selected from any of SEQ ID NOS: 3, 6, 7, 9, 10, 17, 18, 19, 20, 21, 22, 23, 24, 25, 26-36, 70-111 and 121-169. It is envisioned that SBD in any GBS fusion protein in Table 3 can be readily substituted for any other SBD protein as disclosed herein, including, but not limited to SBD1, SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 2 or 112-120.

It is envisioned that SBD1 in any GBS fusion protein selected from SEQ ID NO: 28-36, 70-111 can be readily substituted for any other SBD protein as disclosed herein, including, but not limited to SBD2, SBD3, SBD4, NanH, NanH2, VcNanH, or having an amino acid sequence with at least 80%, 85%, 90%, 95%, 96%, 97%, 98%, 99%, or 100% sequence identity to any of: SEQ ID NOs: 112-120.

In some embodiments, a fusion protein useful in the MAPS-GBS immunogenic complex as disclosed herein comprises an amino acid sequence having at least 80%, or at least 85%, or at least 90%, or at least 95% sequence identity to SEQ ID NO: 15 (AlpC-Rib).

In some embodiments, a fusion protein described herein includes a variant or fragment of a polypeptide listed in Table 1. In some embodiments, a fusion protein described herein includes a polypeptide encoded by a variant or fragment of a gene listed in Table 1. In some embodiments, a fragment included in a fusion protein described herein is close in size to a full-length polypeptide or a polypeptide listed in Table 1. For example, they may lack at most one, two, three, four, five, ten, twenty, or thirty amino acids from one or both termini. In some embodiments, the fragment is 25-50 amino acids in length, or 50-100, or 100-150, or 150-200, or 200-250, or 250-300, or 300-350 amino acids in length. In some embodiments, the fragments result from processing, or partial processing, of signal sequences by an expression host, e.g. E. coli, an insect cell line (e.g., the baculovirus expression system), or a mammalian (e.g., human or Chinese Hamster Ovary) cell line. The fragments described above or sub-fragments thereof (e.g., fragments of 8-50, 8-30, or 8-20 amino acid residues) preferably have one of the biological activities described below, such as increasing the amount of IL-17 released by at least 1.5 fold or 2 fold or more (e.g., either as an absolute measure or relative to a control protein).

The DNA and protein sequence of each gene and polypeptide may be identified by searching for the Locus Tag in a publicly available database, e.g., Entrez Gene (on the NCBI NIH web site on the World Wide Web, at www.ncbi.nlm.nih.gov/sites/entrez?db=gene), in the Streptococcus pneumoniae TIGR4 genome, and the indicated sequences are also included within the scope of the present disclosure.

Certain polypeptides of Table 1, variants thereof, and additional exemplary polypeptides and linkers which constitute components of various embodiments of the fusion proteins are described in greater detail below.

### (f) Alternative GBS Immunogenic Polypeptides.

It is envisioned that at least one, or at least two of the GBS polypeptide antigens in a fusion protein as disclosed herein (e.g., Sip, Ri, AlpC, Alp3/1 etc.) can be substituted for any polypeptide antigen, for example including, but not limited to any antigen disclosed in U.S. Pat. Nos. 10,766,932, 11,235,047, 11,013,793, 11,305,001, 9,499,593, 10,017,548, 10,611,805 or U.S. patent application Ser. No. 14/766,252, 16/616,258, 16/568,646, the contents of each are incorporated herein in their entirety by reference.

Other aspects relate to the use of such a SBD-Antigen fusion protein in methods, compositions and vaccines comprising a MAPS-GBS immunogenic complex comprising for the treatment and or prevention of infection with Group B streptococci (GBS), including the prevention and treatment of common manifestations of GBS infections, including but not limited to bacteremia, pneumonia, meningitis, endocarditis, and osteoarticular infections

It is envisioned that at least one, or at least two of the GBS polypeptide antigens as disclosed herein in GBS fusion protein as disclosed herein can be substituted for any polypeptide antigen, for example including, but not limited to any antigen disclosed in U.S. Pat. Nos. 10,766,932, 11,235,047, 11,013,793, 11,305,001, 9,499,593, 10,017,548, 10,611,805 or U.S. patent application Ser. Nos. 14/766,252, 16/616,258, 16/568,646, the contents of each are incorporated herein in their entirety by reference. In some embodiments, at least one, or at least two of the GBS polypeptide antigens as disclosed herein in GBS fusion protein as disclosed herein can be substituted for any polypeptide antigen selected from any of: EmaA, EmaB, EmaC, EmaD and EmaE, Spb1, Spb2, C protein alpha antigen or Lmb, C5a-ase or an immunogenic fragment thereof. Such GBS immunogenic polypeptides are disclosed in U.S. Pat. No. 7,645,577, which is incorporated herein in its entirety. The polypeptides of EmaA, EmaB, EmaC, EmaD and EmaE are disclosed as SEQ ID NO: 2, 4, 6, 8 and 10, respectively in U.S. Pat. No. 7,645,577, which is incorporated herein in its entirety.

GBS proteins with streptococcal homologs outside of Group B have been previously identified. There is some cross-protection between Group A and Group B streptococci due to cross-reacting surface proteins. The R28 protein of group A streptococcus (GAS) and the Rib protein of group B streptococcus (GBS) are surface molecules that elicit protective immunity to experimental infection. These proteins are members of the same family and cross-react immunologically. In spite of extensive amino acid residue identity, the cross-reactivity between R28 and Rib was found to be limited. Nevertheless, immunization of mice with purified R28 conferred protection against lethal infection with Rib-expressing GBS strains, and immunization with Rib conferred protection against R28-expressing GAS. Thus, R28 and Rib elicited cross-protective immunity. Therefore, it is envisioned that a person of ordinary skill in the art can substitute Rib in a GBS fusion protein as disclosed herein with R28.

In some embodiments, a GBS fusion protein comprising any of Sip, Rib, AlpC, Alp3/1 can further comprise CP1, as described in PCT Application WO2021/17016516 which is incorporated herein in its entirety by reference.

In some embodiments, a fusion protein useful in the MAPS-GBS immunogenic complex as disclosed herein is CP1, further described in PCT Application WO2021/17016516 entitled “Pneumococcal Fusion Protein Vaccines” and filed Sep. 12, 2019, the contents of each of which are incorporated herein by reference in their entirety. Aspects of the CP1 have also been previously described in WO2020/056127, the contents of which are herein incorporated by reference in their entirety, In some embodiments, the fusion protein comprises a complementary affinity molecule described herein (e.g., a biotin-binding moiety described herein) and a polypeptide comprising an amino acid sequence having at least 80%, 85%, 90%, 95%, 96%, 97%, 98%, 99%, or 100% identity to the sequence of SEQ ID NO: 6 or an antigenic fragment thereof.

### (g) Linker or Spacer

In some embodiments, a GBS fusion protein as disclosed herein comprises one or more linkers. In some embodiments, a linker is or comprises one or more amino acids. One or more linkers can be used to join two different polypeptides. For example, a linker can be located between a BBM and a GBS immunogenic polypeptide as disclosed herein (e.g., BBM-linker-[GBS-Ag]), between a SBD and immunogenic polypeptide (e.g., SBD-linker-[GBS-Ag]), or be located between a BBM and a SBD (e.g., BBM-linker-SBD), or located between two GBS immunogenic antigens (e.g., [GBS-Ag1]-linker-[GBS-Ag2]). That is, in any of the GBS fusion proteins disclosed herein, e.g., a SBD-[GBS-Ag]n fusion protein, a BBM-[GBS-Ag]n fusion protein, or a bifunctional SBD-[GBS-Ag]n-BBM fusion protein, one or more linkers can be located between any protein moieties of BBM, GBS-Ag or SBD.

In some embodiments, a linker interposes a structure between two protein moieties. In some embodiments, the structure is or comprises an α-helix. In some embodiments the structure is or comprises a β-strand. In some embodiments, the structure is or comprises a coil/bend. In some embodiments, the structure is or comprises a turn. In some embodiments, a linker decreases steric hindrance between two protein moieties joined by the linker. In some embodiments, a linker decreases unfavorable interactions between two protein moieties joined by the linker. In some embodiments, a linker comprises a mixture of glycine and serine residues. In some embodiments, the linker may additionally comprise threonine, proline, and/or alanine residues. In some embodiment a linker is hydrophilic. In some embodiments a linker is hydrophobic. In some embodiments a linker increases the stability of the fusion protein containing the linker.

In some embodiments, a linker does not interfere with the folding of an antigenic polypeptide to which it is joined. In some embodiments, a linker does not interfere with the antigenicity of an antigenic polypeptide to which it is joined. In some embodiments, a linker does not reduce the antigenicity of an antigenic polypeptide to which it is joined. In some embodiments, a linker does not eliminate the antigenicity of an antigenic polypeptide to which it is joined. In some embodiments the effect of the linker is determined by comparing the polypeptide with the polypeptide joined to the linker.

In some embodiments, a linker does not interfere with the folding of a biotin-binding moiety to which it is joined. In some embodiments, a linker does not interfere with the biotin-binding ability of a biotin-binding moiety to which it is joined. In some embodiments, a linker does not reduce the biotin-binding ability of a biotin-binding moiety to which it is joined. In some embodiments, a linker does not eliminate the biotin-binding ability of a biotin-binding moiety to which it is joined. In some embodiments the effect of the linker is determined by comparing the biotin-binding moiety with the biotin-binding moiety joined to the linker.

In some embodiments, a linker is not antigenic. In some embodiments, a linker does not elicit a T cell response. In some embodiments, a linker does not elicit a B cell response. In some embodiments, a linker does not induce a T cell or a B cell response.

In some embodiments, a linker comprises two or more amino acids. In some embodiments, a linker may be 3-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20, 10-15, 3-10, 3-9, 3-8, 3-7, 3-6, 3-5, 3-4, or 2-3 amino acids in length. In some embodiments, a linker comprises between 10-100, 10-90, 10-80, 10-70, 10-60, 10-50, 10-40, 10-30, 10-20, 10-15 amino acids. In some embodiments, the linker comprises at least 10, 15, 20, 25, 30, 35, 40, 45, 50, 55, 60, 65, 70, 75, 80, 85, 90, or 95 amino acids. In some embodiments, a linker is or comprises 2, 3, 4, 5, 6, 7, 8, 9, 10, 15, 20, 25, 30, 35, 40, 45, 50, 55, 60, 65, 70, 75, 80, 85, 90, 95, or 100 amino acids.

In some embodiments, a linker is a flexible linker. Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties. In some embodiments a linker comprises small non-polar (e.g. Gly) or polar (e.g. Ser or Thr) amino acids. In some embodiments, a linker is a Gly-Ser linker.

In some embodiments, the fusion protein further comprises one or more linkers. In some embodiments, the one or more linkers are selected from GS, or SEQ ID NO:37 (GGGGSSS) and SEQ ID NO:38 (AAA). In some embodiments, the fusion protein comprises an amino acid sequence AAA (SEQ ID NO: 38) residual from a Not I restriction site. In some embodiments, the fusion protein comprises a linker of SEQ ID NO:37 (GGGGSSS) and an amino acid sequence AAA (SEQ ID NO: 38) residual from a Not I restriction site.

In some embodiments, a linker is or comprises an amino acid sequence of GS or GGGGSSS (SEQ ID NO:37). In some embodiments, a linker is or comprises a sequence of (GGGGS)ₙ (SEQ ID NO:39), where n represents the number of repeating GGGGS (SEQ ID NO: 62) units and is 1, 2, 3, 4, 5, 6, 7, 8, 9, 10, 11, 12, 13, 14, 15, 16, 17, 18, 19, 20, 25, 30 or more. In some embodiments, a polypeptide linker may have an amino acid sequence that is or comprises GGGGSGGGGSGGGGS (SEQ ID NO:41) (i.e., (GGGGS)₃ (SEQ ID NO: 41)) or GGGGSGGGGSGGGGSGGGGSGGGGSGGGGS (SEQ ID NO:42) (i.e., (GGGGS)₆ (SEQ ID NO: 42)). In some embodiments, a linker comprises one or more of Gly, Ser, Thr, Ala, Lys, and Glu. In some embodiments, a linker is or comprises KESGSVSSEQLAQFRSLD (SEQ ID NO:43). In some embodiments, a linker is or comprises EGKSSGSGSESKST (SEQ ID NO:44). In some embodiments, a linker is or comprises (Gly)ₙ (SEQ ID NO:45) where n represents the number of repeating Gly residues and is 1, 2, 3, 4, 5, 6, 7, 8, 9, 10, 11, 12, 13, 14, 15, 16, 17, 18, 19, 20, 25, 30 or more. In some embodiments a linker is or comprises GS or GGG. In some embodiments, a linker is or comprises (Gly)₆ (SEQ ID NO:40). In some embodiments, a linker is or comprises (Gly)₈ (SEQ ID NO:46). In some embodiments, a linker is or comprises GSAGSAAGSGEF (SEQ ID NO:47). In some embodiments, a linker is or comprises an amino acid sequence of AAA (SEQ ID NO:38) or GGGSS (SEQ ID NO: 62).

In some embodiments, a linker is a rigid linker. Rigid linkers are useful to keep a fixed distance between domains and to maintain their independent functions. Rigid linkers may also be useful when a spatial separation of the domains is critical to preserve the stability or bioactivity of one or more components in the fusion. In some embodiments, a linker is or comprises (EAAAK)ₙ (SEQ ID NO:48) where n represents the number of repeating EAAAK (SEQ ID NO: 63) units and is 1, 2, 3, 4, 5, 6, 7, 8, 9, 10, 11, 12, 13, 14, 15, 16, 17, 18, 19, 20, 25, 30 or more. In some embodiments, a linker is or comprises A(EAAAK)ₙA, (SEQ ID NO:49) where n represents the number of repeating EAAAK (SEQ ID NO: 63) units and is 1, 2, 3, 4, 5, 6, 7, 8, 9, 10, 11, 12, 13, 14, 15, 16, 17, 18, 19, 20, 25, 30 or more. In some embodiments, a linker is or comprises A(EAAAK)₄A (SEQ ID NO: 61), where n represents the number of repeating EAAAK (SEQ ID NO: 63) units and is 2, 3, 4, or 5. In some embodiments, a linker is or comprises A(EAAAK)₄ALEA(EAAAK)₄A (SEQ ID NO:50). In some embodiments, a linker is or comprises [A(EAAAK)ₙA]ₘ, (SEQ ID NO:51) wherein n is 2, 3, or 4 and m is 1 or 2. In some embodiments, a linker is or comprises AEAAAKEAAAKA (SEQ ID NO:52).

In some embodiments a linker is or comprises (X-Pro)ₙ (SEQ ID NO:53), with X designating any amino acid, where n represents the number of repeating X-Pro units and is 1, 2, 3, 4, 5, 6, 7, 8, 9, 10, 11, 12, 13, 14, 15, 16, 17, 18, 19, 20, 25, 30 or more. In some embodiments a linker is or comprises (Ala-Pro)ₙ (SEQ ID NO:54), where n represents the number of repeating Ala-Pro units and is 1, 2, 3, 4, 5, 6, 7, 8, 9, 10, 11, 12, 13, 14, 15, 16, 17, 18, 19, 20, 25, 30 or more. In some embodiments a linker is or comprises (Ala-Pro)ₙ (SEQ ID NO: 64), where n represents the number of repeating Ala-Pro units and is 5, 6, 7, 8, 9, 10, 11, 12, 13, 14, 15, 16, or 17.

In some embodiments a linker is or comprises (Lys-Pro)ₙ (SEQ ID NO:55), where n represents the number of repeating Lys-Pro units and is 1, 2, 3, 4, 5, 6, 7, 8, 9, 10, 11, 12, 13, 14, 15, 16, 17, 18, 19, 20, 25, 30 or more. In some embodiments a linker is or comprises (Gln-Pro)ₙ (SEQ ID NO:56), where n represents the number of repeating Gln-Pro units and is 1, 2, 3, 4, 5, 6, 7, 8, 9, 10, 11, 12, 13, 14, 15, 16, 17, 18, 19, 20, 25, 30 or more. In some embodiments, a linker is or comprises (Ala-Pro)₇ (SEQ ID NO:57).

In some embodiments a linker is or comprises GAPGGGGGAAAAAGGGGGGAP (GAG linker, SEQ ID NO:58). In some embodiments a linker is or comprises GAPGGGGGAAAAAGGGGGGAPGGGGGAAAAAGGGGGGAP (GAG2 linker, SEQ ID NO:59). In some embodiments a linker is or comprises GAPGGGGGAAAAAGGGGGGAPGGGGGAAAAAGGGGGGAPGGGGGAAAAAGGGGGGAP (GAG3 linker, SEQ ID NO:60).

Suitable linkers or spacers also include those having an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to the above exemplary linkers.

Additional linkers suitable for use with some embodiments may be found in U.S. Patent Publication No. 2012/0232021, filed on Mar. 2, 2012, and [Chen, 2013] the disclosures of which is hereby incorporated by reference in their entireties. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

### (h) Tagged Fusion Proteins

In some embodiments, a GBS fusion protein described herein may comprise a tag. A tag may be N-terminal or C-terminal. For instance, tags may be added to a polypeptide (via additions or modifications on the encoding DNA sequence) to facilitate purification, detection, solubility, or confer other desirable characteristics on the protein. In some embodiments a tag may be a peptide, oligopeptide, or polypeptide that may be used in affinity purification. In some embodiments, a tag is, comprises, or is derived from one or more of polyhistidine (His), Glutathione S-transferase (GST), tandem affinity purification (TAP), FLAG, myc, human influenza hemagglutinin (HA), maltose binding protein (MBP), vesicular Stomatitis viral glycoprotein (VSV-G), thioredoxin, V5, avidin, streptavidin, biotin carboxyl carrier protein (BCCP), Calmodulin, Nus, S tags, lipoprotein D, and galactosidase. In some embodiments, a His tag is or comprises an amino acid sequence of Hₙ, wherein n is an integer between 2 and 10. Exemplary His tags include HBHHHHH (SEQ ID NO:65) and MSYYHHHIIHH (SEQ ID NO:66). In other embodiments, the fusion protein is free of tags such as protein purification tags, and is purified by a method not relying on affinity for a purification tag. In some embodiments, the fusion protein comprises no more than 1, 2, 3, 4, 5, 10, or 20 additional amino acids on one or both termini of a polypeptide of Table 1 or fusion protein of Table 2A or 2B or Table 3.

In some embodiments, a fusion protein described herein may contain a membrane translocating sequence (MTS), to facilitate introduction of the fusion protein into a mammalian cell and subsequent stimulation of the cell-mediated immune response. Exemplary membrane translocating sequences include the hydrophobic region in the signal sequence of Kaposi fibroblast growth factor, the MTS of a synuclein, the third helix of the Antennapedia homeodomain, SN50, integrin 3 h-region, HIV Tat, pAntp, PR-39, abaecin, apidaecin, Bac5, Bac7, P. berghei CS protein, and those MTSs described in U.S. Pat. Nos. 6,248,558, 6,432,680 and 6,248,558.

### IV. Antigenic GBS Polysaccharides

In some embodiments, an GBS-MAPS immunogenic complex described herein includes one or more biotinylated GBS polysaccharides (PS). In some embodiments, a GBS-MAPS immunogenic complex as described herein comprises a biotinylated polysaccharide from the GBS. In some embodiments, an MAPS-GBS immunogenic complex includes one or more biotinylated GBS capsular polysaccharides or biotinylated O-specific polysaccharides (OSP) from, or derived from, one or more GBS subtypes selected from group consisting of serotypes Ia, Ib, II, III, IV, V, VI, VII, VIII, and IX.

In some embodiments, an GBS-MAPS immunogenic complex described herein comprises a GBS polysaccharide that is >60 kDa, or >70 kDa, or >80 kDa, or >90 kDa, or >100 kDa, or >110 kDa, or >120 kDa. In some embodiments, an immunogenic complex described herein comprises an OSP polysaccharide from GBS that is between 90-110 kDa.

In some embodiments, the first (PS1), or second polysaccharide (PS2), or both is isolated from Streptococcus agalactiae. The polysaccharide may be isolated from any encapsulated strain of S. agalactiae, such as 090, A909 (ATCC Accession No. BAA-1138), 515 (ATCC Accession No. BAA-1177), B523, CJB524, MB 4052 (ATCC Accession No. 31574), H36B (ATCC Accession No. 12401), S40, S42, MB 4053 (ATCC Accession No. 31575), M709, 133, 7357, PFEGBST0267, MB 4055 (ATCC Accession No. 31576), 18RS21 (ATCC Accession No. BAA-1175), S16, S20, V8 (ATCC Accession No. 12973), DK21, DK23, UAB, 5401, PFEGBST0708, MB 4082 (ATCC Accession No. 31577), M132, 110, M781 (ATCC Accession No. BAA-22), D136C(3) (ATCC Accession No. 12403), M782, S23, 120, MB 4316 (M-732; ATCC Accession No. 31475), M132, K79, COH1 (ATCC Accession No. BAA-1176), PFEGBST0563, 3139 (ATCC Accession No. 49446), CZ-NI-016, PFEGBST0961, 1169-NT1, CJB111(ATCC Accession No. BAA-23), CJB1 12, 2603 V/R (ATCC Accession No. BAA-611), NCTC 10/81, CJ11, PFEGBST0837, 118754, 114852, 114862, 114866, 118775, B 4589, B 4645, SS1214, CZ-PW-119, 7271, CZ-PW-045, JM9130013, JM9130672, IT-N1-016, IT-PW-62, and IT-PW-64.

Polysaccharides isolated from Streptococcus agalactiae useful in the immunogenic complexes as disclosed herein are disclosed in U.S. Pat. No. 10,226,525, which is incorporated herein in its entirety by reference.

In some embodiments, the PS1 and PS2 for each immunogenic complex of each species is from the same GBS serotype. In some embodiments, the PS1 and PS2 for each MAPS-GBS immunogenic complex for each species is from a different GBS serotype. For example, PS1 and PS2 for a particular species of an MAPS-GBS immunogenic complex can be from, e.g., a specific serotype of Group B Streptococcus (GBS) or Streptococcus agalactiae. In alternative embodiments, PS1 and PS2 for a particular species of an immunogenic complex can be from, e.g., different serotypes of Group B Streptococcus (GBS) or Streptococcus agalactiae.

Aspects of the technology described herein relate to a composition, which is a polyvalent immune composition, and comprises at least one species of MAPS-GBS immunogenic complex. That is, each MAPS-GBS species is distinct from the other MAPS-GBS species in the immunogenic complex. Without being limited to theory, for example, a composition can comprise, e.g., two MAPS-GBS immunogenic complexes as disclosed herein, where the first species of MAPS-GBS immunogenic complex disclosed herein comprises a PS1 and PS2 from Streptococcus agalactiae serotype Ia, and the second species of MAPS-GBS immunogenic complex can comprise a PS1 and PS2 from Streptococcus agalactiae serotype 1b. In such an embodiment, a composition would be considered a two valent (2V) MAPS-GBS immunogenic composition or vaccine. It is envisioned that a MAPS-GBS polyvalent immune composition as disclosed herein comprises MAPS-GBS immunogenic complexes that comprise polysaccharides from at least 2, or at least 3, or at least 4, or at least 5, or at least 6, or at least 7, or at least 8, or at least 9, or at least 10, or 2-5, or 5-6, or 6-7, or 7-8, or 8-9 or 9-10, or more than 10 different serotypes of GBS bacteria.

In some embodiments, as disclosed herein, the first biotinylated polysaccharide (PS1), or second biotinylated polysaccharide (PS2), or both, is from any of serotypes Ia, Ib, II, III, IV, V, VI, VII, VIII, and IX of Streptococcus agalactiae. In some embodiments, as disclosed herein, the first biotinylated polysaccharide, or second biotinylated polysaccharide, or both, is from any of serotypes Ia, Ib, II, III, IV, V or VII of Streptococcus agalactiae (7VMAPS-GBS). In some embodiments, as disclosed herein, the first biotinylated polysaccharide, or second biotinylated polysaccharide, or both, is from any of serotypes Ia, Ib, II, III, V or VII of Streptococcus agalactiae (6VMAPS-GBS).

### Methods of Isolating and Purifying Polysaccharides

In some embodiments, the disclosure provides methods of purifying one or more polysaccharides described herein from GBS from cellular components of bacteria. In some embodiments, methods comprise purifying capsular polysaccharides from one or more cellular components of bacteria. In some embodiments, the cellular components include protein. In some embodiments, the cellular proteins include nucleic acid. In some embodiments, the cellular components include lipids. In some embodiments, the cellular components include polysaccharides. In some embodiments, the cellular components are part of a lysate.

In some embodiments, the polysaccharide purification processes incorporate a series of ethanol precipitations, washes of crude polysaccharide preparations with ethanol, diethyl ether, and/or acetone, and drying under vacuum to furnish purified products. In some embodiments, a phenol extraction step is incorporated for polysaccharide purifications. In some embodiments the purification process employs a CTAB (cetyltrimethyl ammonium bromide) precipitation step in addition to using ethanol and phenol precipitation steps.

### Methods of Biotinylating Polysaccharides

In some embodiments, the disclosure provides methods of biotinylating one or more polysaccharides described herein. In some embodiments, the method comprises reacting purified polysaccharides with 1-cyano-4-dimethylaminopyridinium tetrafluoroborate (CDAP) for activation of hydroxyl groups in the polysaccharides followed by the addition of amine PEG biotin under conditions that result in covalent linkage of biotin to the polysaccharides. In some embodiments, the desired level of biotinylation is achieved by varying the ratio of CDAP to polysaccharide. In some embodiments, the method comprises reacting purified polysaccharides with 1-Ethyl-3-[3-dimethylaminopropyl]carbodiimide Hydrochloride (EDC) and N-hydroxysulfosuccinimide (NHS). In some embodiments, the biotinylated polysaccharides are purified by filtration to remove process residuals such as unreacted biotin, dimethylaminopyridine, acetonitrile, cyanide and unreacted glycine. In some embodiments, the level of polysaccharide biotinylation described herein is optimized to reduce the amount of accessible biotin following MAPS complexation.

### V. Nucleic Acids Encoding the Fusion Proteins or Nucleic Acid Vaccine Compositions

In some embodiments, the present disclosure provides nucleic acids, e.g., DNA, RNA, or analogs thereof, encoding one or more of the polypeptides and/or fusion proteins described herein. An underlying DNA sequence for the polypeptides described herein may be modified in ways that do not affect the sequence of the protein product, and such sequences are included in the invention. In some embodiments, a DNA sequence may be codon-optimized to improve expression in a host such as a bacterial cell line, e.g., E. coli, an insect cell line (e.g., using the baculovirus expression system), or a mammalian (e.g., human or Chinese Hamster Ovary) cell line.

In some embodiments, the present disclosure provides nucleic acids, e.g., DNA, RNA, or analogs thereof, that are at least 70%, 80%, 85%, 90%, 95%, 97%, 98%, 99%, 99.5%, or 100% identical to a nucleic acid sequences that comprise SEQ ID NOS: 16 and any of: 170-174 as provide in Table 1, or a variant or portion thereof. In some embodiments, the nucleic acid is 600-2000, 800-1800, 1000-1600, 1200-1400 nucleotides in length. In some embodiments, the nucleic acid is 600-1600, 800-1800, 1000-2000, 2000-3000, or 3000-4000 nucleotides in length.

In all cases, due to degeneracy in the genetic code, other DNA sequences (including multiple codon-optimized sequences) could be contemplated by those of ordinary skill to encode such polypeptides and fusion proteins.

Nucleic acids encoding polypeptides or fusion proteins of Table 1 or Table 2, or fragments thereof, can be cloned into any of a variety of expression vectors, under the control of a variety of regulatory elements, and fusions can be created with other sequences of interest. Methods of cloning nucleic acids are routine and conventional in the art. For general references describing methods of molecular biology which are mentioned in this application, e.g., isolating, cloning, modifying, labeling, manipulating, sequencing and otherwise treating or analyzing nucleic acids and/or proteins, see, e.g., Sambrook et al, 1989; Ausubel et al, 1995; Davis et al, 1986; Hames et al, 1985; Dracopoli et al, 2018; and Coligan et al, 2018.

### Nucleic Acid-Based Immunogenic Compositions and Vaccines

The present disclosure also provides immunogenic compositions (e.g., vaccine compositions) of, or comprising, one or more nucleic acids encoding fusion proteins described herein. In some embodiments, the immunogenic composition comprises one or more nucleic acids encoding fusion proteins with at least 80%, 85%, 90%, 95%, 96%, 97%, 98%, 99%, 99.5%, or 100% sequence identity to a fusion protein listed in Table 2A, 2B or Table 3. In all cases, due to degeneracy in the genetic code, other DNA sequences (including multiple codon-optimized sequences) could encode such fusion proteins. In some embodiments, these nucleic acids are expressed in the immunized individual, resulting in production of the encoded Streptococcus agalactiae fusion proteins, and the Streptococcus agalactiae fusion proteins so produced have an immunostimulatory or immunoprotective effect in the immunized individual.

Such a nucleic acid-containing immunostimulatory composition may comprise, for example, an origin of replication, and/or a promoter that drives expression of one or more nucleic acids encoding one or more fusion proteins disclosed in Table 3. Such a composition may also comprise a bacterial plasmid vector into which is inserted a promoter (sometimes a strong viral promoter), one or more nucleic acids encoding one or more fusion proteins of disclosed in Table 3, and a polyadenylation/transcriptional termination sequence. In some instances, the nucleic acid is DNA. In some instances, the nucleic acid is RNA.

In some embodiments, an immunogenic composition described herein (e.g., a vaccine composition) includes a fusion protein described herein and additionally one or more, or two or more, known Streptococcus agalactiae antigens. In some instances, the known Streptococcus agalactiae antigens are predominantly antibody targets. In some instances, the known Streptococcus agalactiae antigens are polysaccharides. In some instances, the known Streptococcus agalactiae antigens protect from Streptococcus agalactiae colonization, or from Streptococcus agalactiae-induced sepsis, pneumonia, meningitis, otitis media, sinusitis, or infection of other sites or organs by Streptococcus agalactiae.

### IV. Multivalent MAPS-GBS Immunogenic Compositions and Vaccines

Another aspect of the disclosure provides a composition, e.g., immunogenic composition or vaccine composition that include one or more MAPS-GBS immunogenic complexes described herein. For example, an immunogenic composition, e.g., vaccine composition, can include one or more MAPS-GBS immunogenic complexes described herein. In some embodiments, such compositions can include a plurality of one type of MAPS-GBS immunogenic complex described herein. For example, a composition can include a population of one type of MAPS-GBS immunogenic complex, where all of the MAPS-GBS immunogenic complexes include the same antigenic polypeptide and the same antigenic polysaccharide. Additionally or alternatively, such compositions can include a plurality of more than one type of MAPS-GBS immunogenic complex described herein. For example, a composition can include populations of different types of MAPS-GBS immunogenic complexes. In some embodiments, a composition can include a population of a first type of MAPS-GBS immunogenic complex and a population of a second type of MAPS-GBS immunogenic complex, where the first type and the second type of the MAPS-GBS immunogenic complex have different antigenic polypeptides and/or different antigenic polysaccharides. In some embodiments, a composition can include a population of a first type of MAPS-GBS immunogenic complex and a population of a second type of immunogenic complex, where the first type and the second type of the MAPS-GBS immunogenic complex include the same antigenic polypeptide and different antigenic polysaccharides (e.g., polysaccharides of different serotypes). In some embodiments, MAPS-GBS immunogenic complexes described herein are formulated into a pharmaceutical composition. In some embodiments a pharmaceutical composition may be a vaccine. In some embodiments a pharmaceutical composition comprises a pharmaceutically acceptable carrier. In some embodiments a pharmaceutical composition comprises an adjuvant.

Aspects of the technology disclosed herein also provides immunogenic compositions (e.g., vaccine compositions) of, or comprising, one or more MAPS-GBS immunogenic complexes as described herein. In some embodiments, the immunogenic composition comprises one or more MAPS-GBS immunogenic complexes comprising at least one or more fusion proteins that have at least 80%, 85%, 90%, 95%, 96%, 97%, 98%, 99%, 99.5%, or 100% sequence identity to a fusion protein listed in Table 2A, 2B or Table 3. In some embodiments, the immunogenic composition comprises a fusion protein that is or includes an amino acid sequence having at least 80%, 85%, 90%, 95%, 97%, 98%, 99%, 99.5%, or 100% identity to any one of GBS fusion proteins selected from any of SEQ ID NOS: 3, 6, 7, 9, 10, 17, 18, 19, 20, 21, 22, 23, 24, 25, 26-36, 70-111 and 121-169.

Aspects of the technology disclosed herein also relate to immunogenic compositions (e.g., vaccine compositions) of, or comprising, one or more fusion proteins as described herein. In some embodiments, the immunogenic composition comprises one or more fusion proteins with at least 80%, 85%, 90%, 95%, 96%, 97%, 98%, 99%, 99.5%, or 100% sequence identity to a fusion protein listed in Table 2A, 2B or Table 3. In some embodiments, the immunogenic composition comprises a fusion protein that is or includes an amino acid sequence having at least 80%, 85%, 90%, 95%, 97%, 98%, 99%, 99.5%, or 100% identity to any one of GBS fusion proteins selected from any of SEQ ID NOS: 3, 6, 7, 9, 10, 17, 18, 19, 20, 21, 22, 23, 24, 25, 26-36, 70-111 and 121-169.

In some embodiments, an immunogenic composition may also comprise portions of fusion proteins described herein, for example internal deletion mutants, truncation mutants, and fragments. In some embodiments, the portions of said fusion proteins are immunogenic. The immunogenicity of a portion of a fusion protein is readily determined using the same assays that are used to determine immunogenicity of the full-length fusion protein. In some embodiments, the portion of the fusion protein has substantially the same immunogenicity as the full-length fusion protein. In some embodiments, the immunogenicity is no less than 10%, 20%, 30%, 40%, or 50% that of the fusion proteins of Table 2A, 2B or Table 3.

### Multivalent MAPS-GBS Vaccine Composition

For illustrative purposes only, referring to FIG. 1B, one aspect of the present invention relates to a composition comprising multiple species of MAPS-GBS immunogenic complexes. For example, in some embodiments, each species of MAPS-GBS immunogenic complex comprises; (i) at least a first biotinylated polysaccharide antigen (PS1) comprising at least one sialic acid domain and at least one biotin molecule, (ii) at least a second biotinylated polysaccharide antigen (PS2) comprising at least one sialic acid molecule and at least one biotin molecule, and (iii) at least one bifunctional SBD-[GBS-Ag]n-BBM fusion protein, comprising at least a biotin-biotin moiety (BBM) and a sialic acid binding domain (SBD), and wherein a BBM of at least one fusion protein non-covalently associates with at least one biotin molecule on the first biotinylated polysaccharide antigen (PS1) and the SBD non-covalently associates with at least one sialic acid on the second biotinylated polysaccharide antigen (PS2). If the MAPS-GBS immunogenic complex comprises more than one bifunctional SBD-[GBS-Ag]n-BBM fusion protein, e.g., a first fusion protein (SBD-BBM fusion1) and a second bifunctional fusion protein (e.g., SBD-BBM fusion 2), then the BBM of at least a first fusion protein (SBD-BBM fusion1) non-covalently associates with at least one biotin molecule on the first biotinylated polysaccharide antigen (PS1) and the SBD non-covalently associates with at least one sialic acid on the second biotinylated polysaccharide antigen (PS2), and the SBD of a second SBD-BBM fusion protein (SBD-BBM fusion2) non-covalently associates with at least one sialic acid molecule on the first biotinylated polysaccharide antigen (PS1) and the BBM of the second fusion protein non-covalently associates with at least biotin molecule on the second biotinylated polysaccharide antigen (PS2). This results in the PS1 and PS2 forming a GBS MAPS-complex by the non-covalent association via the first and second bifunctional SBD-BBM fusion proteins.

Accordingly, in some embodiments, a MAPS-GBS immunogenic complex can comprise the following non-covalent associations; PS1-(SBD-BBM fusion protein1)-PS2, where the BBM of the first SBD-BBM fusion protein non-covalently associates with a biotin on the first polysaccharide (PS1), and the SBD of the first SBD-BBM fusion protein non-covalently associates with a sialic acid on the second polysaccharide (PS2), to form an immunogenic complex.

In some embodiment, a MAPS-GBS immunogenic complex comprises at least two GBS fusion proteins, where at least one of these bifunctional fusion proteins is a SBD-[GBS-Ag]n-BBM fusion protein—that is, at least one of the SBD-BBM fusion proteins comprise a GBS polypeptide antigen as disclosed herein. Such a SBD-[GBS-Ag]n-BBM fusion protein can be selected from any fusion protein listed in Table 2A, 2B or Table 3 as disclosed herein. In some embodiments, a MAPS-GBS immunogenic complex can further comprise a GBS fusion protein that is not a bifunctional fusion protein (e.g., not SBD-BBM fusion protein or not a SBD-[GBS-Ag]n-BBM) (e.g., see FIG. 1D). Accordingly, in some embodiments, the second fusion protein in a MAPS GBS immunogenic complex can be selected from any of the biotin-binding moiety (BBM) fusion protein (e.g., BBM-[GBS-Ag]n fusion protein), or (ii) a sialic acid binding domain (SBD) fusion protein (e.g., SBD-[GBS-Ag]n fusion protein). Exemplary fusion proteins for use as a second fusion protein can be selected from any in the list disclosed in Table 2A or 2B or Table 3.

Exemplary configurations of MAPS-GBS immunogenic complexes are shown in Table 4.

TABLE 4
Exemplary MAPS-GBS immunogenic complexes can be as follows:

| | Exemplary MAPS-GBS | Non-covalent | |
|---|---|---|---|
| | immunogenic complex | associations | |
| 1 | [PS1-biotin]-[BBM- | BBM of the first SBD-BBM | Such a MAPS- |
| bifunctional | SBD1]-[s-PS2] | fusion protein ([BBM-SBD1]) | GBS complex |
| fusion | | non-covalently associates with a | can further |
| protein | | biotin on the first | comprise |
| | | polysaccharide (PS1), and the | additional non- |
| | | SBD of the first SBD-BBM | byfinctional |
| | | fusion protein non-covalently | fusion proteins, |
| | | associates with a sialic acid on | e.g., a BBM- |
| | | the second polysaccharide (PS2) | [GBS-Ag]n |
| | | | fusion protein |
| | | | or a SBD- |
| | | | [GBS-Ag]n |
| | | | fusion protein |
| 2 | [PS1-biotin]-[BBM- | BBM of the first SBD-BBM | [BBM-SBD1] |
| bifunctional | SBD1]-[s-PS2] | fusion protein ([BBM-SBD1]) | and [BBM- |
| fusion | [PS1-s]-[SBD-BBM2]- | non-covalently associates with a | SBD2] can be |
| protein | [b-PS2] | biotin on the first biotinylated | a SBD-[GBS- |
| | | polysaccharide (PS1), and the | Ag]n-BBM |
| | | SBD of the first SBD-BBM | fusion protein. |
| | | fusion protein non-covalently | They can be |
| | | associates with a sialic acid on | the same (e.g., |
| | | the second biotinylated | same species), |
| | | polysaccharide (PS2) | or different |
| | | SBD of the second SBD- | (e.g., the GBS |
| | | BBM fusion protein ([BBM- | antigen can be |
| | | SBD2]) non-covalently | different, or in |
| | | associates with a sialic acid on | a different |
| | | the first biotinylated | arrangement |
| | | polysaccharide (PS1), and the | etc.). If they |
| | | BBM of the second SBD-BBM | are different, |
| | | fusion protein non-covalently | one can be |
| | | associates with a biotin on the | SBD-BBM and |
| | | second biotinylated | one can be |
| | | polysaccharide (PS2). | SBD-[GBS- |
| | | | Ag]n-BBM |
| | | | fusion protein. |

In some embodiments, a MAPS-GBS immunogenic complex comprises a plurality of at least 1 species of SBD-BBM fusion proteins—that is, there are plurality of the same SBD-BBM fusion in the complex (e.g. see FIG. 1C(ii)). Such a MAPS-GBS can comprise at least 2, 3, 4, 5, 6, 7,8, 10, 11, 12, 13, 14, 15, 16-20, 21-30, 31-40, 41-50, 51-60, 61-70, 71-80, 81-90, 91-100 or more of the same species of bifunctional SBD-BBM fusion protein that can attach to a first and second polysaccharides. As the number of bifunctional SBD-BBM fusion increases, it can associate with more polysaccharides forming a lager MAPS-GBS complex. For example, referring to FIG. 1E and for simplicity and illustrative purposes only, if a MAPS-GBS complex comprises 5 polysaccharides, a first fusion protein (SBD-BBM fusion protein1) can associate with PS1 and PS2, and a second fusion protein (SBD-BBM fusion protein2) can associate with PS2 and PS3, and a third fusion protein (SBD-BBM fusion protein3) can associate with, e.g., PS3 and PS4, and a fourth fusion protein (SBD-BBM fusion protein4) can associate with, e.g., PS4 and PS5, etc. thereby forming a multi-bifunctional fusion protein and polysaccharide complex. In some embodiments, the additional SBD-BBM fusion proteins can also associate with polysaccharides already existing in the complex, for example, a SBD-BBM fusion protein5 can associate with PS3 and PS5, a SBD-BBM fusion protein6 can associate with PS1 and PS3, strengthening the MAPS-GBS complex. It is envisioned that each MAPS-GBS immunogenic complex comprises a plurality of biotinylated polysaccharides, e.g., at least 5, 10, 15, 20, 25, 30, 35, 40, 45, 50, 55, 60, 65, 75, 85, 90, 95, 100 or more than 100 polysaccharide molecules which are cross linked as using a bifunctional SBD-[GBS-Ag]-BBM fusion protein, or SBD-BBM fusion protein as disclosed herein.

In such an embodiment, the polysaccharides (e.g., PS1, PS2, PS3, PS4, PS5 etc.) can be the same species, e.g., from the same GBS serotype or different GBS serotypes. In some embodiments, the bifunctional SBD-BBM fusion protein can be the same species (e.g., comprise the same GBS antigen in the same configuration), or can be a different species (e.g., comprise the same GBS antigen in different arrangement, or alternatively, be a different GBS antigen).

For example, in some embodiments, all the polysaccharides (e.g., PS1, PS2, PS3, PS4, PS5 etc.) for a species of a MAPS-GBS immunogenic complex can be from a specific serotype of Group B Streptococcus (GBS) or Streptococcus agalactiae, e.g., selected from one of the GBS serotypes Ia, Ib, II, III, IV, V or VII of Streptococcus agalactiae. e.g., see FIG. 1E. In alternative embodiments, a MAPS-GBS immunogenic complex can comprise a plurality of different GBS polysaccharides, e.g., at least 2, 3, 4, 5, 6, 7, 8, 9 or 10 different types of GBS polysaccharides, e.g., selected from serotypes Ia, Ib, II, III, IV, V or VII of Streptococcus agalactiae.

In some embodiments, one species of MAPS-GBS immunogenic complex comprises a plurality of polysaccharides (e.g., PS1, PS2, PS3, PS4, PS5 etc.) that are the same species (e.g., are from the same GBS serotype), and a plurality of bifunctional SBD-BBM fusion proteins that are the same (e.g., comprise the same GBS antigen in the same configuration). In some embodiments, another species of a MAPS-GBS immunogenic complex comprises a plurality of polysaccharides (e.g., PS1, PS2, PS3, PS4, PS5 etc.) that are the same species, e.g., are from the same GBS serotype, and a plurality of bifunctional SBD-BBM fusion proteins that are different species, e.g., comprise the same GBS antigen in different arrangement, or alternatively, comprise one or more different GBS antigens.

In alternative embodiments, another species of a MAPS-GBS immunogenic complex comprises a plurality of polysaccharides (e.g., PS1, PS2, PS3, PS4, PS5 etc.) that are different species, e.g., are from different GBS serotypes, and a plurality of bifunctional SBD-BBM fusion proteins that are the same (e.g., comprise the same GBS antigen in the same configuration). In some embodiments, another species of a MAPS-GBS immunogenic complex comprises a plurality of polysaccharides (e.g., PS1, PS2, PS3, PS4, PS5 etc.) that are different species, e.g., are from more than one GBS serotype, and a plurality of bifunctional SBD-BBM fusion proteins that are different species, e.g., comprise the same GBS antigen in different arrangement, or alternatively, comprise one or more different GBS antigens.

In some embodiments, as disclosed herein, an immunogenic composition, e.g., vaccine composition as disclosed herein comprises multiple different species of MAPS-GBS immunogenic complexes as described herein, e.g., see FIG. 1F. In some embodiments, each MAPS-GBS immunogenic complex species can comprise a different biotinylated polysaccharide selected from any GBS serotypes from the group of: Ia, Ib, II, III, IV, V, VI, VII and VIII.

In some embodiments, each MAPS-GBS immunogenic complex within the composition can comprise the same species of SBD-[GBS-Ag]-BBM fusion protein, or different species within the complex (e.g., see FIGS. 1C(ii) and 1C(iii)) as compared to other species of MAPS-GBS immunogenic complex in the vaccine composition. Stated differently, there can be variability of the type of SBD-[GBS-Ag]-BBM fusion within a species of MAPS-GBS immunogenic complex in a vaccine, where each species of MAPS-GBS complex comprises a different GBS polysaccharide. Alternatively, in some embodiments, there can be variability of the polysaccharide within a species of MAPS-GBS immunogenic complex in a vaccine, where each species of MAPS-GBS complex comprises the same plurality of a SBD-[GBS-Ag]-BBM fusion proteins.

### Exemplary Multivalent Vaccine Composition Comprising Different MAPS-GBS Immunogenic Complexes.

Aspects of the technology described herein relate to a composition, which is a polyvalent immune composition, and comprises at least one species of MAPS-GBS immunogenic complex. That is, each MAPS-GBS species is distinct from the other MAPS-GBS species in the immunogenic complex. Without being limited to theory, for example, a composition can comprise, e.g., two MAPS-GBS immunogenic complexes as disclosed herein, where the first species of MAPS-GBS immunogenic complex disclosed herein comprises a PS1 and PS2 from Streptococcus agalactiae serotype Ia, and the second species of MAPS-GBS immunogenic complex can comprise a PS1 and PS2 from Streptococcus agalactiae serotype 1b. In such an embodiment, a composition would be considered a two valent (2V) MAPS-GBS immunogenic composition or vaccine. It is envisioned that a MAPS-GBS polyvalent immune composition as disclosed herein comprises MAPS-GBS immunogenic complexes that comprise polysaccharides from at least 2, or at least 3, or at least 4, or at least 5, or at least 6, or at least 7, or at least 8, or at least 9, or at least 10, or 2-5, or 5-6, or 6-7, or 7-8, or 8-9 or 9-10, or more than 10 different serotypes of GBS bacteria.

In some embodiments, as disclosed herein, the first biotinylated polysaccharide (PS1), or second biotinylated polysaccharide (PS2), or both, is from any of serotypes Ia, Ib, II, III, IV, V, VI, VII, VIII, and IX of Streptococcus agalactiae. In some embodiments, as disclosed herein, the first biotinylated polysaccharide, or second biotinylated polysaccharide, or both, is from any of serotypes Ia, Ib, II, III, IV, V or VII of Streptococcus agalactiae (7VMAPS-GBS). In some embodiments, as disclosed herein, the first biotinylated polysaccharide, or second biotinylated polysaccharide, or both, is from any of serotypes Ia, Ib, II, III, V or VII of Streptococcus agalactiae (6VMAPS-GBS).

Aspects of the technology described herein relate to multivalent GBS immunogenic compositions or vaccines comprising at least 2, 3, 4, 5, 6, 7 or 8 different MAPS-GBS immunogenic complex species, e.g., see FIG. 1F. In some embodiments, each species of MAPS-GBS immunogenic complex in the immunogenic composition comprises polysaccharides for a different GBS subtype. In some embodiments, the multivalent immune composition as disclosed herein comprises at least 4 species of MAPS-GBS immunogenic complexes, wherein the 4 species of MAPS-GBS immunogenic complexes are selected from any of:
a. a MAPS-GBS (type Ia) immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia or a different serotype selected from any of serotypes Ib, II, III, IV, V, VI, VII, VIII, and IX of Streptococcus agalactiae, and (c) at least one fusion protein, or
b. A MAPS-GBS (type Ib) immunogenic complex comprising (a) a first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib or a different serotype selected from any of serotypes Ia, II, III, IV, V, VI, VII, VIII, and IX of Streptococcus agalactiae, and (c) at least one fusion protein, or
c. an MAPS-GBS (type II) immunogenic complex comprising (a) a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II or a different serotype selected from any of serotypes Ia, Ib, III, IV, V, VI, VII, VIII, and IX of Streptococcus agalactiae, and (c) at least one fusion protein, or
d. an MAPS-GBS (type III) immunogenic complex comprising (a) a first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III or a different serotype selected from any of serotypes Ia, Ib, II, IV, V, VI, VII, VIII, and IX of Streptococcus agalactiae, and (c) at least one fusion protein, or
e. an MAPS-GBS (type IV) immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV or a different serotype selected from any of serotypes Ia, Ib, II, III, V, VI, VII, VIII, and IX of Streptococcus agalactiae, and (c) at least one fusion protein, or
f. an MAPS-GBS (type V) immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V or a different serotype selected from any of serotypes Ia, Ib, II, III, IV, VI, VII, VIII, and IX of Streptococcus agalactiae, and (c) at least one fusion protein, or
g. an MAPS-GBS (type VI) immunogenic complex comprising (a) a first biotinylated polysaccharide antigen comprising at least biotin, one sialic acid and a first polysaccharide antigen from Streptococcus agalactiae serotype VI, and (b) a second biotinylated polysaccharide antigen comprising at least biotin and at least one sialic acid, wherein the second polysaccharide antigen is from Streptococcus agalactiae serotype VI or a different serotype selected from any of serotypes Ia, Ib, II, III, IV, V, VII, VIII, and IX of Streptococcus agalactiae, and (c) at least one fusion protein, or
h. an MAPS-GBS (type VII) immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII or a different serotype selected from any of serotypes Ia, Ib, II, III, IV, V, VI, VIII, and IX of Streptococcus agalactiae, and (c) at least one fusion protein, and
i. an MAPS-GBS (type VIII) immunogenic complex comprising (a) a first biotinylated polysaccharide antigen comprising at least biotin, one sialic acid and a first polysaccharide antigen from Streptococcus agalactiae serotype VIII, and (b) a second biotinylated polysaccharide antigen comprising at least biotin and at least one sialic acid, wherein the second polysaccharide antigen is from Streptococcus agalactiae serotype VIII or a different serotype selected from any of serotypes Ia, Ib, II, III, IV, V, VI, VII, and IX of Streptococcus agalactiae, and (c) at least one fusion protein.

In some embodiments, the polyvalent immune composition as disclosed herein comprises at least 6 species of MAPS-GBS immunogenic complexes (6V-MAPS-GBS), wherein the six species of immunogenic complex are as follows:
i. A first MAPS-GBS immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia or a different serotype selected from any of serotypes Ib, II, III, IV, V, VI, VII, VIII, and IX of Streptococcus agalactiae, and (c) at least one fusion protein,
ii. a second MAPS-GBS immunogenic complex comprising (a) first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib or a different serotype selected from any of serotypes Ia, II, III, IV, V, VI, VII, VIII, and IX of Streptococcus agalactiae, and (c) at least one fusion protein, and (b) at least one fusion protein,
iii. a third MAPS-GBS immunogenic complex comprising a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II or a different serotype selected from any of serotypes Ia, Ib, III, IV, V, VI, VII, VIII, and IX of Streptococcus agalactiae, and (c) at least one fusion protein,
iv. a fourth MAPS-GBS immunogenic complex comprising (a) first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III or a different serotype selected from any of serotypes Ia, Ib, II, IV, V, VI, VII, VIII, and IX of Streptococcus agalactiae, and (c) at least one fusion protein,
v. a firth MAPS-GBS immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V or a different serotype selected from any of serotypes Ia, Ib, II, III, IV, VI, VII, VIII, and IX of Streptococcus agalactiae, and (c) at least one fusion protein, and
vi. a sixth MAPS-GBS immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII or a different serotype selected from any of serotypes Ia, Ib, II, III, IV, V, VI, VIII, and IX of Streptococcus agalactiae, and (c) at least one fusion protein.

In some embodiments, the immunogenic composition or vaccine can further comprises an additional species of MAPS-GBS immunogenic complex to produce 7V-MAPS-GBS vaccine, where the composition further comprises: a seventh MAPS-GBS immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV or a different serotype selected from any of serotypes Ia, Ib, II, III, V, VI, VII, VIII, and IX of Streptococcus agalactiae, and (c) at least one fusion protein, and (b) at least one fusion protein.

In some embodiments, an immunogenic composition (e.g., a vaccine composition) contains one or more fusion proteins described herein, which are not part of a MAPS-GBS complex, in combination with one or more polypeptides from Table 1, or antigenic fragments or variants thereof, in a mixture. In some embodiments, the mixture contains both full-length polypeptides and fragments resulting from processing, or partial processing, of signal sequences by an expression host, e.g. E. coli, an insect cell line (e.g., the baculovirus expression system), or a mammalian cell line (e.g., human or Chinese Hamster Ovary).

In some embodiments, an immunogenic composition contains one or more fusion proteins of any of GBS fusion proteins selected from any of SEQ ID NOS: 3, 6, 7, 9, 10, 17, 18, 19, 20, 21, 22, 23, 24, 25, 26-36, 70-111 and 121-169 in the absence of any other antigens.

In some embodiments, fusion proteins described herein may be non-covalently associated with Streptococcus agalactiae polysaccharides, by biotin to biotin-binding protein interaction, e.g., biotin to rhizavidin protein interaction, and/or by SBD interaction with sialic acid on the GBS polysaccharide, as disclosed herein. In some embodiments, the polysaccharide is a purified PS Streptococcus agalactiae, as disclosed herein in the Examples. In some embodiments, the polysaccharide is a purified lipidated PS Streptococcus agalactiae oligosaccharide.

In some embodiments, a GBS fusion protein as described herein, or disclosed in Table 3 is covalently bound to another molecule. This may, for example, increase the half-life, solubility, bioavailability, or immunogenicity of the fusion protein. Molecules that may be covalently bound to the fusion protein include a carbohydrate, biotin, poly(ethylene glycol) (PEG), polysialic acid, N-propionylated polysialic acid, nucleic acids, polysaccharides, and PLGA. There are many different types of PEG, ranging from molecular weights of below 300 g/mol to over 10,000,000 g/mol. PEG chains can be linear, branched, or with comb or star geometries. In some embodiments, the fusion protein is covalently bound to a moeity that stimulates the immune system. An example of such a moeity is a lipid moeity. In some instances, lipid moieties are recognized by a Toll-like receptor (TLR) such as TLR-2 or TLR-4, and activate the innate immune system.

### VII. Uses of Fusion Proteins

In some embodiments, a GBS fusion protein described herein does not have, or has minimal, hemolytic activity. For example, in some embodiments, the hemolytic activity of a fusion protein described herein can be established by turbidimetry (OD₄₂₀) after incubation of the fusion protein at different dilutions with red blood cells (e.g., sheep erythrocytes), to determine the protein concentration at which 50% of the red blood cells are lysed. In some such embodiments, the hemolytic activity of a fusion protein described herein can be characterized by an OD₄₂₀ of less than 0.4 or lower, including, e.g., less than 0.3, less than 0.25, less than 0.2, or lower, for a given protein concentration.

In some embodiments, polypeptides of Streptococcus agalactiae and fusion proteins described herein, and fragments and variants thereof, are immunogenic. These polypeptides and fusion proteins may be immunogenic in mammals, for example mice, rats, guinea pigs, or humans. An antigenic polypeptide or fusion protein is typically one capable of raising a significant immune response in an assay or in a subject. The immune response may be innate, humoral, cell-mediated, or mucosal (combining elements of innate, humoral and cell-mediated immunity). In some embodiments, an antigenic polypeptide or fusion protein elicits production of a detectable amount of antibody specific to that antigen.

In some embodiments, a fusion protein described herein is an antigen or has antigenic properties. In some embodiments, a fusion protein described herein is a carrier protein or has carrier properties. In some embodiments, a fusion protein described herein is both an antigen and a carrier protein. In some embodiments, a fusion protein described herein has both carrier properties and antigenic properties.

In some embodiments, a GBS fusion protein described herein is an antigen of a MAPS-GBS immunogenic complex (e.g., a Multiple Antigen Presenting System (MAPS) complex) which is a modified immunogenic complex previously described in WO 2012/155007, the entire contents of which are incorporated herein by reference for the purposes indicated herein). In some embodiments, a fusion protein described herein is a carrier protein of an immunogenic complex. In some embodiments, a fusion protein described herein is both a carrier protein and an antigen of an immunogenic complex.

In some embodiments, polypeptides of the fusion proteins described herein have less than 20%, 30%, 40%, 50%, 60% or 70% identity to human auto-antigens and/or gut commensal bacteria (e.g., certain Bacteroides, Clostridium, Fusobacterium, Eubacterium, Ruminococcus, Peptococcus, Peptostreptococcus, Bifidobacterium, Escherichia, and Lactobacillus species). Examples of human autoantigens include insulin, proliferating cell nuclear antigen, cytochrome P450, and myelin basic protein.

A polypeptide included in a fusion protein described herein may comprise one or more immunogenic portions and one or more non-immunogenic portions. The immunogenic portions may be identified by various methods, including protein microarrays, ELISPOT/ELISA techniques, and/or specific assays on different deletion mutants (e.g., fragments) of the polypeptide in question. Immunogenic portions may also be identified by computer algorithms. Some such algorithms, like EpiMatrix (produced by EpiVax), use a computational matrix approach. Other computational tools for identifying antigenic epitopes include PEPVAC (Promiscuous EPitope-based VACcine, hosted by Dana Farber Cancer Institute on the world wide web at immunax.dfci.harvard.edu/PEPVAC), MHCPred (which uses a partial least squares approach and is hosted by The Jenner Institute on the world wide web at jenner. ac.uk/MHCPred), and Immune Epitope Database algorithms on the World Wide Web at tools.immuneepitope.org. An antigenic fragment of a polypeptide described herein comprises at least one immunogenic portion, as measured experimentally or identified by algorithm (for example, the SYFPEITHI algorithm found at syfpeithi.de).

### VIII. Uses of Immunogenic and Vaccine Compositions

In some embodiments, an immunogenic composition or vaccine that includes one or more fusion proteins described herein is characterized in that one or more of the opsonization potential or immune responses to one or more fusion proteins is increased relative to a pre-determined level, as measured by ELISA and/or by a functional antibody assay. In some embodiments, one or more of the opsonization potential or immune response to the one or more fusion proteins is increased by at least 30% or more, including, e.g., at least 40%, at least 50%, at least 60%, at least 70%, at least 80%, at least 90% or more, relative to a predetermined level, as measured by ELISA and/or by a functional antibody assay. In some embodiments, one or more of the opsonization potential or immune responses to the one or more fusion proteins is increased at least 1-fold, 2-fold, 3-fold, 4-fold, or 5-fold relative to a pre-determined level, as measured by ELISA and/or by a functional antibody assay. In some embodiments, the pre-determined level is a pre-immune level (e.g., a level observed when a subject is not immunized, or is immunized in the absence of one or more fusion proteins described herein).

In some embodiments, an immunogenic composition or vaccine that includes one or more fusion proteins described herein, upon administration to a subject, induces an immune response against Streptococcus agalactiae. In some embodiments, the immunogenic composition or vaccine, upon administration to a subject, induces an immune response against one or more serotypes of Streptococcus agalactiae. In some embodiments, the immunogenic composition or vaccine, upon administration to a subject, induces a protective immune response against one or more serotypes of Streptococcus agalactiae. In some embodiments, the immune response is an antibody or B cell response. In some embodiments, the immune response is a T cell response. In some embodiments, the immune response is an innate immune response. In some embodiments, the immune response is a CD4+ T cell response, including Th1, Th2, or Th17 response, or a CD8+ T cell response, or a CD4+ and a CD8+ T cell response, or a CD4−/CD8− T cell response. In some embodiments, the immune response is an antibody or B cell response and a T cell response. In some embodiments, the immune response is an antibody or B cell response, a T cell response, and an innate immune response. In some embodiments, the immune response is a protective immune response.

In some embodiments, the immune response is to the GBS polysaccharide. In some embodiments, the immune response is to the GBS antigenic polypeptide (also referred to as a carrier protein), e.g., to any one or more of antigenic polypeptides Rib, Sip, AlpC, Alp1, Alp3, Alp3/1 in the immunogenic composition. In some embodiments, there is an immune response is to the GBS polysaccharide and to the GBS antigenic polypeptide (also referred to as a carrier protein), e.g., to any one or more of antigenic polypeptides Rib, Sip, AlpC, Alp1, Alp3, Alp3/1 in the fusion protein that is in the MAPS-GBS immunogenic composition.

In some embodiments, an immunogenic complex described herein, upon administration to a subject, induces antibody production against one or more pathogens in the subject at a level greater than a composition comprising an antigenic polysaccharide alone. In some embodiments, an immunogenic complex described herein, upon administration to a subject, induces antibody production against one or more pathogens in the subject at level greater than a composition comprising a polypeptide antigen alone.

In some embodiments, an immunogenic composition or vaccine described herein, upon administration to a subject, induces an immune response against one or more pathogens in the subject at a level greater than a composition comprising an antigenic polysaccharide alone. In some embodiments, an immunogenic composition or vaccine described herein, upon administration to a subject, induces an immune response against one or more pathogens in the subject at a level greater than a composition comprising a polypeptide antigen alone. In some embodiments, an immunogenic complex described herein, upon administration to a subject, induces a protective immune response.

In some embodiments, a MAPS-GBS immunogenic complex described herein that includes one or more antigenic polysaccharides is characterized in that one or more of the opsonization potential, or immune response to one or more antigenic polysaccharides is increased relative to a predetermined level, as measured by ELISA and or by a functional antibody assay. In some embodiments, one or more of the opsonization potential, immune response to the one or more antigenic polysaccharides is increased at least 1-fold, 2-fold, 3-fold, 4-fold, or 5-fold relative to a predetermined level, as measured by ELISA and or by a functional antibody assay. In some embodiments, the predetermined level is a pre-immune level. In some embodiments, the predetermined level is a pre-immune level. In some embodiments, one or more polypeptide antigens are carrier proteins for one or more antigenic polysaccharides.

In some embodiments, a MAPS-GBS immunogenic complex described herein, upon administration to a subject, induces an immune response against one or more pathogens in the subject at a level greater than a composition comprising an antigenic polysaccharide alone. In some embodiments, an immunogenic complex described herein, upon administration to a subject, induces an immune response against one or more pathogens in the subject at a level greater than a composition comprising a polypeptide antigen alone. In some embodiments, an immunogenic complex described herein, upon administration to a subject, induces a protective immune response.

In some embodiments, an immunogenic complex described herein, upon administration to a subject, induces an immune response against Streptococcus agalactiae. In some embodiments, a MAPS-GBS immunogenic complex described herein, upon administration to a subject, induces an immune response against one or more serotypes of Streptococcus agalactiae. In some embodiments, such an immune response may be directed against one or more (e.g., 1, 2, 3, 4, 5, 6, 7, 8, 9, 10 or more) serotypes of Streptococcus agalactiae, wherein an immunogenic complex described herein includes polysaccharide(s) present in at least one or more (e.g., 1, 2, 3, 4, 5, 6, 7, 8, 9, 10 or more) of such serotype(s). In some embodiments, such an immune response may be directed against one or more (e.g., 1, 2, 3, 4, 5, 6, 7, 8, 9, 10 or more) serotypes of Streptococcus agalactiae, wherein an immunogenic complex described herein does not include polysaccharide(s) present in at least one or more (e.g., 1, 2, 3, 4, 5, 6, 7, 8, 9, 10 or more) of such serotype(s). In some embodiments, such an immune response may be directed against two or more (e.g., 2, 3, 4, 5, 6, 7, 8, 9, 10 or more) serotypes of Streptococcus agalactiae, wherein an immunogenic complex described herein (i) includes polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s). In some embodiments, an immunogenic complex described herein, upon administration to a subject, induces a protective immune response against one or more serotypes of Streptococcus agalactiae. In some embodiments, such a protective response may be directed against one or more (e.g., 1, 2, 3, 4, 5, 6, 7, 8, 9, 10 or more) serotypes of Streptococcus agalactiae, wherein an immunogenic complex described herein includes polysaccharide(s) present in at least one or more (e.g., 1, 2, 3, 4, 5, 6, 7, 8, 9, 10 or more) of such serotype(s). In some embodiments, such a protective response may be directed against one or more (e.g., 1, 2, 3, 4, 5, 6, 7, 8, 9, 10 or more) serotypes of Streptococcus agalactiae, wherein an immunogenic complex described herein does not include polysaccharide(s) present in at least one or more (e.g., 1, 2, 3, 4, 5, 6, 7, 8, 9, 10 or more) of such serotype(s). In some embodiments, such a protective response may be directed against two or more (e.g., 2, 3, 4, 5, 6, 7, 8, 9, 10 or more) serotypes of Streptococcus agalactiae, wherein an immunogenic complex described herein (i) includes polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s).

The immunogenic compositions and vaccines comprising MAPS-GBS immunogenic complexes as described herein may be used for prophylactic and/or therapeutic treatment of Streptococcus agalactiae. Accordingly, this application provides a method for immunizing a subject suffering from or susceptible to GBS infection, comprising administering an immunologically effective amount of any of the immunogenic compositions or vaccine formulations described herein. The subject receiving the vaccination may be a male or a female, and may be an infant, child, adolescent, or adult. In some embodiments, the subject being treated is a human. In other embodiments, the subject is a non-human animal. In some embodiments, an immunogenic complex described herein, upon administration to a subject, induces a protective immune response against one or more serotypes of Streptococcus agalactiae.

In prophylactic embodiments, a vaccine composition (e.g., ones as described and/or utilized herein) is administered to a subject to induce an immune response that can help protect against the establishment of Streptococcus agalactiae, for example by protecting against colonization, the first and necessary step in disease. In some embodiments, such an immune response may be directed against one or more (e.g., 1, 2, 3, 4, 5, 6, 7, 8, 9, 10 or more) serotypes of Streptococcus agalactiae, wherein a vaccine composition described herein includes polysaccharide(s) present in at least one or more (e.g., 1, 2, 3, 4, 5, 6, 7, 8, 9, 10 or more) of such serotype(s). In some embodiments, such an immune response may be directed against one or more (e.g., 1, 2, 3, 4, 5, 6, 7, 8, 9, 10 or more) serotypes of Streptococcus agalactiae, wherein a vaccine composition described herein does not include polysaccharide(s) present in at least one or more (e.g., 1, 2, 3, 4, 5, 6, 7, 8, 9, 10 or more) of such serotype(s) (non-vaccine types, NVTs). In some embodiments, such an immune response may be directed against two or more (e.g., 2, 3, 4, 5, 6, 7, 8, 9, 10 or more) serotypes of Streptococcus agalactiae, wherein a vaccine composition described herein (i) comprises a plurality of species of MAPS-GBS immunogenic complexes comprising polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s). Thus, in some aspects, the method inhibits infection by Streptococcus agalactiae in a noncolonized or uninfected subject. In another aspect, the method may reduce the duration of colonization in a subject who is already colonized.

In therapeutic embodiments, the vaccine may be administered to a subject suffering from Streptococcus agalactiae infection, in an amount sufficient to treat the subject. Treating the subject, in this case, refers to reducing Streptococcus agalactiae symptoms and/or bacterial load and/or sequelae in an infected subject. In some embodiments, treating the subject refers to reducing the duration of symptoms or sequelae, or reducing the intensity of symptoms or sequelae. In some embodiments, the vaccine reduces transmissibility of Streptococcus agalactiae from the vaccinated subject. In certain embodiments, the reductions described above are at least 25%, 30%, 40%, 50%, 60%, 70%, 80%, or 90%.

In therapeutic embodiments, the vaccine is administered to a subject postinfection. The vaccine may be administered shortly after infection, e.g. before symptoms or sequelae manifest, or may be administered during or after manifestation of symptoms or sequelae.

In some embodiments, an immunogenic composition and/or vaccine compositions as disclosed herein comprising a plurality of MAPS-GBS immunogenic complexes confer protective immunity, allowing a vaccinated subject to exhibit delayed onset of symptoms or sequelae, or reduced severity of symptoms or sequelae, as the result of his or her exposure to the vaccine. In certain embodiments, the reduction in severity of symptoms or sequelae is at least 25%, 40%, 50%, 60%, 70%, 80%, or 90%. In particular embodiments, vaccinated subjects may display no symptoms or sequelae upon contact with Streptococcus agalactiae, do not become colonized by Streptococcus agalactiae, or both. Protective immunity is typically achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes, that result in sIGA production by B lymphocytes on mucosa-lined tissues of the body. Humoral immunity is typically the result of IgG antibodies and IgM antibodies in serum. Cellular immunity can be achieved through cytotoxic T lymphocytes or through delayed-type hypersensitivity that involves macrophages and T lymphocytes, as well as other mechanisms involving T cells without a requirement for antibodies. In particular, cellular immunity may be mediated by Th1 or Th17 cells.

In some embodiments, an immunogenic composition or vaccine described herein, upon administration to a subject, induces an immune response against Streptococcus agalactiae. In some embodiments, an immunogenic composition or vaccine described herein, upon administration to a subject, induces an immune response against one or more (e.g., 1, 2, 3, 4, 5, 6, 7, 8, 9, 10, or more) serotypes of Streptococcus agalactiae. In some embodiments, an immunogenic composition or vaccine described herein, upon administration to a subject, induces an immune response against all serotypes of Streptococcus agalactiae comprised in such immunogenic composition or vaccine. In some embodiments, an immunogenic complex described herein, upon administration to a subject, induces a protective immune response against one or more (e.g., 1,2,3,4, 5, 6, 7, 8, 9, 10, or more) serotypes of Streptococcus agalactiae. In some embodiments, an immunogenic complex described herein, upon administration to a subject, induces a protective immune response against all serotypes of Streptococcus agalactiae comprised in such immunogenic composition or vaccine.

In some embodiments, the immune response is an antibody or B cell response. In some embodiments, the immune response is a T cell response. In some embodiments, the immune response is an innate immune response. In some embodiments, the immune response is a CD4+ T cell response, including Th1, Th2, or Th17 response, or a CD8+ T cell response, or a CD4+ and CD8+ T cell response, or CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response.

In some embodiments, an immunogenic composition or vaccine described herein, upon administration to a subject, induces an antibody or B cell response against one or more pathogens in the subject at a level greater than a composition comprising an antigenic polysaccharide alone. In some embodiments, an immunogenic composition or vaccine described herein, upon administration to a subject, induces an antibody or B cell response against one or more pathogens in the subject at level greater than a composition comprising a polypeptide antigen alone. In some embodiments, the immune response is a protective immune response.

In some embodiments, an immunogenic composition or vaccine described herein, upon administration to a subject, induces a T cell response against one or more pathogens in the subject at a level greater than a composition comprising an antigenic polysaccharide alone. In some embodiments, an immunogenic composition or vaccine described herein, upon administration to a subject, induces a T cell response against one or more pathogens in the subject at level greater than a composition comprising a polypeptide antigen alone. In some embodiments, the immune response is a protective immune response.

In some embodiments, upon administration to a subject, an immunogenic composition or vaccine described herein treats or prevents infection by Streptococcus agalactiae. In some embodiments, upon administration to a subject, an immunogenic composition or vaccine described herein inhibits or reduces the rate of occurrence of infection by Streptococcus agalactiae. In some embodiments, upon administration to a subject, an immunogenic composition or vaccine described herein reduces the severity of infection by Streptococcus agalactiae. In some embodiments, upon administration to a subject, an immunogenic composition or vaccine described herein inhibits transmission of Streptococcus agalactiae from the subject to another subject.

In some embodiments, an immunogenic composition or vaccine that includes one or more MAPS-GBS immunogenic complexes and/or fusion proteins described herein may be used for prophylactic and/or therapeutic treatment of Streptococcus agalactiae. Accordingly, the present disclosure provides a method for immunizing a subject suffering from or susceptible to Streptococcus agalactiae infection, comprising administering an immunologically effective amount of any immunogenic composition or vaccine that includes one or more MAPS-GBS immunogenic complexes as disclosed herein and comprising one or more fusion proteins as described herein. The subject receiving the immunization may be a male or a female, and may be an infant, child, adolescent, or adult. In some embodiments, the subject being treated is a human. In other embodiments, the subject is a non-human animal.

In some embodiments, upon administration to a subject, an immunogenic composition or vaccine comprising one or more MAPS-GBS immunogenic complexes as disclosed herein and comprising one or more fusion proteins as described herein treats or prevents infection by Streptococcus agalactiae. In some embodiments, upon administration to a subject, the immunogenic composition or vaccine treats or prevents Invasive Streptococcus agalactiae Disease (IPD) due to infection by Streptococcus agalactiae. In some embodiments, upon administration to a subject, the immunogenic composition or vaccine treats or prevents bacteremia due to infection by Streptococcus agalactiae. In some embodiments, upon administration to a subject, the immunogenic composition or vaccine treats or prevents any of: sepsis, pneumonia, and meningitis due to infection by Streptococcus agalactiae. In some embodiments, upon administration to a subject, the immunogenic composition or vaccine treats or prevents organ damage due to infection by Streptococcus agalactiae. In some embodiments, upon administration to a subject, the immunogenic composition or vaccine treats or prevents meningitis due to infection by Streptococcus agalactiae. In some embodiments, upon administration to a subject, the immunogenic composition or vaccine treats or prevents pneumonia due to infection by Streptococcus agalactiae. In some embodiments, upon administration to a subject, the immunogenic composition or vaccine treats or prevents otitis media due to infection by Streptococcus agalactiae. In some embodiments, upon administration to a subject, the immunogenic composition or vaccine treats or prevents sinusitis due to infection by Streptococcus agalactiae.

In some embodiments, upon administration to a female pregnant subject, the immunogenic composition or vaccine treats or prevents any of: sepsis, pneumonia, and meningitis in the baby in utero or post partum, due to infection by Streptococcus agalactiae.

In some embodiments, upon administration to a subject, an immunogenic composition or vaccine comprising a MAPS-GBS immunogenic complex and/or fusion protein as described herein inhibits or reduces the rate of occurrence of infection of a human baby by Streptococcus agalactiae. In some embodiments, upon administration to a subject, including pregnant females, babies and the elderly, the immunogenic composition or vaccine inhibits or reduces the rate of occurrence of Invasive Streptococcus agalactiae Disease (IPD) due to infection by Streptococcus agalactiae. In some embodiments, upon administration to a subject, the immunogenic composition or vaccine inhibits or reduces the rate of occurrence of bacteremia due to infection by Streptococcus agalactiae. In some embodiments, upon administration to a subject, the immunogenic composition or vaccine inhibits or reduces the rate of occurrence of sepsis due to infection by Streptococcus agalactiae. In some embodiments, upon administration to a subject, the immunogenic composition or vaccine inhibits or reduces the rate of occurrence of organ damage due to infection by Streptococcus agalactiae. In some embodiments, upon administration to a subject, the immunogenic composition or vaccine inhibits or reduces the rate of occurrence of meningitis due to infection by Streptococcus agalactiae. In some embodiments, upon administration to a subject, the immunogenic composition or vaccine inhibits or reduces the rate of occurrence of pneumonia due to infection by Streptococcus agalactiae. In some embodiments, upon administration to a subject, the immunogenic composition or vaccine inhibits or reduces the rate of occurrence of otitis media due to infection by Streptococcus agalactiae. In some embodiments, upon administration to a subject, the immunogenic composition or vaccine inhibits or reduces the rate of occurrence of sinusitis due to infection by Streptococcus agalactiae.

In some embodiments, upon administration to a subject, an immunogenic composition or vaccine comprising a MAPS-GBS immunogenic complex and/or fusion protein described herein reduces the severity of infection by Streptococcus agalactiae. In some embodiments, upon administration to a subject, the immunogenic composition or vaccine reduces the severity of Invasive Streptococcus agalactiae Disease (IPD) due to infection by Streptococcus agalactiae. In some embodiments, upon administration to a subject, the immunogenic composition or vaccine reduces the severity of bacteremia due to infection by Streptococcus agalactiae. In some embodiments, upon administration to a subject, the immunogenic composition or vaccine reduces the severity of sepsis due to infection by Streptococcus agalactiae. In some embodiments, upon administration to a subject, the immunogenic composition or vaccine reduces the severity of organ damage due to infection by Streptococcus agalactiae. In some embodiments, upon administration to a subject, the immunogenic composition or vaccine reduces the severity of meningitis due to infection by Streptococcus agalactiae. In some embodiments, upon administration to a subject, the immunogenic composition or vaccine reduces the severity of pneumonia due to infection by Streptococcus agalactiae. In some embodiments, upon administration to a subject, the immunogenic composition or vaccine reduces the severity of otitis media due to infection by Streptococcus agalactiae. In some embodiments, upon administration to a subject, the immunogenic composition or vaccine reduces the severity of sinusitis due to infection by Streptococcus agalactiae.

In some embodiments, upon administration to a subject, an immunogenic composition or vaccine comprising a MAPS-GBS immunogenic complex and/or fusion protein described herein inhibits transmission of Streptococcus agalactiae from the subject to another subject. In some embodiments, upon administration to a subject, the immunogenic composition or vaccine inhibits colonization by Streptococcus agalactiae in the subject. In some embodiments, upon administration to a subject, the immunogenic composition or vaccine inhibits colonization by Streptococcus agalactiae in the nasopharynx of the subject.

In some embodiments, an immunogenic composition or vaccine comprising a MAPS-GBS immunogenic complex comprising one or more fusion proteins as described herein, upon administration to a subject, induces an immune response against Streptococcus agalactiae in the subject at a level greater than a control composition. In some embodiments, the immunogenic composition or vaccine, upon administration to a subject, induces an immune response against one or more serotypes of Streptococcus agalactiae at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition or vaccine comprising a MAPS-GBS immunogenic complex and/or fusion protein described herein, upon administration to a subject, induces an immune response that can help protect against the establishment of Streptococcus agalactiae at a level greater than a control composition. In some embodiments, the immunogenic composition or vaccine protects against colonization at a level greater than a control composition. In some embodiments, the immunogenic composition or vaccine inhibits infection by Streptococcus agalactiae in a non-colonized or uninfected subject at a level greater than a control composition. In some embodiments, the immunogenic composition or vaccine reduces the duration of colonization by Streptococcus agalactiae in a subject who is already colonized at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, a control composition is GBS vaccine, including polysaccharide-protein conjugates that have covalent linkages (e.g., a non-MAPS GBS vaccine), e.g., as disclosed in WO2016178123A1 or a MAPS vaccine comprising a GBS-fusion protein, where the MAPS vaccine does not comprise a bifunctional SBD-BBM fusion protein.

### IX. Antibody Compositions

Some embodiments provide for an antibody composition comprising antibodies raised in a mammal immunized with an immunogenic composition or vaccine comprising either a MAPS-GBS immunogenic complex and/or a fusion protein described herein. In some embodiments, an antibody comprises at least one antibody selected from the group consisting of monoclonal Abs (mAbs) and anti-idiotype antibodies. In some embodiments, an antibody composition comprises an isolated gamma globulin fraction. In some embodiments, an antibody composition comprises polyclonal antibodies. In some embodiments, the antibody composition is administered to a subject. In some embodiments, the antibody composition administered to a subject confers passive immunization.

### X. Vaccine Formulations

In some embodiments, a vaccine composition is a polyvalent or multivalent MAPS-GBS vaccine. In some embodiments, the valency of a vaccine composition refers to the number of species of immunogenic complexes present in the vaccine composition. The valency of a vaccine described herein is not limiting with respect to the total antigens present in said pharmaceutical composition, immunogenic complex, or vaccine, or to the number of pathogen strains for which administration of said pharmaceutical composition, immunogenic complex, immunogenic composition, or vaccine composition may induce an immune-protective response. In a non-limiting example, a 6-valent vaccine composition may comprise more than 6 antigenic components (e.g., peptide and/or polysaccharide components) and may induce an immunoprotective response against more than 6 pathogens, or pathogenic serotypes or strains.

In some embodiments, a vaccine composition comprises between 1-50 species of immunogenic complexes. In some embodiments, a vaccine composition comprises between 1-40 species of immunogenic complexes. In some embodiments, a vaccine composition comprises between 1-35 species of immunogenic complexes. In some embodiments, a vaccine composition comprises between 1-30 species of immunogenic complexes. In some embodiments, a vaccine composition comprises between 1-30 species of immunogenic complexes. In some embodiments, a vaccine composition comprises between 1-24 species of immunogenic complexes. In some embodiments, a vaccine composition comprises between 1-15 species of immunogenic complexes. In some embodiments, a vaccine composition comprises between 1-9 species of immunogenic complexes. In some embodiments, a vaccine composition comprises between 1-5 species of immunogenic complexes. In some embodiments, a vaccine is a polyvalent vaccine.

In some embodiments, a vaccine composition comprises two or more species of MAPS-GBS immunogenic complexes (e.g., in immunogenic compositions) in amounts such that the weight of polysaccharides in the vaccine composition from each MAPS-GBS immunogenic complex is about the same, e.g., present in a w/w ratio of about 1:1. In some embodiments, the weight of polysaccharides in the vaccine contributed by each MAPS-GBS immunogenic complex is about 0.20 ug. In some embodiments, the weight of polysaccharides in the vaccine contributed by each MAPS-GBS immunogenic complex is about 0.25 ug. In some embodiments, the weight of polysaccharides in the vaccine contributed by each MAPS-GBS immunogenic complex is about 0.5 ug. In some embodiments, the weight of polysaccharides in the vaccine contributed by each MAPS-GBS immunogenic complex is about 1 ug. In some embodiments, the weight of polysaccharides in the vaccine contributed by each MAPS-GBS immunogenic complex is about 1.5 ug. In some embodiments, the weight of polysaccharides in the vaccine contributed by each MAPS-GBS immunogenic complex is about 2 ug. In some embodiments, the weight of polysaccharides in the vaccine contributed by each MAPS-GBS immunogenic complex is about 2.5 ug. In some embodiments, the weight of polysaccharides in the vaccine contributed by each MAPS-GBS immunogenic complex is about 3 ug. In some embodiments, the weight of polysaccharides in the vaccine contributed by each MAPS-GBS immunogenic complex is about 3.5 ug. In some embodiments, the weight of polysaccharides in the vaccine contributed by each MAPS-GBS immunogenic complex is about 4 ug. In some embodiments, the weight of polysaccharides in the vaccine contributed by each MAPS-GBS immunogenic complex is about 4.5 ug. In some embodiments, the weight of polysaccharides in the vaccine contributed by each MAPS-GBS immunogenic complex is about 5 ug. In some embodiments, the weight of polysaccharides in the vaccine contributed by each MAPS-GBS immunogenic complex is about 5.5 ug. In some embodiments, the weight of polysaccharides in the vaccine contributed by each MAPS-GBS immunogenic complex is about 6 ug. In some embodiments, the weight of polysaccharides in the vaccine contributed by each MAPS-GBS immunogenic complex is about 7 ug. In some embodiments, the weight of polysaccharides in the vaccine contributed by each MAPS-GBS immunogenic complex is about 8 ug. In some embodiments, the weight of polysaccharides in the vaccine contributed by each MAPS-GBS immunogenic complex is about 9 ug. In some embodiments, the weight of polysaccharides in the vaccine contributed by each MAPS-GBS immunogenic complex is about 10 ug. In some embodiments, the weight of polysaccharides in the vaccine contributed by each MAPS-GBS immunogenic complex is about 11 ug. In some embodiments, the weight of polysaccharides in the vaccine contributed by each MAPS-GBS immunogenic complex is about 12 ug. In some embodiments, the weight of polysaccharides in the vaccine contributed by each MAPS-GBS immunogenic complex is more than 12 ug, e.g., 13 ug, 14 ug, 15 ug, 16 ug, 17 ug, 18 ug, 19 ug, 20 ug, 21 ug, 22 ug, 23 ug, 24 ug, 25 ug, or more.

In some embodiments, a vaccine composition comprises two or more species of immunogenic complexes (e.g., in immunogenic compositions) in amounts such that the weight of polysaccharides in the vaccine composition contributed by each MAPS-GBS immunogenic complex is different, e.g., present in a w/w ratio that is not about 1:1. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:2. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:3. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:4. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:5. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:6. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:7. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:8. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:9. In some embodiments, a vaccine composition comprises two or more species of immunogenic complexes (e.g., in immunogenic compositions) in amounts such that the weight of polysaccharide in the vaccine composition contributed by a first MAPS-GBS immunogenic complex and a second MAPS-GBS immunogenic complex is 1:10. In some embodiments, the vaccine composition comprises a mixture of immunogenic complexes, such that the weight of polysaccharide in a vaccine contributed by an MAPS-GBS immunogenic complex ranges from about 0.20 ug to about 6 ug. In some embodiments, the vaccine composition comprises a mixture of immunogenic complexes, such that the weight of polysaccharide in a vaccine contributed by an MAPS-GBS immunogenic complex ranges from about 0.20 ug to about 12 ug. In some embodiments, the vaccine composition comprises a mixture of immunogenic complexes, such that the weight of polysaccharides in the vaccine contributed by each MAPS-GBS immunogenic complex ranges from about 0.20 ug to about 20 ug. In some embodiments, the vaccine composition comprises a mixture of immunogenic complexes, such that the weight of polysaccharides in the vaccine contributed by each MAPS-GBS immunogenic complex ranges from about 0.20 ug to about 40 ug.

In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is about the same, e.g., present in a w/w protein:PS ratio of about 1:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 2:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 3:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 4:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 5:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 6:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 7:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 8:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 9:1. In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex (e.g., in an immunogenic composition) is present in a w/w protein:PS ratio of about 10:1.

In some embodiments, the combined weight of polysaccharides and polypeptides in the vaccine contributed by each MAPS-GBS immunogenic complex is about 0.20 ug. In some embodiments, the combined weight of polysaccharides and polypeptides in the vaccine contributed by each MAPS-GBS immunogenic complex is about 0.40 ug. In some embodiments, the combined weight of polysaccharides and polypeptides in the vaccine contributed by each MAPS-GBS immunogenic complex is about 1 ug. In some embodiments, the combined weight of polysaccharides and polypeptides in the vaccine contributed by each MAPS-GBS immunogenic complex is about 2 ug. In some embodiments, the combined weight of polysaccharides and polypeptides in the vaccine contributed by each immunogenic composition is about 3 ug. In some embodiments, the combined weight of polysaccharides and polypeptides in the vaccine contributed by each immunogenic composition is about 4 ug. In some embodiments, the combined weight of polysaccharides and polypeptides in the vaccine contributed by each immunogenic composition is about 5 ug. In some embodiments, the combined weight of polysaccharides and polypeptides in the vaccine contributed by each immunogenic composition is about 6 ug. In some embodiments, the combined weight of polysaccharides and polypeptides in the vaccine contributed by each immunogenic composition is about 7 ug. In some embodiments, the combined weight of polysaccharides and polypeptides in the vaccine contributed by each immunogenic composition is about 8 ug. In some embodiments, the combined weight of polysaccharides and polypeptides in the vaccine contributed by each immunogenic composition is about 9 ug. In some embodiments, the combined weight of polysaccharides and polypeptides in the vaccine contributed by each immunogenic composition is about 10 ug. In some embodiments, the combined weight of polysaccharides and polypeptides in the vaccine contributed by each immunogenic composition is about 11 ug. In some embodiments, the combined weight of polysaccharides and polypeptides in the vaccine contributed by each immunogenic composition is about 12 ug. In some embodiments, the combined weight of polysaccharides and polypeptides in the vaccine contributed by each immunogenic composition is about 14 ug. In some embodiments, the combined weight of polysaccharides and polypeptides in the vaccine contributed by each immunogenic composition is about 16 ug. In some embodiments, the combined weight of polysaccharides and polypeptides in the vaccine contributed by each immunogenic composition is about 18 ug. In some embodiments, the combined weight of polysaccharides and polypeptides in the vaccine contributed by each immunogenic composition is about 20 ug. In some embodiments, the combined weight of polysaccharides and polypeptides in the vaccine contributed by each immunogenic composition is about 21 ug. In some embodiments, the combined weight of polysaccharides and polypeptides in the vaccine contributed by each immunogenic composition is about 22 ug. In some embodiments, the combined weight of polysaccharides and polypeptides in the vaccine contributed by each immunogenic composition is about 23 ug. In some embodiments, the combined weight of polysaccharides and polypeptides in the vaccine contributed by each immunogenic composition is about 24 ug. In some embodiments, the combined weight of polysaccharides and polypeptides in the vaccine contributed by each immunogenic composition is about 25 ug. In some embodiments, the combined weight of polysaccharides and polypeptides in the vaccine contributed by each immunogenic composition is about 30 ug. In some embodiments, the combined weight of polysaccharides and polypeptides in the vaccine contributed by each immunogenic composition is about 40 ug. In some embodiments, the combined weight of polysaccharides and polypeptides in the vaccine contributed by each immunogenic composition is about 50 ug. In some embodiments, the combined weight of polysaccharides and polypeptides in the vaccine contributed by each immunogenic composition is about 60 ug. In some embodiments, the combined weight of polysaccharides and polypeptides in the vaccine contributed by each immunogenic composition is about 70 ug. In some embodiments, the combined weight of polysaccharides and polypeptides in the vaccine contributed by each immunogenic composition is about 80 ug. In some embodiments, the combined weight of polysaccharides and polypeptides in the vaccine contributed by each immunogenic composition is about 90 ug. In some embodiments, the combined weight of polysaccharides and polypeptides in the vaccine contributed by each immunogenic composition is about 100 ug. In some embodiments, the combined weight of polysaccharides and polypeptides in the vaccine contributed by each immunogenic composition is about 110 ug.

In some embodiments, a vaccine composition comprises two or more species of immunogenic complexes (e.g., in immunogenic compositions) in amounts such that the combined weight of polysaccharides and polypeptides in the vaccine composition contributed by each MAPS-GBS immunogenic complex is different, e.g., present in a w/w protein:PS ratio that is not about 1:1, e.g., a protein:PS ratio that is 2:1, 3:1, 4:1, 5:1, 6:1, 7:1, 8:1, 9:1, or 10:1. In some embodiments, the vaccine composition comprises a mixture of immunogenic complexes, such that the combined weight of polysaccharides and polypeptides in the vaccine contributed by each MAPS-GBS immunogenic complex ranges from about 0.4 ug to about 110 ug.

Optimal amounts of components for a particular MAPS-GBS vaccine comprising a plurality of MAPS-GBS immunogenic complexes, and/or fusion proteins as described herein can be ascertained by standard studies involving observation of appropriate immune responses in subjects. Following an initial immunization, subjects can receive one or several booster immunizations adequately spaced in time.

The immunogenic composition or vaccine comprising a plurality of MAPS-GBS immunogenic complexes, and/or fusion proteins as described herein, and/or preparations thereof, may be formulated in a unit dosage form for ease of administration and uniformity of dosage. The specific therapeutically effective dose level for any particular subject or organism may depend upon a variety of factors including the severity or degree of risk of infection; the activity of the specific vaccine or vaccine composition employed; other characteristics of the specific vaccine or vaccine composition employed; the age, body weight, general health, sex of the subject, diet of the subject, pharmacokinetic condition of the subject, the time of administration (e.g., with regard to other activities of the subject such as eating, sleeping, receiving other medicines including other vaccine doses, etc.), route of administration, rate of excretion of the specific vaccine or vaccine composition employed; vaccines used in combination or coincidental with the vaccine composition employed; and like factors well known in the medical arts.

An immunogenic composition or vaccine comprising a plurality of MAPS-GBS immunogenic complexes and/or fusion protein as described herein for use in accordance with the present disclosure may be formulated into compositions (e.g., pharmaceutical compositions) according to known techniques. Vaccine preparation is generally described in Vaccine Design (Powell and Newman, 1995). For example, an immunologically amount of a vaccine product can be formulated together with one or more organic or inorganic, liquid or solid, pharmaceutically suitable carrier materials. Preparation of pneumococcal polysaccharide and conjugate vaccines is described, for example, in U.S. Ser. No. 11/395,593, filed Mar. 31, 2006, the contents of which are incorporated herein by reference.

In general, pharmaceutically acceptable carrier(s) include solvents, dispersion media, and the like, which are compatible with pharmaceutical administration. For example, materials that can serve as pharmaceutically acceptable carriers include, but are not limited to sugars such as lactose, glucose, dextrose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; polyols such as glycerol, propylene glycol, and liquid polyethylene glycol; esters such as ethyl oleate and ethyl laurate; agar; buffering agents such as magnesium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as preservatives, and antioxidants can also be present in the composition, according to the judgment of the formulator (Martin, 1975).

Vaccines may be formulated by combining one or more fusion proteins described herein with carriers and/or other optional components by any available means including, for example, conventional mixing, granulating, dissolving, lyophilizing, or similar processes.

Vaccines comprising one or more fusion proteins described herein may be lyophilized up until they are about to be used, at which point they are extemporaneously reconstituted with diluent. In some embodiments, vaccine components or compositions are lyophilized in the presence of one or more other components (e.g., adjuvants), and are extemporaneously reconstituted with saline solution. Alternatively, individual components, or sets of components may be separately lyophilized and/or stored (e.g., in a vaccination kit), the components being reconstituted and either mixed prior to use or administered separately to the subject.

Lyophilization can produce a more stable composition (for instance by preventing or reducing breakdown of polysaccharide antigens). Lyophilizing of vaccines or vaccine components is well known in the art. Typically, a liquid vaccine or vaccine component is freeze dried, often in the presence of an anti-caking agent (such as, for example, sugars such as sucrose or lactose). In some embodiments, the anti-caking agent is present, for example, at an initial concentration of 10-200 mg/ml. Lyophilization typically occurs over a series of steps, for instance a cycle starting at −69° C., gradually adjusting to −24° C. over 3 h, then retaining this temperature for 18 h, then gradually adjusting to −16° C. over 1 h, then retaining this temperature for 6 h, then gradually adjusting to +34° C. over 3 h, and finally retaining this temperature over 9 h.

In some embodiments, a vaccine comprising a MAPS-GBS immunogenic complex and/or fusion protein described herein is a liquid. In some embodiments the liquid is a reconstituted lyophilate. In some embodiments a vaccine has a pH of about 5, about 6, about 7, or about 8. In some embodiments a vaccine has a pH between about 5 and about 7.5. In some embodiments a vaccine has a pH between 5 and 7.5. In some embodiments a vaccine has a pH between about 5.3 and about 6.3. In some embodiments a vaccine has a pH between 5.3 and 6.3. In some embodiments a vaccine has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

Vaccines or vaccine components for use in accordance with the present disclosure may be incorporated into liposomes, cochleates, biodegradable polymers such as poly-lactide, poly-glycolide and poly-lactide-co-glycolides, or immune-stimulating complexes (ISCOMS).

In certain situations, it may be desirable to prolong the effect or release of a vaccine for use in accordance with the present invention, for example, by slowing the absorption of one or more vaccine components. Such delay of absorption may be accomplished, for example, by the use of a liquid suspension of crystalline or amorphous material with poor water solubility. The rate of absorption of the product then depends upon its rate of dissolution, which in turn, may depend upon size and form. Alternatively, or additionally, delayed absorption may be accomplished by dissolving or suspending one or more vaccine components in an oil vehicle. Injectable depot forms can also be employed to delay absorption. Such depot forms can be prepared by forming microcapsule matrices of one or more vaccine components a biodegradable polymer network. Depending upon the ratio of polymer to vaccine component, and the nature of the particular polymer(s) employed, the rate of release can be controlled.

Examples of biodegradable polymers that can be employed in accordance with the present disclosure include, for example, poly(orthoesters) and poly(anhydrides). One particular exemplary polymer is polylactide-polyglycolide.

Depot injectable formulations may also be prepared by entrapping the product in liposomes or microemulsions, which are compatible with body tissues.

Polymeric delivery systems can also be employed in non-depot formulations including, for example, oral formulations. For example, biodegradable, biocompatible polymers such as ethylene vinyl acetate, polyanhydrides, polyglycolic acid, collagen, polyorthoesters, and polylactic acid, etc., can be used in oral formulations. Polysaccharide antigens or conjugates may be formulated with such polymers, for example to prepare particles, microparticles, extrudates, solid dispersions, admixtures, or other combinations in order to facilitate preparation of useful formulations (e.g., oral).

Vaccines comprising one or more fusion proteins described herein for use in accordance with the present disclosure include immunogenic compositions, and may additionally include one or more additional active agents (i.e., agents that exert a biological effect—not inert ingredients). For example, it is common in vaccine preparation to include one or more adjuvants. It will be appreciated that such additional agents may be formulated together with one or more other vaccine components, or may be maintained separately and combined at or near the time of administration. In some embodiments, such additional components may be administered separately from some or all of the other vaccine components, within an appropriate time window for the relevant effect to be achieved.

### (a) Adjuvants

The vaccine formulations and immunogenic compositions comprising a MAPS-GBS immunogenic complex and/or a fusion protein as described herein may include an adjuvant. Adjuvants, generally, are agents that enhance the immune response to an antigen. Adjuvants can be broadly separated into two classes, based on their principal mechanisms of action: vaccine delivery systems and immunostimulatory adjuvants (see, e.g., Singh et al, 2003). In most vaccine formulations, the adjuvant provides a signal to the immune system so that it generates a response to the antigen, and the antigen is required for driving the specificity of the response to the pathogen. Vaccine delivery systems are often particulate formulations, e.g., emulsions, microparticles, immune-stimulating complexes (ISCOMs), nanoparticles, which may be, for example, particles and/or matrices, and liposomes. In contrast, immunostimulatory adjuvants are sometimes from or derived from pathogens and can represent pathogen associated molecular patterns (PAMP), e.g., lipopolysaccharides (LPS), monophosphoryl lipid A (MPL), or CpG-containing DNA, which activate cells of the innate immune system.

Alternatively, adjuvants may be classified as organic and inorganic. Inorganic adjuvants include alum salts such as aluminum phosphate, amorphous aluminum hydroxyphosphate sulfate, and aluminum hydroxide, which are commonly used in human vaccines. Organic adjuvants comprise organic molecules including macromolecules. Non-limiting examples of organic adjuvants include cholera toxin/toxoids, other enterotoxins/toxoids or labile toxins/toxoids of Gram-negative bacteria, interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), and tumor necrosis factor (TNF).

In some embodiments, the vaccine composition as disclosed herein comprises an AddaSO3 adjuvant. In some embodiments, the vaccine composition as disclosed herein comprises a R848 adjuvant. In some embodiments, the vaccine composition as disclosed herein comprises an ODN2395 adjuvant. In some embodiments, the vaccine composition as disclosed herein comprises an Alum phos adjuvant. In some embodiments, the vaccine composition as disclosed herein comprises an AddaSO3 alum adjuvant. In some embodiments, the vaccine composition as disclosed herein comprises a R848 alum adjuvant. In some embodiments, the vaccine composition as disclosed herein comprises an ODN alum adjuvant. In some embodiments, the vaccine composition as disclosed herein comprises an Alum 2PE adjuvant.

In some embodiments, the pharmaceutical composition as disclosed herein comprises an AddaSO3 adjuvant. In some embodiments, the pharmaceutical composition as disclosed herein comprises a R848 adjuvant. In some embodiments, the pharmaceutical composition as disclosed herein comprises an ODN2395 adjuvant. In some embodiments, the pharmaceutical composition as disclosed herein comprises an Alum phos adjuvant. In some embodiments, the pharmaceutical composition as disclosed herein comprises an AddaSO3 alum adjuvant. In some embodiments, the pharmaceutical composition as disclosed herein comprises a R848 alum adjuvant. In some embodiments, the pharmaceutical composition as disclosed herein comprises an ODN alum adjuvant. In some embodiments, the pharmaceutical composition as disclosed herein comprises an Alum 2PE adjuvant.

In some embodiments, the immunogenic composition as disclosed herein comprises an AddaSO3 adjuvant. In some embodiments, the immunogenic composition as disclosed herein comprises a R848 adjuvant. In some embodiments, the immunogenic composition as disclosed herein comprises an ODN2395 adjuvant. In some embodiments, the immunogenic composition as disclosed herein comprises an Alum phos adjuvant. In some embodiments, the immunogenic composition as disclosed herein comprises an AddaSO3 alum adjuvant. In some embodiments, the immunogenic composition as disclosed herein comprises a R848 alum adjuvant. In some embodiments, the immunogenic composition as disclosed herein comprises an ODN alum adjuvant. In some embodiments, the immunogenic composition as disclosed herein comprises an Alum 2PE adjuvant.

In some embodiments, the adjuvant can comprise of at least one of AddaSO3, R848, ODN2395, Alum phosphate, AddaSO3 alum, R848 alum, ODN alum, or Alum 2PE, at least two of AddaSO3, R848, ODN2395, Alum phosphate, AddaSO3 alum, R848 alum, ODN alum, or Alum 2PE, at least three of AddaSO3, R848, ODN2395, Alum phosphate, AddaSO3 alum, R848 alum, ODN alum, or Alum 2PE, at least four of AddaSO3, R848, ODN2395, Alum phosphate, AddaSO3 alum, R848 alum, ODN alum, or Alum 2PE, at least five of AddaSO3, R848, ODN2395, Alum phosphate, AddaSO3 alum, R848 alum, ODN alum, or Alum 2PE, at least six of AddaSO3, R848, ODN2395, Alum phosphate, AddaSO3 alum, R848 alum, ODN alum, or Alum 2PE, at least seven of AddaSO3, R848, ODN2395, Alum phosphate, AddaSO3 alum, R848 alum, ODN alum, or Alum 2PE, or at least eight of AddaSO3, R848, ODN2395, Alum phosphate, AddaSO3 alum, R848 alum, ODN alum, or Alum 2PE.

Adjuvants may also be classified by the response they induce. In some embodiments, the adjuvant induces the generation, proliferation, or activation of Th1 cells or Th2 cells. In other embodiments, the adjuvant induces the generation, proliferation, or activation of B cells. In yet other embodiments, the adjuvant induces the activation of antigen-presenting cells. These categories are not mutually exclusive; in some cases, an adjuvant activates more than one type of cell.

In some embodiments, the adjuvant induces the generation, proliferation, or activation of Th17 cells. The adjuvant may promote the CD4+ or CD8+ T cells to secrete IL-17. In some embodiments, an adjuvant that induces the generation, proliferation, or activation of Th17 cells is one that produces at least a 2-fold, and in some cases a 10-fold, experimental sample to control ratio in the following assay. In the assay, an experimenter compares the IL-17 levels secreted by two populations of cells: (1) cells from animals immunized with the adjuvant and a polypeptide known to induce Th17 generation, proliferation, or activation, and (2) cells from animals treated with the adjuvant and an irrelevant (control) polypeptide. An adjuvant that induces the generation, proliferation, or activation of Th17 cells may cause the cells of population (1) to produce more than 2-fold, or more than 10-fold more IL-17 than the cells of population (2). IL-17 may be measured, for example, by ELISA or ELISPOT. Certain toxins, such as cholera toxin and labile toxin (produced by enterotoxigenic E. coli, or ETEC), activate a Th17 response. Thus, in some embodiments, the adjuvant is a toxin or toxoid. Cholera toxin was successfully used in a mouse model to induce protective immunity in conjunction with certain polypeptides from Table 1. One form of labile toxin is produced by Intercell. Mutant derivates of labile toxin (toxoids) that are active as adjuvants but significantly less toxic can be used as well. Exemplary detoxified mutant derivatives of labile toxin include mutants lacking ADP-ribosyltransferase activity. Particular detoxified mutant derivatives of labile toxin include LTK7 (Douce et al, 1995) and LTK63 (Williams et al, 2004), LT-G192 (Douce et al, 1999), and LTR72 (Giuliani et al, 1998).

In some embodiments, the adjuvant comprises a VLP (virus-like particle). One such adjuvant platform, Alphavirus replicons, induces the activation of Th17 cells using alphavirus and is produced by Alphavax. In some embodiments of the Alphavirus replicon system, alphavirus may be engineered to express an antigen of interest, a cytokine of interest (for example, IL-17 or a cytokine that stimulates IL-17 production), or both, and may be produced in a helper cell line. More detailed information may be found in U.S. Pat. Nos. 5,643,576 and 6,783,939. In some embodiments, a vaccine formulation is administered to a subject in combination with a nucleic acid encoding a cytokine.

Certain classes of adjuvants activate toll-like receptors (TLRs) in order to activate a Th17 response. TLRs are well known proteins that may be found on leukocyte membranes, and recognize foreign antigens (including microbial antigens). Administering a known TLR ligand together with an antigen of interest (for instance, as a fusion protein) can promote the development of an immune response specific to the antigen of interest. One exemplary adjuvant that activates TLRs comprises Monophosphoryl Lipid A (MPL). Traditionally, MPL has been produced as a detoxified lipopolysaccharide (LPS) endotoxin obtained from Gram-negative bacteria, such as S. minnesota. In particular, sequential acid and base hydrolysis of LPS produces an immunoactive lipid A fraction (which is MPL), and lacks the saccharide groups and all but one of the phosphates present in LPS. A number of synthetic TLR agonists (in particular, TLR-4 agonists) are disclosed in Evans et al, 2003. Like MPL adjuvants, these synthetic compounds activate the innate immune system via TLR. Another type of TLR agonist is a synthetic phospholipid dimer, for example E6020 (Ishizaka et al, 2007). Various TLR agonists (including TLR-4 agonists) have been produced and/or sold by, for example, the Infectious Disease Research Institute (IRDI), Corixa, Esai, Avanti Polar Lipids, Inc., and Sigma Aldrich. Another exemplary adjuvant that activates TLRs comprises a mixture of MPL, Trehalose Dicoynomycolate (TDM), and dioctadecyldimethylammonium bromide (DDA). Another TLR-activating adjuvant is R848 (resiquimod).

In some embodiments, the adjuvant is or comprises a saponin. Typically, the saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C.

In some embodiments, combinations of adjuvants are used. Three exemplary combinations of adjuvants are MPL and alum, E6020 and alum, and MPL and an ISCOM.

Adjuvants may be covalently or non-covalently bound to antigens. In some embodiments, the adjuvant may comprise a protein which induces inflammatory responses through activation of antigen-presenting cells (APCs). In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to T cells and initiation of T cell responses (e.g., see Wu et al, 2005).

In some embodiments, an immunogenic composition or vaccine comprising a plurality of MAPS-GBS immunogenic complexes and/or fusion protein as described herein is formulated and/or administered in combination with an adjuvant. In some embodiments, the adjuvant is selected from the group consisting of aluminum phosphate, aluminum hydroxide, and phosphate aluminum hydroxide. In some embodiments, the adjuvant comprises aluminum phosphate. In some embodiments, the adjuvant is aluminum phosphate.

Typically, the same adjuvant or mixture of adjuvants is present in each dose of a vaccine. Optionally, however, an adjuvant may be administered with the first dose of vaccine and not with subsequent doses (i.e., booster shots). Alternatively, a strong adjuvant may be administered with the first dose of vaccine and a weaker adjuvant or lower dose of the strong adjuvant may be administered with subsequent doses. The adjuvant can be administered before the administration of the antigen, concurrent with the administration of the antigen or after the administration of the antigen to a subject (sometimes within 1, 2, 6, or 12 hours, and sometimes within 1, 2, or 5 days). Certain adjuvants are appropriate for human subjects, non-human animals, or both.

Vaccines for use in accordance with the present disclosure may include, or be administered concurrently with, antimicrobial therapy. For example, such vaccines may include or be administered with one or more agents that kills or retards growth of a pathogen. Such agents include, for example, penicillin, vancomycin, erythromycin, azithromycin, and clarithromycin, cefotaxime, ceftriaxone, levoflaxin, gatifloxacin.

Alternatively or additionally, vaccines for use in accordance with the present invention may include, or be administered with, one or more other vaccines or therapies. For example, one or more non-Streptococcus agalactiae antigens may be included in or administered with the vaccines.

### (b) Additional Components and Excipients

In addition to the fusion proteins described herein and the adjuvants described above, a vaccine formulation or immunogenic composition may include one or more additional components.

In some embodiments, the vaccine formulation comprises aluminum phosphate (referred to herein as alum phosphate, or AP). In some embodiments, a vaccine formulation comprising GBS-MAPS aluminum phosphate (referred to herein as alum phosphate, or AP). In some embodiments, the amount of alum phosphate is determined by one of ordinary skill in the art. In some embodiments, the amount of atomic aluminum (in the form of aluminum phosphate) is 250 μg per 500 μl injection (25 μg polysaccharide). In some embodiments, a vaccine formulation or immunogenic composition comprises 250 μg of alum phosphate per 500 μl injection. In some embodiments, the alum phosphate is in a buffer comprising 20 mM Tris, pH 7, 150 mM NaCl.

In some embodiments, the vaccine formulation or immunogenic composition may include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In some embodiments, the vaccine formulation or immunogenic composition includes one or more buffers such as a mixture of sodium bicarbonate and ascorbic acid. In some embodiments, the vaccine formulation may be administered in saline, such as phosphate buffered saline (PBS), or distilled water.

In some embodiments, the vaccine formulation or immunogenic composition includes one or more surfactants, for example, but not limited to, polysorbate 80 (TWEEN 80), polysorbate 20 (TWEEN 20), Polyethylene glycol p-(1,1,3,3-tetramethylbutyl)-phenyl ether (TRITON X-100), and 4-(1,1,3,3-Tetramethylbutyl)phenol polymer with formaldehyde and oxirane (TYLOXAPOL). A surfactant can be ionic or nonionic.

In some embodiments, the vaccine formulation or immunogenic composition includes one or more salts such as sodium chloride, ammonium chloride, calcium chloride, or potassium chloride.

In some embodiments, a preservative is included in the vaccine or immunogenic composition. In other embodiments, no preservative is used. A preservative is most often used in multi-dose vaccine vials, and is less often needed in single-dose vaccine vials. In some embodiments, the preservative is 2-phenoxyethanol, methyl and propyl parabens, benzyl alcohol, and/or sorbic acid.

### XI. Methods of Administration

In some embodiments, an immunogenic composition or vaccine comprising a plurality of MAPS-GBS immunogenic complexes and/or fusion protein as described herein is administered to a subject at risk of developing Streptococcus agalactiae disease, e.g. an infant, a toddler, a juvenile, or an older adult. In some embodiments, the immunogenic composition or vaccine is administered to a subject at elevated risk of developing Streptococcus agalactiae disease, e.g., immunocompromised subjects, subjects having sickle cell disease or other hemoglobinopathies, congenital or acquired asplenia, splenic dysfunction, chronic renal failure or nephrotic syndrome, diseases associated with treatment with immunosuppressive drugs or radiation therapy, including malignant neoplasm, leukemia, lymphomas, Hodgkin's disease, or solid organ transplantation, congenital or acquired immunodeficiency, HIV infection, cerebrospinal fluid leaks, cochlear implant(s), chronic heart disease, chronic lung disease, diabetes mellitus, alcoholism, chronic liver disease, cigarette smoking, asthma, generalized malignancy, multiple myeloma, or solid organ transplantation. It will be appreciated that a subject can be considered at risk for developing a disease without having been diagnosed with any symptoms of the disease. For example, if the subject is known to have been, or to be intended to be, in situations with relatively high risk of infection, that subject will be considered at risk for developing the disease.

Any effective route of administration may be utilized such as, for example, oral, nasal, enteral, parenteral, intramuscular or intravenous, subcutaneous, transdermal, intradermal, rectal, vaginal, topical, ocular, pulmonary, or by contact application. In some embodiments, the immunogenic composition or vaccine may be injected (e.g., via intramuscular, intraperitoneal, intradermal and/or subcutaneous routes); or delivered via the mucosa (e.g., to the oral/alimentary, respiratory, and/or genitourinary tracts). Intranasal administration may be particularly useful in some contexts. In some embodiments, it may be desirable to administer different doses of the immunogenic composition or vaccine by different routes; in some embodiments, it may be desirable to administer different components of one dose via different routes.

In some embodiments, pharmaceutical compositions (e.g., immunogenic compositions or vaccines) are administered intradermally. Conventional technique of intradermal injection, the “Mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced while providing a slight pressure to elevate it under the skin. The liquid is then injected very slowly thereby forming a bleb or bump on the skin surface, followed by slow withdrawal of the needle.

Devices that are specifically designed to administer liquid agents into or across the skin have been described, for example the devices described in WO 99/34850 and EP 1092444, also the jet injection devices described for example in WO 01/13977; U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 97/37705 and WO 97/13537. Other methods of intradermal administration of the immunogenic compositions or vaccines may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

As described above, pharmaceutical compositions (e.g., immunogenic compositions or vaccines) may be administered as a single dose or as multiple doses. It will be appreciated that an administration is a single “dose” so long as all relevant components are administered to a subject within a window of time; it is not necessary that every component be present in a single composition. For example, administration of two different immunogenic compositions or vaccines, within a period of less than 24 h, is considered a single dose. To give but one example, immunogenic compositions or vaccines having different antigenic components may be administered in separate compositions, but as part of a single dose. As noted above, such separate compositions may be administered via different routes or via the same route. Alternatively or additionally, in embodiments wherein an immunogenic composition or vaccine is combined with additional types of active agents, the immunogenic composition or vaccine may be administered via one route, and a second active agent may be administered by the same route or by a different route.

Pharmaceutical compositions (e.g., immunogenic compositions or vaccines) are administered in such amounts and for such time as is necessary to achieve a desired result. In some embodiments of the present invention, the immunogenic composition or vaccine comprises an immunologically effective amount of at least immunogenic composition. The exact amount required to achieve an immunologically effective amount may vary, depending on the immunogenic composition, and from subject to subject, depending on the species, age, and general condition of the subject, the stage of the disease, the particular pharmaceutical mixture, its mode of administration, and the like.

The amount of a MAPS-GBS immunogenic complex and/or a fusion protein as described herein in each pharmaceutical composition (e.g., immunogenic composition or vaccine) dose is selected to allow the vaccine, when administered as described herein, to induce an appropriate immunoprotective response without significant adverse side effects.

In some embodiments, a pharmaceutical composition comprising a MAPS-GBS immunogenic complex and/or a fusion protein as described herein induces a Th1 and/or Th17 cell response upon administration to a subject. In some embodiments, the pharmaceutical composition induces an opsonic/bactericidal response against Streptococcus agalactiae upon administration to a subject. In some embodiments, the pharmaceutical composition comprising a MAPS-GBS immunogenic complex and/or a fusion protein disclosed herein reduces rate of transmission and/or colonization of the mucosal surfaces by Streptococcus pneumoniae upon administration to a subject. In some embodiments, the pharmaceutical composition reduces rate of transmission and/or colonization of the nasopharynx or the lungs by Streptococcus agalactiae upon transmission.

Some embodiments provide for a method of immunizing a subject against Streptococcus agalactiae infection comprising administering to the subject an immunologically effective amount of an immunogenic composition comprising a MAPS-GBS immunogenic complex and/or a fusion protein described herein. Some embodiments provide for a method of immunizing a subject against Streptococcus agalactiae infection comprising administering to the subject an immunologically effective amount of a vaccine composition comprising a fusion protein described herein. Some embodiments provide for a method of immunizing a subject against Streptococcus agalactiae infection comprising administering to the subject an immunologically effective amount of a pharmaceutical composition comprising a fusion protein described herein.

### (a) Combination Prophylaxis or Combination Therapy

In some embodiments, an immunogenic composition or vaccine comprising a plurality of MAPS-GBS immunogenic complexes and/or fusion protein as described herein may be administered in combination with another agent. In some embodiments, the agent is or comprises PCV13. In some embodiments, the agent is or comprises PPSV23. In some embodiments, the agent is or comprises an antibiotic.

### (b) Dosing

In some embodiments, administration of an immunogenic composition or vaccine comprising a plurality of MAPS-GBS immunogenic complexes and/or fusion protein as described herein may involve the delivery of a single dose. In some embodiments, administration may involve an initial dose followed by one or several additional immunization doses, adequately spaced. An immunization schedule is a program for the administration of one or more specified doses of one or more specified MAPS-GBS vaccines, by one or more specified routes of administration, at one or more specified ages of a subject.

In some embodiments, administration of a vaccine (e.g., a vaccine composition) described herein may involve the delivery of a single dose. In some embodiments, administration may involve an initial dose followed by one or several additional immunization doses, adequately spaced. Such additional immunization doses can be referred to as boosters. In some embodiments, a booster (or second or subsequent) immunization dose is administered 2 weeks, or 3 weeks, or about 1 month, or about 2 months, or about 6 months or about 1 year after the preceding dose (where the proceeding dose can be initial dose or a second or third dose, or booster dose).

The present disclosure provides immunization methods that involve administering at least one dose of a vaccine to an infant subject. In some embodiments, the infant subject is 18 months old or younger. In some embodiments, the infant subject is 12 months old or younger. In some embodiments, the infant subject has previously received one or more doses of a conjugated Streptococcus agalactiae polysaccharide vaccine; in other embodiments, the infant subject is naïve to MAPS-GBS vaccines. In some embodiments, the infant subject has previously been infected with, or exposed to infection by Streptococcus agalactiae.

The present disclosure provides immunization methods that involve administering at least one dose of a vaccine to a toddler subject. In some embodiments, the toddler subject is 5 years old or younger. In some embodiments, the toddler subject is 4 years old or younger. In some embodiments, the toddler subject has previously received one or more doses of a conjugated Streptococcus agalactiae polysaccharide vaccine; in other embodiments, the toddler subject is naïve to MAPS-GBS vaccines. In some embodiments, the toddler subject has previously been infected with, or exposed to infection by Streptococcus agalactiae.

The present disclosure provides immunization methods that involve administering at least one dose of a vaccine to a juvenile subject. In some embodiments, the juvenile subject is 18 years old or younger. In some embodiments, the juvenile subject is 15 years old or younger. In some embodiments, the juvenile subject has previously received one or more doses of a conjugated Streptococcus agalactiae polysaccharide vaccine; in other embodiments, the juvenile subject is naïve to MAPS-GBS vaccines. In some embodiments, the juvenile subject has previously been infected with, or exposed to infection by Streptococcus agalactiae.

The present disclosure provides immunization methods that involve administering at least one dose of a vaccine to an adult subject. In some embodiments, the adult subject is older than about 50 years of age. In some embodiments, the adult subject is older than about 65 years of age. In some embodiments, the adult subject has previously received one or more doses of a conjugated Streptococcus agalactiae polysaccharide vaccine; in other embodiments, the adult subject is naïve to MAPS-GBS vaccines. In some embodiments, the adult subject has previously been infected with, or exposed to infection by Streptococcus agalactiae.

Immunization schedules of the present disclosure are provided to induce an immune response (e.g., an immunoprotective response) in a subject sufficient to reduce at least one measure selected from the group consisting of incidence, prevalence, frequency, and/or severity of at least one infection, disease, or disorder, and/or at least one surrogate marker of the infection, disease, or disorder, in a population and/or subpopulation of the subject(s). A supplemental immunization schedule is one which has this effect relative to the standard schedule which it supplements. A supplemental schedule may call for additional administrations and/or supra-immunogenic doses of the immunogenic compositions or vaccines disclosed herein, found in the standard schedule, or for the administration of immunogenic compositions or vaccines not part of the standard schedule. A full immunization schedule of the present invention may comprise both a standard schedule and a supplemental schedule. Exemplary sample immunization schedules are provided for illustrative purposes. Detailed descriptions of methods to assess immunogenic response discussed herein allow one to develop alterations to the sample immunization schedules without undue experimentation.

In one embodiment of the present disclosure, a first administration of a MAPS-GBS vaccine usually occurs when a subject is more than about 2 weeks old, more than about 5 weeks old, more than about 1 year old, more than about 2 years old, more than about 15 years old, or more than about 18 years old.

In one embodiment of the present disclosure, a first administration of a MAPS-GBS vaccine usually occurs when a subject is more than about 50 years old, more than about 55 years old, more than about 60 years old, more than about 65 years old, or more than about 70 years old.

In some embodiments of the disclosure, a single administration of vaccine is employed. It is possible that the purposes of the present invention can be served with a single administration, especially when one or more utilized vaccine polypeptides, polysaccharide(s) and/or conjugate(s) or combinations thereof is/are strong, and in such a situation a single dose schedule is sufficient to induce a lasting immune-protective response.

In some embodiments, it is desirable to administer two or more doses of vaccine, for greater immune-protective efficacy and coverage. Thus, in some embodiments, a number of doses is at least two, at least three or more doses. There is no set maximum number of doses, however it is good clinical practice not to immunize more often than necessary to achieve the desired effect.

Without being bound by theory, a first dose of vaccine administered according to the disclosure may be considered a “priming” dose. In some embodiments, more than one dose is included in an immunization schedule. In such a scenario, a subsequent dose may be considered a “boosting” dose.

A priming dose may be administered to a naïve subject (a subject who has never previously received a MAPS-GBS polysaccharide vaccine). In some embodiments, a priming dose may be administered to a subject who has previously received conjugated polysaccharide vaccine at least five or more years prior to the administration of an initial vaccine dose according to the invention. In other embodiments, a priming dose may be administered to a subject who has previously received a conjugated polysaccharide vaccine at least twenty or more years prior to the administration of a priming vaccine according to the invention.

When an immunization schedule calls for two or more separate doses, the interval between doses is considered. The interval between two successive doses may be the same throughout an immunization schedule, or it may change as the subject ages. In immunization schedules of the present invention, once a first vaccine dose has been administered, there is a first interval before administration of a subsequent dose. A first interval is generally at least about 2 weeks, 1 month, 6 weeks, 2 months, 3 months, 6 months, 9 months, 12 months, or longer. Where more than one subsequent dose(s) are administered, second (or higher) intervals may be provided between such subsequent doses. In some embodiments, all intervals between subsequent doses are of the same length; in other embodiments, second intervals may vary in length. In some embodiments, the interval between subsequent doses may be at least about 12 months, at least about 15 months, at least about 18 months, at least about 21 months or at least about 2 years. In some embodiments, the interval between doses may be up to 3 years, up to about 4 years, or up to about 5 years or 10 years or more. In some embodiments, intervals between subsequent doses may decrease as the subject ages.

It will be appreciated by those skilled in the art that a variety of possible combinations and sub-combinations of the various conditions of timing of the first administration, shortest interval, largest interval and total number of administrations (in absolute terms, or within a stated period) exist, and all of these combinations and sub-combinations should be considered to be within the inventor's contemplation though not explicitly enumerated here.

### (c) Assays for Determining Immune Response

In some embodiments, a method of assessing the immunogenicity of a pharmaceutical composition, immunogenic composition, or vaccine comprising a MAPS-GBS immunogenic complex and/or fusion protein described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), agglutination, motility, cytotoxicity, or adherence; and in vivo assays in animal models of Streptococcus agalactiae disease (e.g. pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the Streptococcus agalactiae pathogens that are the targets of the immunogenic composition. In some embodiments, the immune response is compared to a control composition.

In some embodiments, a method of assessing the potency of a pharmaceutical composition, immunogenic composition, or vaccine comprising a MAPS-GBS immunogenic complex and/or fusion protein described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), internalization, activity neutralization, agglutination, motility, cytotoxicity, or adherence; and in vivo assays in animal models of Streptococcus agalactiae disease (e.g. pneumonia, bacteremia, meningitis, sepsis, otitis media, nasopharyngeal colonization). Parameters include bacterial clearance or reduction from mucosal surfaces or bloodstream, reduction or prevention of bacteremia, meningitis, sepsis, or otitis media, reduction or prevention of colonization of the nasopharynx, reduction of mortality, and passive and active protection following challenge with the Streptococcus agalactiae pathogens that are the targets of the immunogenic composition. In some embodiments, the immune response is compared to a control composition.

Generally speaking, it may be desirable to assess humoral responses, cellular responses, and/or interactions between the two. Where humoral responses are being assessed, antibody titers and/or types (e.g., total IgG, IgG1, IgG2, IgM, IgA, etc.) to specific pathogen antigens (e.g., polypeptides or polysaccharides, either serotype-specific or conserved across two or more serotypes) may be determined, for example before and/or after administration of an initial or a boosting dose of vaccine (and/or as compared with antibody levels in the absence of antigenic stimulation). Cellular responses may be assessed by monitoring reactions such as delayed type hypersensitivity responses, etc. to the antigens. Cellular responses can also be measured directly by evaluating the response of peripheral blood mononuclear cells (PBMCs) monocytes to stimulation with the antigens of interest. Precursor and memory B cell populations may be assessed in enzyme-linked immunospot (ELISpot) assays directed against specific pathogen antigens.

The RIA method detects specific antibodies through incubation of sera with radio-labeled polysaccharides or polypeptides in suspension (e.g., Schiffiman et al, 1980). The antigen-antibody complexes are then precipitated with ammonium sulfate and the radiolabeled pellets assayed for counts per minute (cpm).

In the ELISA detection method, specific antibodies from the sera of vaccinated subjects are quantitated by incubation with antigens (e.g., polypeptides or polysaccharides, either serotype-specific or conserved across two or more serotypes) which have been adsorbed to a solid support (e.g., Koskela and Leinonen (1981); Kojima et al, 1990; Concepcion and Frasch, 2001). The bound antibody is detected using enzyme-conjugated secondary detection antibodies. The ELISA also allows isotyping and subclassing of the immune response (i.e., IgM vs. IgG or IgG1 vs. IgG2) by using isotype- or subclass-specific secondary antibodies and can be adapted to evaluate the avidity of the antibodies (Anttila et al, 1998; Romero-Steiner et al, 2005). Multiplex assays (e.g., Luminex) facilitate simultaneous detection of antibodies to multiple antigens. Antigens are conjugated to spectrally distinct microspheres that are mixed and incubated with serum. The antibodies bound to the antigens on the coated microspheres are detected using a secondary antibody (e.g., R-Phycoerythrin-conjugated goat anti-human IgG).

An approach for assessing functional antibody in serum is the opsonophagocytic assay (OPA) which quantitates only the antibodies that can opsonize the bacteria, leading to ingestion and killing of the bacteria. The standard assay utilizes a human phagocytic effector cell, a source of complement, bacteria, and diluted sera. The assay readout is the serum endpoint titer at which there is ≥50% killing compared to bacteria incubated with complement and human cells alone (Romero-Steiner et al, 1997). This killing OPA can also be multiplexed by utilizing target strains of pathogen that carry different antibiotic resistance markers (Kim et al, 2003). Another type of multiplex opsonic assay is a nonkilling assay in which the uptake by phagocytic effector cells of fluorescent stained encapsulated pathogen or fluorescent microspheres conjugated with antigens from a target pathogen in the presence of diluted sera plus a complement source is evaluated by FC (Martinez et al, 1999). Opsonic activity of serum antibody plus complement can also be evaluated by measuring the oxidative response of phagocytic human effector cells to ingested pathogen (Munro et al. 1985; Ojo-Amaize et al. 1995).

Certain in vivo model systems can be used to evaluate the protection afforded by serum antibodies induced by immunogenic compositions or vaccines comprising a fusion protein described herein. In such passive protection systems, mice or rats are challenged with the pathogen plus diluted sera, and the endpoint titer of the sera which provides protection against pneumonia, bacteremia, colonization of organs or tissues, or mortality is determined (Stack et al. 1998; Saeland et al. 2000).

Immunogenic composition or vaccine efficacy may also be assayed in various model systems such as the mouse challenge model. For instance, BALB/c or C57BL/6 strains of mice may be used. After administering the test immunogenic composition or vaccine to a subject (as a single dose or multiple doses), the experimenter administers a challenge dose of Streptococcus agalactiae. In some cases, a challenge dose administered intranasally is sufficient to cause Streptococcus agalactiae colonization (especially nasal colonization) in an unvaccinated animal, and in some cases a challenge dose administered via aspiration is sufficient to cause sepsis and a high rate of lethality in unvaccinated animals. In some cases, a challenge dose administered via intraperitoneal injection is sufficient to cause sepsis and a high rate of lethality in unvaccinated animals. In some cases, a challenge dose administered via intravenous injection is sufficient to cause sepsis and a high rate of lethality in unvaccinated animals. One can then measure the reduction in colonization or the reduction in lethality in vaccinated animals.

Certain in vivo model systems can be used to evaluate the protection afforded by serum antibodies induced by vaccines of the present invention. In such passive protection systems, mice or rats are challenged with the pathogen plus diluted sera, and the endpoint titer of the sera which provides protection against bacteremia, colonization of organs or tissues, or mortality is determined (Stack et al. 1998; Saeland et al. 2000).

Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the pneumococcal pathogens that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the potency of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), internalization, activity neutralization, agglutination, motility, cytotoxicity, or adherence; and in vivo assays in animal models of pneumococcal disease (e.g. pneumonia, bacteremia, meningitis, sepsis, otitis media, nasopharyngeal colonization). Parameters of in vivo assays include bacterial clearance or reduction from mucosal surfaces or bloodstream, reduction or prevention of bacteremia, meningitis, sepsis, or otitis media, reduction or prevention of colonization of the nasopharynx, reduction of mortality, and passive and active protection following challenge with the pneumococcal pathogens that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the immunogenicity of a vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), agglutination, motility, cytotoxicity, or adherence; and in vivo assays in animal models of pneumococcal disease (e.g. pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the pneumococcal pathogens that are the targets of the immunogenic composition. In some embodiments, the immune response is compared to a control composition. In some embodiments, a control composition may comprise an antigenic polysaccharide present in the vaccine composition and not comprise an antigenic polypeptide present in the vaccine composition. In some embodiments, a control composition may comprise an antigenic polypeptide present in the vaccine composition and not comprise an antigenic polysaccharide present in the vaccine composition. In some embodiments, a control composition may comprise an adjuvant present in the vaccine composition, and not comprise an antigenic polysaccharide and/or an immunogenic polypeptide present in the vaccine composition.

### X. Manufacture of Immunogenic Complexes

In some embodiments, fusion proteins as described herein may be non-covalently associated with Streptococcus agalactiae polysaccharides, by biotin to biotin-binding protein interaction, e.g., biotin to rhizavidin protein interaction, and/or by SBD interaction with sialic acid on the GBS polysaccharide, as disclosed herein. In some embodiments, the polysaccharide is a purified PS Streptococcus agalactiae, as disclosed herein in the Examples. In some embodiments, the polysaccharide is a purified lipidated PS Streptococcus agalactiae oligosaccharide.

In some embodiments, a GBS fusion protein as described herein, or disclosed in Table 3 is covalently bound to another molecule. This may, for example, increase the half-life, solubility, bioavailability, or immunogenicity of the fusion protein. Molecules that may be covalently bound to the fusion protein include a carbohydrate, biotin, poly(ethylene glycol) (PEG), polysialic acid, N-propionylated polysialic acid, nucleic acids, polysaccharides, and PLGA. There are many different types of PEG, ranging from molecular weights of below 300 g/mol to over 10,000,000 g/mol. PEG chains can be linear, branched, or with comb or star geometries. In some embodiments, the fusion protein is covalently bound to a moeity that stimulates the immune system. An example of such a moeity is a lipid moeity. In some instances, lipid moieties are recognized by a Toll-like receptor (TLR) such as TLR-2 or TLR-4, and activate the innate immune system.

The present disclosure includes methods for manufacturing MAPS-GBS immunogenic complexes described herein. In some embodiments, a method of manufacturing MAPS-GBS immunogenic complexes comprises complexing at least one biotinylated polysaccharide with at least one fusion protein as disclosed herein, e.g., a SBD-[GBS-Ag]-BBM fusion protein as disclosed herein.

In some embodiments, a fusion protein e.g., a SBD-[GBS-Ag]-BBM fusion protein and one or more additional components described herein are mixed together using known methods to form a multi-component immunogenic composition. In some embodiments, a fusion protein and one or more additional components described herein are nano-encapsulated using known methods. In some embodiments, a fusion protein and one or more additional components described herein are molded into nano- or micro-particles using known methods. In some embodiments, a fusion protein and one or more additional components described herein are conjugated through a covalent bond using known methods to form a multi-component immunogenic composition. In some embodiments, a fusion protein e.g., a SBD-[GBS-Ag]-BBM fusion protein and one or more additional components described herein are joined non-covalently using known methods to form a multi-component immunogenic composition. Additional methods of combining a fusion protein and one or more additional components are described in, e.g., PCT/US20I2/374I2 and PCT/US2009/44956.

In some embodiments, the average (e.g., the mean) protein (e.g., antigenic protein) to polysaccharide ratio of a plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average protein to polysaccharide ratio of a plurality of immunogenic complexes is approximately 1:1 (w/w). In some embodiments, the average protein to polysaccharide ratio of a plurality of immunogenic complexes is approximately 2:1 (w/w). In some embodiments, the average protein to polysaccharide ratio of a plurality of immunogenic complexes is approximately 3:1 (w/w). In some embodiments, the average protein to polysaccharide ratio of a plurality of immunogenic complexes is approximately 4:1 (w/w). In some embodiments, the average protein to polysaccharide ratio of a plurality of immunogenic complexes is approximately 5:1 (w/w). In some embodiments, the average protein to polysaccharide ratio of a plurality of immunogenic complexes is approximately 6:1 (w/w). In some embodiments, the average protein to polysaccharide ratio of a plurality of immunogenic complexes is approximately 7:1 (w/w). In some embodiments, the average protein to polysaccharide ratio of a plurality of immunogenic complexes is approximately 8:1 (w/w). In some embodiments, the average protein to polysaccharide ratio of a plurality of immunogenic complexes is approximately 9:1 (w/w). In some embodiments, the average protein to polysaccharide ratio of a plurality of immunogenic complexes is approximately 10:1 (w/w). In some embodiments, the average protein:PS ratios are chosen to enhance the polysaccharide immunogenicity potential (carrier function) and/or to elicit protection against, or to inhibit, pneumococcal colonization by any pneumococcus (independent of polysaccharide serotype) through a protein-specific immune response. Immunogenic compositions and vaccines of the invention may comprise mixtures of immunogenic complexes with different average protein to polysaccharide ratios.

In some embodiments, a vaccine or immunogenic composition comprises a plurality of MAPS-GBS immunogenic complexes comprising any one or more of a fusion protein disclosed in Tables 2A, 2B or Table 3, and a GBS polysaccharide, from or derived from Streptococcus agalactiae. In some embodiments, the average ratio of a SBD-[GBS-Ag]-BBM fusion protein as disclosed herein to a polysaccharide from or derived from Streptococcus agalactiae in the plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average ratio of total fusion protein selected from any of: (i) a SBD-[GBS-Ag]-BBM fusion protein, (ii) a SBD-[GBS-Ag]fusion protein, or (iii) BBM-[GBS-Ag] fusion protein as disclosed herein, to a biotinylated polysaccharide from or derived from any subtype of Streptococcus agalactiae in the plurality of MAPS-GBS immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]).

In some embodiments, the average ratio of total fusion protein selected from any of: (i) a SBD-[GBS-Ag]-BBM fusion protein, (ii) a SBD-[GBS-Ag] fusion protein, or (iii) BBM-[GBS-Ag] fusion protein as disclosed herein as disclosed herein to a polysaccharide from or derived from Streptococcus agalactiae in the plurality of MAPS-GBS immunogenic complexes is chosen to enhance the polysaccharide immunogenicity potential (carrier function) and/or to elicit protection against, or to inhibit, pneumococcal colonization by any GBS subtype (independent of polysaccharide serotype) through a protein specific immune response. Immunogenic compositions and vaccines of the invention may comprise mixtures of MAPS-GBE immunogenic complexes with different average protein to polysaccharide ratios.

### XI. Kits

The present disclosure also provides for kits for producing a MAPS-GBS immunogenic complex as disclosed herein which is useful for an investigator to tailor a MAPS-GBS immunogenic complex with their preferred antigens, e.g., for research purposes to assess the effect of an antigen, or a combination of antigens on immune response. Such kits can be prepared from readily available materials and reagents. For example, such kits can comprise any one or more of the following materials: a container comprising a polysaccharide cross-linked with a plurality of first affinity molecules; a container comprising a complementary affinity molecule which associates with the first affinity molecule, wherein the complementary affinity molecule associates with an antigen or carrier protein; a container comprising an antigen; a container comprising a carrier protein; a container comprising an antigen associated with a complementary affinity molecule; a container comprising a carrier protein associated with a complementary affinity molecule.

In another embodiment, the kit comprises a container comprising a GBS polysaccharide; a container comprising biotin, container comprising at least one fusion protein as disclosed herein, e.g., a SBD-[GBS-Ag]-BBM fusion protein; and a container comprising a cross-linking reagent for cross-linking a biotin to the polysaccharide, for example, but not limited to, CDAP (1-cyano-4-dimethylaminopyridinium tetrafluoroborate), and EDC (1-Ethyl-3-[3-dimethylaminopropyl]carbodiimide hydrochloride).

In another embodiment, the kit comprises a container comprising one or more fusion protein e.g., a SBD-[GBS-Ag]-BBM fusion protein, and/or alternatively, a vector encoding a SBD-[GBS-Ag]-BBM fusion protein as disclosed herein, which can optionally comprising a cloning site of inserting a nucleic acid encoding a GBS antigen of interest.

In some embodiments, the kit can comprise at least one co-stimulation factor which can be added to the polymer. In some embodiments, the kit comprises a cross-linking reagent, for example, but not limited to, CDAP (1-cyano-4-dimethylaminopyridinium tetrafluoroborate); EDC (1-Ethyl-3-[3-dimethylaminopropyl]carbodiimide hydrochloride); sodium cyanoborohydride; cyanogen bromide; and ammonium bicarbonate/iodoacetic acid, for linking the co-factor to the polymer.

A variety of kits and components can be prepared for use in the methods described herein, depending upon the intended use of the kit, the particular target antigen and the needs of the user.

In some embodiments, the present invention may be defined in any of the following numbered paragraphs:
1. A fusion protein comprising, in any order, (i) a biotin binding moiety (BBM), (ii) a sialic acid binding domain (SBD) polypeptide and (iii) at least a first polypeptide antigen or antigenic fragment thereof from Group B Streptococcus agalactiae (GBS-Ag).
2. The fusion protein of paragraph 1, wherein the fusion protein comprises in the following order, from N-terminus to C-terminus: (a) a BBM, a first GBS-Ag, a SBD; (b) a SBD, a first GBS-Ag, a BBM; (c) a BBM, a SBD, a first GBS-Ag; or (d) a first GBS-Ag, a BBM, a SBD

The fusion protein of paragraph 1 or 2, further comprising at least a second polypeptide antigen from Group B Streptococcus agalactiae (GBS-Ag), wherein the second GBS-Ag is fused to at least the first GBS-Ag.
3. The fusion protein of paragraph 3, further comprising at least a third polypeptide antigen from Group B Streptococcus agalactiae (GBS-Ag), wherein the third GBS-Ag is fused to either the first GBS-Ag or the second GBS-Ag.
4. The fusion protein of any of paragraphs 1-4, wherein the first, second or third GBS antigen is selected from the group consisting of: Rib, Sip, AlpC, Alp1, Alp3 or Alp3/1.
5. The fusion protein of any of paragraphs 1-5, wherein the fusion protein is selected from:
   a. a fusion protein comprising in order from N-terminus to C terminus: (i) the biotin-binding moiety (BBM), (ii) a Rib polypeptide antigen or an antigenic fragment thereof, or a Sip polypeptide or an antigenic fragment thereof, and (iii) the SBD polypeptide, or
   b. a fusion protein comprising in order from N-terminus to C terminus: (i) the biotin-binding moiety (BBM), (ii) a Rib polypeptide antigen or an antigenic fragment thereof and a Sip polypeptide or an antigenic fragment thereof, and (iii) the SBD polypeptide.
6. The fusion protein of any of paragraphs 1-6, wherein the fusion protein is selected from:
   a. a fusion protein comprising in order from N-terminus to C terminus: (i) a SBD polypeptide, (ii) Rib polypeptide antigen or an antigenic fragment thereof, or Sip polypeptide or an antigenic fragment thereof, and (iii) a biotin-binding moiety (BBM), or
   b. a fusion protein comprising in order from N-terminus to C terminus: (i) a SBD polypeptide, (ii) Rib polypeptide antigen or an antigenic fragment thereof and Sip polypeptide or an antigenic fragment thereof, and (iii) the biotin-binding moiety (BBM).
7. The fusion protein of any of paragraphs 1-7, wherein the BBM is Rhizavidin comprises an amino acid sequence having at least 80%, or at least 85%, or at least 90%, or at least 95% sequence identity to SEQ ID NO: 1.
8. The fusion protein of any of paragraphs 1-8, wherein the SBD is selected from any of: SBD1, SBD2, SBD3, SBD4, NanH, NanH2, VcNanH, or a SBD having an amino acid sequence that has at least 80%, or at least 85%, or at least 90%, or at least 95% sequence identity to any of: SEQ ID NO: 2 or SEQ ID NO: 112-120.
9. The fusion protein of any of paragraphs 1-9, wherein the fusion protein comprises an amino acid sequence having at least 80%, or at least 85%, or at least 90%, or at least 95% sequence identity to any of: SEQ ID NO: 3 (Rhavi-SBD1), SEQ ID NO: 129 (Rhavi-SBD2), SEQ ID NO: 140 (Rhavi-SBD3), or SEQ ID NO: 151 (Rhavi-SBD4).
10. The fusion protein of any of paragraphs 1-10, wherein any of the first, second or third polypeptide antigen comprises an amino acid sequence having at least 80%, or at least 85%, or at least 90%, or at least 95% sequence identity to SEQ ID NO: 4 (Rib).
11. The fusion protein of any of paragraphs 1-11, wherein any of the first, second or third polypeptide antigen comprises an amino acid sequence having at least 80%, or at least 85%, or at least 90%, or at least 95% sequence identity to SEQ ID NO: 5 (Sip).
12. The fusion protein of any of paragraphs 1-12, wherein any of the first, second or third
13. first polypeptide antigen comprises an amino acid sequence having at least 80%, or at least 85%, or at least 90%, or at least 95% sequence identity to any of: SEQ ID NO: 4 (Rib), SEQ ID NO: 5 (Sip), SEQ ID NO: 11 (AlpC), SEQ ID NO: 12 (Alp1); SEQ ID NO: 13 (Alp3), SEQ ID NO: 14 (Alp3/1).
14. The fusion protein of any of paragraphs 1-13, wherein the fusion protein comprises,
   a. an amino acid sequence having at least 80%, or at least 85%, or at least 90%, or at least 95% sequence identity to SEQ ID NO: 1 (Rhavi),
   b. a SBD immunogenic polypeptide, selected from:
      i. an amino acid sequence having at least 80%, or at least 85%, or at least 90%, or at least 95% sequence identity to SEQ ID NO: 2 (SBD1);
      ii. an amino acid sequence having at least 80%, or at least 85%, or at least 90%, or at least 95% sequence identity to SEQ ID NO: 112 (SBD2),
      iii. an amino acid sequence having at least 80%, or at least 85%, or at least 90%, or at least 95% sequence identity to SEQ ID NO: 113 (SBD3),
      iv. an amino acid sequence having at least 80%, or at least 85%, or at least 90%, or at least 95% sequence identity to SEQ ID NO: 114 (SBD4),
      v. an amino acid sequence having at least 80%, or at least 85%, or at least 90%, or at least 95% sequence identity to SEQ ID NO: 115 (NanH),
      vi. an amino acid sequence having at least 80%, or at least 85%, or at least 90%, or at least 95% sequence identity to SEQ ID NO: 116 (NanH2),
      vii. an amino acid sequence having at least 80%, or at least 85%, or at least 90%, or at least 95% sequence identity to SEQ ID NO: 117 (NanH3), or
      viii. an amino acid sequence having at least 80%, or at least 85%, or at least 90%, or at least 95% sequence identity to SEQ ID NO: 120 (VcNanH),
   c. an amino acid sequence having at least 80%, or at least 85%, or at least 90%, or at least 95% sequence identity to SEQ ID NO: 4 (Rib); and
   d. an amino acid sequence having at least 80%, or at least 85%, or at least 90%, or at least 95% sequence identity to SEQ ID NO: 5 (Sip).
15. The fusion protein of any of paragraphs 1-13, comprising, in the following order:
   a. an amino acid sequence having at least 80%, or at least 85%, or at least 90%, or at least 95% sequence identity to SEQ ID NO: 4 (Rib),
   b. an amino acid sequence having at least 80%, or at least 85%, or at least 90%, or at least 95% sequence identity to SEQ ID NO: 5 (Sip),
   c. an amino acid sequence having at least 80%, or at least 85%, or at least 90%, or at least 95% sequence identity to SEQ ID NO: 1 (Rhavi), and
   d. an amino acid sequence at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3) or SEQ ID NO: 120 (VcNanH)
16. The fusion protein of any of paragraphs 1-15, wherein the fusion protein comprises an amino acid sequence having at least 80%, or at least 85%, or at least 90%, or at least 95% sequence identity to SEQ ID NO: 6 (Rhavi-[Rib]-[Sip]-SBD1).
17. The fusion protein of any of paragraphs 1-13, comprising, in the following order:
   i. an amino acid sequence having at least 80%, or at least 85%, or at least 90%, or at least 95% sequence identity to SEQ ID NO: 1 (Rhavi).
   ii. an amino acid sequence having at least 80%, or at least 85%, or at least 90%, or at least 95% sequence identity to SEQ ID NO: 5 (Sip),
   iii. an amino acid sequence having at least 80%, or at least 85%, or at least 90%, or at least 95% sequence identity to SEQ ID NO: 4 (Rib), and
   iv. an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3).
18. The fusion protein of paragraph 17, wherein the fusion protein comprises an amino acid sequence having at least 80%, or at least 85%, or at least 90%, or at least 95% sequence identity to SEQ ID NO: 7 (Rhavi-[Sip]-[Rib]-SBD).
19. The fusion protein of any of paragraphs 1-13, comprising, in the following order:
   a. an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3).
   b. an amino acid sequence having at least 80%, or at least 85%, or at least 90%, or at least 95% sequence identity to SEQ ID NO: 4 (Rib),
   c. an amino acid sequence having at least 80%, or at least 85%, or at least 90%, or at least 95% sequence identity to SEQ ID NO: 5 (Sip), and
   d. an amino acid sequence having at least 80%, or at least 85%, or at least 90%, or at least 95% sequence identity to SEQ ID NO: 1 (Rhavi).
20. The fusion protein of any of paragraphs 1-13, comprising, in the following order:
   a. an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3),
   b. an amino acid sequence having at least 80%, or at least 85%, or at least 90%, or at least 95% sequence identity to SEQ ID NO: 5 (Sip),
   c. an amino acid sequence having at least 80%, or at least 85%, or at least 90%, or at least 95% sequence identity to SEQ ID NO: 4 (Rib), and
   d. an amino acid sequence having at least 80%, or at least 85%, or at least 90%, or at least 95% sequence identity to SEQ ID NO: 1 (Rhavi).
21. The fusion protein of any of paragraphs 1-20, comprising at least one linker sequence or at least one spacer sequence located between any of: the BBM and the SBD; the BBM and at the first polypeptide antigen or an antigenic fragment thereof, the first polypeptide antigen or an antigenic fragment thereof and the second polypeptide antigen or an antigenic fragment thereof, or a polypeptide antigen or an antigenic fragment thereof and a SBD.
22. The fusion protein of any of paragraphs 1-21, wherein the linker sequence or spacer sequence is selected from any of: GGGGSSS, TDPNSSS, SSS, AAA.
23. The fusion protein of any of paragraphs 1-4, wherein the fusion protein is selected from any listed in Table 2A, 2B or Table 3, or comprises an amino acid sequence having at least 80%, or at least 85%, or at least 90%, or at least 95% sequence identity to any of the amino acid sequences listed in Table 3.
24. A fusion protein comprising a sialic acid binding domain (SBD) polypeptide and at least a first polypeptide antigen or an antigenic fragment thereof from Group B Streptococcus agalactiae (GBS-Ag).
25. The fusion protein of paragraph 24, further comprising at least a second polypeptide antigen from Group B Streptococcus agalactiae (GBS-Ag), wherein the second GBS-Ag is fused to at least the first GBS-Ag.
26. The fusion protein of paragraph 25, further comprising at least a third polypeptide antigen from Group B Streptococcus agalactiae (GBS-Ag), wherein the third GBS-Ag is fused to either the first GBS-Ag or the second GBS-Ag.
27. The fusion protein of any of paragraphs 24-26, wherein the first, second or third GBS antigen is selected from the group consisting of: Rib, Sip, AlpC, Alp1, Alp3 or Alp3/1.
28. The fusion protein of any of paragraph 24, wherein the first polypeptide antigen or an antigenic fragment thereof is a Rib polypeptide.
29. The fusion protein of any of paragraph 24, wherein the first polypeptide antigen or an antigenic fragment thereof is a Sip polypeptide.
30. The fusion protein of any of paragraph 25, wherein
   a. the first GBS polypeptide antigen or an antigenic fragment thereof is a Rib polypeptide, and the second GBS polypeptide antigen or antigenic fragment is a Sip polypeptide, or
   b. the first polypeptide antigen or an antigenic fragment thereof is a Sip polypeptide, and the second polypeptide antigen or antigenic fragment is a Rib polypeptide.
31. The fusion protein of any of paragraphs 24-30, wherein the fusion protein is selected from:
   a. a fusion protein comprising in order from N-terminus to C terminus: (i) a SBD polypeptide, (ii) Rib polypeptide antigen or an antigenic fragment thereof, or Sip polypeptide or an antigenic fragment thereof, or
   b. a fusion protein comprising in order from N-terminus to C terminus: (i) a SBD polypeptide, (ii) Rib polypeptide antigen or an antigenic fragment thereof, and Sip polypeptide or an antigenic fragment thereof, wherein the Rib polypeptide antigen or an antigenic fragment thereof and Sip polypeptide or an antigenic fragment thereof can be in any order.
32. The fusion protein of paragraphs 24-30, wherein the fusion protein is selected from:
   a. a fusion protein comprising in order from N-terminus to C terminus: (i) Rib polypeptide antigen or an antigenic fragment thereof, or Sip polypeptide or an antigenic fragment thereof, and (ii) a SBD polypeptide, or
   b. a fusion protein comprising in order from N-terminus to C terminus: (i) Rib polypeptide antigen or an antigenic fragment thereof, and Sip polypeptide or an antigenic fragment thereof, (ii) a SBD polypeptide, wherein the Rib polypeptide antigen or an antigenic fragment thereof and Sip polypeptide or an antigenic fragment thereof can be in any order.
33. The fusion protein of any of paragraphs 24-32, wherein the SBD is selected from any of: SBD1, SBD2, SBD3, SBD4, NanH, NanH2, VcNanH, or a SBD having an amino acid sequence that has at least 80%, or at least 85%, or at least 90%, or at least 95% sequence identity to any of: SEQ ID NO: 2 or SEQ ID NO: 112-120.
34. The fusion protein of any of paragraphs 24-33, wherein the SBD comprises an amino acid sequence having at least 80%, or at least 85%, or at least 90%, or at least 95% sequence identity to SEQ ID NO: 2 or SEQ ID NO: 112.
35. The fusion protein of any of paragraphs 24-34, wherein the first polypeptide antigen comprises an amino acid sequence having at least 80%, or at least 85%, or at least 90%, or at least 95% sequence identity to SEQ ID NO: 4 (Rib).
36. The fusion protein of any of paragraphs 24-29, wherein the first polypeptide antigen comprises an amino acid sequence having at least 80%, or at least 85%, or at least 90%, or at least 95% sequence identity to SEQ ID NO: 5 (Sip).
37. The fusion protein of any of paragraphs 24, comprising at least one linker sequence or at least one spacer sequence located between any of: the SBD and at the first polypeptide antigen or an antigenic fragment thereof, the first polypeptide antigen or an antigenic fragment thereof and the second polypeptide antigen or an antigenic fragment thereof, or a polypeptide antigen or an antigenic fragment thereof and a SBD.
38. The fusion protein of any of paragraphs 1-21, wherein the linker sequence or spacer sequence is selected from any of: GGGGSSS, TDPNSSS, SSS, AAA.
39. The fusion protein of any of paragraphs 24-38, wherein the fusion protein is selected from any SBD fusion proteins listed in Table 2A, 2B or Table 3, or comprises an amino acid sequence having at least 80%, or at least 85%, or at least 90%, or at least 95% sequence identity to any of the amino acid sequences listed in Table 3.
40. An immunogenic composition comprising at least one species of MAPS-GBS immunogenic complex, wherein each species of the MAPS-GBS immunogenic complex comprises;
   a. at least a first biotinylated polysaccharide antigen (PS1) comprising a first polysaccharide antigen, at least one sialic acid molecule and at least one biotin molecule,
   b. at least a second biotinylated polysaccharide antigen (PS2) comprising a second polysaccharide antigen, at least one sialic acid molecule and at least one biotin molecule,
   c. at least one fusion protein, each fusion protein comprises at least a biotin-biotin moiety (BBM) and a sialic acid binding domain (SBD),
wherein a BBM of at least one fusion protein non-covalently associates with at least one biotin molecule on the first biotinylated polysaccharide antigen (PS1) and the SBD non-covalently associates with at least one sialic acid on the second biotinylated polysaccharide antigen (PS2), and/or
wherein a SBD of at least one fusion protein non-covalently associates with at least one sialic acid molecule on the first biotinylated polysaccharide antigen (PS1) and the BBM non-covalently associates with at least biotin molecule on the second biotinylated polysaccharide antigen (PS2),
wherein the first biotinylated polysaccharide antigen, the second biotinylated polysaccharide antigen and the fusion protein form a MAPS-GBS immunogenic complex by the non-covalent association of at least one fusion protein to both the first biotinylated polysaccharide antigen and the second biotinylated polysaccharide antigen.
41. The immune composition of paragraph 40, wherein the PS1 and PS2 are located on the same polysaccharide macromolecule.
42. The immune composition of paragraph 40, wherein the PS1 and PS2 are located on distinct polysaccharide macromolecules.
43. The immune composition of paragraph 41, wherein one polysaccharide macromolecule is from a pathogen, and the other macromolecule is from a distinct pathogen or serotype.
44. The immune composition of paragraph 40, wherein the first or second polysaccharide antigen, or both, is a capsular polysaccharide (CP) from a Group B Streptococcus (GBS) or Streptococcus agalactiae.
45. The immune composition of paragraph 44, wherein the first polysaccharide antigen and the second polysaccharide antigen are the same serotype of GBS.
46. The immune composition of paragraph 44, wherein the first polysaccharide antigen is from one serotype of GBS, and the second polysaccharide antigen is from a different serotype of GBS.
47. The immune composition of any of paragraphs 40-46, wherein the immune composition comprises at least 3, or at least 4, or at least 5 or more than 5 species of immunogenic complex.
48. The immune composition of any of paragraphs 40-47, wherein the immune composition comprises at least 6-10, 11-15, 16-20, 21-25, 26-30, or 31-35 or more than 35 or more species of immunogenic complex.
49. The immune composition of any of paragraphs 40-48, wherein the first biotinylated polysaccharide, or second biotinylated polysaccharide, or both, is from any of serotypes 1a, Ib, II, III, IV, V, VI, VII, VIII, and IX of Streptococcus agalactiae.
50. The immune composition of any of paragraphs 40-49, wherein the first biotinylated polysaccharide, or second biotinylated polysaccharide, or both, is selected from any of serotypes 1a, Ib, II, III, IV, V or VII of Streptococcus agalactiae.
51. The immune composition of any of paragraphs 40-50, comprising at least 4 immunogenic complexes, wherein the at least 4 complexes are selected from any of:
   i. a MAPS-GBS immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia or a different serotype selected from any of serotypes Ib, II, III, IV, V, VI, VII, VIII, and IX of Streptococcus agalactiae, and (c) at least one fusion protein, or
   ii. a MAPS-GBS immunogenic complex comprising (a) a first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib or a different serotype selected from any of serotypes Ia, II, III, IV, V, VI, VII, VIII, and IX of Streptococcus agalactiae, and (c) at least one fusion protein, or
   iii. a MAPS-GBS immunogenic complex comprising (a) a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II or a different serotype selected from any of serotypes Ia, Ib, III, IV, V, VI, VII, VIII, and IX of Streptococcus agalactiae, and (c) at least one fusion protein, or
   iv. a MAPS-GBS immunogenic complex comprising (a) a first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III or a different serotype selected from any of serotypes Ia, Ib, II, IV, V, VI, VII, VIII, and IX of Streptococcus agalactiae, and (c) at least one fusion protein, or
   v. a MAPS-GBS immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV or a different serotype selected from any of serotypes Ia, Ib, II, III, V, VI, VII, VIII, and IX of Streptococcus agalactiae, and (c) at least one fusion protein, or
   vi. a MAPS-GBS immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V or a different serotype selected from any of serotypes Ia, Ib, II, III, IV, VI, VII, VIII, and IX of Streptococcus agalactiae, and (c) at least one fusion protein, or
   vii. a MAPS-GBS immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII or a different serotype selected from any of serotypes Ia, Ib, II, III, IV, V, VI, VIII, and IX of Streptococcus agalactiae, and (c) at least one fusion protein.
52. The immune composition of paragraph 47, wherein the immune composition comprises the immunogenic complexes of:
   i. a first MAPS-GBS immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia or a different serotype selected from any of serotypes Ib, II, III, IV, V, VI, VII, VIII, and IX of Streptococcus agalactiae, and (c) at least one fusion protein,
   ii. a second MAPS-GBS immunogenic complex comprising (a) first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib or a different serotype selected from any of serotypes Ia, II, III, IV, V, VI, VII, VIII, and IX of Streptococcus agalactiae, and (c) at least one fusion protein, and (b) at least one fusion protein,
   iii. a third MAPS-GBS immunogenic complex comprising a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II or a different serotype selected from any of serotypes Ia, Ib, III, IV, V, VI, VII, VIII, and IX of Streptococcus agalactiae, and (c) at least one fusion protein,
   iv. a fourth MAPS-GBS immunogenic complex comprising (a) first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III or a different serotype selected from any of serotypes Ia, Ib, II, IV, V, VI, VII, VIII, and IX of Streptococcus agalactiae, and (c) at least one fusion protein,
   v. a firth MAPS-GBS immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V or a different serotype selected from any of serotypes Ia, Ib, II, III, IV, VI, VII, VIII, and IX of Streptococcus agalactiae, and (c) at least one fusion protein, and
   vi. a sixth MAPS-GBS immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII or a different serotype selected from any of serotypes Ia, Ib, II, III, IV, V, VI, VIII, and IX of Streptococcus agalactiae, and (c) at least one fusion protein.
53. The immune composition of paragraphs 50, further comprising: a seventh MAPS-GBS immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV or a different serotype selected from any of serotypes Ia, Ib, II, III, V, VI, VII, VIII, and IX of Streptococcus agalactiae, and (c) at least one fusion protein, and (b) at least one fusion protein.
54. The immune composition of any of paragraphs 52 or 53, wherein when the first polysaccharide antigen is from a specific Streptococcus agalactiae serotype, the second polysaccharide antigen can be from the same Streptococcus agalactiae serotype, or a different Streptococcus agalactiae serotype.
55. The immune composition of any of paragraphs 49 or 50, wherein when the first polysaccharide antigen and the second polysaccharide antigen are from the same Streptococcus agalactiae serotype.
56. The immune composition of paragraph 54, wherein the immune composition comprises the immunogenic complexes of:
   i. a first MAPS-GBS immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia, and (c) at least one fusion protein,
   ii. a second MAPS-GBS immunogenic complex comprising (a) first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib, and (c) at least one fusion protein,
   iii. a third MAPS-GBS immunogenic complex comprising a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II, and (c) at least one fusion protein, and (b) at least one fusion protein,
   iv. a fourth MAPS-GBS immunogenic complex comprising (a) first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III, and (c) at least one fusion protein,
   v. a firth MAPS-GBS immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V, and (c) at least one fusion protein, and
   vi. a sixth MAPS-GBS immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII, and (c) at least one fusion protein.
57. The immune composition of paragraphs 56, further comprising: a seventh MAPS-GBS immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV, and (c) at least one fusion protein, and (b) at least one fusion protein.
58. The immune composition of any of paragraphs 40-57, wherein the first biotinylated polysaccharide, or second biotinylated polysaccharide, or both, has a sialic acid level of greater than about 20% or about 30%, or about 40%, or about 50% or about 60%.
59. The immune composition of any of paragraphs 40-59, wherein the at least one fusion protein is a fusion protein selected from any of paragraphs 1-23, or selected from any listed in Table 2A, 2B or Table 3, or comprises an amino acid sequence having at least 80%, or at least 85%, or at least 90%, or at least 95% sequence identity to any of the amino acid sequences listed in Table 3.
60. The immune composition of any of paragraphs 40-4, wherein the MAPS-GBS immunogenic complex further comprises at least one fusion protein selected from any of paragraphs 24-39 or a SBD fusion protein selected from any listed in Table 2A, 2B or Table 3, or comprises an amino acid sequence having at least 80%, or at least 85%, or at least 90%, or at least 95% sequence identity to any of the amino acid sequences listed in Table 3.
61. A pharmaceutical composition comprising the immunogenic composition of any of paragraphs 40-60, and a pharmaceutically acceptable carrier.
62. The pharmaceutical composition of paragraph 61, further comprising one or more adjuvants.
63. The pharmaceutical composition of paragraph 62, wherein the one or more adjuvants is or comprises a co-stimulation factor.
64. The pharmaceutical composition of paragraph 61, wherein the one or more adjuvants are selected from the group consisting of aluminum phosphate, aluminum hydroxide, and phosphate aluminum hydroxide.
65. A vaccine comprising the immunogenic composition of any of paragraphs 40-60, or a fusion protein of any of paragraphs 1-39, and a pharmaceutically acceptable carrier.
66. A method of making a multivalent vaccine, comprising mixing four or more species of MAPS-GBS immunogenic complexes of any of paragraphs 40-60 in a single formulation.
67. The method of paragraph 66, comprising mixing six or more species of immunogenic complexes in a single formulation, wherein the six or more immunogenic complexes are disclosed in any of paragraphs 48-54.
68. The method of paragraph 66, wherein each species of the immunogenic complexes comprises least one fusion protein is a fusion protein selected from any of paragraphs 1-23, or selected from any listed in Table 2A, 2B or Table 3, or comprises an amino acid sequence having at least 80%, or at least 85%, or at least 90%, or at least 95% sequence identity to any of the amino acid sequences listed in Table 3.
69. The method of paragraph 68, wherein each species of the MAPS-GBS immunogenic complex can comprises at least one fusion protein selected from any of paragraphs 24-39 or selected from any listed in Table 2A, 2B or Table 3, or comprises an amino acid sequence having at least 80%, or at least 85%, or at least 90%, or at least 95% sequence identity to any of the amino acid sequences listed in Table 3.
70. The method of paragraph 66, wherein each of the species of immunogenic complexes comprises:
   a. at least a first biotinylated polysaccharide antigen comprising a first polysaccharide antigen, at least one sialic acid molecule and at least one biotin molecule,
   b. at least a second biotinylated polysaccharide antigen comprising a second polysaccharide antigen, at least one sialic acid molecule and at least one biotin molecule,
   c. at least one fusion protein, each fusion protein comprises at least a biotin-biotin moiety (BBM) and a sialic acid binding domain (SBD), wherein the fusion protein comprises in order from N-terminus to C terminus:
      (i) a biotin-binding moiety (BBM), (ii) Rib polypeptide antigen or an antigenic fragment thereof, or Sip polypeptide or an antigenic fragment thereof, and (iii) a SBD polypeptide, or
      (i) a biotin-binding moiety (BBM), (ii) a Rib polypeptide antigen or an antigenic fragment thereof and a Sip polypeptide or an antigenic fragment thereof, and (iii) a SBD polypeptide, or
      (i) a SBD polypeptide, (ii) a Rib polypeptide antigen or an antigenic fragment thereof, or a Sip polypeptide or an antigenic fragment thereof, and (iii) a biotin-binding moiety (BBM), or
      (i) a SBD polypeptide, (ii) Rib polypeptide antigen or an antigenic fragment thereof and Sip polypeptide or an antigenic fragment thereof, and (iii) the biotin-binding moiety (BBM),
      wherein a BBM of at least one fusion protein non-covalently associates with at least one biotin molecule on the first biotinylated polysaccharide antigen and the SBD non-covalently associates with at least one sialic acid on the second biotinylated polysaccharide antigen, and/or
      wherein a SBD of at least one fusion protein non-covalently associates with at least one sialic acid molecule on the first biotinylated polysaccharide antigen and the BBM non-covalently associates with at least biotin molecule on the second biotinylated polysaccharide antigen,
      wherein the first biotinylated polysaccharide antigen, the second biotinylated polysaccharide antigen and the fusion protein form a MAPS-GBS immunogenic complex by the non-covalent association of at least one fusion protein to both the first biotinylated polysaccharide antigen and the second biotinylated polysaccharide antigen
71. The method of any of paragraphs 66-70, wherein the BBM is Rhizavidin and comprises an amino acid sequence having at least 80%, or at least 85%, or at least 90%, or at least 95% sequence identity to SEQ ID NO: 1.
72. The method of any of paragraphs 66-71, wherein the SBD comprises an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3).
73. The method of any of paragraphs 66-72, wherein the Rib polypeptide antigen comprises an amino acid sequence having at least 80%, or at least 85%, or at least 90%, or at least 95% sequence identity to SEQ ID NO: 4 (Rib).
74. The method of any of paragraphs 66-73, wherein the Sip polypeptide antigen comprises an amino acid sequence having at least 80%, or at least 85%, or at least 90%, or at least 95% sequence identity to SEQ ID NO: 5 (Sip).
75. The method of any of paragraphs 66-74, wherein the fusion protein is selected from any of the fusion proteins disclosed in paragraphs 14-20 or selected from any listed in Table 2A, 2B or Table 3, or comprises an amino acid sequence having at least 80%, or at least 85%, or at least 90%, or at least 95% sequence identity to any of the amino acid sequences listed in Table 3.
76. The method of any of paragraphs 66-75, wherein the fusion protein comprises at least one linker sequence or at least one spacer sequence located between any of: the BBM and the SBD; the BBM and at the first polypeptide antigen or an antigenic fragment thereof, the first polypeptide antigen or an antigenic fragment thereof and the second polypeptide antigen or an antigenic fragment thereof, or a polypeptide antigen or an antigenic fragment thereof and a SBD polypeptide.
77. The method of any of paragraphs 66-76, wherein the linker sequence or spacer sequence is selected from any of: GGGGSSS, TDPNSSS, SSS and AAA.
78. The method of any of paragraphs 66-77, wherein each species of MAPS-GBS immunogenic complex comprises a first biotinylated polysaccharide antigen, or a second biotinylated polysaccharide antigen, or both the first and second polysaccharide antigen from a serotype of a Group B Streptococcus (GBS).
79. The method of any of paragraphs 66-78, comprising mixing four species of immunogenic complexes, wherein the Streptococcus agalactiae serotype for each species of the four immunogenic complexes is selected from any of: Ia, Ib, II, III, IV, V or VII.
80. The method of any of paragraphs 66-81, comprising mixing six species of immunogenic complexes, wherein the Streptococcus agalactiae serotype for each species of the immunogenic complexes is selected from: Ia, Ib, II, III, V and VII.
81. The method of any of paragraphs 66-80, comprising mixing seven species of immunogenic complexes, wherein the Streptococcus agalactiae serotype for each species of the immunogenic complexes is selected from: Ia, Ib, II, III, IV, V and VII.

### XII. Certain Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastrical, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: In its broadest sense, the term “amino acid”, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Non-standard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kDa tetrameric agents comprised of two identical heavy chain polypeptides (about 50 kDa each) and two identical light chain polypeptides (about 25 kDa each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb©); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR©s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Antigen: The term “antigen”, as used herein, refers to (i) an agent that induces an immune response; and/or (ii) an agent that binds to a T cell receptor (e.g., when presented by an MHC molecule) or to an antibody. In some embodiments, an antigen induces a humoral response (e.g., including production of antigen-specific antibodies); in some embodiments, an antigen induces a cellular response (e.g., involving T cells whose receptors specifically interact with the antigen). In some embodiments, an antigen induces a humoral response and a cellular response. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)), etc. In some embodiments, an antigen is or comprises a polypeptide. In some embodiments, an antigen is or comprises a polysaccharide. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen. In some embodiments, an antigen is a polypeptide or a polysaccharide that, upon administration to a subject, induces a specific and/or clinically relevant immune response to such polypeptide or polysaccharide. In some embodiments, an antigen is selected to induce a specific and/or clinically relevant immune response to such polypeptide or polysaccharide.

Associated with: Two entities are “associated” with one another, as that term is used herein, if the presence, level and/or form of one is correlated with that of the other. In some embodiments, two or more entities are physically “associated” with one another if they interact, directly or indirectly, so that they are and/or remain in physical proximity with one another. In some embodiments, two or more entities that are physically associated with one another are covalently linked to one another. In some embodiments, two or more entities that are physically associated with one another are not covalently linked to one another but are non-covalently associated, for example by means of affinity interactions, electrostatic interactions, hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Carrier protein: As used herein, the term “carrier protein” refers to a protein or peptide that is coupled, complexed, or otherwise associated with a hapten (e.g., a small peptide or lipid) or less immunogenic antigen (e.g., a polysaccharide) and that induces or improves an immune response to such a coupled, or complexed, or otherwise associated hapten (e.g., a small peptide or lipid) or less immunogenic antigen (e.g., a polysaccharide). In some embodiments, such an immune response is or comprises a response to a hapten or less immunogenic antigen that is coupled, complexed, or otherwise associated with such a carrier protein. In some embodiments, such an immune response is or comprises a response to both a carrier protein and a hapten or less immunogenic antigen that is coupled, complexed, or otherwise associated with such a carrier protein. In some embodiments, no significant immune response to a carrier protein itself occurs. In some embodiments, immune response to a carrier protein may be detected; in some such embodiments, immune response to such a carrier protein is strong. In some embodiments, a carrier protein is coupled, complexed, or otherwise associated with one or more other molecules.

Colonization: As used herein, the term “colonization” generally refers to the ability of a microbe to grow at a target site or surface. For example, the term “colonization” refers to the ability of a microbe (e.g., a bacterium) to grow at an anatomical site (e.g., a mucosal membrane, gastrointestinal tract, injury site, organ, etc.) of a host.

Combination therapy: As used herein, the term “combination therapy” refers to those situations in which a subject is exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Derivative: As used herein, the term “derivative”, or grammatical equivalents thereof, refers to a structural analogue of a reference substance. That is, a “derivative” is a substance that shows significant structural similarity with the reference substance, for example sharing a core or consensus structure, but also differs in certain discrete ways. Such a substance would be said to be “derived from” said reference substance. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Fragment: A “fragment” of a material or entity as described herein has a structure that includes a discrete portion of the whole, but lacks one or more moieties found in the whole. In some embodiments, a fragment consists of such a discrete portion. In some embodiments, a fragment includes a discrete portion of the whole which discrete portion shares one or more functional characteristics found in the whole. In some embodiments, a fragment consists of such a discrete portion. In some embodiments, a fragment consists of or comprises a characteristic structural element or moiety found in the whole. In some embodiments, a fragment of a polymer, e.g., a polypeptide or polysaccharide, comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, or 99% of the monomeric units (e.g., residues) found in the whole polymer. The whole material or entity may in some embodiments be referred to as the “parent” of the whole.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98% or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In some embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, at least 99.5%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, inhibit or reduce: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Immunologically effective amount or immunologically effective dose: As used herein, “immunologically effective amount” or “immunologically effective dose” refers to an amount of an antigenic or immunogenic substance, e.g., an antigen, immunogen, immunogenic complex, immunogenic composition, vaccine, or pharmaceutical composition, which when administered to a subject, either in a single dose or as part of a series of doses, that is sufficient to enhance a subject's own immune response against a subsequent exposure to a pathogen. In some embodiments, the pathogen is S. agalactiae (Group B strep). In some embodiments, the immune response is against one or more different serotypes of S. agalactiae (Group B strep). In some embodiments, the immune response is against two or more different serotypes of S. agalactiae (Group B strep). In some embodiments, the immune response is against four or more different serotypes of S. agalactiae (Group B strep). In some embodiments, the immune response is against five or more different serotypes of S. agalactiae i some embodiments, the immune response is against six or more different serotypes of S. agalactiae (Group B strep). In some embodiments, the immune response is against seven or more different serotypes of S. agalactiae (Group B strep). In some embodiments, the immune response is against eight or more different serotypes of S. agalactiae (Group B strep). An immunologically effective amount may vary based on the subject to be treated, the species of the subject, the degree of immune response desired to induce, etc. In some embodiments, an immunologically effective amount is sufficient for treatment or protection of a subject having or at risk of having disease. In some embodiments, an immunologically effective amount refers to a non-toxic but sufficient amount that can be an amount to treat, attenuate, or prevent infection and/or disease (e.g., bacterial infection, S. agalactiae infection, bacterial colonization, S. agalactiae colonization, complications associated with bacterial infection, complications associated with S. agalactiae infection, etc.) in any subject. In some embodiments, an immunologically effective amount is sufficient to induce an immunoprotective response upon administration to a subject.

Immunoprotective response or protective response: As used herein, “immunoprotective response” or “protective response” refers to an immune response that mediates antigen or immunogen-induced immunological memory. In some embodiments, an immunoprotective response is induced by the administration of a substance, e.g., an antigen, immunogen, immunogenic complex, immunogenic composition, vaccine, or pharmaceutical composition to a subject. In some embodiments, immunoprotection involves one or more of active immune surveillance, a more rapid and effective response upon immune activation as compared to a response observed in a naïve subject, efficient clearance of the activating agent or pathogen, followed by rapid resolution of inflammation. In some embodiments, an immunoprotective response is an adaptive immune response. In some embodiments, an immunoprotective response is sufficient to protect an immunized subject from productive infection by a particular pathogen or pathogens to which a vaccine is directed (e.g., S. agalactiae (Group B strep) infection).

Immunization: As used herein, “immunization”, or grammatical equivalents thereof, refers to a process of inducing an immune response to an infectious organism or agent in a subject (“active immunization”), or alternatively, providing immune system components against an infectious organism or agent to a subject (“passive immunization”). In some embodiments, immunization involves the administration of one or more antigens, immunogens, immunogenic complexes, vaccines, immune molecules such as antibodies, immune sera, immune cells such as T cells or B cells, or pharmaceutical compositions to a subject. In some embodiments, immunization is performed by administering an immunologically effective amount of a substance, e.g., an antigen, immunogen, immunogenic complex, immunogenic composition, vaccine, immune molecule such as an antibody, immune serum, immune cell such as a T cell or B cell, or pharmaceutical composition to a subject. In some embodiments, immunization results in an immunoprotective response in the subject. In some embodiments, active immunization is performed by administering to a subject an antigenic or immunogenic substance, e.g., an antigen, immunogen, immunogenic complex, vaccine, or pharmaceutical composition. In some embodiments, passive immunization is performed by administering to a subject an immune system component, e.g., an immune molecule such as an antibody, immune serum, or immune cell such as a T cell or B cell.

Isolated: As used herein, the term “isolated”, or grammatical equivalents thereof, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polysaccharide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide or polysaccharide that is chemically synthesized or is synthesized in a cellular system different from that which produces it in nature is considered to be an “isolated” polypeptide or polysaccharide. Alternatively or additionally, in some embodiments, a polypeptide or polysaccharide that has been subjected to one or more purification techniques may be considered to be an “isolated” polypeptide or polysaccharide to the extent that it has been separated from other components a) with which it is associated in nature; and/or b) with which it was associated when initially produced.

Linker: As used herein, the term “linker” is used to refer to an entity that connects two or more elements to form a multi-element agent. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Plurality: As used herein, the term “plurality” includes at least 2 or more, including, e.g., at least 3, at least 4, at least 5, at least 6, at least 7, at least 8, at least 9, at least 10, or more.

Polysaccharide: The term “polysaccharide” as used herein refers to a polymer macromolecule that is a polymeric carbohydrate molecule composed of long chains of monosaccharide units bound together by glycosidic, phosphodiester, or other linkages, and on hydrolysis give the constituent monosaccharides or oligosaccharides. Polysaccharides range in structure from linear to highly branched. Examples include storage polysaccharides such as starch and glycogen, structural polysaccharides such as cellulose and chitin and microbial polysaccharides, and antigenic polysaccharides found in microorganisms including, but not limited to, capsular polysaccharides (CPS), O polysaccharides (OPS), core O polysaccharides (COPS), and lipopolysaccharides (LPS). Polysaccharides comprise regions of polysaccharide antigens, as this term is disclosed herein.

Polysaccharide Antigen: The term “polysaccharide antigen” as used herein refers to a region or portion of a polysaccharide macromolecule that comprises a plurality of biotin molecules and/or sialic acid molecule. A “polysaccharide antigen” is a region in the polysaccharide macromolecule to which a SBD and/or BBM non-covalently associates with.

Polypeptide: The term “polypeptide”, as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Prevention: The term “prevent” or “prevention”, as used herein in connection with a disease, disorder, and/or medical condition, refers to reducing the risk of developing the disease, disorder and/or condition, and/or a delay of onset, and/or reduction in frequency and/or severity of one or more characteristics or symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete when onset of a disease, disorder or condition has been delayed for a predefined period of time.

Protein: As used herein, the term “protein” encompasses a polypeptide. Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain l-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant: As used herein, the term “recombinant” is intended to refer to polypeptides that are designed, engineered, prepared, expressed, created, manufactured, and/or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, subject, population, sample, sequence or value of interest is compared with a reference or control agent, animal, subject, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a “response” to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Subject or tumor response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of biomarkers in a sample obtained from a subject, cytology, and/or histology. The exact response criteria can be selected in any appropriate manner, provided that when comparing groups of subjects and/or tumors, the groups to be compared are assessed based on the same or comparable criteria for determining response rate. One of ordinary skill in the art will be able to select appropriate criteria.

Risk: As will be understood from context, “risk” of a disease, disorder, and/or condition refers to a likelihood that a particular subject will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments, risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from subjects comparable to a particular subject. In some embodiments, relative risk is 0, 1, 2, 3, 4, 5, 6, 7, 8, 9, 10, or more.

Serotype: As used herein, the term “serotype”, also referred to as a serovar, refers to a distinct variation within a species of bacteria or virus or among immune cells of different subjects. These microorganisms, viruses, or cells are classified together based on their cell surface antigens, allowing the epidemiologic classification of organisms to the sub-species level. A group of serovars with common antigens may be referred to as a serogroup or sometimes serocomplex.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

Susceptible to: A subject who is “susceptible to” a disease, disorder, or condition is at risk for developing the disease, disorder, or condition. In some embodiments, a subject who is susceptible to a disease, disorder, or condition does not display any symptoms of the disease, disorder, or condition. In some embodiments, a subject who is susceptible to a disease, disorder, or condition has not been diagnosed with the disease, disorder, and/or condition. In some embodiments, a subject who is susceptible to a disease, disorder, or condition is a subject who has been exposed to conditions associated with development of the disease, disorder, or condition. In some embodiments, a risk of developing a disease, disorder, and/or condition is a population-based risk (e.g., family members of subjects suffering from the disease, disorder, or condition).

Symptoms are reduced: As used herein, “symptoms are reduced” when one or more symptoms of a particular disease, disorder or condition is reduced in magnitude (e.g., intensity, severity, etc.) and/or frequency, e.g., to a statistically and/or clinically significant or relevant level. For purposes of clarity, a delay in the onset of a particular symptom is considered one form of reducing the frequency of that symptom.

Treatment: As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vaccination: As used herein, the term “vaccination” refers to the administration of a composition intended to generate an immune response, for example to a disease-causing agent. For the purposes of the present invention, vaccination can be administered before, during, and/or after exposure to a disease-causing agent, and in some embodiments, before, during, and/or shortly after exposure to the agent. In some embodiments, vaccination includes multiple administrations, appropriately spaced in time, of a vaccinating composition. In some embodiments, vaccination initiates immunization.

### EXAMPLES

### Example 1

### Incorporation of SBD in the Carrier Protein Significantly Enhances Antibody to GBS Polysaccharide.

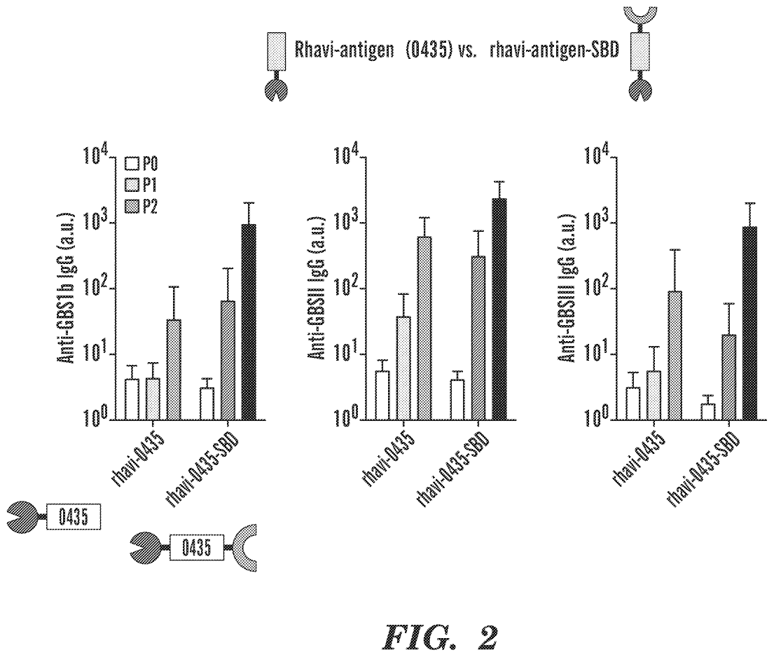

Rabbits were immunized with either a tri-valent GBS MAPS vaccine (3V MAPS-GBS) containing Rhavi-0435 or Rhavi-0435-SBD fusion proteins two times with three weeks intervals. Rabbits were bleed at day 0 (P0), day 21 (p1) or day 42 (p2). The antibody titers against three GBS polysaccharides (Ib, II and III) were measured using ELISA. As shown in FIG. 2, rabbits immunized with MAPS containing Rhavi-0435-SBD made higher antibodies than those immunized with MAPS containing Rhavi-0435.

### Example 2

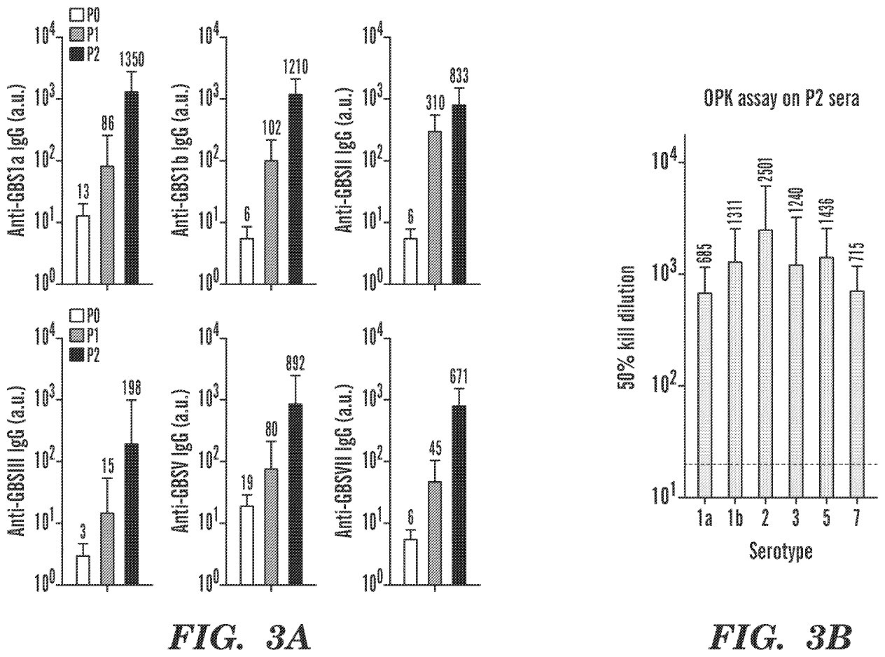

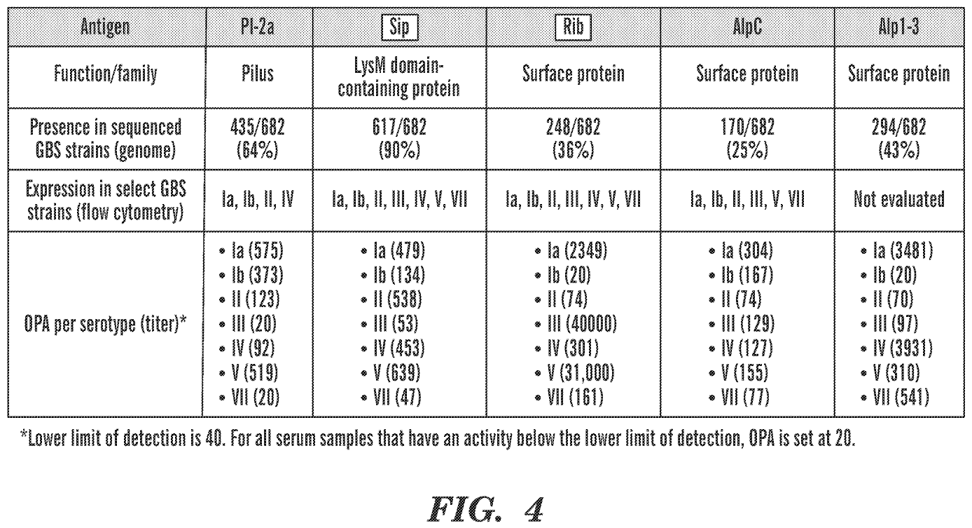

A 6-valent GBS MAPS vaccine induced functional antibody response to all the GBS polysaccharides. Rabbits were immunized with a 6 valent GBS MAPS (MAPS-GBS) containing serotypes Ia, Ib, II, III, V and VII two times with three weeks intervals. Antibody titer against each serotype was measured by ELISA (FIG. 3A). The post-2 immune sera were used in an OPK assay with GBS clinical strains. MAPS-GBS induced high killing titer for all six serotypes (FIG. 3B). OPK titer of pre-immune sera (P0) was below the lower limit of detection (20) for all serotypes.

### Example 3

### Surface Exposure and Function of GBS Antigens

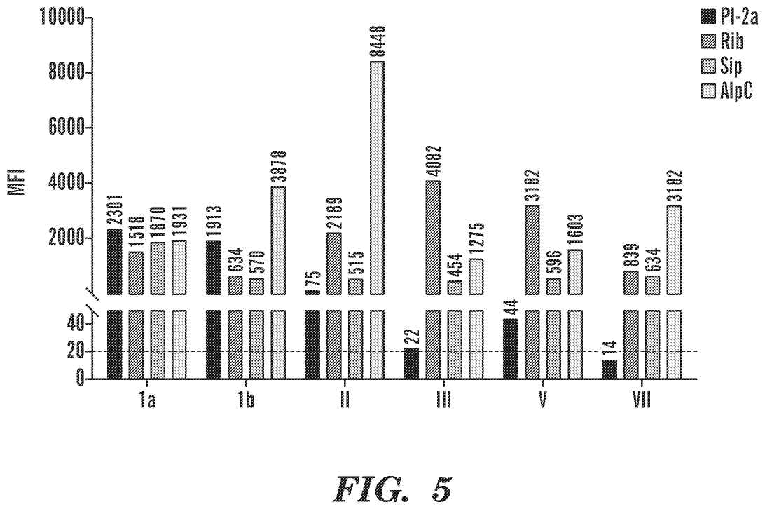

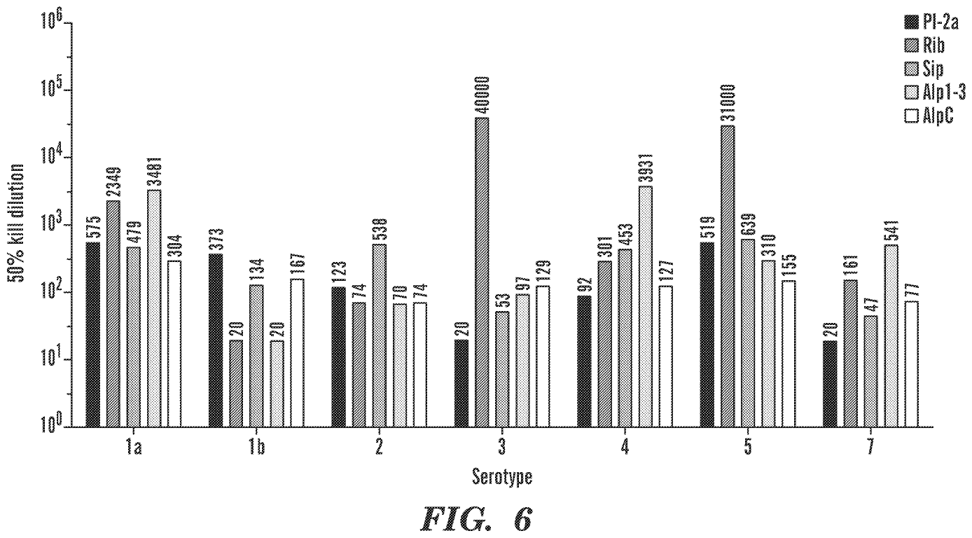

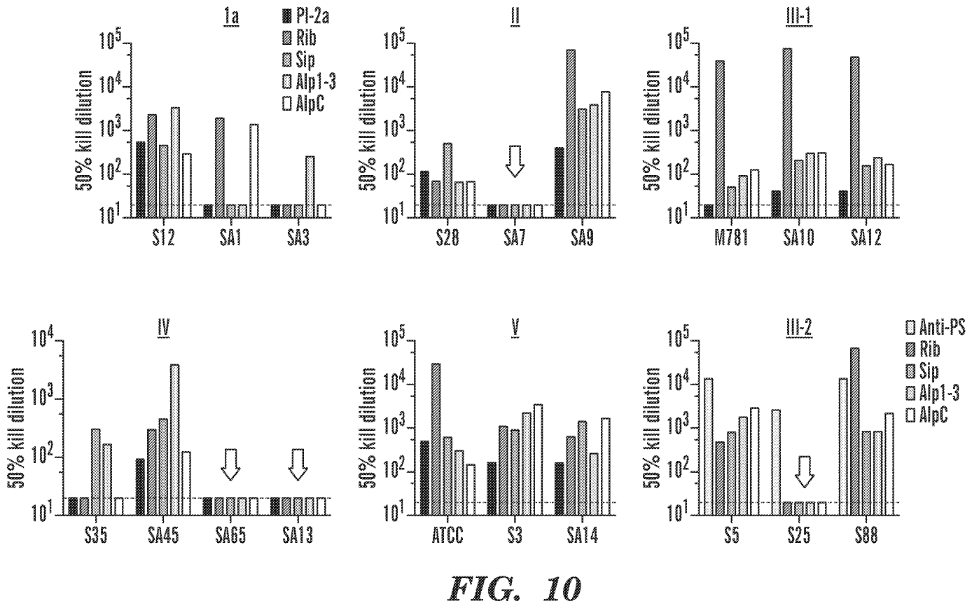

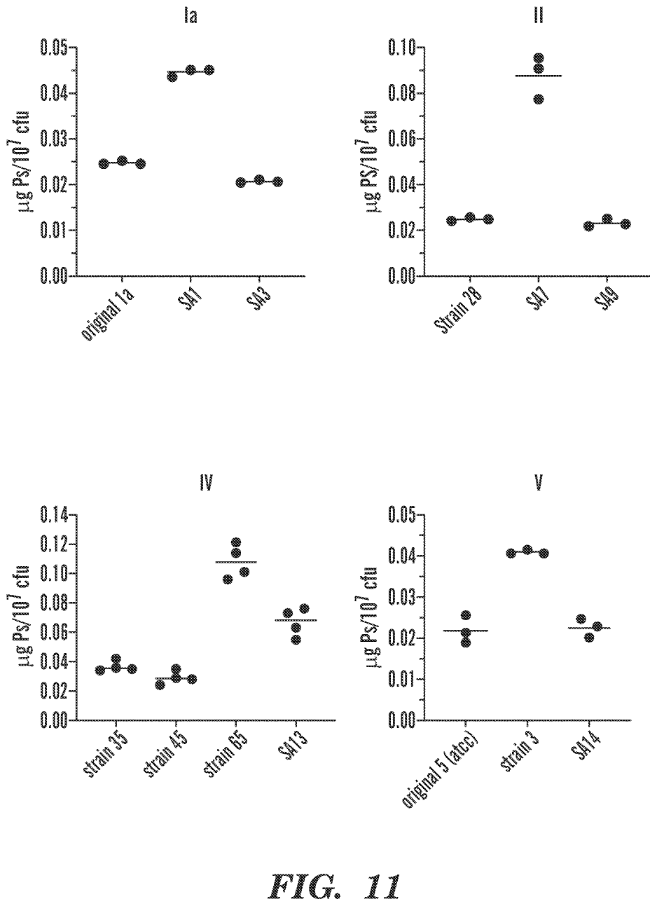

Antisera against PI-2a, Rib, Sip or AlpC were used in a flow cytometry analysis to determine the exposure of each protein on the surface of GBS with six different serotypes. As shown in FIG. 5, Rib sera had the highest surface binding to type III and V, while AlpC sera bound to type Ib, II and VII. These sera with the addition of anti-sera against Alp1-3 fusion protein, were used in OPK assay against GBS clinical strains (7 serotypes). Rib sera had high OPK titer against type III and V, consistent with the flow cytometry analysis (FIG. 6). The other sera had different levels of OPK titers depending on the serotype. Thus, it suggests that the expression patterns of these proteins are different among clinical strains. More clinical strains were tested in the OPK assay using these protein sera. As shown in FIG. 10, some clinical strains are completely resistant to the killing of protein sera, implying that a combination of anti-PS and anti-protein sera is necessary for developing the MAPS-GBS vaccine. One possible explanation of the resistance of protein sera by these clinical strains can be explained by their high capsular polysaccharide expression as shown in FIG. 11, where the resistant strains have 2-4 fold higher PS content.

### Example 4

### Protection Against Invasive Disease by Passive Transfer

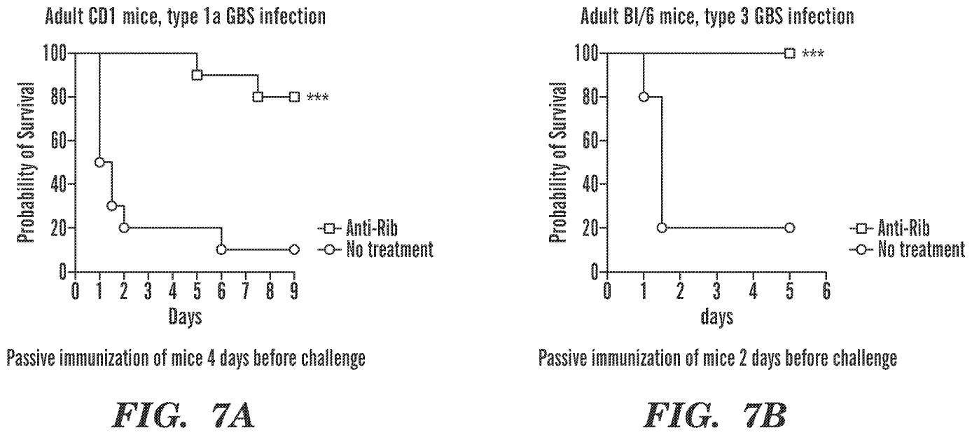

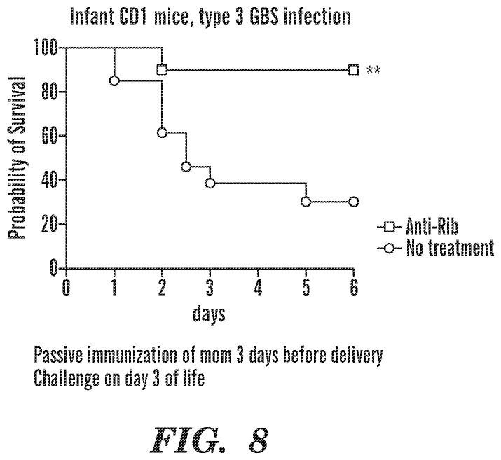

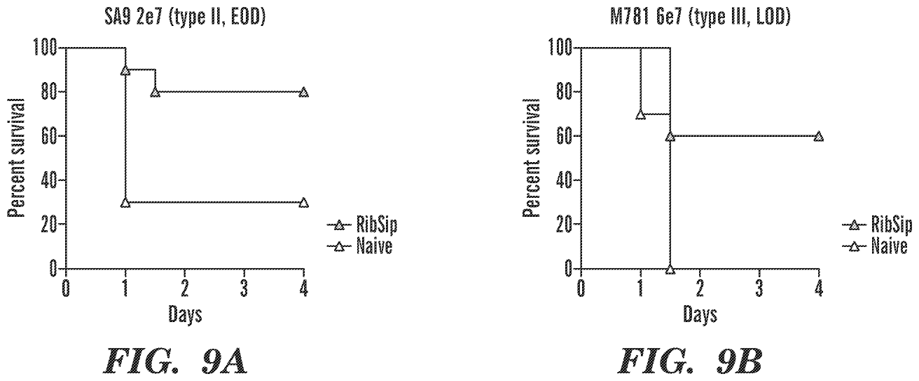

Anti-Rib sera was tested for protection against invasive infection in animal models. Passive transfer of Rib sera protected adult CD1 (type Ia) and BL6 mice (type III) (FIG. 7A-7B). Passive transfer of Rib sera to the pregnant mom also protected against invasive infection of the offspring by type III GBS (infant mice, FIG. 8). Furthermore, antiserum against a fusion protein of Rib and Sip was tested for protection against two more clinical strains. As shown in FIG. 9A-9B, passive transfer of this serum protected against serotype II and serotype III infection in CD1 infection models.

### Example 5

### Synergistic Killing Effect by Combination of Protein and PS Antisera

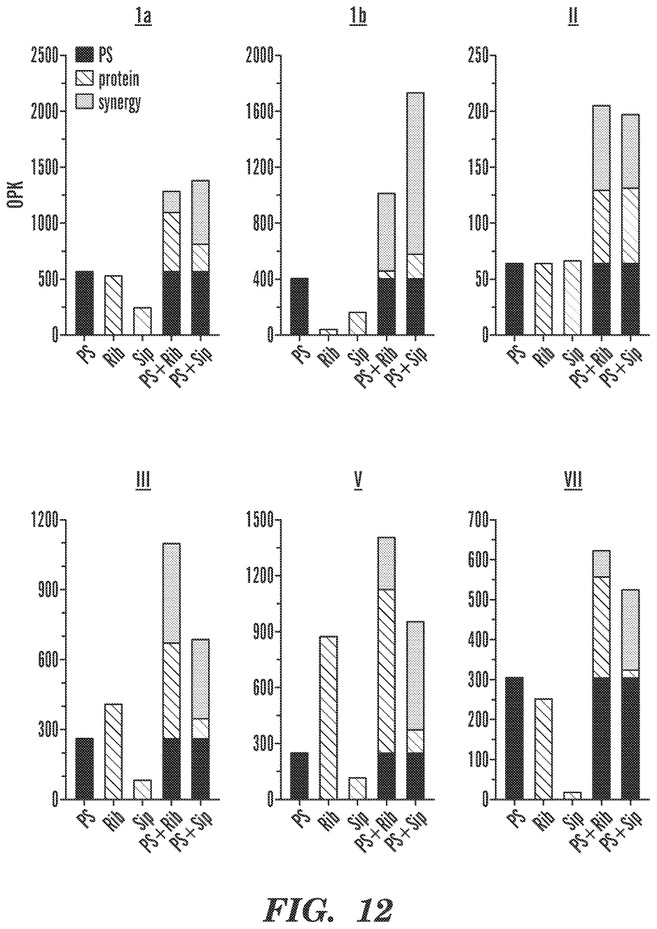

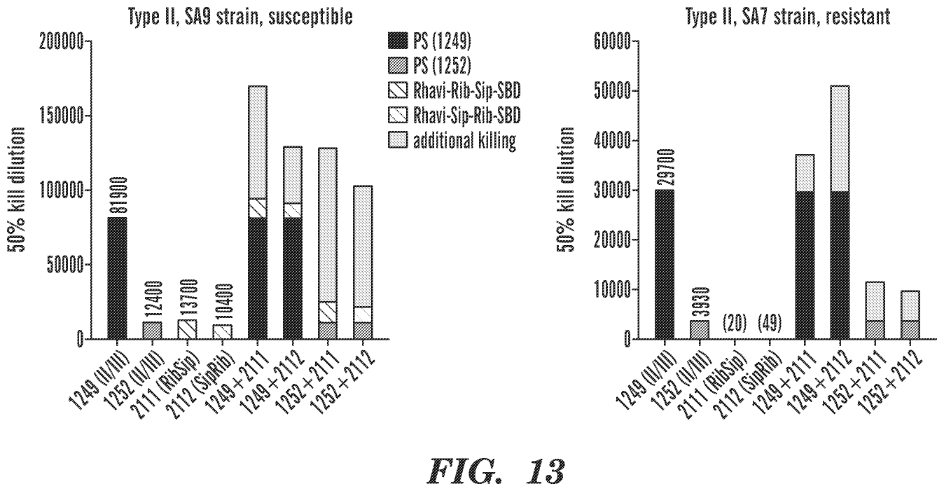

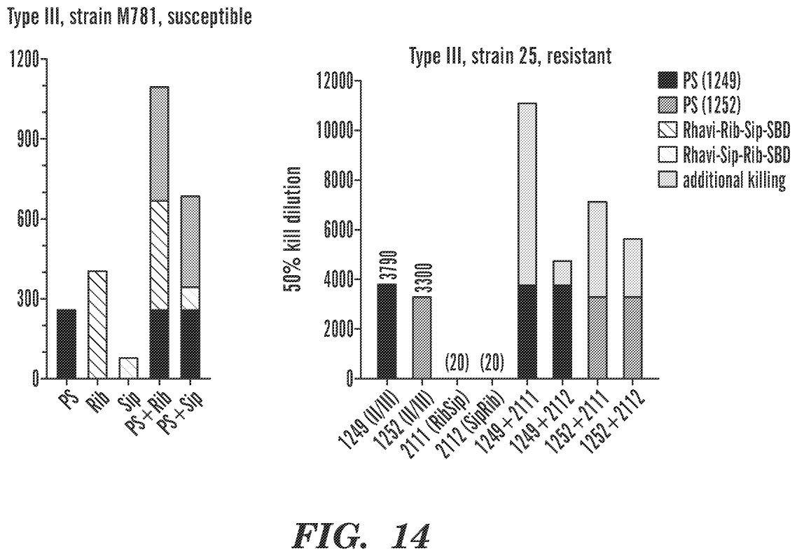

Combinations of anti-antigenic polysaccharides (anti-PS) and antibodies to the antigenic polypeptides (anti-Rib or anti-Sip) resulted in synergy (e.g., the combined effect is greater than the sum of each individually) (FIG. 12). The synergy effect was present in all six serotypes we tested (Ia, Ib, II, III, V and VII). Since there are strains that are resistant to the killing by anti-protein sera, we further tested whether a synergy effect can be observed in these resistant strains. As shown in FIG. 13 and FIG. 14, pairs of strains (sensitive or resistant) from the same serotype were tested in the killing assay with either individual serum or a combination of sera. Surprisingly, the synergy effect can be observed even in the resistant strains.

### Example 6

### Effect of Dose and Immunization Schedule on PS Immunogenicity.

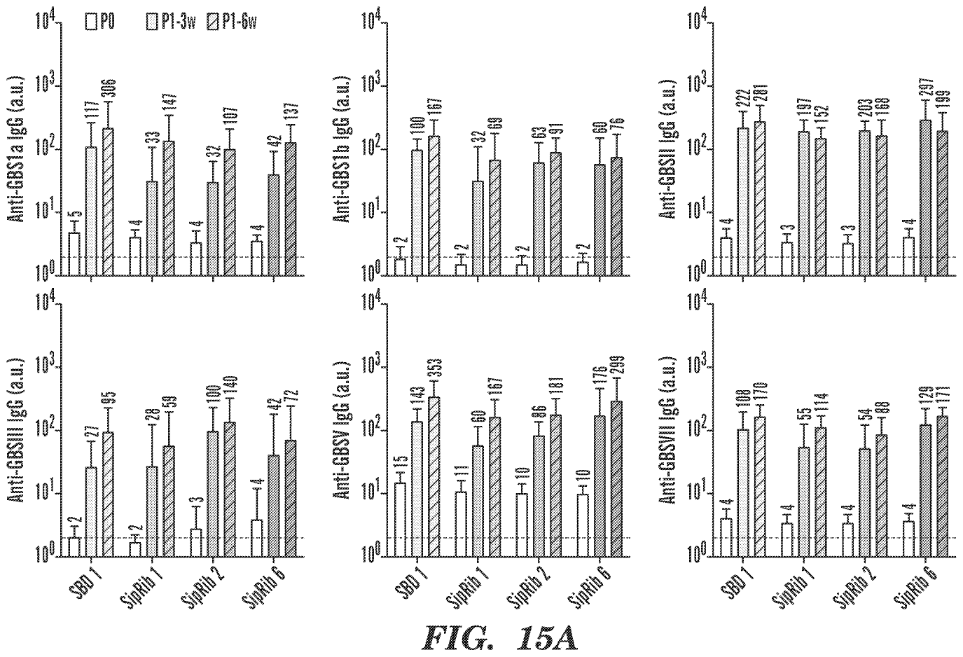

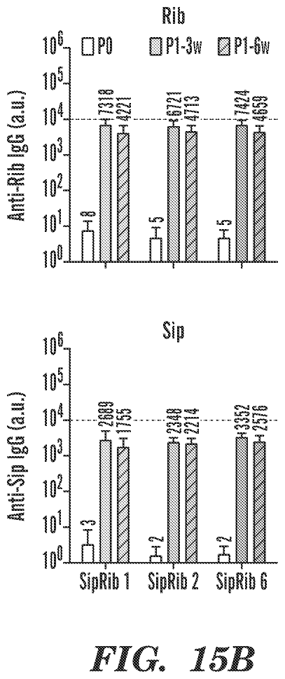

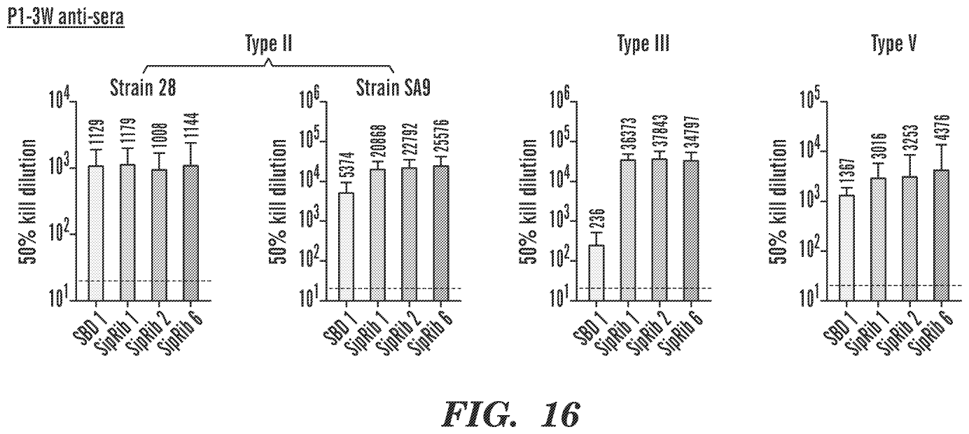

A MAPS-GBS immunogenic complex comprising Rhavi-SBD and Rhavi-Sip-Rib-SBD were used to immunize rabbits. Anti-PS and anti-Rib, anti-Sip ELISA titers were analyzed at 3 weeks or 6 weeks after one immunization. As shown in FIG. 15A, the anti-PS titer was higher at 6 weeks post one immunization, and 1-2 ug per dose is optimal for Rhavi-Sip-Rib-SBD MAPS. In contrast, anti-protein antibody stayed flat at 3 weeks or 6 weeks post immunization (FIG. 15B). Assessment of the killing titer of antisera from the immunization of Rhavi-Sip-Rib-SBD MAPS-GBS against serotypes II (strains 28 and SA9), III, and V GBS stains was also performed. As shown in FIG. 16, Rhavi-Sip-Rib-SBD MAPS induced higher killing activity against all three serotypes.

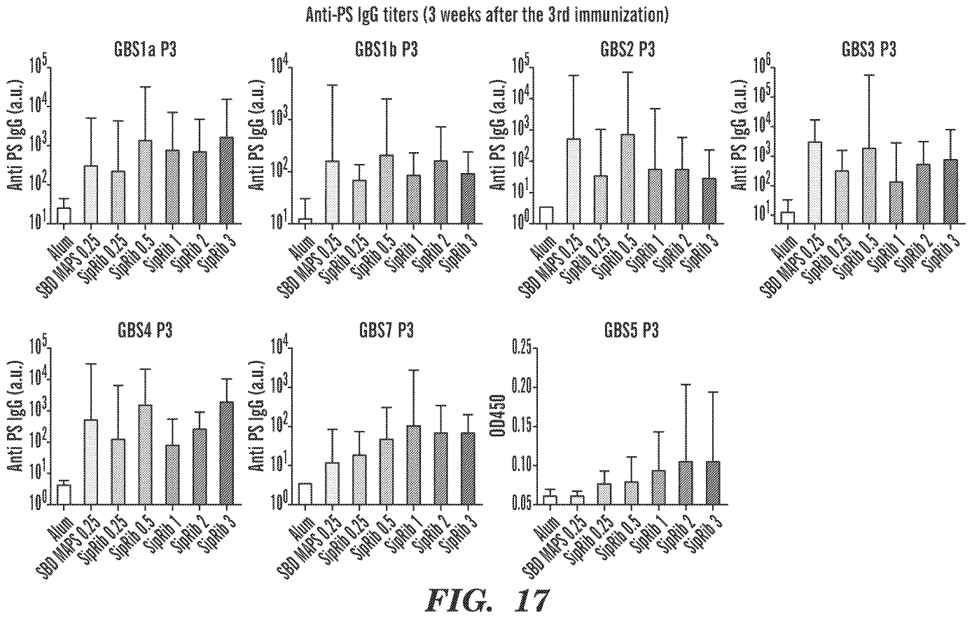

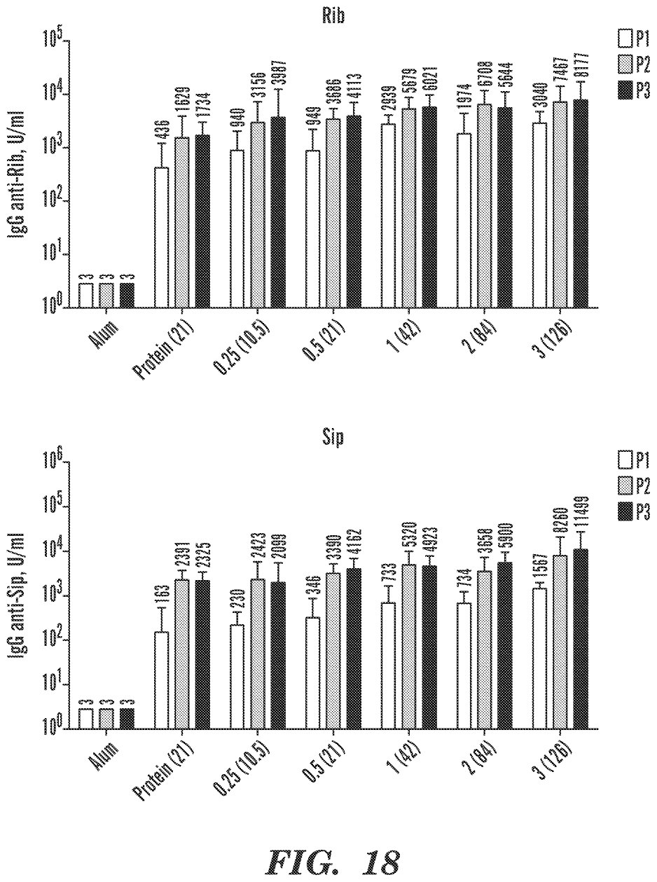

A seven-valent Rhavi-Sip-Rib-SBD MAPS was used to confirm the optimal dose to induce anti-PS antibody. As shown in FIG. 17, the seven valent vaccine induced robust antibody responses to all PS after one injection for all the doses tested. The antibody response to Rib and Sip proteins were also measured by ELISA. As shown in FIG. 18, antibody response was peaked after two immunizations.

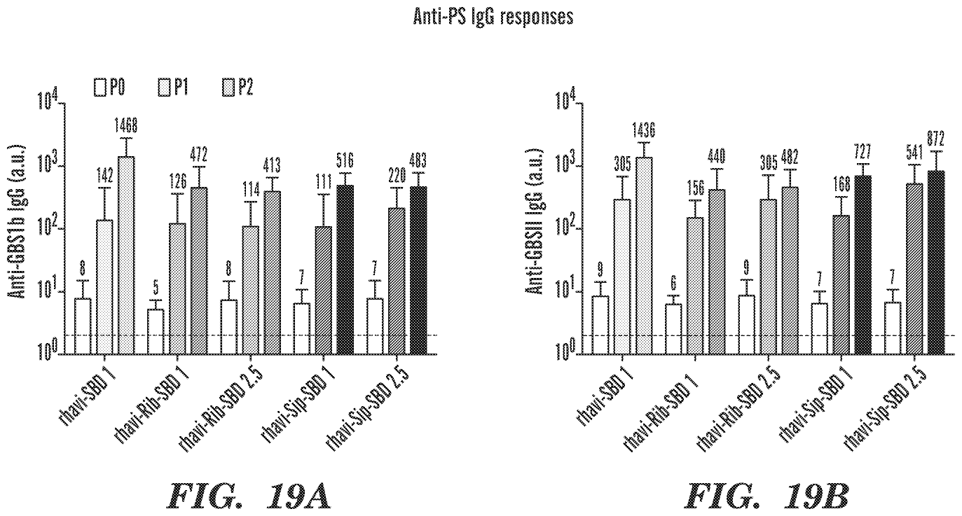

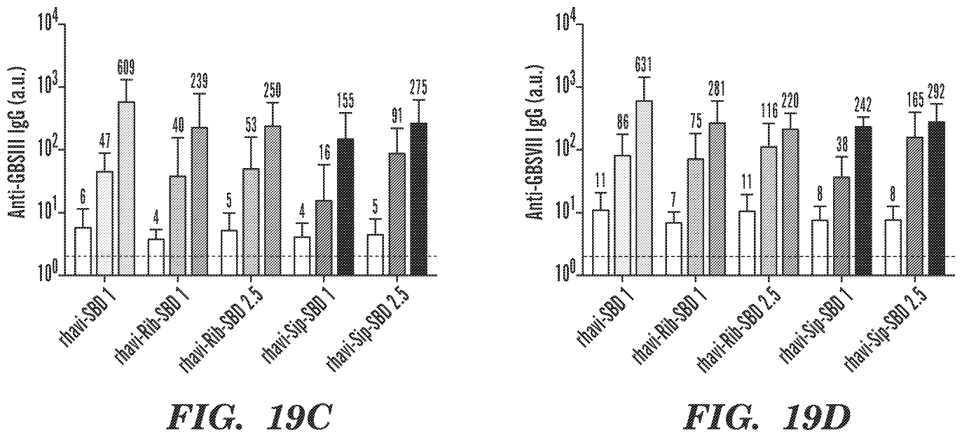

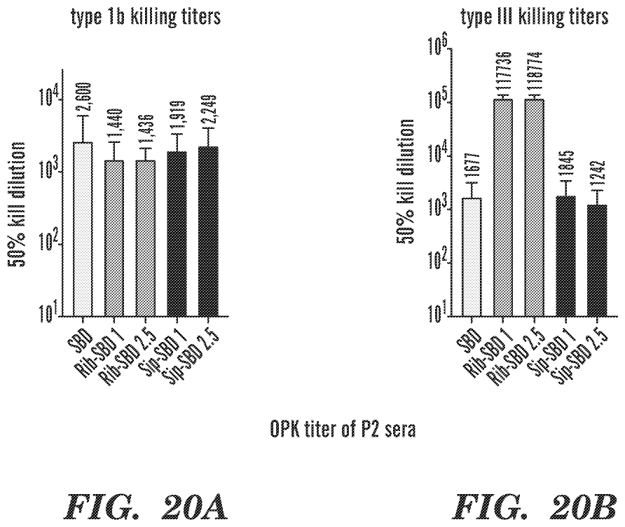

Two new fusion proteins were used to test the dose response of Rhavi-Rib-SBD and Rhavi-Sip SBD MAPS in rabbits. As shown in FIG. 19A-19D, the anti-PS antibody responses were similar between two proteins with different doses. The rabbit sera post two immunizations were used in an OPK assay to measure the titer against two different serotypes. As shown in FIG. 20A-20B, Rib containing MAPS generated much higher killing titer against type III strain, while there was no difference between other fusion proteins and doses.

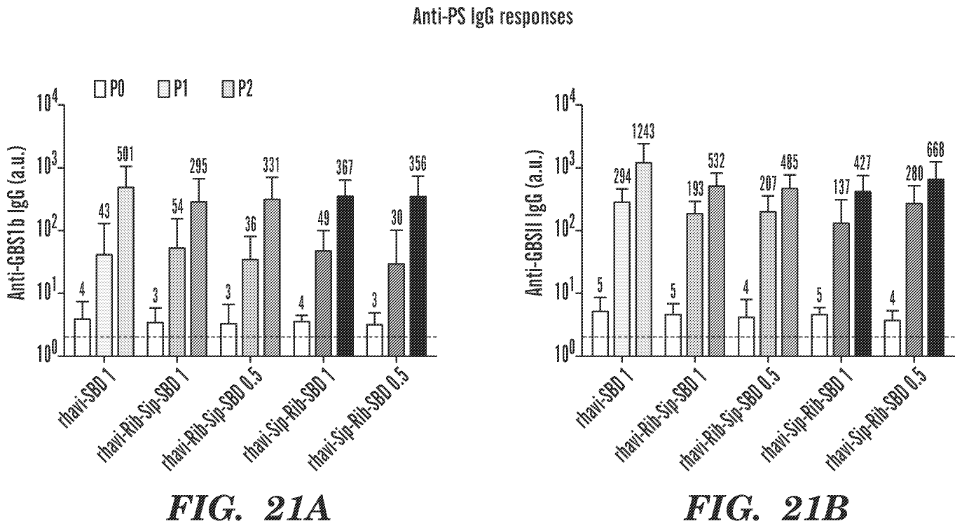

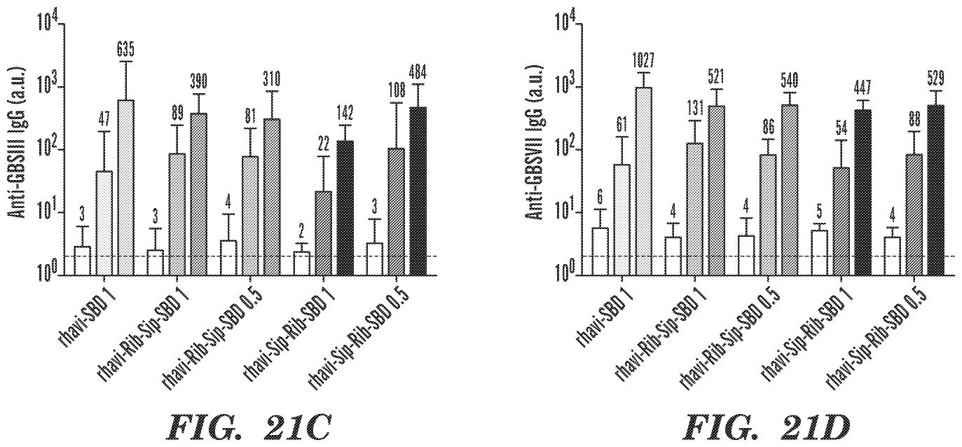

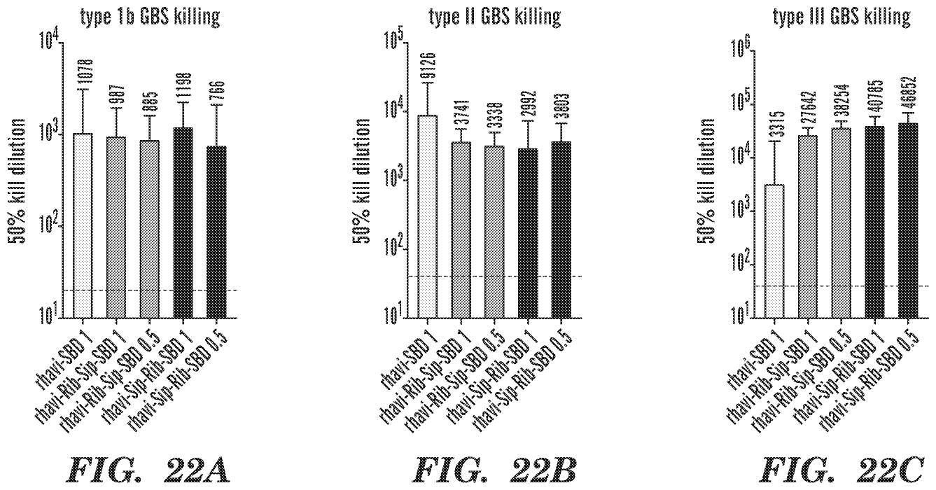

A comparison between the immunogenicity of MAPS-GBS comprising Rhavi-Rib-Sip-SBD and Rhavi-Sip-Rib-SBD in rabbits was carried out at 1 and 0.5 microgram dose. Both fusion proteins induced high anti-PS responses, comparable to that induced by Rhavi-SBD fusion protein (FIG. 21). The sera were tested in OPK assay for three serotypes of clinical strains. Both Rhavi-Rib-Sip-SBD and Rhavi-Sip-Rib-SBD induced high killing titers against all three strains (FIG. 22). The protein response was compared between these two fusion protein MAPS complexes, and the results showed that they made similar anti-Rib and anti-Sip antibody (FIG. 23).

### Example 7

### Rib-Sip-Rhavi-SBD MAPS is Better for Inducing PS Antibody

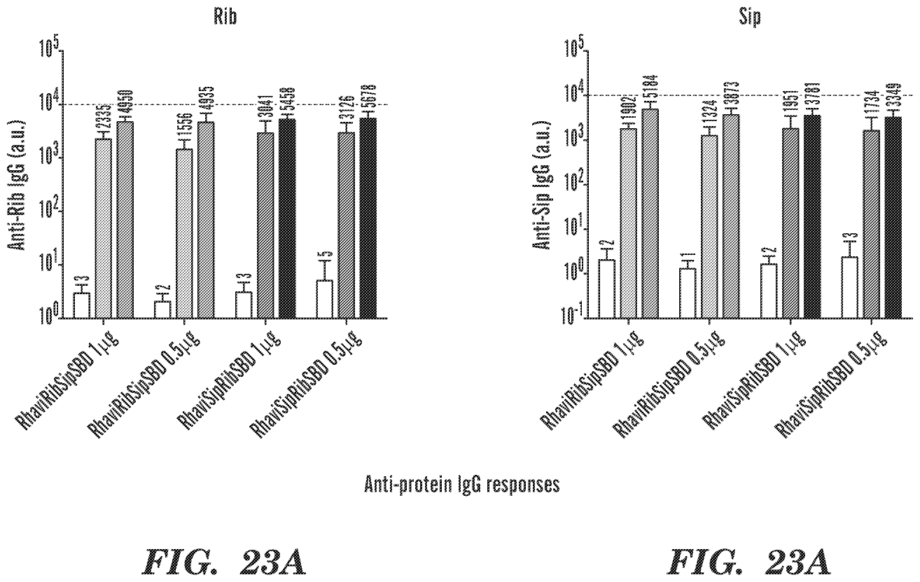

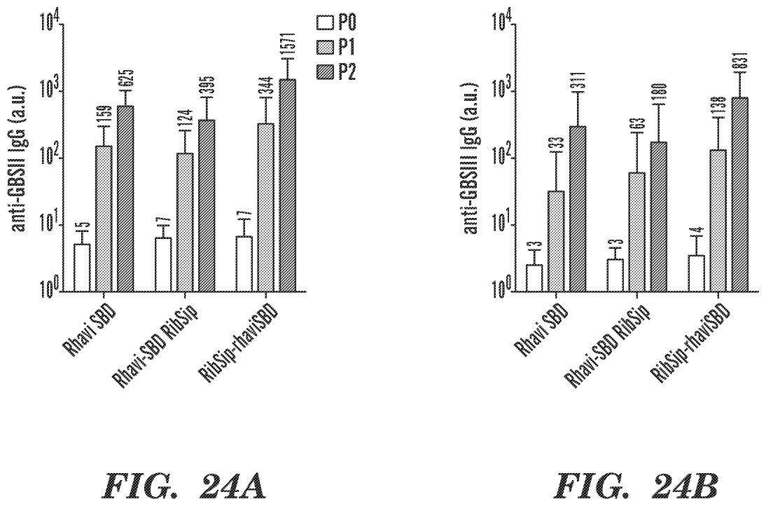

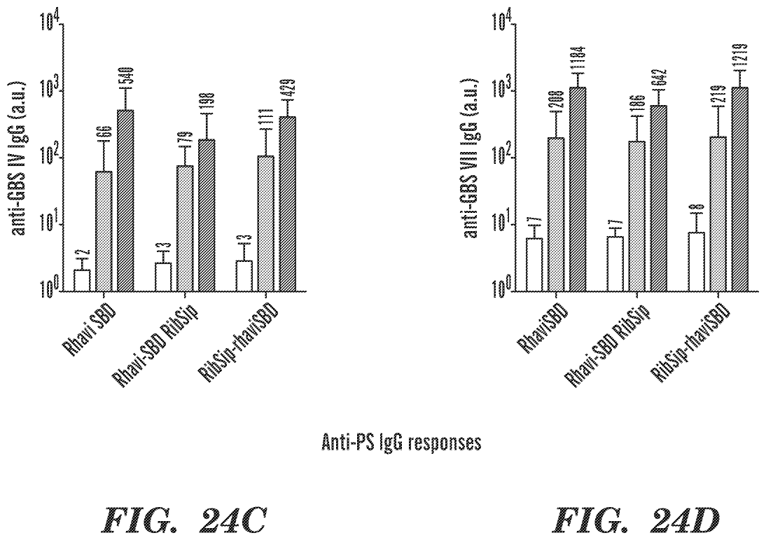

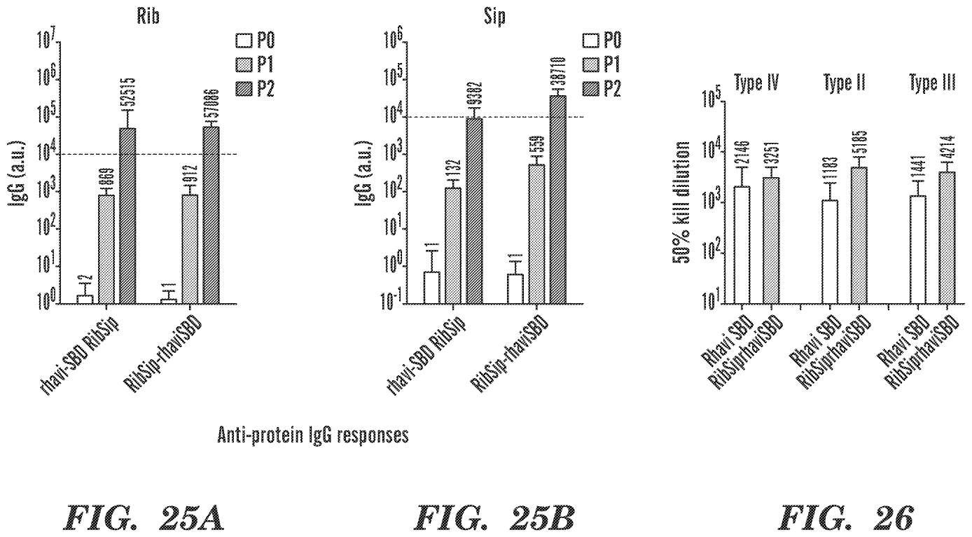

Three fusion proteins (Rhavi-SBD, Rhavi-SBD-Rib-Sip, and Rib-Sip-Rhavi-SBD) were used to generate MAPS and the complexes were used to immunize rabbits. As shown in FIG. 24A-24D, Rib-Sip-Rhavi-SBD MAPS made significantly higher antibody to all four PS included in the MAPS. It also generated higher anti-Rib and anti-Sip antibody responses comparing to Rhavi-SBD-Rib-Sip (FIG. 25A-25B). The killing titer of Rib-Sip-Rhavi-SBD MAPS sera are comparable or higher than those induced by Rhavi-SBD MAPS (FIG. 26).

### Example 8

### Protection by MAPS-GBS in Animal Models

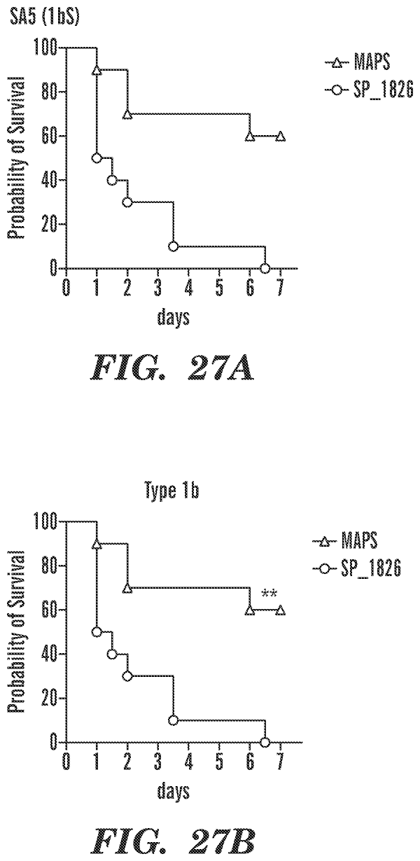

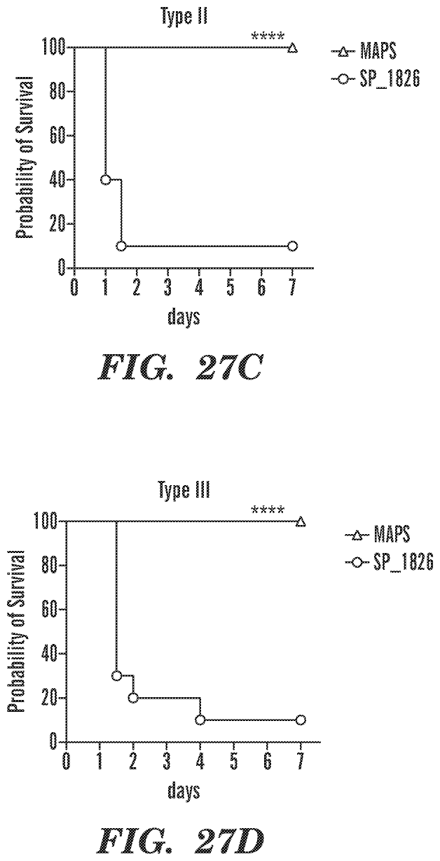

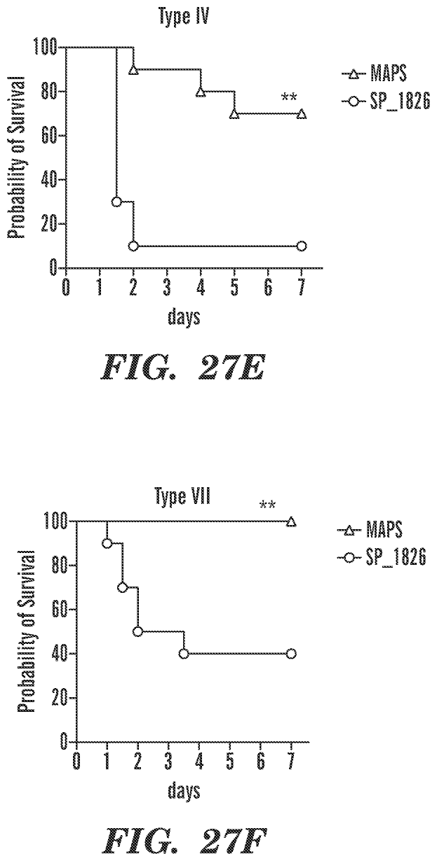

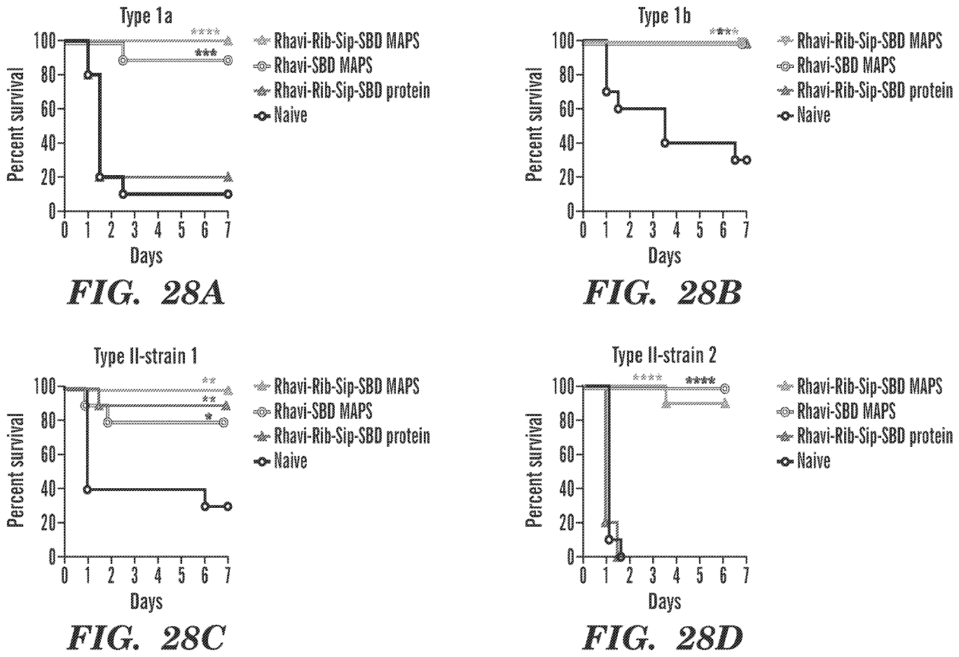

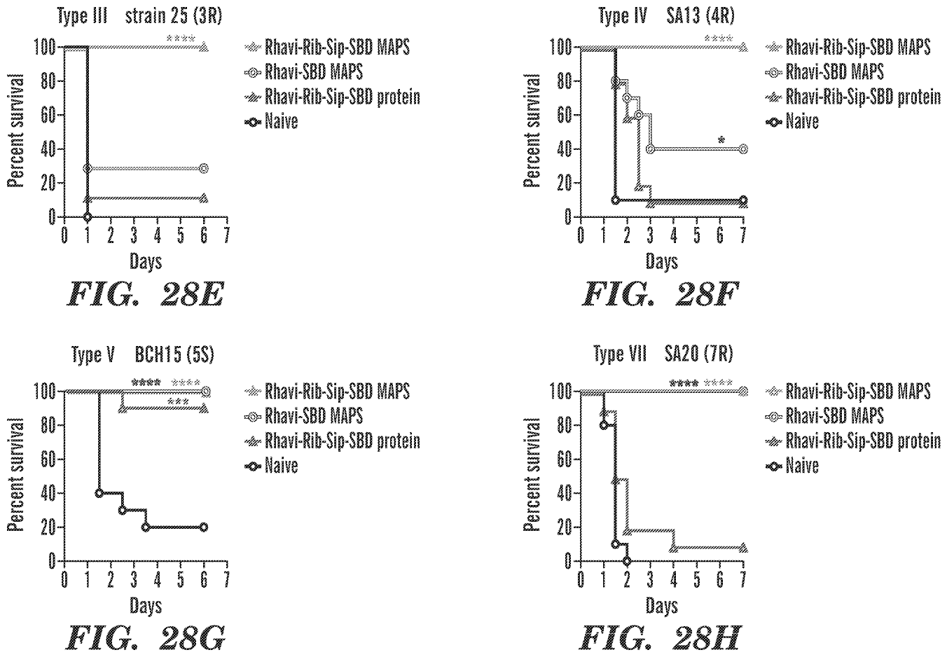

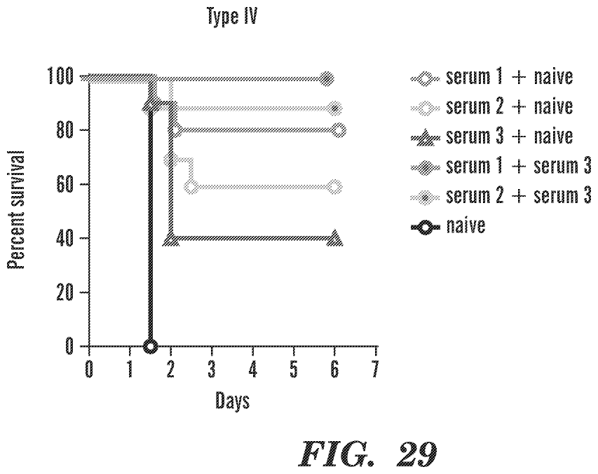

CD1 mice were immunized with a 7V MAPS-GBS immunogenic complexes comprising a fusion protein rhavi-Rib-Sip-SBD three times. Mice were infected with serotypes Ib, II, III, IV and VII strains. MAPS-GBS showed better protection than the negative control (irrelevant protein), as shown in FIG. 27A-27F. A passive transfer experiment using rabbits received 7V MAPS-GBS immunogenic complexes comprising a fusion protein selected from Rhavi-Rib-Sip-SBD, or Rhavi-SBD, or administration of the Rhavi-Rib-Sip-SBD protein alone showed that MAPS-GBS complexes made with Rhavi-Rib-Sip-SBD generated better or similar protection against a few serotypes of GBS strains (FIG. 28A-28H). It was also demonstrated that a combination of anti-PS and anti-Rib/Sip sera can indeed induced a synergistic protective effect against a type IV infection (FIG. 29). Mice that received both sera had a much better survival rate than those that received the same volume of single serum.

1. A fusion protein comprising, in any order, (i) a biotin binding moiety (BBM), (ii) a sialic acid binding domain (SBD) polypeptide and (iii) at least a first polypeptide antigen or antigenic fragment thereof from Group B Streptococcus agalactiae (GBS-Ag).

2. The fusion protein of claim 1, wherein the fusion protein comprises in the following order, from N-terminus to C-terminus:
a BBM, a first GBS-Ag, a SBD;
a SBD, a first GBS-Ag, a BBM;
a BBM, a SBD, a first GBS-Ag; or
a first GBS-Ag, a BBM, a SBD

3. The fusion protein of claim 1 or 2, further comprising at least a second polypeptide antigen from Group B Streptococcus agalactiae (GBS-Ag), wherein the second GBS-Ag is fused to at least the first GBS-Ag.

4. The fusion protein of claim 3, further comprising at least a third polypeptide antigen from Group B Streptococcus agalactiae (GBS-Ag), wherein the third GBS-Ag is fused to either the first GBS-Ag or the second GBS-Ag.

5. The fusion protein of any of claims 1-4, wherein the first, second or third GBS antigen is selected from the group consisting of: Rib, Sip, AlpC, Alp1, Alp3 or Alp3/1.

6. The fusion protein of any of claims 1-5, wherein the fusion protein is selected from:
a. a fusion protein comprising in order from N-terminus to C terminus: (i) the biotin-binding moiety (BBM), (ii) a Rib polypeptide antigen or an antigenic fragment thereof, or a Sip polypeptide or an antigenic fragment thereof, and (iii) the SBD polypeptide, or
b. a fusion protein comprising in order from N-terminus to C terminus: (i) the biotin-binding moiety (BBM), (ii) a Rib polypeptide antigen or an antigenic fragment thereof and a Sip polypeptide or an antigenic fragment thereof, and (iii) the SBD polypeptide.

7. The fusion protein of any of claims 1-6, wherein the fusion protein is selected from:
a. a fusion protein comprising in order from N-terminus to C terminus: (i) a SBD polypeptide, (ii) Rib polypeptide antigen or an antigenic fragment thereof, or Sip polypeptide or an antigenic fragment thereof, and (iii) a biotin-binding moiety (BBM), or
b. a fusion protein comprising in order from N-terminus to C terminus: (i) a SBD polypeptide, (ii) Rib polypeptide antigen or an antigenic fragment thereof and Sip polypeptide or an antigenic fragment thereof, and (iii) the biotin-binding moiety (BBM).

8. The fusion protein of any of claims 1-7, wherein the BBM is Rhizavidin comprises an amino acid sequence having at least 80%, or at least 85%, or at least 90%, or at least 95% sequence identity to SEQ ID NO: 1.

9. The fusion protein of any of claims 1-8, wherein the SBD is selected from any of: SBD1, SBD2, SBD3, SBD4, NanH, NanH2, VcNanH, or a SBD having an amino acid sequence that has at least 80%, or at least 85%, or at least 90%, or at least 95% sequence identity to any of: SEQ ID NO: 2 or SEQ ID NO: 112-120.

10. The fusion protein of any of claims 1-9, wherein the fusion protein comprises an amino acid sequence having at least 80%, or at least 85%, or at least 90%, or at least 95% sequence identity to any of: SEQ ID NO: 3 (Rhavi-SBD1), SEQ ID NO: 129 (Rhavi-SBD2), SEQ ID NO: 140 (Rhavi-SBD3), or SEQ ID NO: 151 (Rhavi-SBD4).

11. The fusion protein of any of claims 1-10, wherein any of the first, second or third polypeptide antigen comprises an amino acid sequence having at least 80%, or at least 85%, or at least 90%, or at least 95% sequence identity to SEQ ID NO: 4 (Rib).

12. The fusion protein of any of claims 1-11, wherein any of the first, second or third polypeptide antigen comprises an amino acid sequence having at least 80%, or at least 85%, or at least 90%, or at least 95% sequence identity to SEQ ID NO: 5 (Sip).

13. The fusion protein of any of claims 1-12, wherein any of the first, second or third or polypeptide comprises an amino acid sequence having at least 80%, or at least 85%, or at least 90%, or at least 95% sequence identity to any of: SEQ ID NO: 4 (Rib), SEQ ID NO: 5 (Sip), SEQ ID NO: 11 (AlpC), SEQ ID NO: 12 (Alp1); SEQ ID NO: 13 (Alp3), SEQ ID NO: 14 (Alp3/1).

14. The fusion protein of any of claims 1-13, wherein the fusion protein comprises,
a. an amino acid sequence having at least 80%, or at least 85%, or at least 90%, or at least 95% sequence identity to SEQ ID NO: 1 (Rhavi),
b. a SBD immunogenic polypeptide, selected from:
i. an amino acid sequence having at least 80%, or at least 85%, or at least 90%, or at least 95% sequence identity to SEQ ID NO: 2 (SBD1);
ii. an amino acid sequence having at least 80%, or at least 85%, or at least 90%, or at least 95% sequence identity to SEQ ID NO: 112 (SBD2),
iii. an amino acid sequence having at least 80%, or at least 85%, or at least 90%, or at least 95% sequence identity to SEQ ID NO: 113 (SBD3),
iv. an amino acid sequence having at least 80%, or at least 85%, or at least 90%, or at least 95% sequence identity to SEQ ID NO: 114 (SBD4),
v. an amino acid sequence having at least 80%, or at least 85%, or at least 90%, or at least 95% sequence identity to SEQ ID NO: 115 (NanH),
vi. an amino acid sequence having at least 80%, or at least 85%, or at least 90%, or at least 95% sequence identity to SEQ ID NO: 116 (NanH2),
vii. an amino acid sequence having at least 80%, or at least 85%, or at least 90%, or at least 95% sequence identity to SEQ ID NO: 117 (NanH3), or
viii. an amino acid sequence having at least 80%, or at least 85%, or at least 90%, or at least 95% sequence identity to SEQ ID NO: 120 (VcNanH),
c. an amino acid sequence having at least 80%, or at least 85%, or at least 90%, or at least 95% sequence identity to SEQ ID NO: 4 (Rib); and
d. an amino acid sequence having at least 80%, or at least 85%, or at least 90%, or at least 95% sequence identity to SEQ ID NO: 5 (Sip).

15. The fusion protein of any of claims 1-13, comprising, in the following order:
a. an amino acid sequence having at least 80%, or at least 85%, or at least 90%, or at least 95% sequence identity to SEQ ID NO: 4 (Rib),
b. an amino acid sequence having at least 80%, or at least 85%, or at least 90%, or at least 95% sequence identity to SEQ ID NO: 5 (Sip),
c. an amino acid sequence having at least 80%, or at least 85%, or at least 90%, or at least 95% sequence identity to SEQ ID NO: 1 (Rhavi), and
d. an amino acid sequence at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3) or SEQ ID NO: 120 (VcNanH)

16. The fusion protein of any of claims 1-15, wherein the fusion protein comprises an amino acid sequence having at least 80%, or at least 85%, or at least 90%, or at least 95% sequence identity to SEQ ID NO: 6 (Rhavi-[Rib]-[Sip]-SBD1).

17. The fusion protein of any of claims 1-13, comprising, in the following order:
i. an amino acid sequence having at least 80%, or at least 85%, or at least 90%, or at least 95% sequence identity to SEQ ID NO: 1 (Rhavi).
ii. an amino acid sequence having at least 80%, or at least 85%, or at least 90%, or at least 95% sequence identity to SEQ ID NO: 5 (Sip),
iii. an amino acid sequence having at least 80%, or at least 85%, or at least 90%, or at least 95% sequence identity to SEQ ID NO: 4 (Rib), and
iv. an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3).

18. The fusion protein of claim 17, wherein the fusion protein comprises an amino acid sequence having at least 80%, or at least 85%, or at least 90%, or at least 95% sequence identity to SEQ ID NO: 7 (Rhavi-[Sip]-[Rib]-SBD).

19. The fusion protein of any of claims 1-13, comprising, in the following order:
a. an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3).
b. an amino acid sequence having at least 80%, or at least 85%, or at least 90%, or at least 95% sequence identity to SEQ ID NO: 4 (Rib),
c. an amino acid sequence having at least 80%, or at least 85%, or at least 90%, or at least 95% sequence identity to SEQ ID NO: 5 (Sip), and
d. an amino acid sequence having at least 80%, or at least 85%, or at least 90%, or at least 95% sequence identity to SEQ ID NO: 1 (Rhavi).

20. The fusion protein of any of claims 1-13, comprising, in the following order:
a. an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3),
b. an amino acid sequence having at least 80%, or at least 85%, or at least 90%, or at least 95% sequence identity to SEQ ID NO: 5 (Sip),
c. an amino acid sequence having at least 80%, or at least 85%, or at least 90%, or at least 95% sequence identity to SEQ ID NO: 4 (Rib), and
d. an amino acid sequence having at least 80%, or at least 85%, or at least 90%, or at least 95% sequence identity to SEQ ID NO: 1 (Rhavi).

21. The fusion protein of any of claims 1-20, comprising at least one linker sequence or at least one spacer sequence located between any of: the BBM and the SBD; the BBM and at the first polypeptide antigen or an antigenic fragment thereof, the first polypeptide antigen or an antigenic fragment thereof and the second polypeptide antigen or an antigenic fragment thereof, or a polypeptide antigen or an antigenic fragment thereof and a SBD.

22. The fusion protein of any of claims 1-21, wherein the linker sequence or spacer sequence is selected from any of: GGGGSSS, TDPNSSS, SSS, AAA.

23. The fusion protein of any of claims 1-4, wherein the fusion protein is selected from any listed in Table 2A, 2B or Table 3, or comprises an amino acid sequence having at least 80%, or at least 85%, or at least 90%, or at least 95% sequence identity to any of the amino acid sequences listed in Table 3.

24. A fusion protein comprising a sialic acid binding domain (SBD) polypeptide and at least a first polypeptide antigen or an antigenic fragment thereof from Group B Streptococcus agalactiae (GBS-Ag).

25. The fusion protein of claim 24, further comprising at least a second polypeptide antigen from Group B Streptococcus agalactiae (GBS-Ag), wherein the second GBS-Ag is fused to at least the first GBS-Ag.

26. The fusion protein of claim 25, further comprising at least a third polypeptide antigen from Group B Streptococcus agalactiae (GBS-Ag), wherein the third GBS-Ag is fused to either the first GBS-Ag or the second GBS-Ag.

27. The fusion protein of any of claims 24-26, wherein the first, second or third GBS antigen is selected from the group consisting of: Rib, Sip, AlpC, Alp1, Alp3 or Alp3/1.

28. The fusion protein of any of claim 24, wherein the first polypeptide antigen or an antigenic fragment thereof is a Rib polypeptide.

29. The fusion protein of any of claim 24, wherein the first polypeptide antigen or an antigenic fragment thereof is a Sip polypeptide.

30. The fusion protein of any of claim 25, wherein
c. the first GBS polypeptide antigen or an antigenic fragment thereof is a Rib polypeptide, and the second GBS polypeptide antigen or antigenic fragment is a Sip polypeptide, or
d. the first polypeptide antigen or an antigenic fragment thereof is a Sip polypeptide, and the second polypeptide antigen or antigenic fragment is a Rib polypeptide.

31. The fusion protein of any of claims 24-30, wherein the fusion protein is selected from:
a. a fusion protein comprising in order from N-terminus to C terminus: (i) a SBD polypeptide, (ii) Rib polypeptide antigen or an antigenic fragment thereof, or Sip polypeptide or an antigenic fragment thereof, or
b. a fusion protein comprising in order from N-terminus to C terminus: (i) a SBD polypeptide, (ii) Rib polypeptide antigen or an antigenic fragment thereof, and Sip polypeptide or an antigenic fragment thereof, wherein the Rib polypeptide antigen or an antigenic fragment thereof and Sip polypeptide or an antigenic fragment thereof can be in any order.

32. The fusion protein of claims 24-30, wherein the fusion protein is selected from:
a. a fusion protein comprising in order from N-terminus to C terminus: (i) Rib polypeptide antigen or an antigenic fragment thereof, or Sip polypeptide or an antigenic fragment thereof, and (ii) a SBD polypeptide, or
b. a fusion protein comprising in order from N-terminus to C terminus: (i) Rib polypeptide antigen or an antigenic fragment thereof, and Sip polypeptide or an antigenic fragment thereof, (ii) a SBD polypeptide, wherein the Rib polypeptide antigen or an antigenic fragment thereof and Sip polypeptide or an antigenic fragment thereof can be in any order.

33. The fusion protein of any of claims 24-32, wherein the SBD is selected from any of: SBD1, SBD2, SBD3, SBD4, NanH, NanH2, VcNanH, or a SBD having an amino acid sequence that has at least 80%, or at least 85%, or at least 90%, or at least 95% sequence identity to any of: SEQ ID NO: 2 or SEQ ID NO: 112-120.

34. The fusion protein of any of claims 24-33, wherein the SBD comprises an amino acid sequence having at least 80%, or at least 85%, or at least 90%, or at least 95% sequence identity to SEQ ID NO: 2 or SEQ ID NO: 112.

35. The fusion protein of any of claims 24-34, wherein the first polypeptide antigen comprises an amino acid sequence having at least 80%, or at least 85%, or at least 90%, or at least 95% sequence identity to SEQ ID NO: 4 (Rib).

36. The fusion protein of any of claims 24-29, wherein the first polypeptide antigen comprises an amino acid sequence having at least 80%, or at least 85%, or at least 90%, or at least 95% sequence identity to SEQ ID NO: 5 (Sip).

37. The fusion protein of any of claim 24, comprising at least one linker sequence or at least one spacer sequence located between any of: the SBD and at the first polypeptide antigen or an antigenic fragment thereof, the first polypeptide antigen or an antigenic fragment thereof and the second polypeptide antigen or an antigenic fragment thereof, or a polypeptide antigen or an antigenic fragment thereof and a SBD.

38. The fusion protein of any of claims 1-21, wherein the linker sequence or spacer sequence is selected from any of: GGGGSSS, TDPNSSS, SSS, AAA.

39. The fusion protein of any of claims 24-38, wherein the fusion protein is selected from any SBD fusion proteins listed in Table 2A, 2B or Table 3, or comprises an amino acid sequence having at least 80%, or at least 85%, or at least 90%, or at least 95% sequence identity to any of the amino acid sequences listed in Table 3.

40. An immunogenic composition comprising at least one species of MAPS-GBS immunogenic complex, wherein each species of the MAPS-GBS immunogenic complex comprises;
a. at least a first biotinylated polysaccharide antigen (PS1) comprising a first polysaccharide antigen, at least one sialic acid molecule and at least one biotin molecule,
b. at least a second biotinylated polysaccharide antigen (PS2) comprising a second polysaccharide antigen, at least one sialic acid molecule and at least one biotin molecule,
c. at least one fusion protein, each fusion protein comprises at least a biotin-biotin moiety (BBM) and a sialic acid binding domain (SBD),
wherein a BBM of at least one fusion protein non-covalently associates with at least one biotin molecule on the first biotinylated polysaccharide antigen (PS1) and the SBD non-covalently associates with at least one sialic acid on the second biotinylated polysaccharide antigen (PS2), and/or
wherein a SBD of at least one fusion protein non-covalently associates with at least one sialic acid molecule on the first biotinylated polysaccharide antigen (PS1) and the BBM non-covalently associates with at least biotin molecule on the second biotinylated polysaccharide antigen (PS2),
wherein the first biotinylated polysaccharide antigen, the second biotinylated polysaccharide antigen and the fusion protein form a MAPS-GBS immunogenic complex by the non-covalent association of at least one fusion protein to both the first biotinylated polysaccharide antigen and the second biotinylated polysaccharide antigen.

41. The immune composition of claim 40, wherein the PS1 and PS2 are located on the same polysaccharide macromolecule.

42. The immune composition of claim 40, wherein the PS1 and PS2 are located on distinct polysaccharide macromolecules.

43. The immune composition of claim 42, wherein one polysaccharide macromolecule is from a pathogen, and the other macromolecule is from a distinct pathogen or serotype.

44. The immune composition of claim 40, wherein the first or second polysaccharide antigen, or both, is a capsular polysaccharide (CP) from a Group B Streptococcus (GBS) or Streptococcus agalactiae.

45. The immune composition of claim 44, wherein the first polysaccharide antigen and the second polysaccharide antigen are the same serotype of GBS.

46. The immune composition of claim 43, wherein the first polysaccharide antigen is from one serotype of GBS, and the second polysaccharide antigen is from a different serotype of GBS.

47. The immune composition of any of claims 40-46, wherein the immune composition comprises at least 3, or at least 4, or at least 5 or more than 5 species of immunogenic complex.

48. The immune composition of any of claims 40-47, wherein the immune composition comprises at least 6-10, 11-15, 16-20, 21-25, 26-30, or 31-35 or more than 35 or more species of immunogenic complex.

49. The immune composition of any of claims 40-48, wherein the first biotinylated polysaccharide, or second biotinylated polysaccharide, or both, is from any of serotypes 1a, Ib, II, III, IV, V, VI, VII, VIII, and IX of Streptococcus agalactiae.

50. The immune composition of any of claims 40-49, wherein the first biotinylated polysaccharide, or second biotinylated polysaccharide, or both, is selected from any of serotypes 1a, Ib, II, III, IV, V or VII of Streptococcus agalactiae.

51. The immune composition of any of claims 40-50, comprising at least 4 immunogenic complexes, wherein the at least 4 complexes are selected from any of:
i. a MAPS-GBS immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia or a different serotype selected from any of serotypes Ib, II, III, IV, V, VI, VII, VIII, and IX of Streptococcus agalactiae, and (c) at least one fusion protein, or
ii. a MAPS-GBS immunogenic complex comprising (a) a first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib or a different serotype selected from any of serotypes Ia, II, III, IV, V, VI, VII, VIII, and IX of Streptococcus agalactiae, and (c) at least one fusion protein, or
iii. a MAPS-GBS immunogenic complex comprising (a) a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II or a different serotype selected from any of serotypes Ia, Ib, III, IV, V, VI, VII, VIII, and IX of Streptococcus agalactiae, and (c) at least one fusion protein, or
iv. a MAPS-GBS immunogenic complex comprising (a) a first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III or a different serotype selected from any of serotypes Ia, Ib, II, IV, V, VI, VII, VIII, and IX of Streptococcus agalactiae, and (c) at least one fusion protein, or
v. a MAPS-GBS immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV or a different serotype selected from any of serotypes Ia, Ib, II, III, V, VI, VII, VIII, and IX of Streptococcus agalactiae, and (c) at least one fusion protein, or
vi. a MAPS-GBS immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V or a different serotype selected from any of serotypes Ia, Ib, II, III, IV, VI, VII, VIII, and IX of Streptococcus agalactiae, and (c) at least one fusion protein, or
vii. a MAPS-GBS immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII or a different serotype selected from any of serotypes Ia, Ib, II, III, IV, V, VI, VIII, and IX of Streptococcus agalactiae, and (c) at least one fusion protein.

52. The immune composition of claim 40-50, wherein the immune composition comprises the immunogenic complexes of:
i. a first MAPS-GBS immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia or a different serotype selected from any of serotypes Ib, II, III, IV, V, VI, VII, VIII, and IX of Streptococcus agalactiae, and (c) at least one fusion protein,
ii. a second MAPS-GBS immunogenic complex comprising (a) first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib or a different serotype selected from any of serotypes Ia, II, III, IV, V, VI, VII, VIII, and IX of Streptococcus agalactiae, and (c) at least one fusion protein, and (b) at least one fusion protein,
iii. a third MAPS-GBS immunogenic complex comprising a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II or a different serotype selected from any of serotypes Ia, Ib, III, IV, V, VI, VII, VIII, and IX of Streptococcus agalactiae, and (c) at least one fusion protein,
iv. a fourth MAPS-GBS immunogenic complex comprising (a) first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III or a different serotype selected from any of serotypes Ia, Ib, II, IV, V, VI, VII, VIII, and IX of Streptococcus agalactiae, and (c) at least one fusion protein,
v. a firth MAPS-GBS immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V or a different serotype selected from any of serotypes Ia, Ib, II, III, IV, VI, VII, VIII, and IX of Streptococcus agalactiae, and (c) at least one fusion protein, and
vi. a sixth MAPS-GBS immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII or a different serotype selected from any of serotypes Ia, Ib, II, III, IV, V, VI, VIII, and IX of Streptococcus agalactiae, and (c) at least one fusion protein.

53. The immune composition of claim any of claims 40-50, further comprising: a seventh MAPS-GBS immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV or a different serotype selected from any of serotypes Ia, Ib, II, III, V, VI, VII, VIII, and IX of Streptococcus agalactiae, and (c) at least one fusion protein, and (b) at least one fusion protein.

54. The immune composition of any of claim 52 or 53, wherein when the first polysaccharide antigen is from a specific Streptococcus agalactiae serotype, the second polysaccharide antigen can be from the same Streptococcus agalactiae serotype, or a different Streptococcus agalactiae serotype.

55. The immune composition of any of claim 49 or 50, wherein when the first polysaccharide antigen and the second polysaccharide antigen are from the same Streptococcus agalactiae serotype.

56. The immune composition of claim 54, wherein the immune composition comprises the immunogenic complexes of:
i. a first MAPS-GBS immunogenic complex comprising (a) a first biotinylated polysaccharide antigen comprising at least biotin, one sialic acid and a first polysaccharide antigen from Streptococcus agalactiae serotype Ia, (b) a second biotinylated polysaccharide antigen comprising at least biotin and at least one sialic acid, wherein the second polysaccharide antigen is from Streptococcus agalactiae serotype Ia, and (c) at least one fusion protein,
ii. a second MAPS-GBS immunogenic complex comprising (a) first biotinylated polysaccharide antigen comprising at least biotin, one sialic acid and a first polysaccharide antigen from Streptococcus agalactiae serotype Ib, (b) a second biotinylated polysaccharide antigen comprising at least biotin and at least one sialic acid, wherein the second polysaccharide antigen is from Streptococcus agalactiae serotype Ib, and (c) at least one fusion protein,
iii. a third MAPS-GBS immunogenic complex comprising a first biotinylated polysaccharide antigen comprising at least biotin, one sialic acid and a first polysaccharide antigen from Streptococcus agalactiae serotype II, (b) a second biotinylated polysaccharide antigen comprising at least biotin and at least one sialic acid, wherein the second polysaccharide antigen is from Streptococcus agalactiae serotype II, and (c) at least one fusion protein, and (b) at least one fusion protein,
iv. a fourth MAPS-GBS immunogenic complex comprising (a) first biotinylated polysaccharide antigen comprising at least biotin, one sialic acid and a first polysaccharide antigen from Streptococcus agalactiae serotype III, (b) a second biotinylated polysaccharide antigen comprising at least biotin and at least one sialic acid, wherein the second polysaccharide antigen is from Streptococcus agalactiae serotype III, and (c) at least one fusion protein,
v. a firth MAPS-GBS immunogenic complex comprising (a) a first biotinylated polysaccharide antigen comprising at least biotin, one sialic acid and a first polysaccharide antigen from Streptococcus agalactiae serotype V, (b) a second biotinylated polysaccharide antigen comprising at least biotin and at least one sialic acid, wherein the second polysaccharide antigen is from Streptococcus agalactiae serotype V, and (c) at least one fusion protein, and
vi. a sixth MAPS-GBS immunogenic complex comprising (a) a first biotinylated polysaccharide antigen comprising at least biotin, one sialic acid and a first polysaccharide antigen from Streptococcus agalactiae serotype VII, and (b) a second biotinylated polysaccharide antigen comprising at least biotin and at least one sialic acid, wherein the second polysaccharide antigen is from Streptococcus agalactiae serotype VII, and (c) at least one fusion protein.

57. The immune composition of claim 56, further comprising: a seventh MAPS-GBS immunogenic complex comprising (a) a first biotinylated polysaccharide antigen comprising at least biotin, one sialic acid and a first polysaccharide antigen from Streptococcus agalactiae serotype IV, (b) a second biotinylated polysaccharide antigen comprising at least biotin and at least one sialic acid, wherein the second polysaccharide antigen is from Streptococcus agalactiae serotype IV, and (c) at least one fusion protein, and (b) at least one fusion protein.

58. The immune composition of any of claims 40-57, wherein the first biotinylated polysaccharide, or second biotinylated polysaccharide, or both, has a sialic acid level of greater than about 20% or about 30%, or about 40%, or about 50% or about 60%.

59. The immune composition of any of claims 40-58, wherein the at least one fusion protein is a fusion protein selected from any of claims 1-23, or selected from any listed in Table 2A, 2B or Table 3, or comprises an amino acid sequence having at least 80%, or at least 85%, or at least 90%, or at least 95% sequence identity to any of the amino acid sequences listed in Table 3.

60. The immune composition of any of claims 40-59, wherein the MAPS-GBS immunogenic complex further comprises at least one fusion protein selected from any of claims 24-39 or a SBD fusion protein selected from any listed in Table 2A, 2B or Table 3, or comprises an amino acid sequence having at least 80%, or at least 85%, or at least 90%, or at least 95% sequence identity to any of the amino acid sequences listed in Table 3.

61. A pharmaceutical composition comprising the immunogenic composition of any of claims 40-60, and a pharmaceutically acceptable carrier.

62. The pharmaceutical composition of claim 61, further comprising one or more adjuvants.

63. The pharmaceutical composition of claim 62, wherein the one or more adjuvants is or comprises a co-stimulation factor.

64. The pharmaceutical composition of claim 61, wherein the one or more adjuvants are selected from the group consisting of aluminum phosphate, aluminum hydroxide, and phosphate aluminum hydroxide.

65. A vaccine comprising the immunogenic composition of any of claims 40-60, or a fusion protein of any of claims 1-39, and a pharmaceutically acceptable carrier.

66. A method of making a multivalent vaccine, comprising mixing four or more species of MAPS-GBS immunogenic complexes of any of claims 40-60 in a single formulation.

67. The method of claim 66, comprising mixing six or more species of immunogenic complexes in a single formulation, wherein the six or more immunogenic complexes are disclosed in any of claims 48-54.

68. The method of claim 66, wherein each species of the immunogenic complexes comprises least one fusion protein is a fusion protein selected from any of claims 1-23, or selected from any listed in Table 2A, 2B or Table 3, or comprises an amino acid sequence having at least 80%, or at least 85%, or at least 90%, or at least 95% sequence identity to any of the amino acid sequences listed in Table 3.

69. The method of claim 68, wherein each species of the MAPS-GBS immunogenic complex can comprises at least one fusion protein selected from any of claims 24-39 or selected from any listed in Table 2A, 2B or Table 3, or comprises an amino acid sequence having at least 80%, or at least 85%, or at least 90%, or at least 95% sequence identity to any of the amino acid sequences listed in Table 3.

70. The method of claim 66, wherein each of the species of immunogenic complexes comprises:
a. at least a first biotinylated polysaccharide antigen comprising a first polysaccharide antigen, at least one sialic acid molecule and at least one biotin molecule,
b. at least a second biotinylated polysaccharide antigen comprising a second polysaccharide antigen, at least one sialic acid molecule and at least one biotin molecule,
c. at least one fusion protein, each fusion protein comprises at least a biotin-biotin moiety (BBM) and a sialic acid binding domain (SBD), wherein the fusion protein comprises in order from N-terminus to C terminus:
(i) a biotin-binding moiety (BBM), (ii) Rib polypeptide antigen or an antigenic fragment thereof, or Sip polypeptide or an antigenic fragment thereof, and (iii) a SBD polypeptide, or
(i) a biotin-binding moiety (BBM), (ii) a Rib polypeptide antigen or an antigenic fragment thereof and a Sip polypeptide or an antigenic fragment thereof, and (iii) a SBD polypeptide, or
(i) a SBD polypeptide, (ii) a Rib polypeptide antigen or an antigenic fragment thereof, or a Sip polypeptide or an antigenic fragment thereof, and (iii) a biotin-binding moiety (BBM), or
(i) a SBD polypeptide, (ii) Rib polypeptide antigen or an antigenic fragment thereof and Sip polypeptide or an antigenic fragment thereof, and (iii) the biotin-binding moiety (BBM),
wherein a BBM of at least one fusion protein non-covalently associates with at least one biotin molecule on the first biotinylated polysaccharide antigen and the SBD non-covalently associates with at least one sialic acid on the second biotinylated polysaccharide antigen, and/or
wherein a SBD of at least one fusion protein non-covalently associates with at least one sialic acid molecule on the first biotinylated polysaccharide antigen and the BBM non-covalently associates with at least biotin molecule on the second biotinylated polysaccharide antigen,
wherein the first biotinylated polysaccharide antigen, the second biotinylated polysaccharide antigen and the fusion protein form a MAPS-GBS immunogenic complex by the non-covalent association of at least one fusion protein to both the first biotinylated polysaccharide antigen and the second biotinylated polysaccharide antigen

71. The method of any of claims 66-70, wherein the BBM is Rhizavidin and comprises an amino acid sequence having at least 80%, or at least 85%, or at least 90%, or at least 95% sequence identity to SEQ ID NO: 1.

72. The method of any of claims 66-71, wherein the SBD comprises an amino acid sequence having at least 80%, or at least 85%, or at least 90%, or at least 95% sequence identity to any one of: SEQ ID NO: 2 (SBD1), SEQ ID NO: 112 (SBD2), SEQ ID NO: 113 (SBD3), SEQ ID NO: 114 (SBD4), SEQ ID NO: 115 (NanH), SEQ ID NO: 116 (NanH2) or SEQ ID NO: 117 (NanH3).

73. The method of any of claims 66-72, wherein the Rib polypeptide antigen comprises an amino acid sequence having at least 80%, or at least 85%, or at least 90%, or at least 95% sequence identity to SEQ ID NO: 4 (Rib).

74. The method of any of claims 66-73, wherein the Sip polypeptide antigen comprises an amino acid sequence having at least 80%, or at least 85%, or at least 90%, or at least 95% sequence identity to SEQ ID NO: 5 (Sip).

75. The method of any of claims 66-74, wherein the fusion protein is selected from any of the fusion proteins disclosed in claims 14-20 or selected from any listed in Table 2A, 2B or Table 3, or comprises an amino acid sequence having at least 80%, or at least 85%, or at least 90%, or at least 95% sequence identity to any of the amino acid sequences listed in Table 3.

76. The method of any of claims 66-75, wherein the fusion protein comprises at least one linker sequence or at least one spacer sequence located between any of: the BBM and the SBD; the BBM and at the first polypeptide antigen or an antigenic fragment thereof, the first polypeptide antigen or an antigenic fragment thereof and the second polypeptide antigen or an antigenic fragment thereof; or a polypeptide antigen or an antigenic fragment thereof and a SBD polypeptide.

77. The method of any of claims 66-76, wherein the linker sequence or spacer sequence is selected from any of: GGGGSSS, TDPNSSS, SSS and AAA.

78. The method of any of claims 66-77, wherein each species of MAPS-GBS immunogenic complex comprises a first biotinylated polysaccharide antigen, or a second biotinylated polysaccharide antigen, or both the first and second polysaccharide antigen from a serotype of a Group B Streptococcus (GBS).

79. The method of any of claims 66-78, comprising mixing four species of immunogenic complexes, wherein the Streptococcus agalactiae serotype for each species of the four immunogenic complexes is selected from any of: Ia, Ib, II, III, IV, V or VII.

80. The method of any of claims 66-81, comprising mixing six species of immunogenic complexes, wherein the Streptococcus agalactiae serotype for each species of the immunogenic complexes is selected from: Ia, Ib, II, III, V and VII.

81. The method of any of claims 66-80, comprising mixing seven species of immunogenic complexes, wherein the Streptococcus agalactiae serotype for each species of the immunogenic complexes is selected from: Ia, Ib, II, III, IV, V and VII.

82. Use of immune composition of any of claims 40-60, or the vaccine of claim 65 to induce an immune response to a subject.

83. Use of a vaccine composition of claim 65, or a fusion protein of any of claims 1-39 to induce an immune response in a subject.

84. A method to induce an immune response to a subject, comprising administering to the subject a pharmaceutical composition comprising the immune composition of any of claims 61-64, or the vaccine of claim 65.

85. A method to induce an immune response in a subject, comprising administering a vaccine comprising a fusion protein of any of claims 1-39.

86. The method of claim 84 or 85, wherein the immune response is an antibody or B cell response.

87. The method of claim 84 or 85, wherein the immune response is a CD4+ T cell response, including Th1, Th2, or Th17 response, or a CD8+ T cell response, or CD4+/CD8+ T cell response.

88. The method of claim 84 or 85, wherein the immune response is:
an antibody or B cell response; and
a T cell response.

89. The method of claim 84 or 85, wherein the immune response is to: at least the first antigenic polysaccharide, or the second antigenic polysaccharides, or both the first and second antigenic polysaccharide, or at least one polypeptide antigen.

90. The method of claim 84 or 85, wherein the immune response is an antibody or B cell response to at least the first antigenic polysaccharide and/or second antigenic polysaccharide, and a CD4+ T cell response, including Th 1, Th2, or Th 17 response, or a CD8+ T cell response, or CD4+/CD8+ T cell response to at least one polypeptide antigen.

91. The method of claim 84 or 85, wherein the immune response is an antibody or B cell response to at least the first antigenic polysaccharide and/or the second antigenic polysaccharide, and an antibody or B cell response and a CD4+ T cell response, including Th1, Th2, or Th 17 response, or a CD8+ T cell response, or CD4+/CD8+ T cell response to at least one polypeptide antigen.

92. The method of claim 84 or 85, wherein the immune response protects the subject from at least one serotype of Streptococcus agalactiae.

93. The method of claim 89, wherein the immune response protects the subject from at least one serotype of Streptococcus agalactiae selected from serotypes: Ia, Ib, II, III, V and VII.

94. The immunogenic composition of any of claims 40-60, wherein herein the immunogenic composition, upon administration to a subject, elicits (i) an immune response to the at least the first antigenic polysaccharide or the second antigenic polysaccharide, or both the first and second antigenic polysaccharide and (ii) an immune response to at least one of the polypeptide antigens, in the subject.

95. The immunogenic composition of claim 94, wherein the immune response to the at least first antigenic polysaccharide or the second antigenic polysaccharide, or both, and/or at least one of the one polypeptide antigens comprises an antibody or B cell response.

96. The immunogenic composition of claim 94, wherein the immune response to the at least first antigenic polysaccharide or the second antigenic polysaccharide, or both, and/or at least one of the one polypeptide antigens comprises a T cell response.

97. The immunogenic composition of claim 94, wherein the immune response to the at least first antigenic polysaccharide or the second antigenic polysaccharide, or both, and/or at least one of the one polypeptide antigens comprises a CD4+ T cell response, including Th1, Th2, or Th17 response, or a CD8+ T cell response, or a CD4+/CD8+ T cell response.

98. The immunogenic composition of claim 94, wherein the immune response to the at least first antigenic polysaccharide or the second antigenic polysaccharide, or both, and/or at least one of the one polypeptide antigens comprises:
an antibody or B cell response; and
a T cell response.

99. The immunogenic composition of any of claims 40-60, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) an antibody or B cell response to at least one of polypeptide antigens, in the subject.

100. The immunogenic composition of any of claims 40-60, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) an antibody or B cell response and a CD4+ T cell response, including Th1, Th2, or Th17 response, or a CD8+ T cell response, or a CD4+/CD8+ T cell response to at least one of polypeptide antigens, in the subject.

101. The immunogenic composition of any of claims 40-60, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) a CD4+ T cell response, including Th1, Th2, or Th17 response, or a CD8+ T cell response, or a CD4+/CD8+ T cell response to at least one of polypeptide antigens, in the subject.

102. The fusion protein of any of claims 1-39, wherein a biotin-binding moiety (BBM) comprises an amino acid sequence of at least 80%, or 90%, or 95% sequence identity to SEQ ID NO: 1 that has any one or more of the amino acid modifications: N80, T108, N118, S119A, N138A.

103. The fusion protein of any of claims 1-39, wherein a biotin-binding moiety comprises an amino acid sequence of at least 80%, or 90%, or 95%, 98%, 99% or 100% sequence identity to SEQ ID NO: 8, wherein the biotin binding moiety has at least one or more of the amino acid modifications: N80, T108, N118, S119A, N138A.

104. The fusion protein of any of claims 1-39, wherein the fusion protein comprising a biotin-binding moiety having an amino acid sequence of at least 80%, or 90%, or 95% sequence identity to SEQ ID NO: 2 that has any one or more of the amino acid modifications: N80, T108, N118, S119A, N138A.

105. The fusion protein of any of claims 1-39, wherein a biotin-binding moiety comprises an amino acid sequence of at least 80%, or 90%, or 95%, 98%, 99% or 100% sequence identity to SEQ ID NO: 52, wherein the biotin binding moiety has at least one or more of the amino acid modifications: N80, T108, N118, S119A, N138A.

106. An expression vector comprising a nucleic acid encoding a fusion protein of any of claims 1-39, wherein the expression vector comprises a promoter operatively linked to a nucleic acid sequence encoding the fusion protein, wherein the nucleic acid sequence comprises, in any order,
(i) a nucleic acid sequence encoding a Rhizavidin protein comprising amino acids of SEQ ID NO: 1 or a protein having at least 80% sequence identity to SEQ ID NO: 1,
(ii) a nucleic acid sequence encoding a at least one sialic acid binding domain (SBD) selected from any of: SBD1, SBD2, SBD3, SBD4, NanH, NanH2, NanH3, or VcNanH, or a SBD having an amino acid sequence that has at least 80%, or at least 85%, or at least 90%, or at least 95% sequence identity to any of: SEQ ID NO: 2 or SEQ ID NO: 112-120, and
(iii) a nucleic acid sequence encoding a polypeptide having an amino acid sequence of any of: SEQ ID NO: 4 (Rib), SEQ ID NO: 5 (Sip), SEQ ID NO: 11 (AlpC), SEQ ID NO: 12 (Alp1); SEQ ID NO: 13 (Alp3), SEQ ID NO: 14 (Alp3/1), or having an amino acid sequence at least 80%, or at least 85%, or at least 90%, or at least 95% sequence identity to any of: SEQ ID NO: 4, 5 or 11-14.

107. The expression vector of claim 106, wherein the nucleic encoding a Rhizavidin protein comprises a nucleic acid sequence having at least 80%, or at least 85%, or at least 90%, or at least 95% sequence identity to SEQ ID NO: 16 or a codon optimized variant thereof.

108. The expression vector of claim 106, wherein the nucleic encoding a SBD protein comprises a nucleic acid sequence having at least 80%, or at least 85%, or at least 90%, or at least 95% sequence identity to any of: SEQ ID NO: 170-174, or codon optimized variants thereof.

109. A cell comprising the expression vector of any of claims 107-109.

110. A method of manufacturing a fusion protein of any of claims 1-39, using the expression vector of any of claims 107 to 109.


SEQUENCE LISTING (181 sequences)

SEQ ID NO: 1 - protein, 135 aa, Rhizobium sp.
  REGION 1..135: note = Rhavi
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 2 - protein, 185 aa, unidentified
  REGION 1..185: note = SBD1
VIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 3 - protein, 328 aa, synthetic construct
  REGION 1..328: note = Rhavi-SBD1
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 4 - protein, 175 aa, Streptococcus agalactiae
  REGION 1..175: note = RIB
AEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 5 - protein, 405 aa, unidentified
DTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 6 - protein, 914 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 7 - protein, 914 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 8 - protein, 135 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGNATEIVTSWNLAYEGGSGPAIEQGQDTFQYVPTTENKSLLKD

SEQ ID NO: 9 - protein, 922 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 10 - protein, 918 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 11 - protein, 180 aa, unidentified
ASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 12 - protein, 180 aa, unidentified
AEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLPVPDKDKLE

SEQ ID NO: 13 - protein, 181 aa, unidentified
ASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGR

SEQ ID NO: 14 - protein, 361 aa, synthetic construct
ASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLPVPDKDKLE

SEQ ID NO: 15 - protein, 358 aa, synthetic construct
ASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 16 - DNA, 405 bp, Rhizobium sp.
  source 1..405: mol_type = unassigned DNA
tttgatgcgagtaactttaaagatttcagctctattgcgagcgccagtagctcttggcagaatcagagtggcagcaccatgattatccaggtggattctttcggcaacgttagtggccagtatgttaatcgtgcgcagggcacgggttgccagaactctccgtacccgctgaccggccgcgtgaatggcacgtttatcgccttcagcgttggctggaacaattctaccgaaaactgtaatagtgcaaccggctggacgggttatgcgcaggtgaacggtaacaataccgaaattgttacgagctggaatctggcctatgaaggcggttctggcccggcaatcgaacagggtcaggatacctttcagtacgttccgaccacggaaaacaaaagcctgctgaaagat

SEQ ID NO: 17 - protein, 318 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 18 - protein, 548 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 19 - protein, 726 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 20 - protein, 726 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 21 - protein, 772 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 22 - protein, 772 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 23 - protein, 922 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 24 - protein, 918 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 25 - protein, 364 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 26 - protein, 594 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 27 - protein, 323 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 28 - protein, 515 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 29 - protein, 373 aa, synthetic construct
MASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 30 - protein, 505 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 31 - protein, 735 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 32 - protein, 505 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 33 - protein, 735 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 34 - protein, 678 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVL

SEQ ID NO: 35 - protein, 678 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 36 - protein, 689 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 37 - protein, 7 aa, synthetic construct
GGGGSSS

SEQ ID NO: 38 - skipped

SEQ ID NO: 39 - protein, 5 aa, synthetic construct
  REGION 1..>5: note = The entire sequence of amino acids 1-5 can be repeated one or more times.
GGGGS

SEQ ID NO: 40 - protein, 6 aa, synthetic construct
GGGGGG

SEQ ID NO: 41 - protein, 15 aa, synthetic construct
GGGGSGGGGSGGGGS

SEQ ID NO: 42 - protein, 30 aa, synthetic construct
GGGGSGGGGSGGGGSGGGGSGGGGSGGGGS

SEQ ID NO: 43 - protein, 18 aa, synthetic construct
KESGSVSSEQLAQFRSLD

SEQ ID NO: 44 - protein, 14 aa, synthetic construct
EGKSSGSGSESKST

SEQ ID NO: 45 - skipped

SEQ ID NO: 46 - protein, 8 aa, synthetic construct
GGGGGGGG

SEQ ID NO: 47 - protein, 12 aa, synthetic construct
GSAGSAAGSGEF

SEQ ID NO: 48 - protein, 5 aa, synthetic construct
  REGION 1..>5: note = The entire sequence of amino acids 1-5 can be repeated one or more times.
EAAAK

SEQ ID NO: 49 - protein, 7 aa, synthetic construct
  REGION 2..>6: note = The entire sequence of amino acids 2-6 can be repeated one or more times.
AEAAAKA

SEQ ID NO: 50 - protein, 46 aa, synthetic construct
AEAAAKEAAAKEAAAKEAAAKALEAEAAAKEAAAKEAAAKEAAAKA

SEQ ID NO: 51 - protein, 7 aa, synthetic construct
  REGION 2..>6: note = The entire sequence of amino acids 2-6 can be repeated one or more times.
  REGION 1..>7: note = The entire sequence of amino acids 1-7 can be repeated one or more times.
AEAAAKA

SEQ ID NO: 52 - protein, 12 aa, synthetic construct
AEAAAKEAAAKA

SEQ ID NO: 53-56 - skipped (4 entries)

SEQ ID NO: 57 - protein, 14 aa, synthetic construct
APAPAPAPAPAPAP

SEQ ID NO: 58 - protein, 21 aa, synthetic construct
GAPGGGGGAAAAAGGGGGGAP

SEQ ID NO: 59 - protein, 39 aa, synthetic construct
GAPGGGGGAAAAAGGGGGGAPGGGGGAAAAAGGGGGGAP

SEQ ID NO: 60 - protein, 57 aa, synthetic construct
GAPGGGGGAAAAAGGGGGGAPGGGGGAAAAAGGGGGGAPGGGGGAAAAAGGGGGGAP

SEQ ID NO: 61 - skipped

SEQ ID NO: 62 - protein, 5 aa, synthetic construct
GGGGS

SEQ ID NO: 63 - protein, 5 aa, synthetic construct
EAAAK

SEQ ID NO: 64 - skipped

SEQ ID NO: 65 - protein, 6 aa, synthetic construct
HHHHHH

SEQ ID NO: 66 - protein, 10 aa, synthetic construct
MSYYHHHHHH

SEQ ID NO: 67-69 - skipped (3 entries)

SEQ ID NO: 70 - protein, 919 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 71 - protein, 693 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 72 - protein, 918 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 73 - protein, 866 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 74 - protein, 866 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 75 - protein, 547 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 76 - protein, 777 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 77 - protein, 777 aa, synthetic construct; identical to SEQ ID NO: 76

SEQ ID NO: 78 - protein, 781 aa, synthetic construct
MASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 79 - protein, 724 aa, synthetic construct
MASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 80 - protein, 724 aa, synthetic construct
MASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 81 - protein, 697 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 82 - protein, 927 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 83 - protein, 697 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 84 - protein, 927 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 85 - protein, 683 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSAAAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVL

SEQ ID NO: 86 - protein, 870 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGR

SEQ ID NO: 87 - protein, 693 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 88 - protein, 923 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 89 - protein, 692 aa, synthetic construct
MASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 90 - protein, 927 aa, synthetic construct
MASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 91 - protein, 870 aa, synthetic construct
MASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRAAAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 92 - protein, 870 aa, synthetic construct
MASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAASTIPGSAATLNTSITKNIQNGNAYIDLYDVKLGKIDPLQLIVLEQGFTAKYVFRQGTKYYGDVSQLQSTGRASLTYNIFGEDGLPHVKTDGQIDIVSVALTIYDSTTLRDKIEEVRTNANDPKWTEESRTEVLTGLDTIKTDIDNNPKTQTDIDSKIVEVNELEKLLVLSVPDKDKSGRGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 93 - protein, 495 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVL

SEQ ID NO: 94 - protein, 687 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 95 - protein, 545 aa, synthetic construct
MASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 96 - protein, 677 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVL

SEQ ID NO: 97 - protein, 907 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVL

SEQ ID NO: 98 - protein, 677 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 99 - protein, 907 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 100 - protein, 861 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 101 - protein, 1091 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 102 - protein, 865 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 103 - protein, 865 aa, synthetic construct; identical to SEQ ID NO: 102

SEQ ID NO: 104 - protein, 869 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVL

SEQ ID NO: 105 - protein, 1099 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVL

SEQ ID NO: 106 - protein, 865 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 107 - protein, 1095 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAADTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 108 - protein, 865 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 109 - protein, 1095 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 110 - protein, 864 aa, synthetic construct
MASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 111 - protein, 1099 aa, synthetic construct
MASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKSGRAEVISGSAATLNSALVKNVSGGKAYIDIYDVKNGKIDPLNLIVLNPSNYSANYYIKQGGRIFTSVNQLQTPGTATITYNILDENGNPYTKSDGQIDIVSLVTTVYDTTELRNNINKVIENANDPKWSDDSRKDVLSKIEVIKNDIDNNPKTQSDIDNKIVEVNELEKLLVLGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 112 - protein, 192 aa, unidentified
IPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 113 - protein, 191 aa, unidentified
SPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 114 - protein, 189 aa, unidentified
EETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 115 - protein, 381 aa, Salmonella sp.
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 116 - protein, 404 aa, Salmonella sp.
MKKFIKILKVLSMAIVLSACNINGIFASNLNTTNEPQKTTVFNKNDNTWNAQYFRIPSLQTLADGTMLAFSDIRYNGAEDHAYIDIGAAKSTDNGQTWDYKTVMENDRIDSTFSRVMDSTTVVTDTGRIILIAGSWNKNGNWASSTTSLRSDWSVQMVYSDDNGETWSDKVDLTTNKARIKNQPSNTIGWLAGVGSGIVMSDGTIVMPIQIALRENNANNYYSSVIYSKDNGETWTMGNKVPDPKTSENMVIELDGALIMSSRNDGKNYRASYISYDMGSTWEVYDPLHNKISTGNGSGCQGSFIKVTAKDGHRLGFISAPKNTKGGYVRDNITVYMIDFDDLSKGIRELCSPYPEDGNSSGGGYSCLSFNDGKLSILYEANGNIEYKDLTDYYLSIENNKKLK

SEQ ID NO: 117 - protein, 396 aa, unidentified
  REGION 1..396: note = The region is from Trypanosoma cruzi.
GSSRVELFKRQSSKVPFEKDGKVTERVVHSFRLPALVNVDGVMVAIADARYETSFDNSLIDTVAKYSVDDGETWETQIAIKNSRASSVSRVVDPTVIVKGNKLYVLVGSYNSSRSYWTSHGDARDWDILLAVGEVTKSTAGGKITASIKWGSPVSLKEFFPAEMEGMHTNQFLGGAGVAIVASNGNLVYPVQVTNKKKQVFSKIFYSEDEGKTWKFGKGRSAFGCSEPVALEWEGKLIINTRVDYRRRLVYESSDMGNTWLEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTPA

SEQ ID NO: 118 - protein, 1035 aa, unidentified
MSYFRNRDIDIERNSMNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 119 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 120 - protein, 192 aa, Vibrio cholerae
ALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 121 - protein, 929 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 122 - protein, 925 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 123 - protein, 928 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 124 - protein, 924 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 125 - protein, 934 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNLEHHHHHH

SEQ ID NO: 126 - protein, 930 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNLEHHHHHH

SEQ ID NO: 127 - protein, 506 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 128 - protein, 736 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 129 - protein, 335 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 130 - protein, 779 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 131 - protein, 779 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 132 - protein, 921 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 133 - protein, 921 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 134 - protein, 513 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 135 - protein, 743 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 136 - protein, 929 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATGGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 137 - protein, 925 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATGGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 138 - protein, 371 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 139 - protein, 601 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 140 - protein, 334 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 141 - protein, 778 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 142 - protein, 778 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 143 - protein, 920 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 144 - protein, 920 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 145 - protein, 512 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 146 - protein, 742 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 147 - protein, 928 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 148 - protein, 924 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 149 - protein, 370 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 150 - protein, 600 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 151 - protein, 332 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 152 - protein, 776 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 153 - protein, 776 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 154 - protein, 918 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 155 - protein, 918 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 156 - protein, 510 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 157 - protein, 740 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 158 - protein, 926 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 159 - protein, 922 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 160 - protein, 368 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 161 - protein, 598 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 162 - protein, 510 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 163 - protein, 740 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 164 - protein, 517 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 165 - protein, 747 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 166 - protein, 516 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 167 - protein, 746 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 168 - protein, 522 aa, synthetic construct
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNLEHHHHHH

SEQ ID NO: 169 - protein, 752 aa, synthetic construct
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNLEHHHHHH

SEQ ID NO: 170 - DNA, 555 bp, unidentified
  source 1..555: mol_type = unassigned DNA
gtgatagaaaaagaagatgttgaaaccaatgcttcaaatggtcagagagttgatttatcaagtgaactagataaactaaagaaacttgaaaacgcaacagttcacatggagtttaagccagatgccaaggccccagcattctataatctcttttctgtgtcaagtgctactaaaaaagatgagtacttcactatggcagtttacaataatactgctactctagaggggcgtggttcggatgggaaacagttttacaataattacaacgatgcacccttaaaagttaaaccaggtcagtggaattctgtgactttcacagttgaaaaaccgacagcagaactacctaaaggccgagtgcgcctctacgtaaacggggtattatctcgaacaagtctgagatctggcaatttcattaaagatatgccagatgtaacgcatgtgcaaatcggagcaaccaagcgtgccaacaatacggtttgggggtcaaatctacagattcggaatctcactgtgtataatcgtgctttaacaccagaagaggtacaaaaacgtagt

SEQ ID NO: 171 - DNA, 576 bp, unidentified
  source 1..576: mol_type = unassigned DNA
attccggaaggcattctgatggaaaagaacaatgttgatatagccgaagggcagggatacagcctggaccaggaggctggcgccaaatatgtaaaggctatgacccagggtacaattatcctatcctataaatcaacaagtgaaaatggaatccagtctctgttcagcgtaggaaacagtaccgcgggcaatcaggacaggcatttccacatctatataacaaactctggcggcataggcatagaacttagaaacacggacggtgtcttcaattatacattggacagaccggcatcagtgcgtgctctgtataaaggagaacgggtattcaatacagttgcattaaaagccgatgcagcaaataaacagtgcagattatttgcaaatggcgagcttctcgcaacactggataaagatgcgtttaagtttatcagtgacattacaggcgtagataacgttacattaggcggcacgaagaggcaggggaagattgcatatccgttcgggggaacgattggtgacattaaggtatatagtaatgcactgtccgacgaagaattgatacaggcaacagga

SEQ ID NO: 172 - DNA, 576 bp, unidentified
  source 1..576: mol_type = unassigned DNA
attcccgaaggcatcctgatggagaaaaataacgttgatattgccgagggccagggctattctttggatcaagaggccggtgccaagtatgtaaaagcaatgacacaagggaccattattctgtcatacaaatcgacctcagagaatggcatccaatcgttattctcagtcgggaatagtactgctggtaatcaggaccgtcattttcatatctatattacaaactcggggggcattggaatcgagcttcgtaacacagatggcgtattcaactatactttggaccgcccggcgtctgttcgcgcgctgtataaaggggagcgcgtgttcaatacggttgctttgaaggcggacgccgccaataagcagtgtcgcttgttcgcaaatggcgaattgttagctacgcttgacaaggacgcatttaagtttatttccgacatcactggggtggacaatgtaacgttgggtggaaccaaacgccagggcaaaattgcatacccgttcggagggacaatcggcgacattaaagtctatagcaacgcgctgagtgacgaagagcttatccaggctaccgga

SEQ ID NO: 173 - DNA, 573 bp, unidentified
  source 1..573: mol_type = unassigned DNA
tctcctatttttcaaggaggttcatatcaactgaacaataagagtatagatatcagctctttgttattagataaattgtctggagagagtcagacagtagtaatgaaatttaaagcagataaaccaaactctcttcaagctttgtttggcctatctaatagtaaagcaggctttaaaaataattacttttcaattttcatgagagattctggtgagataggtgtagaaataagagacgcccaaaagggaataaattatttattttccagaccagcttcattatggggaaaacataaaggacaggcagttgaaaatacactagtatttgtatctgattctaaagataaaacatacacaatgtatgttaatggaatagaagtgttctctgaaacagttgatacatttttgccaatttcaaatataaatggtatagataaggcaacactaggagctgttaatcgtgaaggtaaggaacattacctcgcaaaaggaagtattgatgaaatcagtctatttaacaaagcaattagtgatcaggaagtttcaactattcccttgtcaaatccatttcag

SEQ ID NO: 174 - DNA, 567 bp, unidentified
  source 1..567: mol_type = unassigned DNA
gaagaaactcctgtcttagaaaaaaataatgttactttaacagggggcggagaaaatgttactaaagagttaaaggataaatttactagcggtgactttactgtagtgattaagtacaatcagtcaagtgagaaaggcttacaagctctgtttggaatatctaattccaaacccggtcaacaaaatagttatgtagatgtgttccttagagacaatggtgagttggggatggaagcgcgtgatacttcttccaataaaaataacctagtatccagacctgcttcagtttggggtaagtacaaacaagaggctgtgactaacactgttgcagtagtagcagattcagtcaaaaaaacatattctttatacgcaaatggtacaaaagtagtagaaaagaaagtggataatttcctaaacatcaaggatattaaaggtattgattactatatgcttgggggagtgaaacgtgcaggaaaaacggcgtttggttttaacggaacactagaaaatatcaaattctttaatagtgcattggatgaagaaactgttaaaaagatgacaacaaac

SEQ ID NO: 175 - protein, 17 aa, synthetic construct
MKKVAAFVALSLLMAGC

SEQ ID NO: 176 - protein, 21 aa, synthetic construct
MNSKKLCCICVLFSLLAGCAS

SEQ ID NO: 177 - protein, 19 aa, synthetic construct
MRYSKLTMLIPCALLLSAC

SEQ ID NO: 178 - protein, 24 aa, synthetic construct
MFVTSKKMTAAVLAITLAMSLSAC

SEQ ID NO: 179 - protein, 18 aa, synthetic construct
MIKRVLVVSMVGLSLVGC

SEQ ID NO: 180 - protein, 4 aa, synthetic construct
VSDP

SEQ ID NO: 181 - protein, 4 aa, synthetic construct
AQDP